SEQUENCE LISTING (335 sequences)

SEQ ID NO: 1 - RNA, 3127 nt, Homo sapiens
  source 1..3127: mol_type = mRNA
gagatacccggccagacaccctcacctgcggtgcccagctgcccaggctgaggcaagagaaggccagaaaccatgcccatggggtctctgcaaccgctggccaccttgtacctgctggggatgctggtcgcttcctgcctcggacggctcagctggtatgacccagatttccaggcaaggctcacccgttccaactcgaagtgccagggccagctggaggtctacctcaaggacggatggcacatggtttgcagccagagctggggccggagctccaagcagtgggaggaccccagtcaagcgtcaaaagtctgccagcggctgaactgtggggtgcccttaagccttggccccttccttgtcacctacacacctcagagctcaatcatctgctacggacaactgggctccttctccaactgcagccacagcagaaatgacatgtgtcactctctgggcctgacctgcttagaaccccagaagacaacacctccaacgacaaggcccccgcccaccacaactccagagcccacagctcctcccaggctgcagctggtggcacagtctggcggccagcactgtgccggcgtggtggagttctacagcggcagcctggggggtaccatcagctatgaggcccaggacaagacccaggacctggagaacttcctctgcaacaacctccagtgtggctccttcttgaagcatctgccagagactgaggcaggcagagcccaagacccaggggagccacgggaacaccagcccttgccaatccaatggaagatccagaactcaagctgtacctccctggagcattgcttcaggaaaatcaagccccagaaaagtggccgagttcttgccctcctttgctcaggtttccagcccaaggtgcagagccgtctggtggggggcagcagcatctgtgaaggcaccgtggaggtgcgccagggggctcagtgggcagccctgtgtgacagctcttcagccaggagctcgctgcggtgggaggaggtgtgccgggagcagcagtgtggcagcgtcaactcctatcgagtgctggacgctggtgacccaacatcccgggggctcttctgtccccatcagaagctgtcccagtgccacgaactttgggagagaaattcctactgcaagaaggtgtttgtcacatgccaggatccaaaccccgcaggcctggccgcaggcacggtggcaagcatcatcctggccctggtgctcctggtggtgctgctggtcgtgtgcggcccccttgcctacaagaagctagtgaagaaattccgccagaagaagcagcgccagtggattggcccaacgggaatgaaccaaaacatgtctttccatcgcaaccacacggcaaccgtccgatcccatgctgagaaccccacagcctcccacgtggataacgaatacagccaacctcccaggaactcccacctgtcagcttatccagctctggaaggggctctgcatcgctcctccatgcagcctgacaactcctccgacagtgactatgatctgcatggggctcagaggctgtaaagaactgggatccatgagcaaaaagccgagagccagacctgtttgtcctgagaaaactgtccgctcttcacttgaaatcatgtccctatttctaccccggccagaacatggacagaggccagaagccttccggacaggcgctgctgccccgagtggcaggccagctcacactctgctgcacaacagctcggccgcccctccacttgtggaagctgtggtgggcagagccccaaaacaagcagccttccaactagagactcgggggtgtctgaagggggccccctttccctgcccgctggggagcggcgtctcagtgaaatcggctttctcctcagactctgtccctggtaaggagtgacaaggaagctcacagctgggcgagtgcattttgaatagttttttgtaagtagtgcttttcctccttcctgacaaatcgagcgctttggcctcttctgtgcagcatccacccctgcggatccctctggggaggacaggaaggggactcccggagacctctgcagccgtggtggtcagaggctgctcacctgagcacaaagacagctctgcacattcaccgcagctgccagccaggggtctgggtgggcaccaccctgacccacagcgtcaccccactccctctgtcttatgactcccctccccaaccccctcatctaaagacaccttcctttccactggctgtcaagcccacagggcaccagtgccacccagggcccggcacaaaggggcgcctagtaaaccttaaccaacttggttttttgcttcacccagcaattaaaagtcccaagctgaggtagtttcagtccatcacagttcatcttctaacccaagagtcagagatggggctggtcatgttcctttggtttgaataactcccttgacgaaaacagactcctctagtacttggagatcttggacgtacacctaatcccatggggcctcggcttccttaactgcaagtgagaagaggaggtctacccaggagcctcgggtctgatcaagggagaggccaggcgcagctcactgcggcggctccctaagaaggtgaagcaacatgggaacacatcctaagacaggtcctttctccacgccatttgatgctgtatctcctgggagcacaggcatcaatggtccaagccgcataataagtctggaagagcaaaagggagttactaggatatggggtgggctgctcccagaatctgctcagctttctgcccccaccaacaccctccaaccaggccttgccttctgagagcccccgtggccaagcccaggtcacagatcttcccccgaccatgctgggaatccagaaacagggaccccatttgtcttcccatatctggtggaggtgagggggctcctcaaaagggaactgagaggctgctcttagggagggcaaaggttcgggggcagccagtgtctcccatcagtgccttttttaataaaagctctttcatctatagtttggccaccatacagtggcctcaaagcaaccatggcctacttaaaaaccaaaccaaaaataaagagtttagttgaggagaaa

SEQ ID NO: 2 - RNA, 6685 nt, Homo sapiens
  source 1..6685: mol_type = mRNA
ggggccgggaaggggcctgagcagggaagggacacaggcgtcctgaaacaaagctttgggttgagggcagaggcgaggagccagagaagtgaactcctcctttccgtggccgctcttgtcatttttctcttttctcttttcaaatgggctatttgtgtgttaatttcatttggttcttgggaataacgactcaccgcgttgatttaaagaaagaactaaaattccagatggcaaactcaatgaatggcagaaaccctggtggtcgaggaggaaatccccgaaaaggtcgaattttgggtattattgatgctattcaggatgcagttggaccccctaagcaagctgccgcagatcgcaggaccgtggagaagacttggaagctcatggacaaagtggtaagactgtgccaaaatcccaaacttcagttgaaaaatagcccaccatatatacttgatattttgcctgatacatatcagcatttacgacttatattgagtaaatatgatgacaaccagaaacttgcccaactcagtgagaatgagtactttaaaatctacattgatagccttatgaaaaagtcaaaacgggcaataagactctttaaagaaggcaaggagagaatgtatgaagaacagtcacaggacagacgaaatctcacaaaactgtcccttatcttcagtcacatgctggcagaaatcaaagcaatctttcccaatggtcaattccagggagataactttcgtatcacaaaagcagatgctgctgaattctggagaaagttttttggagacaaaactatcgtaccatggaaagtattcagacagtgccttcatgaggtccaccagattagctctggcctggaagcaatggctctaaaatcaacaattgatttaacttgcaatgattacatttcagtttttgaatttgatatttttaccaggctgtttcagccttggggctctattttgcggaattggaatttcttagctgtgacacatccaggttacatggcatttctcacatatgatgaagttaaagcacgactacagaaatatagcaccaaacccggaagctatattttccggttaagttgcactcgattgggacagtgggccattggctatgtgactggggatgggaatatcttacagaccatacctcataacaagcccttatttcaagccctgattgatggcagcagggaaggattttatctttatcctgatgggaggagttataatcctgatttaactggattatgtgaacctacacctcatgaccatataaaagttacacaggaacaatatgaattatattgtgaaatgggctccacttttcagctctgtaagatttgtgcagagaatgacaaagatgtcaagattgagccttgtgggcatttgatgtgcacctcttgccttacggcatggcaggagtcggatggtcagggctgccctttctgtcgttgtgaaataaaaggaactgagcccataatcgtggacccctttgatccaagagatgaaggctccaggtgttgcagcatcattgacccctttggcatgccgatgctagacttggacgacgatgatgatcgtgaggagtccttgatgatgaatcggttggcaaacgtccgaaagtgcactgacaggcagaactcaccagtcacatcaccaggatcctctccccttgcccagagaagaaagccacagcctgacccactccagatcccacatctaagcctgccacccgtgcctcctcgcctggatctaattcagaaaggcatagttagatctccctgtggcagcccaacgggttcaccaaagtcttctccttgcatggtgagaaaacaagataaaccactcccagcaccacctcctcccttaagagatcctcctccaccgccacctgaaagacctccaccaatcccaccagacaatagactgagtagacacatccatcatgtggaaagcgtgccttccagagacccgccaatgcctcttgaagcatggtgccctcgggatgtgtttgggactaatcagcttgtgggatgtcgactcctaggggagggctctccaaaacctggaatcacagcgagttcaaatgtcaatggaaggcacagtagagtgggctctgacccagtgcttatgcggaaacacagacgccatgatttgcctttagaaggagctaaggtcttttccaatggtcaccttggaagtgaagaatatgatgttcctccccggctttctcctcctcctccagttaccaccctcctccctagcataaagtgtactggtccgttagcaaattctctttcagagaaaacaagagacccagtagaggaagatgatgatgaatacaagattccttcatcccaccctgtttccctgaattcacaaccatctcattgtcataatgtaaaacctcctgttcggtcttgtgataatggtcactgtatgctgaatggaacacatggtccatcttcagagaagaaatcaaacatccctgacttaagcatatatttaaagggagatgtttttgattcagcctctgatcccgtgccattaccacctgccaggcctccaactcgggacaatccaaagcatggttcttcactcaacaggacgccctctgattatgatcttctcatccctccattaggtgaagatgcttttgatgccctccctccatctctcccacctcccccacctcctgcaaggcatagtctcattgaacattcaaaacctcctggctccagtagccggccatcctcaggacaggatctttttcttcttccttcagatccctttgttgatctagcaagtggccaagttcctttgcctcctgctagaaggttaccaggtgaaaatgtcaaaactaacagaacatcacaggactatgatcagcttccttcatgttcagatggttcacaggcaccagccagaccccctaaaccacgaccgcgcaggactgcaccagaaattcaccacagaaaaccccatgggcctgaggcggcattggaaaatgtcgatgcaaaaattgcaaaactcatgggagagggttatgcctttgaagaggtgaagagagccttagagatagcccagaataatgtcgaagttgcccggagcatcctccgagaatttgccttccctcctccagtatccccacgtctaaatctatagcagccagaactgtagacaccaaaatggaaagcaatcgatgtattccaagagtgtggaaataaagagaactgagatggaattcaagagagaagtgtctcctcctcgtgtagcagcttgagaagaggcttgggagtgcagcttctcaaaggagaccgatgcttgctcaggatgtcgacagctgtggcttccttgtttttgctagccatatttttaaatcagggttgaactgacaaaaataatttaaagacgtttacttcccttgaactttgaacctgtgaaatgctttaccttgtttacagtttggcaaagttgcagtttgttcttgtttttagtttagttttgttttggtgttttgatacctgtactgtgttcttcacagaccctttgtagcgtggtcaggtctgctgtaacatttcccaccaactctcttgctgtccacatcaacagctaaatcatttattcatatggatctctaccatccccatgccttgcccaggtccagttccatttctctcattcacaagatgctttgaaggttctgattttcaactgatcaaactaatgcaaaaaaaaaaaagtatgtattcttcactactgagtttcttctttggaaaccatcactattgagagatgggaaaaacctgaatgtataaagcatttatttgtcaataaactgccttttgtaaggggttttcacataacatagaggagcttcccttttttgtttaagttttgtaacctttaatcctccatattctcatgtctgtcatcccaggggtgtcacaactgtaaaaatctacaatattagaaagcagctactacagatgtggaagagagaacacttgtataagacaatgctgtactgaagttatataacaaggtcccctgatacttatgcctgcattactttgagggatgctgaatgagaggaccatctccctgaaatatattaataattttcagataaaattatgaacataacataccttttctcaagtggaattagagttcatttcccatttaagtgattacactctatctcctaagagctgtgggaaccagttcatattgcagtggaaacattaactcttcgttgcactgttgagtgtcaaggctactgtggcaatgttttttgcaaagttctcagtaattttctctctgcaatcaaattagagttcatatttaatttgcagattctctcattcattgctccacactgctttatcaaatcaggtgaaagaaattaatgtagttttgcctattttacaaaatggtggttggttctttaaacaccatgttattactcttaataaatctacaaatatttattgagcatctatgatatgcttggtgctgttaggcactgtgtttttaaagcacaaactctcaaaagctgattccagcgtaatgttttatttttgtttttctgtgttttttatgacaaagtctgtctgatagacaaagcctggaaggtttcccccagggctttagaatttagaattagctggttttcatttcaaatgggaaattaatggagaaatgagataaaatcatctctgtgtaaatgcttgaacccacagaaagttttctaaatatggactaattgtgggtttgtgcatatttctgtgtctcctcttttcacttcacacccaccatgatataaaaagatgcatcagagagtgagcctgccttcaggacctgaaaggacatgactgaacatctagtagatctggtggaaattaatgttatacaaaagacacgtagactaagttgaccctgggagaggagttaatacattcctttgcaaagtgacatgtgccctttttttgacagtctggctttcatggtatgagatgaggactcacaggccttgggtaatttctgcttttgtatgaattttttcagatggttctgagctggttatgtccctggaggggaatttttcatttgccttagatgttcttcccatgagtaggtacatgagtgaagtttcttacacttttaaaattgcattggcaaattttcttttgctttatgaacccctgttttgtcattatgatttttcacttgttttaacagtttcagatgtcaccttggaatagcactgtttattttggcctttttatgattgctgatgataggtacagtctaaaggccaaaagttgccacttcattgtgataacattgttgcaacattcctaatatattagagaatgatgtaaaataagaatgttagagaacatagcacattctcattcagtttataaacaatatttgaaaatttccagtgagaattaaaaaaggactagtgatgatatagcaaacctggacctgaaatttgggggtggggggtggggggatatctgttttgcagagaaaatttaccagcctccagcaaaatctctgcatcttgtactcaatagcaaccattagtattgtcatttaaaaagaatgaatcaaaaattcatacgtaatggaatgaaaagaggaaaactttctattaaacactgtcaagaaagacccattgctatctatcacctatgaaattaaacctaaaaaaatttttttttattgtaaaagcgactgctcgttgatgtctggaagtgatccagactttgtttcactgtgatccaaaaagttagacattccttccatttctcaagatttggtaacaatttttttttaagtcagcatttcatataatacataaaattaagacaaattaacttactgatatgtggccaatatcaacccttctcacccaacccaaacatgttctggagatagaaggacttatcctcaattatattccagtaaatttatctccataatctgttttatcccctaatgtatgatatatctcaccaaatttttctagaattgaataaaagcagtttttgcttaacttgaatttccttccacaaatacaaccttcagctcttccctctctgtttcccttttgcccacattgcaaatagtcttcataatagaaccctcactgcaacttaaaagggtcccatttcttaactcagcatttcatataccaagaacacttgcaaagatttcttcataatgtgattttttaatttttcagttccagaagcatttcctacagttcgaacgttattttttctctcagattttcaacttgaagctttgatttgcactgaattaacttggataaaattttagtttctctcttcagctaacagatttggaagcaaattacagtatacttgaataattcaggtactacctgttgacctggaattttagtgattctgggaaagttttccagacaaagacaccaatagagtgaagggttttttccccaattactattttattgttttttaaaatgcctttttaaaacttgtgtgatgtggcaaaatagtcatatgttcaattaatttatattttattcattccttttcaattctggttattatgtaacctcaagtactttatctgttcttttaaagtattttataaaatgaacgttaactaaa

SEQ ID NO: 3 - RNA, 2176 nt, Homo sapiens
  source 1..2176: mol_type = mRNA
agtccctgtcctgcgcccgcgcgccccgggagcctacccagcacgcgctccgcgcccactggttccctccagccgccgccgtccagccgagtccccactccggagtcgccgctgccgcggggacatggtcctctgcgttcaggggtgagcacccccttgtaagctcagggctactgttgggtgtcagggaacaaagttttagactgctgcgctccaaagcgggcacacacatgtacctagaacacaccagccactgtccccaccatgatgatgacacagccatggacacacccctgcccagacctcgtcctttgctggctgtggagcggactgggcagcggcccctgtgggccccgtccctggaactgcccaagccagtcatgcagcccttgcctgctggggccttcctcgaggaggtggcagagggtaccccagcccagacagagagtgagccaaaggtgctggacccagaggaggatctgctgtgcatagccaagaccttctcctaccttcgggaatctggctggtattggggttccattacggccagcgaggcccgacaacacctgcagaagatgccagaaggcacgttcttagtacgtgacagcacgcaccccagctacctgttcacgctgtcagtgaaaaccactcgtggccccaccaatgtacgcattgagtatgccgactccagcttccgtctggactccaactgcttgtccaggccacgcatcctggcctttccggatgtggtcagccttgtgcagcactatgtggcctcctgcactgctgatacccgaagcgacagccccgatcctgctcccaccccggccctgcctatgcctaaggaggatgcgcctagtgacccagcactgcctgctcctccaccagccactgctgtacacctaaaactggtgcagccctttgtacgcagaagcagtgcccgcagcctgcaacacctgtgccgccttgtcatcaaccgtctggtggccgacgtggactgcctgccactgccccggcgcatggccgactacctccgacagtaccccttccagctctgactgtacggggcaatctgcccaccctcacccagtcgcaccctggaggggacatcagccccagctggacttgggcccccactgtccctcctccaggcatcctggtgcctgcatacctctggcagctggcccaggaagagccagcaagagcaaggcatgggagaggggaggtgtcacacaacttggaggtaaatgcccccaggccgcatgtggcttcattatactgagccatgtgtcagaggatggggagacaggcaggaccttgtctcacctgtgggctgggcccagacctccactcgcttgcctgccctggccacctgaactgtatgggcactctcagccctggtttttcaatccccagggtcgggtaggacccctactggcagccagcctctgtttctgggaggatgacatgcagaggaactgagatcgacagtgactagtgaccccttgttgaggggtaagccaggctaggggactgcacaattatacactatttatttatttattctccttggggttggtgtcaggggcgagccaaccccacctctatgccctgagccctggtagtccagagaccccaactctgccctggcttctctggttcttccctgtggaaagcccatcctgagacatcttgctggaaccaaggcaatcctggatgtcctggtactgacccacccgtctgtgaatgtgtccactctcttctgcccccagccatatttggggaggatggacaactacaataggtaagaaaatgcagccggagcctcagtccccagcagagcctgtgtctcaccccctcacaggacagagctgtatctgcatagagctggtctcactgtggcgcaggccccggggggagtgcctgtgctgtcaggaagagggggtgctggtttgagggccgccactgcagttctgctaggtctgcttcctgcccaggaaggtgcctgcacatgagaggagagaaatacacgtctgataagacttcatgaaataataattatagcaaagaacagtttggtggtcttttctcttccactgatttttctgtaatgacattatacctttattacctctttattttattacctctataataaaatgatacctttcatgta

SEQ ID NO: 4 - RNA, 2737 nt, Homo sapiens
  source 1..2737: mol_type = mRNA
agctcgggctccagctccagcgccggcgcttcagctgcgaccgcgagccctctcaagcaaggtagagaccccgcgagccccccagcgccccaccctcccccatcggaaaaggacaggggtagggaagccgaggacccacgggctgcgttcgtgctgctgggtcgggaaggaggaagcgtgacagctggagcgggcctaccctctgaagaggtccaagcaacggaagtactactacgaagctgcctttctggccatccttgagaaaaatagacagatggccaaggagaggggcctaataagccccagtgattttgcccagctgcaaaaatacatggaatactccaccaaaaaggtcagtgatgtcctaaagctcttcgaggatggcgagatggctaaatatgtccaaggagatgccattgggtacgagggattccagcaattcctgaaaatctatctcgaagtggataatgttcccagacacctaagcctggcactgtttcaatcctttgagactggtcactgcttaaatgagacaaatgtgacaaaagatgtggtgtgtctcaatgatgtttcctgctacttttcccttctggagggtggtcggccagaagacaagttagaattcaccttcaagctgtacgacacggacagaaatgggatcctggacagctcagaagtggacaaaattatcctacagatgatgcgagtggctgaatacctggattgggatgtgtctgagctgaggccgattcttcaggagatgatgaaagagattgactatgatggcagtggctctgtctctcaagctgagtgggtccgggctggggccaccaccgtgccactgctagtgctgctgggtctggagatgactctgaaggacgacggacagcacatgtggaggcccaagaggttccccagaccagtctactgcaatctgtgcgagtcaagcattggtcttggcaaacagggactgagctgtaacctctgtaagtacactgttcacgaccagtgtgccatgaaagccctgccttgtgaagtcagcacctatgccaagtctcggaaggacattggtgtccaatcacatgtgtgggtgcgaggaggctgtgagtccgggcgctgcgaccgctgtcagaaaaagatccggatctaccacagtctgaccgggctgcattgtgtatggtgccacctagagatccacgatgactgcctgcaagcggtgggccatgagtgtgactgtgggctgctccgggatcacatcctgcctccatcttccatctatcccagtgtcctggcctctggaccggatcgtaaaaatagcaaaacaagccagaagaccatggatgatttaaatttgagcacctctgaggctctgcggattgaccctgttcctaacacccacccacttctcgtctttgtcaatcctaagagtggcgggaagcaggggcaaagggtgctctggaagttccagtatatattaaaccctcgacaggtgttcaacctcctaaaggatggtcctgagatagggctccgattattcaaggatgttcctgatagccggattttggtgtgtggtggagacggcacagtaggctggattctagagaccattgacaaagctaacttgccagttttgcctcctgttgctgtgttgcccctgggtactggaaatgatctggctcgatgcctaagatggggaggaggttatgaaggacagaatctggcaaagatcctcaaggatttagagatgagtaaagtggtacatatggatcgatggtctgtggaggtgatacctcaacaaactgaagaaaaaagtgacccagtcccctttcaaatcatcaataactacttctctattggcgtggatgcctctattgctcatcgattccacatcatgcgagagaaatatccggagaagttcaacagcagaatgaagaacaagctatggtacttcgaatttgccacatctgaatccatcttctcaacatgcaaaaagctggaggagtctttgacagttgagatctgtgggaaaccgctggatctgagcaacctgtccctagaaggcatcgcagtgctaaacatccctagcatgcatggtggctccaacctctggggtgataccaggagaccccatggggatatctatgggatcaaccaggccttaggtgctacagctaaagtcatcaccgaccctgatatcctgaaaacctgtgtaccagacctaagtgacaagagactggaagtggttgggctggagggtgcaattgagatgggccaaatctataccaagctcaagaatgctggacgtcggctggccaagtgctctgagatcaccttccacaccacaaaaacccttcccatgcaaattgacggagaaccctggatgcagacgccctgtacaatcaagatcacccacaagaaccagatgcccatgctcatgggcccacccccccgctccaccaatttctttggcttcttgagctaagggggacacccttggcctccaagccagccttgaacccacctccctgtccctggactctactcccgaggctctgtacattgctgccacatactcctgccagcttgggggagtgttccttcaccctcacagtatttattatcctgcaccacctcactgttccccatgcgcacacacatacacacaccccaaaacacatacattgaaagtgcctcatctgaataaaatgacttgtgtttcccctttgggatctgctaa

SEQ ID NO: 5 - RNA, 4072 nt, Homo sapiens
  source 1..4072: mol_type = mRNA
caaactaggacttgctcagcagaggccgccagcccggagctggatccagagcccggccttggggaccccagctcccacctgcgccctgccttccagatcagccaaccgcctgccatggagactttctttaggagacatttccgggggaaggtgccaggccctggagaggggcagcagcggcccagcagcgtggggctgcccacaggcaaggcccggcgtcgctcccccgctgggcaggcctcctcctcactggcacagcggcgccgctccagcgcccagctccagggctgcctcctgagttgcggggtgagggcccagggttccagccgccggcgctccagcactgtgcccccttcctgcaacccccgcttcatcgtggataaggtgctcactccacagcctaccaccgtgggggcccagcttctgggtgcacccctgctgttgaccgggcttgtgggcatgaatgaggaggagggtgtccaggaggatgtggtagccgaggcatcgagcgccatccagccaggcaccaagacaccagggccacccccacctcggggcgcccagccgctgttgcccctaccccgctacctgcgccgagcctcctcccacctgctccccgcggatgccgtatatgaccacgctctctggggcctgcacggctactatcggcgcctcagccagcggcggccctcaggccagcaccctggccctgggggccgaagagcctcaggcaccaccgccggcaccatgctgcccacccgtgtgcgcccactgtcccgcaggcgccaggtagccctacggcgcaaggcggccggaccccaggcctggagcgccctgctcgcgaaagccatcaccaagtcgggcctccagcacctggccccccctccgcccacccctggggccccgtgcagcgagtcagagcggcagatccggagtacagtggactggagcgagtcagcgacatatggggagcacatctggttcgagaccaacgtgtccggggacttctgctacgttggggagcagtactgtgtagccaggatgctgaagtcagtgtctcgaagaaagtgcgcagcctgcaagattgtggtgcacacgccctgcatcgagcagctggagaagataaatttccgctgtaagccgtccttccgtgaatcaggctccaggaatgtccgcgagccaacctttgtacggcaccactgggtacacagacgacgccaggacggcaagtgtcggcactgtgggaagggattccagcagaagttcaccttccacagcaaggagattgtggccatcagctgctcgtggtgcaagcaggcataccacagcaaggtgtcctgcttcatgctgcagcagatcgaggagccgtgctcgctgggggtccacgcagccgtggtcatcccgcccacctggatcctccgcgcccggaggccccagaatactctgaaagcaagcaagaagaagaagagggcatccttcaagaggaagtccagcaagaaagggcctgaggagggccgctggagacccttcatcatcaggcccaccccctccccgctcatgaagcccctgctggtgtttgtgaaccccaagagtgggggcaaccagggtgcaaagatcatccagtctttcctctggtatctcaatccccgacaagtcttcgacctgagccagggagggcccaaggaggcgctggagatgtaccgcaaagtgcacaacctgcggatcctggcgtgcgggggcgacggcacggtgggctggatcctctccaccctggaccagctacgcctgaagccgccaccccctgttgccatcctgcccctgggtactggcaacgacttggcccgaaccctcaactggggtgggggctacacagatgagcctgtgtccaagatcctctcccacgtggaggaggggaacgtggtacagctggaccgctgggacctccacgctgagcccaaccccgaggcagggcctgaggaccgagatgaaggcgccaccgaccggttgcccctggatgtcttcaacaactacttcagcctgggctttgacgcccacgtcaccctggagttccacgagtctcgagaggccaacccagagaaattcaacagccgctttcggaataagatgttctacgccgggacagctttctctgacttcctgatgggcagctccaaggacctggccaagcacatccgagtggtgtgtgatggaatggacttgactcccaagatccaggacctgaaaccccagtgtgttgttttcctgaacatccccaggtactgtgcgggcaccatgccctggggccaccctggggagcaccacgactttgagccccagcggcatgacgacggctacctcgaggtcattggcttcaccatgacgtcgttggccgcgctgcaggtgggcggacacggcgagcggctgacgcagtgtcgcgaggtggtgctcaccacatccaaggccatcccggtgcaggtggatggcgagccctgcaagcttgcagcctcacgcatccgcatcgccctgcgcaaccaggccaccatggtgcagaaggccaagcggcggagcgccgcccccctgcacagcgaccagcagccggtgccagagcagttgcgcatccaggtgagtcgcgtcagcatgcacgactatgaggccctgcactacgacaaggagcagctcaaggaggcctctgtgccgctgggcactgtggtggtcccaggagacagtgacctagagctctgccgtgcccacattgagagactccagcaggagcccgatggtgctggagccaagtccccgacatgccagaaactgtcccccaagtggtgcttcctggacgccaccactgccagccgcttctacaggatcgaccgagcccaggagcacctcaactatgtgactgagatcgcacaggatgagatttatatcctggaccctgagctgctgggggcatcggcccggcctgacctcccaacccccacttcccctctccccacctcaccctgctcacccacgccccggtcactgcaaggggatgctgcaccccctcaaggtgaagagctgattgaggctgccaagaggaacgacttctgtaagctccaggagctgcaccgagctgggggcgacctcatgcaccgagacgagcagagtcgcacgctcctgcaccacgcagtcagcactggcagcaaggatgtggtccgctacctgctggaccacgcccccccagagatccttgatgcggtggaggaaaacggggagacctgtttgcaccaagcagcggccctgggccagcgcaccatctgccactacatcgtggaggccggggcctcgctcatgaagacagaccagcagggcgacactccccggcagcgggctgagaaggctcaggacaccgagctggccgcctacctggagaaccggcagcactaccagatgatccagcgggaggaccaggagacggctgtgtagcgggccgcccacgggcagcaggagggacaatgcggccaggggacgagcgccttccttgcccacctcactgccacattccagtgggacggccacggggggacctaggccccagggaaagagccccatgccgccccctaaggagccgcccagacctagggctggactcaggagctgggggggcctcacctgttcccctgaggaccccgccggacccggaggctcacagggaacaagacacggctgggttggatatgcctttgccggggttctggggcagggcgctccctggccgcagcagatgccctcccaggagtggaggggctggagagggggaggccttcgggaagaggcttcctgggccccctggtcttcggccgggtccccagcccccgctcctgccccaccccacctcctccgggcttcctcccggaaactcagcgcctgctgcacttgcctgccctgccttgcttggcacccgctccggcgaccctccccgctcccctgtcatttcatcgcggactgtgcggcctgggggtggggggcgggactctcacggtgacatgtttacagctgggtgtgactcagtaaagtggatttttttttc

SEQ ID NO: 6 - RNA, 9421 nt, Homo sapiens
  source 1..9421: mol_type = mRNA
gcgcgggcggcggcggcggcgagagcagaggacgagccgggacgcggcgccgcggcaccagggcgcgcagccgggccggcccgaccccaccggccatacggtggagccatcgaagcccccacccacaggctgacagaggcaccgttcaccagagggctcaacaccgggatctatgtttaagttttaactctcgcctccaaagaccacgataattccttccccaaagcccagcagccccccagccccgcgcagccccagcctgcctcccggcgcccagatgcccgccatgccctccagcggccccggggacaccagcagctctgctgcggagcgggaggaggaccgaaaggacggagaggagcaggaggagccgcgtggcaaggaggagcgccaagagcccagcaccacggcacggaaggtggggcggcctgggaggaagcgcaagcaccccccggtggaaagcggtgacacgccaaaggaccctgcggtgatctccaagtccccatccatggcccaggactcaggcgcctcagagctattacccaatggggacttggagaagcggagtgagccccagccagaggaggggagccctgctggggggcagaagggcggggccccagcagagggagagggtgcagctgagaccctgcctgaagcctcaagagcagtggaaaatggctgctgcacccccaaggagggccgaggagcccctgcagaagcgggcaaagaacagaaggagaccaacatcgaatccatgaaaatggagggctcccggggccggctgcggggtggcttgggctgggagtccagcctccgtcagcggcccatgccgaggctcaccttccaggcgggggacccctactacatcagcaagcgcaagcgggacgagtggctggcacgctggaaaagggaggctgagaagaaagccaaggtcattgcaggaatgaatgctgtggaagaaaaccaggggcccggggagtctcagaaggtggaggaggccagccctcctgctgtgcagcagcccactgaccccgcatcccccactgtggctaccacgcctgagcccgtggggtccgatgctggggacaagaatgccaccaaagcaggcgatgacgagccagagtacgaggacggccggggctttggcattggggagctggtgtgggggaaactgcggggcttctcctggtggccaggccgcattgtgtcttggtggatgacgggccggagccgagcagctgaaggcacccgctgggtcatgtggttcggagacggcaaattctcagtggtgtgtgttgagaagctgatgccgctgagctcgttttgcagtgcgttccaccaggccacgtacaacaagcagcccatgtaccgcaaagccatctacgaggtcctgcaggtggccagcagccgcgcggggaagctgttcccggtgtgccacgacagcgatgagagtgacactgccaaggccgtggaggtgcagaacaagcccatgattgaatgggccctggggggcttccagccttctggccctaagggcctggagccaccagaagaagagaagaatccctacaaagaagtgtacacggacatgtgggtggaacctgaggcagctgcctacgcaccacctccaccagccaaaaagccccggaagagcacagcggagaagcccaaggtcaaggagattattgatgagcgcacaagagagcggctggtgtacgaggtgcggcagaagtgccggaacattgaggacatctgcatctcctgtgggagcctcaatgttaccctggaacaccccctcttcgttggaggaatgtgccaaaactgcaagaactgctttctggagtgtgcgtaccagtacgacgacgacggctaccagtcctactgcaccatctgctgtgggggccgtgaggtgctcatgtgcggaaacaacaactgctgcaggtgcttttgcgtggagtgtgtggacctcttggtggggccgggggctgcccaggcagccattaaggaagacccctggaactgctacatgtgcgggcacaagggtacctacgggctgctgcggcggcgagaggactggccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgagaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtcttcatgaatgagaaagaggacatcttatggtgcactgaaatggaaagggtatttggtttcccagtccactatactgacgtctccaacatgagccgcttggcgaggcagagactgctgggccggtcatggagcgtgccagtcatccgccacctcttcgctccgctgaaggagtattttgcgtgtgtgtaagggacatgggggcaaactgaggtagcgacacaaagttaaacaaacaaacaaaaaacacaaaacataataaaacaccaagaacatgaggatggagagaagtatcagcacccagaagagaaaaaggaatttaaaacaaaaaccacagaggcggaaataccggagggctttgccttgcgaaaagggttggacatcatctcctgatttttcaatgttattcttcagtcctatttaaaaacaaaaccaagctcccttcccttcctcccccttcccttttttttcggtcagaccttttattttctactcttttcagaggggttttctgtttgtttgggttttgtttcttgctgtgactgaaacaagaaggttattgcagcaaaaatcagtaacaaaaaatagtaacaataccttgcagaggaaaggtgggagagaggaaaaaaggaaattctatagaaatctatatattgggttgtttttttttttgttttttgttttttttttttgggtttttttttttactatatatcttttttttgttgtctctagcctgatcagataggagcacaagcaggggacggaaagagagagacactcaggcggcagcattccctcccagccactgagctgtcgtgccagcaccattcctggtcacgcaaaacagaacccagttagcagcagggagacgagaacaccacacaagacatttttctacagtatttcaggtgcctaccacacaggaaaccttgaagaaaatcagtttctagaagccgctgttacctcttgtttacagtttatatatatatgatagatatgagatatatatataaaaggtactgttaactactgtacaacccgacttcataatggtgctttcaaacagcgagatgagtaaaaacatcagcttccacgttgccttctgcgcaaagggtttcaccaaggatggagaaagggagacagcttgcagatggcgcgttctcacggtgggctcttccccttggtttgtaacgaagtgaaggaggagaacttgggagccaggttctccctgccaaaaagggggctagatgaggtggtcgggcccgtggacagctgagagtgggattcatccagactcatgcaataaccctttgattgttttctaaaaggagactccctcggcaagatggcagagggtacggagtcttcaggcccagtttctcactttagccaattcgagggctccttgtggtgggatcagaactaatccagagtgtgggaaagtgacagtcaaaaccccacctggagcaaataaaaaaacatacaaaacgtactggtgctttcctgtctaagtcgccttttgtgtgttcttttatgaggcccccaccatccacccctctgtgcacaaggcggctcccggcccctggaatgtgatgtttttggtcatctccaaaggctgcagttttatactgggaggctgatgacaccttttattataattattcttatggttctggttataattatgtttgttttaagattttctttaagaaaacaaaaacccaacacccttccctttaggtttcaaaccaaggtgcgggagcagggtgcttctgagttagcggtggctctggtgccctggctcccacccctcgggccaggtgggccactgggcatggtgacagctgggcggccagggagtccccaaggtctgccttctgggcttgtcactcttctcttctacttcccctcctcttgagtcgggtgtgtcagggctggagggaggaccggccgtctctctccttgtgtgtgtgattggagtggtgtgtatttcttttctagtacttggtctgatgatagctgtgctcttacctcgagtcagcagcacctggagccccaagtgctcaacactttgttctacttcctacaaggcaggcggcctggcgtcagtagtaggaagggatggcattgttggctccagggtgagggcccgacgggcaggtgcttgcctgggtagatggggtgtagatatgtggcatatagagatatatattttataatgggaggggggatggcacctcctgggactggtagagctgggaggtgtgtcggcaagcacatggccccaagtccacgtccctagaaagatggggcctacagtgataggcgctctataaatacatagatagggtcatgtttaagaaatattatggggtggggagtgggggaagcaggcttagtgtgctgagcctcggtccccactgcagacctaaaccccacaccaatcagtcaatctcacacgcatacacaggagcatatttctgtactgatgcataatgaatgcactcacacacacacacacacgccagggggctcccagcactcttctcccagggttcaggactttgtggagtcagcccgtgcagctatttacctctgttgccctgattgcaggtagactgtcaatcccagaagttgctaggtgctgaggcaggaaaaggaggaggtttcttttagcaggtgctctcctctagcaggtagtgttctatctcttttgcccctggtgatgtcaaaagattcttctgttagcaacctgtgatactgacctgcaactctaggcaccagtcaagtcagccctcaactccccctacctattctccccaactgaaatcaaattgacatccaggtctgtcttggccagggggcttccattggggaaaaaaaatgggacagtgaacctcacctattgaataaaccaaattagtaattagtgacaaaaccaaaccaaggctttggaccccctgctcattactcaggagtaaattcacacagctggtggccggtctcacccctttttggggtgcttggtcctggaccccaatctactgtctcctcgacagattggtgtttaagatcatgcccagttgcttcctgtcacctcttgacaggtggtgactgggctgctgtggggctggagcagcgaggaagggagcctctgccccctcagtggagtccctgtgctaactgtggtgctcacccagctcctccttgctcccgcggggctctgtccttcgggcctggagtccaccagcgccactccaggagggctgggggaggaggggctgcctcaggcagtccaaggtgctccctcccccgctggccaatgctaaggtcttcccggctcccagggtcaggacagagtcttctaaggctcaggctctcccaagccactgcttagtgacagttagcctttcccagcggcccaagctccaccggaactgggcctggtgctccaaccaaagggttggtgttgggacttgggaagagacttggtagccaagggcatggcaagcctgggggattcctgtgggccggcttgctcgaggagggagtgctgggcacagaggagcccttatggctgacagcttgagagcacttgttcctccagatctagccacttgaccaggccctagaatggaccagatgccccaggggtcatggcccccccccccccccccttgcagggacgagagttaacaatcaccagcttttcttccggcccagcctgtgtgcttggtctcacagtagaatttaggagagtgtggggctttgggattgtgccaacttggccaagctgagatatgcttaatccacctcaacgctggggcctctggggacaggtattgggtcaccttccaaggggtgctgaacttcagcaggctgtagataaggcagaggttagaggcagtggagtgggggaaccaagaaggcctagggaaaattgccaggcatattccctgtggaattttctttccgaagagtaggcccatttcccccttagaaacaagggttctgggggctgatgacagcttcccctgcaggtgccaggtggcgcatgatgccaaagagatccgtggctcttgcctgggggaggtagccaggtgctcgtgctggggacacagtccttggcctcctgcctaagcaggaagctagtttccagcaggcggctggtactaaccccccgccccacgggagcgacaggctggtgcaccggcagactggggggccgcaggcgggcactagcaggggtctgcagtccatagatgatgacatgaatctgttgctgtccttttgtctctaggccccagagacttcttgctattgaacatcctattggaaagcaaactttccaagagagcacccgggacctgttagcttttgaaacaggcctgggtgtgattgactggtagagatgcctgtgtgatttttggcgcattgcaggagtgtggattgggacaggcaccccagcaggaaggaaggctgacaccaaaggggtctcacagggcccagctccccttgcttcccgataattggccaccaggccatggcgactctggggaagcctagatgggaacacccaccttctccagcagtagaaggaatatttcagcagagccatctgggagatgttcttataggaaaactgcagtcattcatttcatagttttgcttatataaatagagtacatttgggaggcagttttgaattcctgttgctactctgcttggtgcggagttgcagtggccccaagtgtctttcagagctgttgcttttaggatccccctcaagctgtgggttagggccagggcagggagggggtgctgcctggcactgtgccctgaagagggctgcttgcttcctttggtcgggagcacggcagagccaacattctggtgggaaccgagtttctagccaggcccctgttttggtcccgggaggtgagtaatctgagtttgtcattgcgtattctccacgctgcctgctctcagtgctggagactagagagcgaccctggagttgtcctctgggagtcctgcctgtttgccagggagcagctgaaacctccccgctcccaatcccgggggtggaaaaggtgtagtggaggaatggtctggagctgggatggcccttctggccatgccctgctgagccaggattaaggaaagtgtcagccgcctccttaccaggccctgtgctggggtcaggaggaccgggaggagagggaaggggtgagaggatggactctgctgttgtagtgtgagctctggaatctttacgttttggtaaaggcaggccagggtctttgaggatcgagttggggaaccttgcctctgtgtttccgagacccagctaaggtgggcctttggtgggtgacctgtctctgacccgaggcagcagagttttgggggagggagaaacaagcaaggagcagggaggacaaggacacaagatccctttgctggtggtgacttactcacttcccccaccagagggcctgggactcccgaaactcccgtaactggaaagctccctggctcagctccactggggggtgaggagggcggctccctcagccccagcctagcggcctccaaccctgtgcctgagaacatgagcagcggtctaaagcatcccctagtcccatttcacttcacagaccgatttggttgcacagccctagaattcaacagacaacaaaaatgctgaaattccttagaatacgcagagggaggaggtgactcaccaaggtgctaaaacattttattaaaaatatatattgttaaattaagtctgctgtctggacaatgactgtttgttttgttttcttgtagtggagtttaaaagagactattattttactctgatatattattattaaaaaggcattttaactttgtacttgaaaactaaatgagcgatttcatgtgttttagctgaggcattgaagtagcgggtgcttacttatttgtgggaaatcatgtattcatactgaagaataaactccgtagctgatttggtaataacttttactgttacagacgatttcgctttgaccccggtggcagagcacttttactccacattccacgcagctaaatgcaggacttcccagactcttccctccccagccctctttcctttctcttaagttcctcttttctcctttattattattatttttttttcctggttccagcatcctttcaccactgtattttttttttttttttttttttttttttttttttttttttttttttttttttagggttgcttctctatttattgacaataataatgtacatttcacagtctggttctaggacattcagggaagggcgagtgggggcccgaggggtccctgccccacccgcctggtgtcctgttgtatctctgtgtaagtgatgctcgtgacatagctgtttatgaaataattaaagaggtcaatgcgtacaacagatgtagaaagtctgtcagttccgccttcatcacatttttttttgtgcccagaagaatataataaagctcctttctaatgtacttgtgctggagaacacttgaataaatggactgtttttgtgcaaaaagaaaaa

SEQ ID NO: 7 - RNA, 2631 nt, Homo sapiens
  source 1..2631: mol_type = mRNA
agttgagccctgaggccctgctggcccctgggcgcaggcccagctcaggcccccagggatggacgtcgtggaccctgacattttcaatagagacccccgggaccactatgacctgctacagcggctgggtggcggcacgtatggggaagtctttaaggctcgagacaaggtgtcaggggacctggtggcactgaagatggtgaagatggagcctgatgatgatgtctccacccttcagaaggaaatcctcatattgaaaacttgccggcacgccaacatcgtggcctaccatgggagttatctctggttgcagaaactctggatctgcatggaattctgtggggctggttctctccaggacatctaccaagtgacaggctccctgtcagagctccagattagctatgtctgccgggaagtgctccagggactggcctatttgcactcacagaagaagatacacagggacatcaagggagctaacatcctcatcaatgatgctggggaggtcagattggctgactttggcatctcggcccagattggggctacactggccagacgcctctctttcattgggacaccctactggatggctccggaagtggcagctgtggccctgaagggaggatacaatgagctgtgtgacatctggtccctgggcatcacggccatcgaactggccgagctacagccaccgctctttgatgtgcaccctctcagagttctcttcctcatgaccaagagtggctaccagcctccccgactgaaggaaaaaggcaaatggtcggctgccttccacaacttcatcaaagtcactctgactaagagtcccaagaaacgacccagcgccaccaagatgctcagtcatcaactggtatcccagcctgggctgaatcgaggcctgatcctggatcttcttgacaaactgaagaatcccgggaaaggaccctccattggggacattgaggatgaggagcccgagctaccccctgctatccctcggcggatcagatccacccaccgctccagctctctggggatcccagatgcagactgctgtcggcggcacatggagttcaggaagctccgaggaatggagaccagacccccagccaacaccgctcgcctacagcctcctcgagacctcaggagcagcagccccaggaagcaactgtcagagtcgtctgacgatgactatgacgacgtggacatccccacccctgcagaggacacacctcctccacttccccccaagcccaagttccgttctccatcagacgagggtcctgggagcatgggggatgatgggcagctgagcccgggggtgctggtccggtgtgccagtgggcccccaccaaacagcccccgtcctgggcctcccccatccaccagcagcccccacctcaccgcccattcagaaccctcactctggaacccaccctcccgggagcttgacaagcccccacttctgccccccaagaaggaaaagatgaagagaaagggatgtgcccttctcgtaaagttgttcaatggctgccccctccggatccacagcacggccgcctggacacatccctccaccaaggaccagcacctgctcctgggggcagaggaaggcatcttcatcctgaaccggaatgaccaggaggccacgctggaaatgctctttcctagccggactacgtgggtgtactccatcaacaacgttctcatgtctctctcaggaaagaccccccacctgtattctcatagcatccttggcctgctggaacggaaagagaccagagcaggaaaccccatcgctcacattagcccccaccgcctactggcaaggaagaacatggtttccaccaagatccaggacaccaaaggctgccgggcgtgctgtgtggcggagggtgcgagctctgggggcccgttcctgtgcggtgcattggagacgtccgttgtcctgcttcagtggtaccagcccatgaacaaattcctgcttgtccggcaggtgctgttcccactgccgacgcctctgtccgtgttcgcgctgctgaccgggccaggctctgagctgcccgctgtgtgcatcggcgtgagccccgggcggccggggaagtcggtgctcttccacacggtgcgctttggcgcgctctcttgctggctgggcgagatgagcaccgagcacaggggacccgtgcaggtgacccaggtagaggaagatatggtgatggtgttgatggatggctctgtgaagctggtgaccccggaggggtccccagtccggggacttcgcacacctgagatccccatgaccgaagcggtggaggccgtggctatggttggaggtcagcttcaggccttctggaagcatggagtgcaggtgtgggctctaggctcggatcagctgctacaggagctgagagaccctaccctcactttccgtctgcttggctcccccaggcctgtagtggtggagacacgcccagtggatgatcctactgctcccagcaacctctacatccaggaatgagtccctaggggggtgtcaggaactagtccttgcaccccctcccccatagacacactagtggtcatggcatgtcctcatctcccaataaacatgactttagcctctgc

SEQ ID NO: 8 - RNA, 2635 nt, Homo sapiens
  source 1..2635: mol_type = mRNA
agtcccagtggcggaggctacgaaacttgggggagtgcacagaagaacttcgggagcgcacgcgggaccagggaccaggctgagactcggggcgccagtccgggcaggggcagcgggagccggccgggtagggtgcagcctgaggcttgttcagcagaacaggtgcaagccacattgttgccaagacctgcctgaagccggattctccccactgcctccttcaaccccgcctcttcctcctcctgtgggactgctcccccctcctgtgaggctagatagatgccctgtatccaagcccaatatgggacaccagcaccgagtccgggaccccgtgaccacctggcaagcgaccccctgacccctgagttcatcaagcccaccatggacctggccagccccgaggcagcccccgctgcccccactgccctgcccagcttcagcaccttcatggacggctacacaggagagtttgacaccttcctctaccagctgccaggaacagtccagccatgctcctcagcctcctcctcggcctcctccacatcctcgtcctcagccacctcccctgcctctgcctccttcaagttcgaggacttccaggtgtacggctgctaccccggccccctgagcggcccagtggatgaggccctgtcctccagtggctctgactactatggcagcccctgctcggccccgtcgccctccacgcccagcttccagccgccccagctctctccctgggatggctccttcggccacttctcgcccagccagacttacgaaggcctgcgggcatggacagagcagctgcccaaagcctctgggcccccacagcctccagccttcttttccttcagtcctcccaccggccccagccccagcctggcccagagccccctgaagttgttcccctcacaggccacccaccagctgggggagggagagagctattccatgcctacggccttcccaggtttggcacccacttctccacaccttgagggctcggggatactggatacacccgtgacctcaaccaaggcccggagcggggccccaggtggaagtgaaggccgctgtgctgtgtgtggggacaacgcttcatgccagcattatggtgtccgcacatgtgagggctgcaagggcttcttcaagcgcacagtgcagaaaaacgccaagtacatctgcctggctaacaaggactgccctgtggacaagaggcggcgaaaccgctgccagttctgccgcttccagaagtgcctggcggtgggcatggtgaaggaagttgtccgaacagacagcctgaaggggcggcggggccggctaccttcaaaacccaagcagcccccagatgcctcccctgccaatctcctcacttccctggtccgtgcacacctggactcagggcccagcactgccaaactggactactccaagttccaggagctggtgctgccccactttgggaaggaagatgctggggatgtacagcagttctacgacctgctctccggttctctggaggtcatccgcaagtgggcggagaagatccctggctttgctgagctgtcaccggctgaccaggacctgttgctggagtcggccttcctggagctcttcatcctccgcctggcgtacaggtctaagccaggcgagggcaagctcatcttctgctcaggcctggtgctacaccggctgcagtgtgcccgtggcttcggggactggattgacagtatcctggccttctcaaggtccctgcacagcttgcttgtcgatgtccctgccttcgcctgcctctctgcccttgtcctcatcaccgaccggcatgggctgcaggagccgcggcgggtggaggagctgcagaaccgcatcgccagctgcctgaaggagcacgtggcagctgtggcgggcgagccccagccagccagctgcctgtcacgtctgttgggcaaactgcccgagctgcggaccctgtgcacccagggcctgcagcgcatcttctacctcaagctggaggacttggtgccccctccacccatcattgacaagatcttcatggacacgctgcccttctgacccctgcctgggaacacgtgtgcacatgcgcactctcatatgccaccccatgtgcctttagtccacggacccccagagcacccccaagcctgggcttgagctgcagaatgactccaccttctcacctgctccaggaggtttgcagggagctcaagcccttggggagggggatgccttcatgggggtgaccccacgatttgtcttatcccccccagcctggccccggcctttatgttttttgtaagataaaccgtttttaacacatagcgccgtgctgtaaataagcccagtgctgctgtaaatacaggaagaaagagcttgaggtgggagcggggctgggaggaagggatgggccccgccttcctgggcagcctttccagcctcctgctggctctctcttcctaccctccttccacatgtacataaactgtcactctaggaagaagacaaatgacagattctgacatttatatttgtgtattttcctggatttatagtatgtgacttttctgattaatatatttaatatattgaataaaaaatagacatgtagttggaa

SEQ ID NO: 9 - RNA, 3470 nt, Homo sapiens
  source 1..3470: mol_type = mRNA
gcatgcgccgcagcgccagcgctctccccggatcgtgcggggcctgagcctctccgccggcgcaggctctgctcgcgccagctcgctcccgcagccatgcccaccaccatcgagcgggagttcgaagagttggatactcagcgtcgctggcagccgctgtacttggaaattcgaaatgagtcccatgactatcctcatagagtggccaagtttccagaaaacagaaatcgaaacagatacagagatgtaagcccatatgatcacagtcgtgttaaactgcaaaatgctgagaatgattatattaatgccagtttagttgacatagaagaggcacaaaggagttacatcttaacacagggtccacttcctaacacatgctgccatttctggcttatggtttggcagcagaagaccaaagcagttgtcatgctgaaccgcattgtggagaaagaatcggttaaatgtgcacagtactggccaacagatgaccaagagatgctgtttaaagaaacaggattcagtgtgaagctcttgtcagaagatgtgaagtcgtattatacagtacatctactacaattagaaaatatcaatagtggtgaaaccagaacaatatctcactttcattatactacctggccagattttggagtccctgaatcaccagcttcatttctcaatttcttgtttaaagtgagagaatctggctccttgaaccctgaccatgggcctgcggtgatccactgtagtgcaggcattgggcgctctggcaccttctctctggtagacacttgtcttgttttgatggaaaaaggagatgatattaacataaaacaagtgttactgaacatgagaaaataccgaatgggtcttattcagaccccagatcaactgagattctcatacatggctataatagaaggagcaaaatgtataaagggagattctagtatacagaaacgatggaaagaactttctaaggaagacttatctcctgcctttgatcattcaccaaacaaaataatgactgaaaaatacaatgggaacagaataggtctagaagaagaaaaactgacaggtgaccgatgtacaggactttcctctaaaatgcaagatacaatggaggagaacagtgagagtgctctacggaaacgtattcgagaggacagaaaggccaccacagctcagaaggtgcagcagatgaaacagaggctaaatgagaatgaacgaaaaagaaaaaggtggttatattggcaacctattctcactaagatggggtttatgtcagtcattttggttggcgcttttgttggctggacactgttttttcagcaaaatgccctataaacaattaattttgcccagcaagcttctgcactagtaactgacagtgctacattaatcataggggtttgtctgcagcaaacgcctcatatcccaaaaacggtgcagtagaatagacatcaaccagataagtgatatttacagtcacaagcccaacatctcaggactcttgactgcaggttcctctgaaccccaaactgtaaatggctgtctaaaataaagacattcatgtttgttaaaaactggtaaattttgcaactgtattcatacatgtcaaacacagtatttcacctgaccaacattgagatatcctttatcacaggatttgtttttggaggctatctggattttaacctgcacttgatataagcaataaatattgtggttttatctacgttattggaaagaaaatgacatttaaataatgtgtgtaatgtataatgtactattgacatgggcatcaacacttttattcttaagcatttcagggtaaatatattttataagtatctatttaatcttttgtagttaactgtactttttaagagctcaatttgaaaaatctgttactaaaaaaataaattgtatgtcgattgaattgtactggatacattttccatttttctaaagagaagtttgatatgagcagttagaagttggaataagcaatttctactatatattgcatttcttttatgttttacagttttccccattttaaaaagaaaagcaaacaaagaaacaaaagtttttcctaaaaatatctttgaaggaaaattctccttactgggatagtcaggtaaacagttggtcaagactttgtaaagaaattggtttctgtaaatcccattattgatatgtttatttttcatgaaaatttcaatgtagttggggtagattatgatttaggaagcaaaagtaagaagcagcattttatgattcataatttcagtttactagactgaagttttgaagtaaacacttttcagtttctttctacttcaataaatagtatgattatatgcaaaccttacattgtcattttaacttaatgaatattttttaaagcaaactgtttaatgaatttaactgctcatttgaatgctagctttcctcagatttcaacattccattcagtgtttaatttgtcttacttaaacttgaaattgttgttacaaatttaattgctaggaggcatggatagcatacattattatggatagcataccttatttcagtggttttcaaactatgctcattggatgtccaggtgggtcaagaggttactttcaaccacagcatctctgccttgtctctttatatgccacataagatttctgcataaggcttaagtattttaaagggggcagttatcatttaaaaacagtttggtcgggcgcggtggctcatgcctgtaatcccagcactttgggaggctgaagtgggcagatcacctgaggtcaggagttcaagaccagcctggccaacgtggtgaaacaccatctctactaaaaatgcaaaaattagctgggcatggtggagggcacctgtaatctcagctactcaggaggctgaggtaggagaattgcttgaacccaggagatggaggttgcagtgagctgagatcacgtcactgcactccagccagggcgacagagcgagactccatctcaaaagaaacaaacaaaaaaaacagtttgggccgggtgtggtggctcacgcttgtaatcccagcacttcggaaggccaaggcgggcggatcacgaggtcaagagatggagactgtcctggccaacatggtgaaatcccttctttactaaaaatacaaaaattatctgggcgtggtggtgcatgcctgtagtcccagctccttgggaggctaaggcaggagaatcacttgaacccgggaggcagaggttgcagtgagccgagattgcaccactgcactccagcctggcaacagagcaagacttcgtctcaaaaaaaaaaaaaaaaaaagtttgaaaaccattggtatagatagatattttgaattgatttgcatagtctccttgaatgtgttaaattatgttgaaagtatgaaagcaggatgtaggtggtactacatattaaataagatttatataaca

SEQ ID NO: 10 - RNA, 9697 nt, Homo sapiens
  source 1..9697: mol_type = mRNA
aaacagaaggtgggccggggcggggagaaacagaactcggtcaatttcccagtttgtcgggtctttaaaaatacaggcccctaaagcactaagggcatgccctcggtgaaacaggggagcgcttctgctgaatgagattaaagcgacagaaaagggaaaggagagcgcgggcaacgggatctaaagggagatagagacgcgggcctctgagggctggcaaacattcagcagcacaccctctcaagattgtttacttgcctttgctcctgttgagttacaacgcttggaagcaggagatgggctcagcagcagccaataggacatgatccaggaagagcagtaagggactgagctgctgaattcaactagagggcagccttgtggatggccccgaagcaagcctgatggaacaggatagaaccaaccatgttgagggcaacagactaagtccattcctgataccatcacctcccatttgccagacagaacctctggctacaaagctccagaatggaagcccactgcctgagagagctcatccagaagtaaatggagacaccaagtggcactctttcaaaagttattatggaataccctgtatgaagggaagccagaatagtcgtgtgagtcctgactttacacaagaaagtagagggtattccaagtgtttgcaaaatggaggaataaaacgcacagttagtgaaccttctctctctgggctccttcagatcaagaaattgaaacaagaccaaaaggctaatggagaaagacgtaacttcggggtaagccaagaaagaaatccaggtgaaagcagtcaaccaaatgtctccgatttgagtgataagaaagaatctgtgagttctgtagcccaagaaaatgcagttaaagatttcaccagtttttcaacacataactgcagtgggcctgaaaatccagagcttcagattctgaatgagcaggaggggaaaagtgctaattaccatgacaagaacattgtattacttaaaaacaaggcagtgctaatgcctaatggtgctacagtttctgcctcttccgtggaacacacacatggtgaactcctggaaaaaacactgtctcaatattatccagattgtgtttccattgcggtgcagaaaaccacatctcacataaatgccattaacagtcaggctactaatgagttgtcctgtgagatcactcacccatcgcatacctcagggcagatcaattccgcacagacctctaactctgagctgcctccaaagccagctgcagtggtgagtgaggcctgtgatgctgatgatgctgataatgccagtaaactagctgcaatgctaaatacctgttcctttcagaaaccagaacaactacaacaacaaaaatcagtttttgagatatgcccatctcctgcagaaaataacatccagggaaccacaaagctagcgtctggtgaagaattctgttcaggttccagcagcaatttgcaagctcctggtggcagctctgaacggtatttaaaacaaaatgaaatgaatggtgcttacttcaagcaaagctcagtgttcactaaggattccttttctgccactaccacaccaccaccaccatcacaattgcttctttctccccctcctcctcttccacaggttcctcagcttccttcagaaggaaaaagcactctgaatggtggagttttagaagaacaccaccactaccccaaccaaagtaacacaacacttttaagggaagtgaaaatagagggtaaacctgaggcaccaccttcccagagtcctaatccatctacacatgtatgcagcccttctccgatgctttctgaaaggcctcagaataattgtgtgaacaggaatgacatacagactgcagggacaatgactgttccattgtgttctgagaaaacaagaccaatgtcagaacacctcaagcataacccaccaatttttggtagcagtggagagctacaggacaactgccagcagttgatgagaaacaaagagcaagagattctgaagggtcgagacaaggagcaaacacgagatcttgtgcccccaacacagcactatctgaaaccaggatggattgaattgaaggcccctcgttttcaccaagcggaatcccatctaaaacgtaatgaggcatcactgccatcaattcttcagtatcaacccaatctctccaatcaaatgacctccaaacaatacactggaaattccaacatgcctggggggctcccaaggcaagcttacacccagaaaacaacacagctggagcacaagtcacaaatgtaccaagttgaaatgaatcaagggcagtcccaaggtacagtggaccaacatctccagttccaaaaaccctcacaccaggtgcacttctccaaaacagaccatttaccaaaagctcatgtgcagtcactgtgtggcactagatttcattttcaacaaagagcagattcccaaactgaaaaacttatgtccccagtgttgaaacagcacttgaatcaacaggcttcagagactgagccattttcaaactcacaccttttgcaacataagcctcataaacaggcagcacaaacacaaccatcccagagttcacatctccctcaaaaccagcaacagcagcaaaaattacaaataaagaataaagaggaaatactccagacttttcctcacccccaaagcaacaatgatcagcaaagagaaggatcattctttggccagactaaagtggaagaatgttttcatggtgaaaatcagtattcaaaatcaagcgagttcgagactcataatgtccaaatgggactggaggaagtacagaatataaatcgtagaaattccccttatagtcagaccatgaaatcaagtgcatgcaaaatacaggtttcttgttcaaacaatacacacctagtttcagagaataaagaacagactacacatcctgaactttttgcaggaaacaagacccaaaacttgcatcacatgcaatattttccaaataatgtgatcccaaagcaagatcttcttcacaggtgctttcaagaacaggagcagaagtcacaacaagcttcagttctacagggatataaaaatagaaaccaagatatgtctggtcaacaagctgcgcaacttgctcagcaaaggtacttgatacataaccatgcaaatgtttttcctgtgcctgaccagggaggaagtcacactcagacccctccccagaaggacactcaaaagcatgctgctctaaggtggcatctcttacagaagcaagaacagcagcaaacacagcaaccccaaactgagtcttgccatagtcagatgcacaggccaattaaggtggaacctggatgcaagccacatgcctgtatgcacacagcaccaccagaaaacaaaacatggaaaaaggtaactaagcaagagaatccacctgcaagctgtgataatgtgcagcaaaagagcatcattgagaccatggagcagcatctgaagcagtttcacgccaagtcgttatttgaccataaggctcttactctcaaatcacagaagcaagtaaaagttgaaatgtcagggccagtcacagttttgactagacaaaccactgctgcagaacttgatagccacaccccagctttagagcagcaaacaacttcttcagaaaagacaccaaccaaaagaacagctgcttctgttctcaataattttatagagtcaccttccaaattactagatactcctataaaaaatttattggatacacctgtcaagactcaatatgatttcccatcttgcagatgtgtagagcaaattattgaaaaagatgaaggtcctttttatacccatctaggagcaggtcctaatgtggcagctattagagaaatcatggaagaaaggtttggacagaagggtaaagctattaggattgaaagagtcatctatactggtaaagaaggcaaaagttctcagggatgtcctattgctaagtgggtggttcgcagaagcagcagtgaagagaagctactgtgtttggtgcgggagcgagctggccacacctgtgaggctgcagtgattgtgattctcatcctggtgtgggaaggaatcccgctgtctctggctgacaaactctactcggagcttaccgagacgctgaggaaatacggcacgctcaccaatcgccggtgtgccttgaatgaagagagaacttgcgcctgtcaggggctggatccagaaacctgtggtgcctccttctcttttggttgttcatggagcatgtactacaatggatgtaagtttgccagaagcaagatcccaaggaagtttaagctgcttggggatgacccaaaagaggaagagaaactggagtctcatttgcaaaacctgtccactcttatggcaccaacatataagaaacttgcacctgatgcatataataatcagattgaatatgaacacagagcaccagagtgccgtctgggtctgaaggaaggccgtccattctcaggggtcactgcatgtttggacttctgtgctcatgcccacagagacttgcacaacatgcagaatggcagcacattggtatgcactctcactagagaagacaatcgagaatttggaggaaaacctgaggatgagcagcttcacgttctgcctttatacaaagtctctgacgtggatgagtttgggagtgtggaagctcaggaggagaaaaaacggagtggtgccattcaggtactgagttcttttcggcgaaaagtcaggatgttagcagagccagtcaagacttgccgacaaaggaaactagaagccaagaaagctgcagctgaaaagctttcctccctggagaacagctcaaataaaaatgaaaaggaaaagtcagccccatcacgtacaaaacaaactgaaaacgcaagccaggctaaacagttggcagaacttttgcgactttcaggaccagtcatgcagcagtcccagcagccccagcctctacagaagcagccaccacagccccagcagcagcagagaccccagcagcagcagccacatcaccctcagacagagtctgtcaactcttattctgcttctggatccaccaatccatacatgagacggcccaatccagttagtccttatccaaactcttcacacacttcagatatctatggaagcaccagccctatgaacttctattccacctcatctcaagctgcaggttcatatttgaattcttctaatcccatgaacccttaccctgggcttttgaatcagaatacccaatatccatcatatcaatgcaatggaaacctatcagtggacaactgctccccatatctgggttcctattctccccagtctcagccgatggatctgtataggtatccaagccaagaccctctgtctaagctcagtctaccacccatccatacactttaccagccaaggtttggaaatagccagagttttacatctaaatacttaggttatggaaaccaaaatatgcagggagatggtttcagcagttgtaccattagaccaaatgtacatcatgtagggaaattgcctccttatcccactcatgagatggatggccacttcatgggagccacctctagattaccacccaatctgagcaatccaaacatggactataaaaatggtgaacatcattcaccttctcacataatccataactacagtgcagctccgggcatgttcaacagctctcttcatgccctgcatctccaaaacaaggagaatgacatgctttcccacacagctaatgggttatcaaagatgcttccagctcttaaccatgatagaactgcttgtgtccaaggaggcttacacaaattaagtgatgctaatggtcaggaaaagcagccattggcactagtccagggtgtggcttctggtgcagaggacaacgatgaggtctggtcagacagcgagcagagctttctggatcctgacattgggggagtggccgtggctccaactcatgggtcaattctcattgagtgtgcaaagcgtgagctgcatgccacaacccctttaaagaatcccaataggaatcaccccaccaggatctccctcgtcttttaccagcataagagcatgaatgagccaaaacatggcttggctctttgggaagccaaaatggctgaaaaagcccgtgagaaagaggaagagtgtgaaaagtatggcccagactatgtgcctcagaaatcccatggcaaaaaagtgaaacgggagcctgctgagccacatgaaacttcagagcccacttacctgcgtttcatcaagtctcttgccgaaaggaccatgtccgtgaccacagactccacagtaactacatctccatatgccttcactcgggtcacagggccttacaacagatatatatgatatcacccccttttgttggttacctcacttgaaaagaccacaaccaacctgtcagtagtatagttctcatgacgtgggcagtggggaaaggtcacagtattcatgacaaatgtggtgggaaaaacctcagctcaccagcaacaaaagaggttatcttaccatagcacttaattttcactggctcccaagtggtcacagatggcatctaggaaaagaccaaagcattctatgcaaaaagaaggtggggaagaaagtgttccgcaatttacatttttaaacactggttctattattggacgagatgatatgtaaatgtgatcccccccccccgcttacaactctacacatctgtgaccacttttaataatatcaagtttgcatagtcatggaacacaaatcaaacaagtactgtagtattacagtgacaggaatcttaaaataccatctggtgctgaatatatgatgtactgaaatactggaattatggctttttgaaatgcagtttttactgtaatcttaacttttatttatcaaaatagctacaggaaacatgaatagcaggaaaacactgaatttgtttggatgttctaagaaatggtgctaagaaaatggtgtctttaatagctaaaaatttaatgcctttatatcatcaagatgctatcagtgtactccagtgcccttgaataataggggtaccttttcattcaagtttttatcataattacctattcttacacaagcttagtttttaaaatgtggacattttaaaggcctctggattttgctcatccagtgaagtccttgtaggacaataaacgtatatatgtacatatatacacaaacatgtatatgtgcacacacatgtatatgtataaatattttaaatggtgttttagaagcactttgtctacctaagctttgacaacttgaacaatgctaaggtactgagatgtttaaaaaacaagtttactttcattttagaatgcaaagttgatttttttaaggaaacaaagaaagcttttaaaatatttttgcttttagccatgcatctgctgatgagcaattgtgtccatttttaacacagccagttaaatccaccatggggcttactggattcaagggaatacgttagtccacaaaacatgttttctggtgctcatctcacatgctatactgtaaaacagttttatacaaaattgtatgacaagttcattgctcaaaaatgtacagttttaagaattttctattaactgcaggtaataattagctgcatgctgcagactcaacaaagctagttcactgaagcctatgctattttatggatcataggctcttcagagaactgaatggcagtctgcctttgtgttgataattatgtacattgtgacgttgtcatttcttagcttaagtgtcctctttaacaagaggattgagcagactgatgcctgcataagatgaataaacagggttagttccatgtgaatctgtcagttaaaaagaaacaaaaacaggcagctggtttgctgtggtggttttaaatcattaatttgtataaagaagtgaaagagttgtatagtaaattaaattgtaaacaaaacttttttaatgcaatgctttagtattttagtactgtaaaaaaattaaatatatacatatatatatatatatatatatatatatatatgagtttgaagcagaattcacatcatgatggtgctactcagcctgctacaaatatatcataatgtgagctaagaattcattaaatgtttgagtgatgttcctacttgtcatatacctcaacactagtttggcaataggatattgaactgagagtgaaagcattgtgtaccatcatttttttccaagtccttttttttattgttaaaaaaaaaagcataccttttttcaatacttgatttcttagcaagtataacttgaacttcaacctttttgttctaaaaattcagggatatttcagctcatgctctccctatgccaacatgtcacctgtgtttatgtaaaattgttgtaggttaataaatatattctttgtcagggatttaacccttttattttgaatcccttctattttacttgtacatgtgctgatgtaactaaaactaattttgtaaatctgttggctctttttattgtaaagaaaagcattttaaaagtttgaggaatcttttgactgtttcaagcaggaaaaaaaaattacatgaaaatagaatgcactgagttgataaagggaaaaattgtaaggcaggagtttggcaagtggctgttggccagagacttacttgtaactctctaaatgaagtttttttgatcctgtaatcactgaaggtacatactccatgtggacttcccttaaacaggcaaacacctacaggtatggtgtgcaacagattgtacaattacattttggcctaaatacatttttgcttactagtatttaaaataaattcttaatcagaggaggcctttgggttttattggtcaaatctttgtaagctggcttttgtctttttaaaaaatttcttgaatttgtggttgtgtccaatttgcaaacatttccaaaaatgtttgctttgcttacaaaccacatgattttaatgttttttgtataccataatatctagccccaaacatttgattactacatgtgcattggtgattttgatcatccattcttaatatttgatttctgtgtcacctactgtcatttgttaaactgctggccaacaagaacaggaagtatagtttggggggttggggagagtttacataaggaagagaagaaattgagtggcatattgtaaatatcagatctataattgtaaatataaaacctgcctcagttagaatgaatggaaagcagatctacaatttgctaatataggaatatcaggttgactatatagccatacttgaaaatgcttctgagtggtgtcaactttacttgaatgaatttttcatcttgattgacgcacagtgatgtacagttcacttctgaagctagtggttaacttgtgtaggaaacttttgcagtttgacactaagataacttctgtgtgcatttttctatgcttttttaaaaactagtttcatttcattttcatgagatgtttggtttataagatctgaggatggttataaatactgtaagtattgtaatgttatgaatgcaggttatttgaaagctgtttattattatatcattcctgataatgctatgtgagtgtttttaataaaatttatatttatttaatgcactctaa

SEQ ID NO: 11 - RNA, 2734 nt, Homo sapiens
  source 1..2734: mol_type = mRNA
aggactcggccccatggagacgccgccggccgccgctggcgcatggcgggtaggagctgtggcgcggggccttccaggagtctgagctatgagtggcccctgtggagagaagcctgtcctggaagccagccccaccatgagtctgtgggaatttgaggacagccacagccgtcagggcaccccaaggccgggtcaagagctggccgctgaggaggcctcggccctggaactgcagatgaaggtggacttcttccggaagctgggctattcatccacggagatccacagcgtcctgcagaagctgggcgtccaggcagacaccaacacggtgctgggtgagctggtgaaacacgggacagccaccgagcgggagcgccagacctcaccggacccctgccctcagctccctctagtcccgcggggtggtggcacccctaaggctcccaacctggagcctccactcccagaagaggaaaaggagggcagcgacctgagaccagtggtcatcgatgggagcaacgtggccatgagccatgggaacaaggaggtcttctcctgccggggcatcctgctggcagtgaactggtttctggagcggggccacacagacatcacagtgtttgtgccatcctggaggaaggagcagcctcggcccgacgtgcccatcacagaccagcacatcctgcgggaactggagaagaagaagatcctggtgttcacaccatcacgacgcgtgggtggcaagcgggtggtgtgctatgacgacagattcattgtgaagctggcctacgagtctgacgggatcgtggtttccaacgacacataccgtgacctccaaggcgagcggcaggagtggaagcgcttcatcgaggagcggctgctcatgtactccttcgtcaatgacaagcttgccatcggccccttctcagcaattagaagtccctgcatggctcccacctccagagagttctttgcaccctctgcaggtttatgccccctgatgacccactgggccggcacgggcccagcctggacaacttcctgcgtaagaagccactcactttggagcacaggaagcagccgtgtccctatggaaggaaatgcacctatgggatcaagtgccgattcttccacccagagcggccaagctgcccccagcgctctgtggcagatgagctccgtgccaatgctctcctctcaccccccagagccccaagcaaggacaaaaatggccggcggccttcaccttcatcccagtccagctctctgctaacagagagtgagcagtgcagcctggatgggaagaagctgggggcccaggcatccccagggtcccgccaagagggtctaacacagacctatgccccatcaggcaggagcctcgcacctagcgggggcagtggcagcagctttgggcccacagactggctcccacagacgctggactcactcccgtacgtctcccaggattgcctggactcgggcattggctccctggagagccagatgtcggaactttggggggttcgaggaggaggccctggtgagccgggcccaccccgagccccttacacgggctacagtccctatggatctgagctcccagccaccgcagccttctctgcctttggccgggccatgggtgctggccacttcagtgtccctgccgactacccacccgcgccccctgcctttccacctcgagagtactggtctgaaccatacccactgcccccacccacatcagtccttcaggagcccccagtgcagagcccaggggctggcaggagcccgtggggcagggcaggcagcctggccaaggagcaggccagcgtgtatactaagctgtgtggtgtgtttcccccgcacctggtggaggctgtgatggggcgcttcccacagctcctggacccccagcagctggctgccgagatcctctcctacaagtcccagcaccccagtgagtaagctgcctgtggctggcaagggcagcacccccagcctccaagggccgtcaggctgggctttgggccattgagcagcccattcccagccctgaggcccaccccagaggctggacagagggaggattcaagtcgggaaggaaacccacaaaccaaagatactgtaggattggttctggcccatgcagcacctctagctgtctgcctcagtgggtcagaagcgatcaccctgttgatacacattgtatctctgtagtttaaggagacgctgccggtaacggcgtcggtccgtggctgaggcccaaaccgtcttttctctcagagggtggggagggaggtgggggcagcagaggcctgggctgggtgccctgtgcacgccaccccacttccgccctacccctgggacgttggccttggctggctagttgggcaccgtgtgcctgccctccaagggcctcctctacgccaatgaggcctcatctgtgctctcgctgggcacgtggcttcatgtcagtaagcaagatgcttcttaataacccaccttctgccccactctattccttatcctgctgcccctgtaggggtcaagggccctccgtctacaccctcttcttctcctccatcctttattcagagtcatctcgcccttccccatgggtgggggaacctgtgtttgtttgtgtgcacatgtaaattttaaatattttaagcagaaagtccttacctcctgtaacacatcaataaagtacaatcattgtgagccctttcaa

SEQ ID NO: 12 - RNA, 20 nt, synthetic construct
  source 1..20: mol_type = other RNA
  modified_base 1..3: mod_base = OTHER; note = 2'O-methyl nucleotide phosphorothioate
cataccagctgagccgtccg

SEQ ID NO: 13 - RNA, 20 nt, synthetic construct
  source 1..20: mol_type = other RNA
  modified_base 1..3: mod_base = OTHER; note = 2'O-methyl nucleotide phosphorothioate
aaaatatcaagtatatatgg

SEQ ID NO: 14 - RNA, 20 nt, synthetic construct
  source 1..20: mol_type = other RNA
  modified_base 1..3: mod_base = OTHER; note = 2'O-methyl nucleotide phosphorothioate
gccaaaggtgctggacccag

SEQ ID NO: 15 - RNA, 20 nt, synthetic construct
  source 1..20: mol_type = other RNA
  modified_base 1..3: mod_base = OTHER; note = 2'O-methyl nucleotide phosphorothioate
tccggatctttttctgacag

SEQ ID NO: 16 - RNA, 20 nt, synthetic construct
  source 1..20: mol_type = other RNA
  modified_base 1..3: mod_base = OTHER; note = 2'O-methyl nucleotide phosphorothioate
ggtagccctacggcgcaagg

SEQ ID NO: 17 - RNA, 20 nt, synthetic construct
  source 1..20: mol_type = other RNA
  modified_base 1..3: mod_base = OTHER; note = 2'O-methyl nucleotide phosphorothioate
tgacacgccaaaggaccctg

SEQ ID NO: 18 - RNA, 20 nt, synthetic construct
  source 1..20: mol_type = other RNA
  modified_base 1..3: mod_base = OTHER; note = 2'O-methyl nucleotide phosphorothioate
agagggtgcacatcaaagag

SEQ ID NO: 19 - RNA, 20 nt, synthetic construct
  source 1..20: mol_type = other RNA
  modified_base 1..3: mod_base = OTHER; note = 2'O-methyl nucleotide phosphorothioate
catggacctggccagccccg

SEQ ID NO: 20 - RNA, 20 nt, synthetic construct
  source 1..20: mol_type = other RNA
  modified_base 1..3: mod_base = OTHER; note = 2'O-methyl nucleotide phosphorothioate
ttgacatagaagaggcacaa

SEQ ID NO: 21 - RNA, 20 nt, synthetic construct
  source 1..20: mol_type = other RNA
  modified_base 1..3: mod_base = OTHER; note = 2'O-methyl nucleotide phosphorothioate
aaagagcaagagattctgaa

SEQ ID NO: 22 - RNA, 20 nt, synthetic construct
  source 1..20: mol_type = other RNA
  modified_base 1..3: mod_base = OTHER; note = 2'O-methyl nucleotide phosphorothioate
cagctccctctagtcccgcg

SEQ ID NO: 23 - RNA, 22 nt, synthetic construct
  source 1..22: mol_type = other RNA
gtattcatcatcatcttcctct

SEQ ID NO: 24 - RNA, 22 nt, synthetic construct
  source 1..22: mol_type = other RNA
tttaacttcatcatatgtgaga

SEQ ID NO: 25 - RNA, 22 nt, synthetic construct
  source 1..22: mol_type = other RNA
tttataaactgaatgagaatgt

SEQ ID NO: 26 - RNA, 22 nt, synthetic construct
  source 1..22: mol_type = other RNA
ttcatcatcatcttcctctact

SEQ ID NO: 27 - RNA, 22 nt, synthetic construct
  source 1..22: mol_type = other RNA
tttgactttttcataaggctat

SEQ ID NO: 28 - RNA, 22 nt, synthetic construct
  source 1..22: mol_type = other RNA
tgaatactttccatggtacgat

SEQ ID NO: 29 - RNA, 22 nt, synthetic construct
  source 1..22: mol_type = other RNA
ttaaagtactcattctcactga

SEQ ID NO: 30 - RNA, 22 nt, synthetic construct
  source 1..22: mol_type = other RNA
tataattgaggataagtccttc

SEQ ID NO: 31 - RNA, 22 nt, synthetic construct
  source 1..22: mol_type = other RNA
ttagaaaactttctgtgggttc

SEQ ID NO: 32 - RNA, 22 nt, synthetic construct
  source 1..22: mol_type = other RNA
taatatattaggaatgttgcaa

SEQ ID NO: 33 - RNA, 22 nt, synthetic construct
  source 1..22: mol_type = other RNA
tgaaataagggcttgttatgag

SEQ ID NO: 34 - RNA, 22 nt, synthetic construct
  source 1..22: mol_type = other RNA
tttagaaaactttctgtgggtt

SEQ ID NO: 35 - RNA, 22 nt, synthetic construct
  source 1..22: mol_type = other RNA
tttattaagagtaataacatgg

SEQ ID NO: 36 - RNA, 22 nt, synthetic construct
  source 1..22: mol_type = other RNA
tttgatttcttctctgaagatg

SEQ ID NO: 37 - RNA, 22 nt, synthetic construct
  source 1..22: mol_type = other RNA
tcttcatacattctctccttgc

SEQ ID NO: 38 - RNA, 22 nt, synthetic construct
  source 1..22: mol_type = other RNA
taaatatatgcttaagtcaggg

SEQ ID NO: 39 - RNA, 22 nt, synthetic construct
  source 1..22: mol_type = other RNA
tcttgtattcatcatcatcttc

SEQ ID NO: 40 - RNA, 22 nt, synthetic construct
  source 1..22: mol_type = other RNA
tacattatgacaatgagatggt

SEQ ID NO: 41 - RNA, 22 nt, synthetic construct
  source 1..22: mol_type = other RNA
ttaattgaacatatgactattt

SEQ ID NO: 42 - RNA, 22 nt, synthetic construct
  source 1..22: mol_type = other RNA
tttattgacaaataaatgcttt

SEQ ID NO: 43 - RNA, 22 nt, synthetic construct
  source 1..22: mol_type = other RNA
tatgacaatgagatggttgtga

SEQ ID NO: 44 - RNA, 22 nt, synthetic construct
  source 1..22: mol_type = other RNA
ttctaaaggcaaatcatggcgt

SEQ ID NO: 45 - RNA, 22 nt, synthetic construct
  source 1..22: mol_type = other RNA
ttacattatgacaatgagatgg

SEQ ID NO: 46 - RNA, 22 nt, synthetic construct
  source 1..22: mol_type = other RNA
tgtgaattcagggaaacagggt

SEQ ID NO: 47 - RNA, 22 nt, synthetic construct
  source 1..22: mol_type = other RNA
tgcagatcatagtcactgtcgg

SEQ ID NO: 48 - RNA, 22 nt, synthetic construct
  source 1..22: mol_type = other RNA
ttgattttcctgaagcaatgct

SEQ ID NO: 49 - RNA, 22 nt, synthetic construct
  source 1..22: mol_type = other RNA
tgattttcctgaagcaatgctc

SEQ ID NO: 50 - RNA, 22 nt, synthetic construct
  source 1..22: mol_type = other RNA
ttttggttcattcccgttgggc

SEQ ID NO: 51 - RNA, 22 nt, synthetic construct
  source 1..22: mol_type = other RNA
tccaagtactagaggagtctgt

SEQ ID NO: 52 - RNA, 22 nt, synthetic construct
  source 1..22: mol_type = other RNA
tgacacatgtcatttctgctgt

SEQ ID NO: 53 - RNA, 22 nt, synthetic construct
  source 1..22: mol_type = other RNA
ttattcaaaccaaaggaacatg

SEQ ID NO: 54 - RNA, 22 nt, synthetic construct
  source 1..22: mol_type = other RNA
tgcagatcatagtcactgtcgg

SEQ ID NO: 55 - RNA, 22 nt, synthetic construct
  source 1..22: mol_type = other RNA
ttcgttatccacgtgggaggct

SEQ ID NO: 56 - RNA, 22 nt, synthetic construct
  source 1..22: mol_type = other RNA
tgattgagctctgaggtgtgta

SEQ ID NO: 57 - RNA, 22 nt, synthetic construct
  source 1..22: mol_type = other RNA
ttggaaggctgcttgttttggg

SEQ ID NO: 58 - RNA, 22 nt, synthetic construct
  source 1..22: mol_type = other RNA
tagaagatgaactgtgatggac

SEQ ID NO: 59 - RNA, 22 nt, synthetic construct
  source 1..22: mol_type = other RNA
ttggtttttaagtaggccatgg

SEQ ID NO: 60 - RNA, 22 nt, synthetic construct
  source 1..22: mol_type = other RNA
ttttattaaaaaaggcactgat

SEQ ID NO: 61 - RNA, 22 nt, synthetic construct
  source 1..22: mol_type = other RNA
ttcactagcttcttgtaggcaa

SEQ ID NO: 62 - RNA, 22 nt, synthetic construct
  source 1..22: mol_type = other RNA
ttggaaggctgcttgttttggg

SEQ ID NO: 63 - RNA, 22 nt, synthetic construct
  source 1..22: mol_type = other RNA
ttgggttagaagatgaactgtg

SEQ ID NO: 64 - RNA, 22 nt, synthetic construct
  source 1..22: mol_type = other RNA
tattcaaaccaaaggaacatga

SEQ ID NO: 65 - RNA, 22 nt, synthetic construct
  source 1..22: mol_type = other RNA
tattcaaaatgcactcgcccag

SEQ ID NO: 66 - RNA, 22 nt, synthetic construct
  source 1..22: mol_type = other RNA
tgggttagaagatgaactgtga

SEQ ID NO: 67 - RNA, 22 nt, synthetic construct
  source 1..22: mol_type = other RNA
aagatgaactgtgatggactga

SEQ ID NO: 68 - RNA, 22 nt, synthetic construct
  source 1..22: mol_type = other RNA
tcaaaccaaaggaacatgacca

SEQ ID NO: 69 - RNA, 22 nt, synthetic construct
  source 1..22: mol_type = other RNA
tgagcttccttgtcactcctta

SEQ ID NO: 70 - RNA, 22 nt, synthetic construct
  source 1..22: mol_type = other RNA
tagggacatgatttcaagtgaa

SEQ ID NO: 71 - RNA, 22 nt, synthetic construct
  source 1..22: mol_type = other RNA
aaaactattcaaaatgcactcg

SEQ ID NO: 72 - RNA, 22 nt, synthetic construct
  source 1..22: mol_type = other RNA
aaactattcaaaatgcactcgc

SEQ ID NO: 73 - RNA, 22 nt, synthetic construct
  source 1..22: mol_type = other RNA
ataaataaataaatagtgtata

SEQ ID NO: 74 - RNA, 22 nt, synthetic construct
  source 1..22: mol_type = other RNA
tgagacacaggctctgctgggg

SEQ ID NO: 75 - RNA, 22 nt, synthetic construct
  source 1..22: mol_type = other RNA
taaaggtataatgtcattacag

SEQ ID NO: 76 - RNA, 22 nt, synthetic construct
  source 1..22: mol_type = other RNA
tcacgtactaagaacgtgcctt

SEQ ID NO: 77 - RNA, 22 nt, synthetic construct
  source 1..22: mol_type = other RNA
tgcagatacagctctgtcctgt

SEQ ID NO: 78 - RNA, 22 nt, synthetic construct
  source 1..22: mol_type = other RNA
taaaataaagaggtaataaagg

SEQ ID NO: 79 - RNA, 22 nt, synthetic construct
  source 1..22: mol_type = other RNA
tatcattttattatagaggtaa

SEQ ID NO: 80 - RNA, 22 nt, synthetic construct
  source 1..22: mol_type = other RNA
taggagaaggtcttggctatgc

SEQ ID NO: 81 - RNA, 22 nt, synthetic construct
  source 1..22: mol_type = other RNA
tttcatgaagtcttatcagacg

SEQ ID NO: 82 - RNA, 22 nt, synthetic construct
  source 1..22: mol_type = other RNA
tctatgcagatacagctctgtc

SEQ ID NO: 83 - RNA, 22 nt, synthetic construct
  source 1..22: mol_type = other RNA
taaaataaagaggtaataaagg

SEQ ID NO: 84 - RNA, 22 nt, synthetic construct
  source 1..22: mol_type = other RNA
tgaagtcttatcagacgtgtat

SEQ ID NO: 85 - RNA, 22 nt, synthetic construct
  source 1..22: mol_type = other RNA
tagaggtaataaaataaagagg

SEQ ID NO: 86 - RNA, 22 nt, synthetic construct
  source 1..22: mol_type = other RNA
ttcatgaagtcttatcagacgt

SEQ ID NO: 87 - RNA, 22 nt, synthetic construct
  source 1..22: mol_type = other RNA
gaaggtaggagaaggtcttggc

SEQ ID NO: 88 - RNA, 22 nt, synthetic construct
  source 1..22: mol_type = other RNA
agaggtaataaaataaagaggt

SEQ ID NO: 89 - RNA, 22 nt, synthetic construct
  source 1..22: mol_type = other RNA
taaagaggtaataaaggtataa

SEQ ID NO: 90 - RNA, 22 nt, synthetic construct
  source 1..22: mol_type = other RNA
tattatttcatgaagtcttatc

SEQ ID NO: 91 - RNA, 22 nt, synthetic construct
  source 1..22: mol_type = other RNA
tcttatcagacgtgtatttctc

SEQ ID NO: 92 - RNA, 22 nt, synthetic construct
  source 1..22: mol_type = other RNA
tcattacagaaaaatcagtgga

SEQ ID NO: 93 - RNA, 22 nt, synthetic construct
  source 1..22: mol_type = other RNA
aaaggtataatgtcattacaga

SEQ ID NO: 94 - RNA, 22 nt, synthetic construct
  source 1..22: mol_type = other RNA
ttatcagacgtgtatttctctc

SEQ ID NO: 95 - RNA, 22 nt, synthetic construct
  source 1..22: mol_type = other RNA
tgtcattacagaaaaatcagtg

SEQ ID NO: 96 - RNA, 22 nt, synthetic construct
  source 1..22: mol_type = other RNA
tattagcgaagaacatctggag

SEQ ID NO: 97 - RNA, 22 nt, synthetic construct
  source 1..22: mol_type = other RNA
ttggcatcaatcatcacagggt

SEQ ID NO: 98 - RNA, 22 nt, synthetic construct
  source 1..22: mol_type = other RNA
tgctttatggagtttgacctcg

SEQ ID NO: 99 - RNA, 22 nt, synthetic construct
  source 1..22: mol_type = other RNA
atcaatcatcacagggttggac

SEQ ID NO: 100 - RNA, 22 nt, synthetic construct
  source 1..22: mol_type = other RNA
tcttccacagcattcattcctg

SEQ ID NO: 101 - RNA, 22 nt, synthetic construct
  source 1..22: mol_type = other RNA
tttggcatcaatcatcacaggg

SEQ ID NO: 102 - RNA, 22 nt, synthetic construct
  source 1..22: mol_type = other RNA
agggattcttctcttcttctgg

SEQ ID NO: 103 - RNA, 22 nt, synthetic construct
  source 1..22: mol_type = other RNA
ttcaatcatgggcttgttctgc

SEQ ID NO: 104 - RNA, 22 nt, synthetic construct
  source 1..22: mol_type = other RNA
tcaatcatgggcttgttctgca

SEQ ID NO: 105 - RNA, 22 nt, synthetic construct
  source 1..22: mol_type = other RNA
tagcgaagaacatctggagccg

SEQ ID NO: 106 - RNA, 22 nt, synthetic construct
  source 1..22: mol_type = other RNA
atgaagacaggaaaatgctggt

SEQ ID NO: 107 - RNA, 22 nt, synthetic construct
  source 1..22: mol_type = other RNA
ttattagcgaagaacatctgga

SEQ ID NO: 108 - RNA, 22 nt, synthetic construct
  source 1..22: mol_type = other RNA
ttcattcctgcaatgaccttgg

SEQ ID NO: 109 - RNA, 22 nt, synthetic construct
  source 1..22: mol_type = other RNA
tagggattcttctcttcttctg

SEQ ID NO: 110 - RNA, 22 nt, synthetic construct
  source 1..22: mol_type = other RNA
ttccatcaaagagagacagcac

SEQ ID NO: 111 - RNA, 22 nt, synthetic construct
  source 1..22: mol_type = other RNA
tctttgtagggattcttctctt

SEQ ID NO: 112 - RNA, 22 nt, synthetic construct
  source 1..22: mol_type = other RNA
gtttgtttaactttgtgtcgct

SEQ ID NO: 113 - RNA, 22 nt, synthetic construct
  source 1..22: mol_type = other RNA
tcaataatctccttgaccttgg

SEQ ID NO: 114 - RNA, 22 nt, synthetic construct
  source 1..22: mol_type = other RNA
ttctcattcatgaagacaggaa

SEQ ID NO: 115 - RNA, 22 nt, synthetic construct
  source 1..22: mol_type = other RNA
tttctcattcatgaagacagga

SEQ ID NO: 116 - RNA, 22 nt, synthetic construct
  source 1..22: mol_type = other RNA
tcttctcttcttctggtggctc

SEQ ID NO: 117 - RNA, 22 nt, synthetic construct
  source 1..22: mol_type = other RNA
ttagcgaagaacatctggagcc

SEQ ID NO: 118 - RNA, 22 nt, synthetic construct
  source 1..22: mol_type = other RNA
ttatcattcacagtggatgcca

SEQ ID NO: 119 - RNA, 22 nt, synthetic construct
  source 1..22: mol_type = other RNA
ttgctgaacttggctatcctgc

SEQ ID NO: 120 - RNA, 22 nt, synthetic construct
  source 1..22: mol_type = other RNA
tttaactttgtgtcgctacctc

SEQ ID NO: 121 - RNA, 22 nt, synthetic construct
  source 1..22: mol_type = other RNA
ttgtttaactttgtgtcgctac

SEQ ID NO: 122 - RNA, 22 nt, synthetic construct
  source 1..22: mol_type = other RNA
tttgtttaactttgtgtcgcta

SEQ ID NO: 123 - RNA, 22 nt, synthetic construct
  source 1..22: mol_type = other RNA
ttaacaaaaatatttaatgctg

SEQ ID NO: 124 - RNA, 22 nt, synthetic construct
  source 1..22: mol_type = other RNA
taacaaaaatatttaatgctgc

SEQ ID NO: 125 - RNA, 22 nt, synthetic construct
  source 1..22: mol_type = other RNA
ttaatataatcattctcagcat

SEQ ID NO: 126 - RNA, 22 nt, synthetic construct
  source 1..22: mol_type = other RNA
tctgcaaagtcttctgctggtg

SEQ ID NO: 127 - RNA, 22 nt, synthetic construct
  source 1..22: mol_type = other RNA
tactgtataatacgacttcaca

SEQ ID NO: 128 - RNA, 22 nt, synthetic construct
  source 1..22: mol_type = other RNA
tttagaggaaagtcctgtacat

SEQ ID NO: 129 - RNA, 22 nt, synthetic construct
  source 1..22: mol_type = other RNA
ttttatgttaatatcatctcct

SEQ ID NO: 130 - RNA, 22 nt, synthetic construct
  source 1..22: mol_type = other RNA
taatacgacttcacatcttctg

SEQ ID NO: 131 - RNA, 22 nt, synthetic construct
  source 1..22: mol_type = other RNA
tctgtatactagaatctccctt

SEQ ID NO: 132 - RNA, 22 nt, synthetic construct
  source 1..22: mol_type = other RNA
taattgtagtagatgtactgta

SEQ ID NO: 133 - RNA, 22 nt, synthetic construct
  source 1..22: mol_type = other RNA
atgagaatctcagttgatctgg

SEQ ID NO: 134 - RNA, 22 nt, synthetic construct
  source 1..22: mol_type = other RNA
tttaacacgactgtgatcatat

SEQ ID NO: 135 - RNA, 22 nt, synthetic construct
  source 1..22: mol_type = other RNA
tcgaatttccaagtacagcggc

SEQ ID NO: 136 - RNA, 22 nt, synthetic construct
  source 1..22: mol_type = other RNA
taatataatcattctcagcatt

SEQ ID NO: 137 - RNA, 22 nt, synthetic construct
  source 1..22: mol_type = other RNA
tctgttttctggaaacttggcc

SEQ ID NO: 138 - RNA, 22 nt, synthetic construct
  source 1..22: mol_type = other RNA
ttaaccgattctttctccacaa

SEQ ID NO: 139 - RNA, 22 nt, synthetic construct
  source 1..22: mol_type = other RNA
tcattaaaaacacttaacacat

SEQ ID NO: 140 - RNA, 22 nt, synthetic construct
  source 1..22: mol_type = other RNA
taaaggttctgcaaagtcttct

SEQ ID NO: 141 - RNA, 22 nt, synthetic construct
  source 1..22: mol_type = other RNA
tctgacaagagcttcacactga

SEQ ID NO: 142 - RNA, 22 nt, synthetic construct
  source 1..22: mol_type = other RNA
atacgacttcacatcttctgac

SEQ ID NO: 143 - RNA, 22 nt, synthetic construct
  source 1..22: mol_type = other RNA
ttctatgtcaactaaactggca

SEQ ID NO: 144 - RNA, 22 nt, synthetic construct
  source 1..22: mol_type = other RNA
ttctagacctattctgttccca

SEQ ID NO: 145 - RNA, 22 nt, synthetic construct
  source 1..22: mol_type = other RNA
tacgacttcacatcttctgaca

SEQ ID NO: 146 - RNA, 22 nt, synthetic construct
  source 1..22: mol_type = other RNA
tataatacgacttcacatcttc

SEQ ID NO: 147 - RNA, 22 nt, synthetic construct
  source 1..22: mol_type = other RNA
tatatttacaattatagatctg

SEQ ID NO: 148 - RNA, 22 nt, synthetic construct
  source 1..22: mol_type = other RNA
ttatatttacaattatagatct

SEQ ID NO: 149 - RNA, 22 nt, synthetic construct
  source 1..22: mol_type = other RNA
tttattctctgaaactaggtgt

SEQ ID NO: 150 - RNA, 22 nt, synthetic construct
  source 1..22: mol_type = other RNA
ttgtttgaacaagaaacctgta

SEQ ID NO: 151 - RNA, 22 nt, synthetic construct
  source 1..22: mol_type = other RNA
aagaattcaaatatgaacctgc

SEQ ID NO: 152 - RNA, 22 nt, synthetic construct
  source 1..22: mol_type = other RNA
taacattacaatacttacagta

SEQ ID NO: 153 - RNA, 22 nt, synthetic construct
  source 1..22: mol_type = other RNA
taaaatgaaagtaaacttgttt

SEQ ID NO: 154 - RNA, 22 nt, synthetic construct
  source 1..22: mol_type = other RNA
tcatattgagtcttgacaggtg

SEQ ID NO: 155 - RNA, 22 nt, synthetic construct
  source 1..22: mol_type = other RNA
aagtattgaaaaaaggtatgct

SEQ ID NO: 156 - RNA, 22 nt, synthetic construct
  source 1..22: mol_type = other RNA
tttattaaaaacactcacatag

SEQ ID NO: 157 - RNA, 22 nt, synthetic construct
  source 1..22: mol_type = other RNA
ttatgtgttgaaaaactggtga

SEQ ID NO: 158 - RNA, 22 nt, synthetic construct
  source 1..22: mol_type = other RNA
ttactatacaactctttcactt

SEQ ID NO: 159 - RNA, 22 nt, synthetic construct
  source 1..22: mol_type = other RNA
tttgttgaaaatgaaatctagt

SEQ ID NO: 160 - RNA, 22 nt, synthetic construct
  source 1..22: mol_type = other RNA
tatacaactctttcacttcttt

SEQ ID NO: 161 - RNA, 22 nt, synthetic construct
  source 1..22: mol_type = other RNA
tcacatttacatatcatctcgt

SEQ ID NO: 162 - RNA, 22 nt, synthetic construct
  source 1..22: mol_type = other RNA
tttagatgtaaaactctggcta

SEQ ID NO: 163 - RNA, 22 nt, synthetic construct
  source 1..22: mol_type = other RNA
ttataaaccaaacatctcatga

SEQ ID NO: 164 - RNA, 22 nt, synthetic construct
  source 1..22: mol_type = other RNA
ttaagtaatacaatgttcttgt

SEQ ID NO: 165 - RNA, 22 nt, synthetic construct
  source 1..22: mol_type = other RNA
tttatcaactcagtgcattcta

SEQ ID NO: 166 - RNA, 22 nt, synthetic construct
  source 1..22: mol_type = other RNA
ttaactgacagattcacatgga

SEQ ID NO: 167 - RNA, 22 nt, synthetic construct
  source 1..22: mol_type = other RNA
ttaaaaagacaaaagccagctt

SEQ ID NO: 168 - RNA, 22 nt, synthetic construct
  source 1..22: mol_type = other RNA
ttagatgtaaaactctggctat

SEQ ID NO: 169 - RNA, 22 nt, synthetic construct
  source 1..22: mol_type = other RNA
tatattagcaaattgtagatct

SEQ ID NO: 170 - RNA, 22 nt, synthetic construct
  source 1..22: mol_type = other RNA
taagctaagaaatgacaacgtc

SEQ ID NO: 171 - RNA, 22 nt, synthetic construct
  source 1..22: mol_type = other RNA
taagattacagtaaaaactgca

SEQ ID NO: 172 - RNA, 22 nt, synthetic construct
  source 1..22: mol_type = other RNA
tttaatttactatacaactctt

SEQ ID NO: 173 - RNA, 22 nt, synthetic construct
  source 1..22: mol_type = other RNA
tttgaatactgattttcaccat

SEQ ID NO: 174 - RNA, 22 nt, synthetic construct
  source 1..22: mol_type = other RNA
ttatgtatcaagtacctttgct

SEQ ID NO: 175 - RNA, 22 nt, synthetic construct
  source 1..22: mol_type = other RNA
tcttataaaccaaacatctcat

SEQ ID NO: 176 - RNA, 22 nt, synthetic construct
  source 1..22: mol_type = other RNA
ttccatagggacacggctgctt

SEQ ID NO: 177 - RNA, 22 nt, synthetic construct
  source 1..22: mol_type = other RNA
tctgttagcagagagctggact

SEQ ID NO: 178 - RNA, 22 nt, synthetic construct
  source 1..22: mol_type = other RNA
ttcacaatgaatctgtcgtcat

SEQ ID NO: 179 - RNA, 22 nt, synthetic construct
  source 1..22: mol_type = other RNA
tacagagatacaatgtgtatca

SEQ ID NO: 180 - RNA, 22 nt, synthetic construct
  source 1..22: mol_type = other RNA
tgtatcaacagggtgatcgctt

SEQ ID NO: 181 - RNA, 22 nt, synthetic construct
  source 1..22: mol_type = other RNA
ttaggattgacaaagacgagaa

SEQ ID NO: 182 - RNA, 22 nt, synthetic construct
  source 1..22: mol_type = other RNA
tcagcttgagagacagagccac

SEQ ID NO: 183 - RNA, 22 nt, synthetic construct
  source 1..22: mol_type = other RNA
ttactcatctctaaatccttga

SEQ ID NO: 184 - RNA, 22 nt, synthetic construct
  source 1..22: mol_type = other RNA
tctggtacacaggttttcagga

SEQ ID NO: 185 - RNA, 22 nt, synthetic construct
  source 1..22: mol_type = other RNA
tttagcactgcgatgccttcta

SEQ ID NO: 186 - RNA, 22 nt, synthetic construct
  source 1..22: mol_type = other RNA
ataataaatactgtgagggtga

SEQ ID NO: 187 - RNA, 22 nt, synthetic construct
  source 1..22: mol_type = other RNA
tagtagtacttccgttgcttgg

SEQ ID NO: 188 - RNA, 22 nt, synthetic construct
  source 1..22: mol_type = other RNA
ttcgtagtagtacttccgttgc

SEQ ID NO: 189 - RNA, 22 nt, synthetic construct
  source 1..22: mol_type = other RNA
tctgggaacattatccacttcg

SEQ ID NO: 190 - RNA, 22 nt, synthetic construct
  source 1..22: mol_type = other RNA
tgagaagatggattcagatgtg

SEQ ID NO: 191 - RNA, 22 nt, synthetic construct
  source 1..22: mol_type = other RNA
taataaatactgtgagggtgaa

SEQ ID NO: 192 - RNA, 22 nt, synthetic construct
  source 1..22: mol_type = other RNA
tcttttgtcacatttgtctcat

SEQ ID NO: 193 - RNA, 22 nt, synthetic construct
  source 1..22: mol_type = other RNA
tagattttcaggaattgctgga

SEQ ID NO: 194 - RNA, 22 nt, synthetic construct
  source 1..22: mol_type = other RNA
tttgcatgttgagaagatggat

SEQ ID NO: 195 - RNA, 22 nt, synthetic construct
  source 1..22: mol_type = other RNA
tttcatggcacactggtcgtga

SEQ ID NO: 196 - RNA, 22 nt, synthetic construct
  source 1..22: mol_type = other RNA
tcaaatttaaatcatccatggt

SEQ ID NO: 197 - RNA, 22 nt, synthetic construct
  source 1..22: mol_type = other RNA
atagattttcaggaattgctgg

SEQ ID NO: 198 - RNA, 22 nt, synthetic construct
  source 1..22: mol_type = other RNA
atatgtaccactttactcatct

SEQ ID NO: 199 - RNA, 22 nt, synthetic construct
  source 1..22: mol_type = other RNA
ttgcatgttgagaagatggatt

SEQ ID NO: 200 - RNA, 22 nt, synthetic construct
  source 1..22: mol_type = other RNA
tttaaatcatccatggtcttct

SEQ ID NO: 201 - RNA, 22 nt, synthetic construct
  source 1..22: mol_type = other RNA
tttactcatctctaaatccttg

SEQ ID NO: 202 - RNA, 22 nt, synthetic construct
  source 1..22: mol_type = other RNA
taaatcatccatggtcttctgg

SEQ ID NO: 203 - RNA, 22 nt, synthetic construct
  source 1..22: mol_type = other RNA
tagtcaatctctttcatcatct

SEQ ID NO: 204 - RNA, 22 nt, synthetic construct
  source 1..22: mol_type = other RNA
ttagctttgtcaatggtctcta

SEQ ID NO: 205 - RNA, 22 nt, synthetic construct
  source 1..22: mol_type = other RNA
ttgacgaaggagtacatgagca

SEQ ID NO: 206 - RNA, 22 nt, synthetic construct
  source 1..22: mol_type = other RNA
tgaatctgtcgtcatagcacac

SEQ ID NO: 207 - RNA, 22 nt, synthetic construct
  source 1..22: mol_type = other RNA
tactgacatgaagccacgtgcc

SEQ ID NO: 208 - RNA, 22 nt, synthetic construct
  source 1..22: mol_type = other RNA
tagataagcattataattccta

SEQ ID NO: 209 - RNA, 22 nt, synthetic construct
  source 1..22: mol_type = other RNA
ttaatcagagacttcaggcggt

SEQ ID NO: 210 - RNA, 20 nt, synthetic construct
  source 1..20: mol_type = other RNA
actgcgtgccctgccaagaa

SEQ ID NO: 211 - RNA, 22 nt, synthetic construct
  source 1..22: mol_type = other RNA
ttaagaatcttttcaaacacta

SEQ ID NO: 212 - protein, 466 aa, synthetic construct
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGGGGSGGGGSGGGGSDIELTQSPAIMSASPGEKVTMTCSASSSVSYMHWYQQKSGTSPKRWIYDTSKLASGVPGRFSGSGSGNSYSLTISSVEAEDDATYYCQQWSGYPLTFGAGTKLEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 213 - protein, 813 aa, synthetic construct
DIQMTQSPSSLSASVGDRVTITCRASENIYSYVAWYQQKPGKAPKLLIYNAKSLASGVPSRFSGSGSGTDFTLTISSLQPEDFATYYCQHHYVSPWTFGGGTKLEIKGGGGSGGGGSGGGGSQVQLQESGPGLVKPSETLSLTCTVSGFSLTSYGVSWIRQPAGKGLEWIGVIWEDGSTNYHSALISRVTMSVDTSKNQFSLKLSSVTAADTAVYYCARPHYGSSYVGAMEYWGAGTTVTVSSATTTPAPRPPTPAPTIASQPLSLRPEACFMYVAAAAFVLLFFVGCGVLLSRKRRRQHGQLWFPEGFKVSEASKKKRREPLGEDSVGLKPLKNAMVSKLSQLQTELLAALLESGLSKEALLQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMTCRDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 214 - RNA, 103 nt, synthetic construct
  source 1..103: mol_type = other RNA
ggggccactagggacaggatgttttagagctagaaatagcaagttaaaataaggctagtccgttatcaacttgaaaaagtggcaccgagtcggtgcttttttt

SEQ ID NO: 215 - DNA, 20 bp, Adeno-associated virus 1
  source 1..20: mol_type = other DNA
ggggccactagggacaggat

SEQ ID NO: 216 - RNA, 20 nt, synthetic construct
  source 1..20: mol_type = other RNA
gcacctgaataccacgcctg

SEQ ID NO: 217 - RNA, 20 nt, synthetic construct
  source 1..20: mol_type = other RNA
cgcctgcgatgtagtcgatg

SEQ ID NO: 218 - RNA, 20 nt, synthetic construct
  source 1..20: mol_type = other RNA
caggacgggcgagatgtccc

SEQ ID NO: 219 - RNA, 20 nt, synthetic construct
  source 1..20: mol_type = other RNA
ctgaatctttggagtacctg

SEQ ID NO: 220 - RNA, 20 nt, synthetic construct
  source 1..20: mol_type = other RNA
ggccacggagcgagacatct

SEQ ID NO: 221 - RNA, 20 nt, synthetic construct
  source 1..20: mol_type = other RNA
aagtcaacttcaatgtcgga

SEQ ID NO: 222 - RNA, 20 nt, synthetic construct
  source 1..20: mol_type = other RNA
gcttggaggcctgatcagcg

SEQ ID NO: 223 - RNA, 20 nt, synthetic construct
  source 1..20: mol_type = other RNA
cttatctcttcgcagcgagg

SEQ ID NO: 224 - RNA, 20 nt, synthetic construct
  source 1..20: mol_type = other RNA
cacacattactccaacattg

SEQ ID NO: 225 - RNA, 20 nt, synthetic construct
  source 1..20: mol_type = other RNA
ttccgcaaaatagagcccca

SEQ ID NO: 226 - RNA, 20 nt, synthetic construct
  source 1..20: mol_type = other RNA
tgcacagaactatcgtacca

SEQ ID NO: 227 - RNA, 20 nt, synthetic construct
  source 1..20: mol_type = other RNA
gcaataagactctttaaaga

SEQ ID NO: 228 - RNA, 20 nt, synthetic construct
  source 1..20: mol_type = other RNA
caaagagattacgaatgcct

SEQ ID NO: 229 - RNA, 20 nt, synthetic construct
  source 1..20: mol_type = other RNA
caaggcaccccaggtttcca

SEQ ID NO: 230 - RNA, 20 nt, synthetic construct
  source 1..20: mol_type = other RNA
ttacgaatgccttggaaacc

SEQ ID NO: 231 - RNA, 20 nt, synthetic construct
  source 1..20: mol_type = other RNA
cagagacgcatctgaccctc

SEQ ID NO: 232 - RNA, 20 nt, synthetic construct
  source 1..20: mol_type = other RNA
catgcagttctcacacactg

SEQ ID NO: 233 - RNA, 20 nt, synthetic construct
  source 1..20: mol_type = other RNA
gtgtgagaactgcatggaga

SEQ ID NO: 234 - RNA, 20 nt, synthetic construct
  source 1..20: mol_type = other RNA
tctcatttcaggaaaccact

SEQ ID NO: 235 - RNA, 20 nt, synthetic construct
  source 1..20: mol_type = other RNA
agtcatacaccttaaccaag

SEQ ID NO: 236 - RNA, 20 nt, synthetic construct
  source 1..20: mol_type = other RNA
ttcaaggaaaccagttgagg

SEQ ID NO: 237 - RNA, 20 nt, synthetic construct
  source 1..20: mol_type = other RNA
gagccttgcctggaaatctg

SEQ ID NO: 238 - RNA, 20 nt, synthetic construct
  source 1..20: mol_type = other RNA
aagcgtcaaaagtctgccag

SEQ ID NO: 239 - RNA, 20 nt, synthetic construct
  source 1..20: mol_type = other RNA
cgttccaactcgaagtgcca

SEQ ID NO: 240 - RNA, 20 nt, synthetic construct
  source 1..20: mol_type = other RNA
gagcgactgggacacggtga

SEQ ID NO: 241 - RNA, 20 nt, synthetic construct
  source 1..20: mol_type = other RNA
gctgcgcaagaagggcccta

SEQ ID NO: 242 - RNA, 20 nt, synthetic construct
  source 1..20: mol_type = other RNA
ttgttctggccagcagcccc

SEQ ID NO: 243 - RNA, 20 nt, synthetic construct
  source 1..20: mol_type = other RNA
cttccagagccacatcatcg

SEQ ID NO: 244 - RNA, 20 nt, synthetic construct
  source 1..20: mol_type = other RNA
gggactcaccagagagaggt

SEQ ID NO: 245 - RNA, 20 nt, synthetic construct
  source 1..20: mol_type = other RNA
cggtcgaaatagaagcccta

SEQ ID NO: 246 - RNA, 20 nt, synthetic construct
  source 1..20: mol_type = other RNA
aaaaggatattgtgcaactg

SEQ ID NO: 247 - RNA, 20 nt, synthetic construct
  source 1..20: mol_type = other RNA
tgtgcatatttattacatcg

SEQ ID NO: 248 - RNA, 20 nt, synthetic construct
  source 1..20: mol_type = other RNA
tttgtgaagatcttgaccaa

SEQ ID NO: 249 - RNA, 20 nt, synthetic construct
  source 1..20: mol_type = other RNA
tgtcatgctgaaccgcattg

SEQ ID NO: 250 - RNA, 20 nt, synthetic construct
  source 1..20: mol_type = other RNA
ccactctatgaggatagtca

SEQ ID NO: 251 - RNA, 20 nt, synthetic construct
  source 1..20: mol_type = other RNA
ttgacatagaagaggcacaa

SEQ ID NO: 252 - RNA, 20 nt, synthetic construct
  source 1..20: mol_type = other RNA
gagtactacactcagcagca

SEQ ID NO: 253 - RNA, 20 nt, synthetic construct
  source 1..20: mol_type = other RNA
tcacgcacaagaaacgtcca

SEQ ID NO: 254 - RNA, 20 nt, synthetic construct
  source 1..20: mol_type = other RNA
aggtctcggtgaaaccacct

SEQ ID NO: 255 - RNA, 20 nt, synthetic construct
  source 1..20: mol_type = other RNA
agcattatccaaagagtccg

SEQ ID NO: 256 - RNA, 20 nt, synthetic construct
  source 1..20: mol_type = other RNA
atattaattcttaccagtgg

SEQ ID NO: 257 - RNA, 20 nt, synthetic construct
  source 1..20: mol_type = other RNA
agctttaaatcaaggttcat

SEQ ID NO: 258 - RNA, 20 nt, synthetic construct
  source 1..20: mol_type = other RNA
atcccgagccctaaggtgca

SEQ ID NO: 259 - RNA, 20 nt, synthetic construct
  source 1..20: mol_type = other RNA
ggcagcgcggaggacagcgt

SEQ ID NO: 260 - RNA, 20 nt, synthetic construct
  source 1..20: mol_type = other RNA
ctcagggggctactaccacc

SEQ ID NO: 261 - RNA, 20 nt, synthetic construct
  source 1..20: mol_type = other RNA
gtcaccgacgagaccagaag

SEQ ID NO: 262 - RNA, 20 nt, synthetic construct
  source 1..20: mol_type = other RNA
gtcgtggacttcgtactgct

SEQ ID NO: 263 - RNA, 20 nt, synthetic construct
  source 1..20: mol_type = other RNA
taatttttaggcaagtgtcg

SEQ ID NO: 264 - RNA, 20 nt, synthetic construct
  source 1..20: mol_type = other RNA
ttagctgttagacttgaata

SEQ ID NO: 265 - RNA, 20 nt, synthetic construct
  source 1..20: mol_type = other RNA
cgagagccgtcaacttgcgt

SEQ ID NO: 266 - RNA, 20 nt, synthetic construct
  source 1..20: mol_type = other RNA
cggcttcaactgcaaaggtg

SEQ ID NO: 267 - RNA, 20 nt, synthetic construct
  source 1..20: mol_type = other RNA
tatgaaaaagcagagcgact

SEQ ID NO: 268 - RNA, 20 nt, synthetic construct
  source 1..20: mol_type = other RNA
tctggcgggcgagctcacgc

SEQ ID NO: 269 - RNA, 20 nt, synthetic construct
  source 1..20: mol_type = other RNA
ctcacgctggttaccgccta

SEQ ID NO: 270 - RNA, 20 nt, synthetic construct
  source 1..20: mol_type = other RNA
aaagattacgaacttccctg

SEQ ID NO: 271 - RNA, 20 nt, synthetic construct
  source 1..20: mol_type = other RNA
gttaaaaacagacatgccta

SEQ ID NO: 272 - RNA, 20 nt, synthetic construct
  source 1..20: mol_type = other RNA
atgcctaaggaggttgtacc

SEQ ID NO: 273 - RNA, 20 nt, synthetic construct
  source 1..20: mol_type = other RNA
ctccaggtatcccatcgaaa

SEQ ID NO: 274 - RNA, 20 nt, synthetic construct
  source 1..20: mol_type = other RNA
caccaaatacgatagatcag

SEQ ID NO: 275 - RNA, 20 nt, synthetic construct
  source 1..20: mol_type = other RNA
tggcggcgtgaatggcaaga

SEQ ID NO: 276 - RNA, 20 nt, synthetic construct
  source 1..20: mol_type = other RNA
taggatggtagcacacaacc

SEQ ID NO: 277 - RNA, 20 nt, synthetic construct
  source 1..20: mol_type = other RNA
cagcagcagagccccgacgg

SEQ ID NO: 278 - RNA, 20 nt, synthetic construct
  source 1..20: mol_type = other RNA
cggcgtgcgaacggaatgtg

SEQ ID NO: 279 - RNA, 20 nt, synthetic construct
  source 1..20: mol_type = other RNA
tatagacgctgcccgacgtc

SEQ ID NO: 280 - RNA, 20 nt, synthetic construct
  source 1..20: mol_type = other RNA
tccaaagaagggtactgtgg

SEQ ID NO: 281 - RNA, 20 nt, synthetic construct
  source 1..20: mol_type = other RNA
acagtacccttctttggaat

SEQ ID NO: 282 - RNA, 20 nt, synthetic construct
  source 1..20: mol_type = other RNA
gcgacgggcgcatctacgtg

SEQ ID NO: 283 - RNA, 20 nt, synthetic construct
  source 1..20: mol_type = other RNA
cccgacctccataagtcctg

SEQ ID NO: 284 - RNA, 20 nt, synthetic construct
  source 1..20: mol_type = other RNA
ggggtcctcgaagcgcacga

SEQ ID NO: 285 - RNA, 20 nt, synthetic construct
  source 1..20: mol_type = other RNA
tgctctgtttagaagatgac

SEQ ID NO: 286 - RNA, 20 nt, synthetic construct
  source 1..20: mol_type = other RNA
atattcttttctagttaaag

SEQ ID NO: 287 - RNA, 20 nt, synthetic construct
  source 1..20: mol_type = other RNA
cctgtaaagaaacaaaagac

SEQ ID NO: 288 - RNA, 20 nt, synthetic construct
  source 1..20: mol_type = other RNA
tggagaaagacgtaacttcg

SEQ ID NO: 289 - RNA, 20 nt, synthetic construct
  source 1..20: mol_type = other RNA
tctgccctgaggtatgcgat

SEQ ID NO: 290 - RNA, 20 nt, synthetic construct
  source 1..20: mol_type = other RNA
attccgcttggtgaaaacga

SEQ ID NO: 291 - RNA, 20 nt, synthetic construct
  source 1..20: mol_type = other RNA
caggcacaatagaaacaacg

SEQ ID NO: 292 - RNA, 20 nt, synthetic construct
  source 1..20: mol_type = other RNA
ccatttgtaatgctgacttg

SEQ ID NO: 293 - RNA, 20 nt, synthetic construct
  source 1..20: mol_type = other RNA
ctgggtcacttgtgccgtgg

SEQ ID NO: 294 - RNA, 20 nt, synthetic construct
  source 1..20: mol_type = other RNA
gtcagggttctggatatctg

SEQ ID NO: 295 - RNA, 20 nt, synthetic construct
  source 1..20: mol_type = other RNA
tggatttagagtctctcagc

SEQ ID NO: 296 - RNA, 20 nt, synthetic construct
  source 1..20: mol_type = other RNA
ctgcggctgtggtccagctg

SEQ ID NO: 297 - RNA, 20 nt, synthetic construct
  source 1..20: mol_type = other RNA
acaaaactgtgctagacatg

SEQ ID NO: 298 - RNA, 20 nt, synthetic construct
  source 1..20: mol_type = other RNA
ttcttccccagcccaggtaa

SEQ ID NO: 299 - RNA, 20 nt, synthetic construct
  source 1..20: mol_type = other RNA
cgtcatgagcagattaaacc

SEQ ID NO: 300 - RNA, 20 nt, synthetic construct
  source 1..20: mol_type = other RNA
gagagcgcctgcgacccgag

SEQ ID NO: 301 - RNA, 20 nt, synthetic construct
  source 1..20: mol_type = other RNA
ccagcgggtgaagtacacca

SEQ ID NO: 302 - RNA, 20 nt, synthetic construct
  source 1..20: mol_type = other RNA
ggagcgcttttcgccgccag

SEQ ID NO: 303 - RNA, 20 nt, synthetic construct
  source 1..20: mol_type = other RNA
tgaggcctggaccttatgca

SEQ ID NO: 304 - RNA, 20 nt, synthetic construct
  source 1..20: mol_type = other RNA
cctggtggagtgaaccatga

SEQ ID NO: 305 - RNA, 20 nt, synthetic construct
  source 1..20: mol_type = other RNA
caagcacttaggttcccctg

SEQ ID NO: 306 - RNA, 20 nt, synthetic construct
  source 1..20: mol_type = other RNA
ggtctccctacaattcagcg

SEQ ID NO: 307 - RNA, 20 nt, synthetic construct
  source 1..20: mol_type = other RNA
cacagcgcgtgactgcaatg

SEQ ID NO: 308 - RNA, 20 nt, synthetic construct
  source 1..20: mol_type = other RNA
tctggggcaccaattctagg

SEQ ID NO: 309 - RNA, 20 nt, synthetic construct
  source 1..20: mol_type = other RNA
gagccatgcttggcttacga

SEQ ID NO: 310 - RNA, 20 nt, synthetic construct
  source 1..20: mol_type = other RNA
gtacaagtacttatctcatg

SEQ ID NO: 311 - RNA, 20 nt, synthetic construct
  source 1..20: mol_type = other RNA
gagataacaacataacaaca

SEQ ID NO: 312 - RNA, 20 nt, synthetic construct
  source 1..20: mol_type = other RNA
catattccatagtctttggg

SEQ ID NO: 313 - RNA, 20 nt, synthetic construct
  source 1..20: mol_type = other RNA
ctgccccttagcaacttagg

SEQ ID NO: 314 - RNA, 20 nt, synthetic construct
  source 1..20: mol_type = other RNA
tgtttaaaaatatgttgaca

SEQ ID NO: 315 - RNA, 20 nt, synthetic construct
  source 1..20: mol_type = other RNA
ccaggaatggaaactcacgc

SEQ ID NO: 316 - RNA, 20 nt, synthetic construct
  source 1..20: mol_type = other RNA
gaggccgctgaattaacccg

SEQ ID NO: 317 - RNA, 20 nt, synthetic construct
  source 1..20: mol_type = other RNA
atacacgcacacttgcagaa

SEQ ID NO: 318 - RNA, 20 nt, synthetic construct
  source 1..20: mol_type = other RNA
gagcagacagaaacccaggg

SEQ ID NO: 319 - RNA, 20 nt, synthetic construct
  source 1..20: mol_type = other RNA
tgagtctccaaacagaacag

SEQ ID NO: 320 - RNA, 20 nt, synthetic construct
  source 1..20: mol_type = other RNA
taatatcactgacttcacgg

SEQ ID NO: 321 - RNA, 20 nt, synthetic construct
  source 1..20: mol_type = other RNA
tacacacaatgtaagcagca

SEQ ID NO: 322 - RNA, 20 nt, synthetic construct
  source 1..20: mol_type = other RNA
gggagctcaattcgaaacca

SEQ ID NO: 323 - RNA, 20 nt, synthetic construct
  source 1..20: mol_type = other RNA
ttggacaggtgagacagtcg

SEQ ID NO: 324 - RNA, 20 nt, synthetic construct
  source 1..20: mol_type = other RNA
aagctcactcagatagtgtg

SEQ ID NO: 325 - RNA, 20 nt, synthetic construct
  source 1..20: mol_type = other RNA
caggagaaccaccttacacg

SEQ ID NO: 326 - RNA, 20 nt, synthetic construct
  source 1..20: mol_type = other RNA
ggacagaccctgattcacaa

SEQ ID NO: 327 - RNA, 20 nt, synthetic construct
  source 1..20: mol_type = other RNA
acatggcagtctatgaacag

SEQ ID NO: 328 - RNA, 20 nt, synthetic construct
  source 1..20: mol_type = other RNA
cctatagagagtactacttg

SEQ ID NO: 329 - RNA, 20 nt, synthetic construct
  source 1..20: mol_type = other RNA
ccaaccgggtcttcattacg

SEQ ID NO: 330 - RNA, 20 nt, synthetic construct
  source 1..20: mol_type = other RNA
tcaagcgtagagttccgagt

SEQ ID NO: 331 - RNA, 20 nt, synthetic construct
  source 1..20: mol_type = other RNA
tcatgcaattatggacccag

SEQ ID NO: 332 - RNA, 20 nt, synthetic construct
  source 1..20: mol_type = other RNA
cgggaaagtgactggccatg

SEQ ID NO: 333 - RNA, 20 nt, synthetic construct
  source 1..20: mol_type = other RNA
tgagattgaaatcaaatcgg

SEQ ID NO: 334 - RNA, 20 nt, synthetic construct
  source 1..20: mol_type = other RNA
tatgcaatattcatcacgcg

SEQ ID NO: 335 - RNA, 20 nt, synthetic construct
  source 1..20: mol_type = other RNA
aatgtgttaaatcaaatgca

(19) United States
(12) Patent Application Publication
(10) Pub. No.: US 2025/0222103 A1
(43) Pub. Date: Jul. 10, 2025

(54) IMMUNE CELLS WITH COMBINATION GENE PERTURBATIONS

(71) Applicant: Arsenal Biosciences, Inc., South San Francisco, CA (US)

(72) Inventors: Adam Litterman, San Francisco, CA (US); Brenal Singh, San Francisco, CA (US); John Gagnon, San Francisco, CA (US); David Detomaso, Alameda, CA (US); Ashley Cass, San Francisco, CA (US); Levi Gray-Rupp, Philadelphia, PA (US); Samuel Williams, Burlingame, CA (US)

(21) Appl. No.: 19/080,727

(22) Filed: Mar. 14, 2025

Related U.S. Application Data
(63) Continuation of application No. PCT/US23/74352, filed on Sep. 15, 2023.
(60) Provisional application No. 63/376,041, filed on Sep. 16, 2022.

(51) Int. Cl. A61K 40/31; A61K 40/11; A61P 35/00; C12N 9/22; C12N 15/11; C12N 15/113

(52) U.S. Cl. CPC A61K 40/31; A61K 40/11; A61P 35/00; C12N 9/22; C12N 15/11; C12N 15/113; C12N 2310/14; C12N 2310/20; C12N 2310/531

(57) ABSTRACT

Provided herein are recombinant nucleic acids that reduce expression of CD5, CBLB, CISH, DGKA, DGKZ, DNMT3A, MAP4K1, NR4A1, PTPN2, TET2, or ZC3H12A and cells comprising such recombinant nucleic acids. Also provided are methods of making and using such cells.

### CROSS REFERENCE TO RELATED APPLICATIONS

This application is continuation of International Application No. PCT/US2023/074352 filed on Sep. 15, 2023, which claims the benefit of and priority to U.S. Provisional Application No. 63/376,041, filed on Sep. 16, 2022, the disclosure of which is hereby incorporated by reference in its entirety for all purposes.

### SEQUENCE LISTING

The instant application contains a Sequence Listing which has been submitted electronically and is hereby incorporated by reference in its entirety. Said XML copy, created on Dec. 8, 2023, is named ANB-216USWOC1_SL.xml, and is 352,953 bytes in size.

### BACKGROUND

Cancer is a disease characterized by uncontrollable growth of cells. Many approaches to treating cancer have been tried, including drugs and radiation therapies. Recent cancer treatments have sought to use the body's own immune cells to attack cancer cells. One promising approach uses T cells that are taken from a patient and genetically engineered to produce chimeric antigen receptors, or CARs, receptor proteins that give the T cells a new ability to target a specific protein. The receptors are chimeric because they combine antigen-binding and T-cell activating functions into a single receptor.

Immunotherapy using CAR-T cells is promising because the modified T cells have the potential to recognize cancer cells in order to more effectively target and destroy them. After the T cells are engineered with the CARs, the resulting CAR-T cells are introduced into patients to attack tumor cells. Once CAR-T cells are infused into a patient, they come in contact with their targeted antigen on a cell. The CAR-T cells bind to the antigen and become activated. Upon antigen engagement, CAR T cells can proliferate exponentially, initiate antitumor cytokine production, and target tumor cell killing.

However, there remain some limitations to CAR T cell-based immunotherapy. In particular, CAR-T cells can lack peripheral survival, can have reduced expansion and effector function, are susceptible to suppression and exhaustion, and may not result in memory T cell persistence. Thus, additional therapies targeting T cell intrinsic pathways are needed to address these roadblocks for CAR-T therapy.

Individual perturbation of the genes DNMT3A, TET2, CD5, DGKA, DGKZ, MAP4K1, CBLB, FAS, PTPN2, NR4A1, ZC3H12A, and CISH have been reported to enhance T cell expansion or T cell mediated target cell killing, features which have been reported to be predictive of T cell potency in CAR T and other therapeutic T cell applications. While the impact of individual gene perturbations has been established, it is unknown if the coincident perturbation of any two of these genes would provide a more significant impact on T cell expansion or T cell mediated target cell killing than by the individual component genes.

### SUMMARY

In one aspect, provided herein are one or more recombinant nucleic acids comprising at least one sequence as set forth in SEQ ID NOs: 12-207.

In some embodiments, the nucleic acid is a guide RNA.

In some embodiments, further comprising a protein comprising a nuclease domain, wherein the nucleic acid and protein form a ribonucleoprotein (RNP) complex.

In some embodiments, the nuclease domain comprises a CRISPR-associated endonuclease (Cas), optionally a Cas9 nuclease.

In some embodiments, the ribonucleoprotein (RNP) complex reduces expression of one or more of CD5, CBLB, CISH, DGKA, DGKZ, DNMT3A, MAP4K1, NR4A1, PTPN2, TET2, and/or ZC3H12A in a cell by at least 50%, 55%, 60%, 65%, 75%, 80%, 85%, 90%, 95%, or 99% as compared to a control cell that does not comprise the RNP complex.

In some embodiments, comprising a first nucleic acid and a second nucleic acid, wherein the first and second nucleic acids are distinct.

In some embodiments, comprising a sequence set forth in SEQ ID NO: 12 and a sequence set forth in SEQ ID NO: 13.

In some embodiments, comprising a sequence set forth in SEQ ID NO: 21 and a sequence set forth in SEQ ID NO: 13.

In some embodiments, comprising a sequence set forth in SEQ ID NO: 21 and a sequence set forth in SEQ ID NO: 12.

In some embodiments, comprising a sequence set forth in SEQ ID NO: 12 and a sequence set forth in SEQ ID NO: 14.

In some embodiments, comprising a sequence set forth in SEQ ID NO: 13 and a sequence set forth in SEQ ID NO: 20.

In some embodiments, comprising a sequence set forth in SEQ ID NO: 21 and a sequence set forth in SEQ ID NO: 20.

In some embodiments, comprising a sequence set forth in SEQ ID NO: 20 and a sequence set forth in SEQ ID NO: 22.

In some embodiments, comprising a sequence set forth in SEQ ID NO: 17 and a sequence set forth in SEQ ID NO: 13.

In some embodiments, comprising a sequence set forth in SEQ ID NO: 20 and a sequence set forth in SEQ ID NO: 14.

In some embodiments, comprising a sequence set forth in SEQ ID NO: 21 and a sequence set forth in SEQ ID NO: 22.

In some embodiments, the nucleic acid is a short hairpin RNA (shRNA). In some embodiments, the shRNA reduces the expression of one or more of CD5, CBLB, CISH, DGKA, DNMT3A, PTPN2, TET2, and/or ZC3H12A in a cell by at least 50%, 55%, 60%, 65%, 75%, 80%, 85%, 90%, 95%, or 99% as compared to a control cell that does not comprise the shRNA. In some embodiments, the first and second nucleic acids are distinct.

In one aspect, provided herein are one or more recombinant nucleic acids comprising a nucleic acid sequence at least 15 nucleotides in length complementary to an mRNA encoding human CD5 comprising the sequence set forth in SEQ ID NO: 1.

In one aspect, provided herein are one or more recombinant nucleic acids comprising a nucleic acid sequence at least 15 nucleotides in length complementary to an mRNA encoding human CBLB comprising the sequence set forth in SEQ ID NO: 2.

In one aspect, provided herein are one or more recombinant nucleic acids comprising a nucleic acid sequence at least 15 nucleotides in length complementary to an mRNA encoding human CISH comprising the sequence set forth in SEQ ID NO: 3.

In one aspect, provided herein are one or more recombinant nucleic acids comprising a nucleic acid sequence at least 15 nucleotides in length complementary to an mRNA encoding DGKA comprising the sequence set forth in SEQ ID NO: 4.

In one aspect, provided herein are one or more recombinant nucleic acids comprising a nucleic acid sequence at least 15 nucleotides in length complementary to an mRNA encoding DGKZ comprising the sequence set forth in SEQ ID NO: 5.

In one aspect, provided herein are one or more recombinant nucleic acids comprising a nucleic acid sequence at least 15 nucleotides in length complementary to an mRNA encoding DNMT3A comprising the sequence set forth in SEQ ID NO: 6.

In one aspect, provided herein are one or more recombinant nucleic acids comprising a nucleic acid sequence at least 15 nucleotides in length complementary to an mRNA encoding MAP4K1 comprising the sequence set forth in SEQ ID NO: 7.

In one aspect, provided herein are one or more recombinant nucleic acids comprising a nucleic acid sequence at least 15 nucleotides in length complementary to an mRNA encoding NR4A1 comprising the sequence set forth in SEQ ID NO: 8.

In one aspect, provided herein are one or more recombinant nucleic acids comprising a nucleic acid sequence at least 15 nucleotides in length complementary to an mRNA encoding TET2 comprising the sequence set forth in SEQ ID NO: 10.

In one aspect, provided herein are one or more recombinant nucleic acids comprising a nucleic acid sequence at least 15 nucleotides in length complementary to an mRNA encoding PTPN2 comprising the sequence set forth in SEQ ID NO: 9.

In one aspect, provided herein are one or more recombinant nucleic acids comprising a nucleic acid sequence at least 15 nucleotides in length complementary to an mRNA encoding ZC3H12A comprising the sequence set forth in SEQ ID NO: 11.

In one aspect, provided herein are one or more recombinant nucleic acids comprising at least two or more nucleic acids selected from the group consisting of: (1) a nucleic acid sequence at least 15 nucleotides in length complementary to an mRNA encoding human CD5 comprising the sequence set forth in SEQ ID NO: 1; (2) a nucleic acid sequence at least 15 nucleotides in length complementary to an mRNA encoding human CBLB comprising the sequence set forth in SEQ ID NO: 2; (3) a nucleic acid sequence at least 15 nucleotides in length complementary to an mRNA encoding human CISH comprising the sequence set forth in SEQ ID NO: 3; (4) a nucleic acid sequence at least 15 nucleotides in length complementary to an mRNA encoding DGKA comprising the sequence set forth in SEQ ID NO: 4; (5) a nucleic acid sequence at least 15 nucleotides in length complementary to an mRNA encoding DGKZ comprising the sequence set forth in SEQ ID NO: 5; (6) a nucleic acid sequence at least 15 nucleotides in length complementary to an mRNA encoding DNMT3A comprising the sequence set forth in SEQ ID NO: 6; (7) a nucleic acid sequence at least 15 nucleotides in length complementary to an mRNA encoding MAP4K1 comprising the sequence set forth in SEQ ID NO: 7; (9) a nucleic acid sequence at least 15 nucleotides in length complementary to an mRNA encoding TET2 comprising the sequence set forth in SEQ ID NO: 10; (10) a nucleic acid sequence at least 15 nucleotides in length complementary to an mRNA encoding human Protein Tyrosine Phosphatase Non-Receptor Type 2 (PTPN2) comprising the sequence set forth in SEQ ID NO: 9; (11) a nucleic acid sequence at least 15 nucleotides in length complementary to an mRNA encoding NR4A1 comprising the sequence set forth in SEQ ID NO: 8; or (12) a nucleic acid sequence at least 15 nucleotides in length complementary to an mRNA encoding ZC3H12A comprising the sequence set forth in SEQ ID NO: 11.

In some embodiments, the nucleic acid sequence is at least 16, 17, 18, 19, 20, 21, or 22 nucleotides in length.

In some embodiments, the nucleic acid is a short hairpin RNA (shRNA), a small interfering RNA (siRNA), a double stranded RNA (dsRNA), or an antisense oligonucleotide.

In some embodiments, the nucleic acid is an shRNA.

In some embodiments, the nucleic acid reduces expression of CD5, CBLB, CISH, DGKA, DGKZ, DNMT3A, MAP4K1, NR4A1, PTPN2, TET2, and/or ZC3H12A in a cell by at least 50%, 55%, 60%, 65%, 75%, 80%, 85%, 90%, 95%, or 99% as compared to a control cell that does not comprise the nucleic acid.

In some embodiments, the nucleic acid sequence is an shRNA complementary to the mRNA encoding CD5 and comprises a sequence set forth in any one of SEQ ID NOs: 47-72. In some embodiments, the nucleic acid sequence is an shRNA complementary to the mRNA encoding CBLB and comprises a sequence set forth in any one of SEQ ID NOs: 23-46. In some embodiments, the nucleic acid sequence is an shRNA complementary to the mRNA encoding CISH and comprises a sequence set forth in any one of SEQ ID NOs: 73-95. In some embodiments, the nucleic acid sequence is an shRNA complementary to the mRNA encoding DGKA and comprises a sequence set forth in any one of SEQ ID NOs: 181-204. In some embodiments, the nucleic acid sequence is an shRNA complementary to the mRNA encoding DNMT3A and comprises a sequence set forth in any one of SEQ ID NOs: 96-122. In some embodiments, the nucleic acid sequence is an shRNA complementary to the mRNA encoding TET2 and comprises a sequence set forth in any one of SEQ ID NOs: 147-175. In some embodiments, the nucleic acid sequence is an shRNA complementary to the mRNA encoding PTPN2 and comprises a sequence set forth in any one of SEQ ID NOs: 123-146. In some embodiments, the nucleic acid sequence is an shRNA complementary to the mRNA encoding ZC3H12A and comprises a sequence set forth in any one of SEQ ID NOs: 176-180 and 205-207.

In some embodiments, comprising at least a nucleic acid sequence at least 15 nucleotides in length complementary to an mRNA encoding human CD5 comprising the sequence set forth in SEQ ID NO: 1 and a nucleic acid sequence at least 15 nucleotides in length complementary to an mRNA encoding human CBLB comprising the sequence set forth in SEQ ID NO: 2.

In some embodiments, comprising at least a nucleic acid sequence at least 15 nucleotides in length complementary to an mRNA encoding TET2 comprising the sequence set forth in SEQ ID NO: 9 and a nucleic acid sequence at least 15 nucleotides in length complementary to an mRNA encoding human CBLB comprising the sequence set forth in SEQ ID NO: 2.

In some embodiments, comprising at least a nucleic acid sequence at least 15 nucleotides in length complementary to an mRNA encoding TET2 comprising the sequence set forth in SEQ ID NO: 9 and a nucleic acid sequence at least 15 nucleotides in length complementary to an mRNA encoding human CD5 comprising the sequence set forth in SEQ ID NO: 1.

In some embodiments, comprising at least a nucleic acid sequence at least 15 nucleotides in length complementary to an mRNA encoding human CD5 comprising the sequence set forth in SEQ ID NO: 1 and a nucleic acid sequence at least 15 nucleotides in length complementary to an mRNA encoding human CISH comprising the sequence set forth in SEQ ID NO: 3.

In some embodiments, the recombinant nucleic acid further comprises one or more of: a nucleotide sequence encoding a priming receptor comprising a first extracellular antigen-binding domain that specifically binds to a first antigen; a nucleotide sequence encoding a chimeric antigen receptor (CAR) comprising a second extracellular antigen-binding domain that specifically binds to a second antigen; or a nucleotide sequence encoding a T cell receptor (TCR).

In some embodiments, the first antigen and the second antigen are distinct.

In some embodiments, the recombinant nucleic acid comprises, in a 5′ to 3′ direction the TCR; the nucleic acid disclosed herein.

In some embodiments, the recombinant nucleic acid comprises, in a 5′ to 3′ direction the nucleic acid disclosed herein; the TCR.

In some embodiments, the recombinant nucleic acid comprises, in a 5′ to 3′ direction the CAR; the nucleic disclosed herein; and the priming receptor.

In some embodiments, the nucleic acid comprises, in a 5′ to 3′ direction the priming receptor; the nucleic acid disclosed herein; and the CAR.

In some embodiments, the recombinant nucleic acid further comprises a 5′ homology directed repair arm and/or a 3′ homology directed repair arm complementary to an insertion site in a host cell chromosome.

In some embodiments, the recombinant nucleic acid comprises the 5′ homology directed repair arm and the 3′ homology directed repair arm.

In some embodiments, the recombinant nucleic acid is incorporated into an expression cassette or an expression vector.

In some embodiments, the expression cassette or the expression vector further comprises a constitutive promoter upstream of the recombinant nucleic acid.

In some embodiments, comprising a first nucleic acid and a second nucleic acid, wherein the first nucleic acid and the second nucleic acid are encoded on a single nucleic acid.

In some embodiments, the first nucleic acid comprises the 5′ homology directed repair arm and the second nucleic acid comprises the 3′ homology directed repair arm.

In some embodiments, the first nucleic acid and the second nucleic acid are encoded on different nucleic acids.

In some embodiments, the first nucleic acid and the second nucleic acid are incorporated into a single expression cassette or a single expression vector.

In some embodiments, the expression cassette or the expression vector further comprises a constitutive promoter upstream of the first nucleic acid and/or upstream of the second nucleic acid.

In some embodiments, the expression vector is a non-viral vector.

In one aspect, provided herein are expression vectors comprising the one or more recombinant nucleic acid(s) disclosed herein.

In some embodiments, the expression vector is a non-viral vector.

In some embodiments, the 5′ and 3′ ends of the recombinant nucleic acid(s) comprise one or more nucleotide sequences that are homologous to genomic sequences flanking an insertion site in a genome of a primary cell.

In some embodiments, the insertion site is located at a T Cell Receptor Alpha Constant (TRAC) locus or a genomic safe harbor (GSH) locus.

In some embodiments, the GSH locus is the GS94 locus.

In one aspect, provided herein are immune cells comprising at least one or more nucleic acids selected from the group consisting of: (1) a nucleic acid sequence at least 15 nucleotides in length complementary to an mRNA encoding human CD5 comprising the sequence set forth in SEQ ID NO: 1; (2) a nucleic acid sequence at least 15 nucleotides in length complementary to an mRNA encoding human CBLB comprising the sequence set forth in SEQ ID NO: 2; (3) a nucleic acid sequence at least 15 nucleotides in length complementary to an mRNA encoding human CISH comprising the sequence set forth in SEQ ID NO: 3; (4) a nucleic acid sequence at least 15 nucleotides in length complementary to an mRNA encoding DGKA comprising the sequence set forth in SEQ ID NO: 4; (5) a nucleic acid sequence at least 15 nucleotides in length complementary to an mRNA encoding DGKZ comprising the sequence set forth in SEQ ID NO: 5; (6) a nucleic acid sequence at least 15 nucleotides in length complementary to an mRNA encoding DNMT3A comprising the sequence set forth in SEQ ID NO: 6; (7) a nucleic acid sequence at least 15 nucleotides in length complementary to an mRNA encoding MAP4K1 comprising the sequence set forth in SEQ ID NO: 7; (9) a nucleic acid sequence at least 15 nucleotides in length complementary to an mRNA encoding TET2 comprising the sequence set forth in SEQ ID NO: 10; (10) a nucleic acid sequence at least 15 nucleotides in length complementary to an mRNA encoding human Protein Tyrosine Phosphatase Non-Receptor Type 2 (PTPN2) comprising the sequence set forth in SEQ ID NO: 9; (11) a nucleic acid sequence at least 15 nucleotides in length complementary to an mRNA encoding NR4A1 comprising the sequence set forth in SEQ ID NO: 8; or (12) a nucleic acid sequence at least 15 nucleotides in length complementary to an mRNA encoding ZC3H12A comprising the sequence set forth in SEQ ID NO: 11.

In some embodiments, the one or more nucleic acids are an shRNA, an siRNA, a dsRNA, or an antisense oligonucleotide.

In some embodiments, the one or more nucleic acids are shRNA.

In some embodiments, the shRNA is complementary to the mRNA encoding CD5 and comprises a sequence set forth in any one of SEQ ID NOs: 47-72. In some embodiments, the shRNA is complementary to the mRNA encoding CBLB and comprises a sequence set forth in any one of SEQ ID NOs: 23-46. In some embodiments, the shRNA is complementary to the mRNA encoding CISH and comprises a sequence set forth in any one of SEQ ID NOs: 73-95. In some embodiments, the shRNA is complementary to the mRNA encoding DGKA and comprises a sequence set forth in any one of SEQ ID NOs: 181-204. In some embodiments, the shRNA is complementary to the mRNA encoding DNMT3A and comprises a sequence set forth in any one of SEQ ID NOs: 96-122. In some embodiments, the shRNA is complementary to the mRNA encoding TET2 and comprises a sequence set forth in any one of SEQ ID NOs: 147-175. In some embodiments, the shRNA is complementary to the mRNA encoding PTPN2 and comprises a sequence set forth in any one of SEQ ID NOs: 123-146. In some embodiments, the shRNA is complementary to the mRNA encoding ZC3H12A and comprises a sequence set forth in any one of SEQ ID NOs: 176-180 and 205-207.

In some embodiments, the cell further comprises a deletion of at least a first target gene selected from the group consisting of CD5, CBLB, CISH, DGKA, DGKZ, DNMT3A, MAP4K1, NR4A1, PTPN2, TET2, and ZC3H12A.

In some embodiments, further comprising deletion of at least a second target gene selected from the group consisting of CD5, CBLB, CISH, DGKA, DGKZ, DNMT3A, MAP4K1, NR4A1, PTPN2, TET2, and ZC3H12A, and wherein the first target gene and the second target gene are distinct.

In some embodiments, the at least first or second target gene(s) are deleted via CRISPR-Cas9 gene editing.

In some embodiments, expression of at least one or more target gene selected from the group consisting of CD5, CBLB, CISH, DGKA, DGKZ, DNMT3A, MAP4K1, NR4A1, PTPN2, TET2, and ZC3H12A in the immune cell is reduced by at least 50%, 55%, 60%, 65%, 75%, 80%, 85%, 90%, 95%, or 99% as compared to a control cell that does not comprise the nucleic acids or does comprise the target gene.

In one aspect, provided herein are immune cells comprising a deletion of at least a first target gene selected from the group consisting of CD5, CBLB, CISH, DGKA, DGKZ, DNMT3A, MAP4K1, NR4A1, PTPN2, TET2, and ZC3H12A.

In some embodiments, further comprising deletion of at least a second target gene selected from the group consisting of CD5, CBLB, CISH, DGKA, DGKZ, DNMT3A, MAP4K1, NR4A1, PTPN2, TET2, and ZC3H12A, and wherein the first target gene and the second target gene are distinct.

In some embodiments, the at least first or second target gene(s) are deleted via CRISPR-Cas9 gene editing.

In some embodiments, expression of the at least first or at least second target gene in the immune cell is reduced by at least 50%, 55%, 60%, 65%, 75%, 80%, 85%, 90%, 95%, or 99% as compared to a control cell that does not comprise the deletion of the at least first or at least second target gene.

In one aspect, provided herein are immune cells comprising a first guide RNA, wherein the first guide RNA comprises a sequence set forth in SEQ ID NOs: 12-22.

In some embodiments, further comprising a second guide RNA comprising a sequence set forth in SEQ ID NOs: 12-22.

In some embodiments, further comprising a protein comprising a nuclease domain, wherein the nucleic acid and protein form a ribonucleoprotein (RNP) complex.

In some embodiments, the nuclease domain comprises a CRISPR-associated endonuclease (Cas), optionally a Cas9 nuclease.

In one aspect, provided herein are immune cells comprising one or more nucleic acids comprising a first shRNA and a second shRNA, wherein the first shRNA and second shRNA each comprise a sequence set forth in any one of SEQ ID NOs: 23-207.

In some embodiments, the first or second nucleic acid reduces expression of CD5, CBLB, CISH, DGKA, DGKZ, DNMT3A, MAP4K1, NR4A1, PTPN2, TET2, or ZC3H12A in the immune cell by at least 50%, 55%, 60%, 65%, 75%, 80%, 85%, 90%, 95%, or 99% as compared to a control cell that does not comprise the first nucleic acid.

In some embodiments, expression of CD5, CBLB, CISH, DGKA, DGKZ, DNMT3A, MAP4K1, NR4A1, PTPN2, TET2, and/or ZC3H12A in the immune cell is reduced by at least 50%, 55%, 60%, 65%, 75%, 80%, 85%, 90%, 95%, or 99% as compared to a control cell that does not comprise the first or second nucleic acid.

In some embodiments, expression of CD5, CBLB, CISH, DGKA, DGKZ, DNMT3A, MAP4K1, NR4A1, PTPN2, TET2, and/or ZC3H12A is determined by a nucleic acid assay or a protein assay.

In some embodiments, the nucleic acid assay comprises at least one of polymerase chain reaction (PCR), quantitative PCR (qPCR), RT-qPCR, microarray, gene array, or RNAseq.

In some embodiments, the protein assay comprises at least one of immunoblotting, fluorescence activated cell sorting, flow-cytometry, magnetic-activated cell sorting, or affinity-based cell separation.

In some embodiments, the cell further comprises one or more of: a priming receptor comprising a first extracellular antigen-binding domain that specifically binds to a first antigen; a chimeric antigen receptor (CAR) comprising a second extracellular antigen-binding domain that specifically binds to a second antigen; or a T cell receptor (TCR).

In some embodiments, the immune cell is a primary human immune cell.

In some embodiments, the primary immune cell is a natural killer (NK) cell, a natural killer T (NKT) cell, a T cell, a γδ T cell, a CD8+ T cell, a CD4+ T cell, a primary T cell, a T cell progenitor, or an induced pluripotent stem cell (iPSC).

In some embodiments, the primary immune cell is a primary T cell.

In some embodiments, the primary immune cell is a primary human T cell.

In some embodiments, the immune cell is virus-free.

In some embodiments, the immune cell is a viable, virus-free, primary cell.

In some embodiments, the immune cell is an autologous immune cell.

In some embodiments, the immune cell is an allogeneic immune cell.

In one aspect, provided herein are primary immune cells comprising at least one recombinant nucleic acid(s) comprising a first nucleic acid comprising a sequence as set forth in SEQ ID NOs: 12-207; and wherein the primary immune cell does not comprise a viral vector for introducing the recombinant nucleic acid(s) into the primary immune cell.

In one aspect, provided herein are viable, virus-free, primary cells comprising one or more ribonucleoprotein complex(es) (RNP), wherein the RNP comprises a nuclease domain and a guide RNA, wherein the guide RNA comprises a first nucleic acid comprising a sequence as set forth in SEQ ID NOs: 12-22.

In some embodiments, further comprising a second different nucleic acid comprising a sequence as set forth in SEQ ID NO: 12-207.

In one aspect, provided herein are primary immune cells comprising a ribonucleoprotein complex (RNP)-recombinant nucleic acid(s) complex, wherein the RNP comprises a nuclease domain and a guide RNA, wherein the recombinant nucleic acid(s) comprises at least a first nucleic acid comprising: (1) a nucleic acid sequence at least 15 nucleotides in length complementary to an mRNA encoding human CD5 comprising the sequence set forth in SEQ ID NO: 1; (2) a nucleic acid sequence at least 15 nucleotides in length complementary to an mRNA encoding human CBLB comprising the sequence set forth in SEQ ID NO: 2; (3) a nucleic acid sequence at least 15 nucleotides in length complementary to an mRNA encoding human CISH comprising the sequence set forth in SEQ ID NO: 3; (4) a nucleic acid sequence at least 15 nucleotides in length complementary to an mRNA encoding DGKA comprising the sequence set forth in SEQ ID NO: 4; (5) a nucleic acid sequence at least 15 nucleotides in length complementary to an mRNA encoding DGKZ comprising the sequence set forth in SEQ ID NO: 5; (6) a nucleic acid sequence at least 15 nucleotides in length complementary to an mRNA encoding DNMT3A comprising the sequence set forth in SEQ ID NO: 6; (7) a nucleic acid sequence at least 15 nucleotides in length complementary to an mRNA encoding MAP4K1 comprising the sequence set forth in SEQ ID NO: 7; (9) a nucleic acid sequence at least 15 nucleotides in length complementary to an mRNA encoding TET2 comprising the sequence set forth in SEQ ID NO: 10; (10) a nucleic acid sequence at least 15 nucleotides in length complementary to an mRNA encoding human Protein Tyrosine Phosphatase Non-Receptor Type 2 (PTPN2) comprising the sequence set forth in SEQ ID NO: 9; (11) a nucleic acid sequence at least 15 nucleotides in length complementary to an mRNA encoding NR4A1 comprising the sequence set forth in SEQ ID NO: 8; or (12) a nucleic acid sequence at least 15 nucleotides in length complementary to an mRNA encoding ZC3H12A comprising the sequence set forth in SEQ ID NO: 11, and wherein the 5′ and 3′ ends of the recombinant nucleic acid(s) comprise nucleotide sequences that are homologous to genomic sequences flanking an insertion site in the genome of the primary cell.

In one aspect, provided herein are viable, virus-free, primary cells comprising a ribonucleoprotein complex (RNP)-recombinant nucleic acid(s) complex, wherein the RNP comprises a nuclease domain and a guide RNA, wherein recombinant nucleic acid(s) comprises at least a first nucleic acid: (1) a nucleic acid sequence at least 15 nucleotides in length complementary to an mRNA encoding human CD5 comprising the sequence set forth in SEQ ID NO: 1; (2) a nucleic acid sequence at least 15 nucleotides in length complementary to an mRNA encoding human CBLB comprising the sequence set forth in SEQ ID NO: 2; (3) a nucleic acid sequence at least 15 nucleotides in length complementary to an mRNA encoding human CISH comprising the sequence set forth in SEQ ID NO: 3; (4) a nucleic acid sequence at least 15 nucleotides in length complementary to an mRNA encoding DGKA comprising the sequence set forth in SEQ ID NO: 4; (5) a nucleic acid sequence at least 15 nucleotides in length complementary to an mRNA encoding DGKZ comprising the sequence set forth in SEQ ID NO: 5; (6) a nucleic acid sequence at least 15 nucleotides in length complementary to an mRNA encoding DNMT3A comprising the sequence set forth in SEQ ID NO: 6; (7) a nucleic acid sequence at least 15 nucleotides in length complementary to an mRNA encoding MAP4K1 comprising the sequence set forth in SEQ ID NO: 7; (9) a nucleic acid sequence at least 15 nucleotides in length complementary to an mRNA encoding TET2 comprising the sequence set forth in SEQ ID NO: 10; (10) a nucleic acid sequence at least 15 nucleotides in length complementary to an mRNA encoding human Protein Tyrosine Phosphatase Non-Receptor Type 2 (PTPN2) comprising the sequence set forth in SEQ ID NO: 9; (11) a nucleic acid sequence at least 15 nucleotides in length complementary to an mRNA encoding NR4A1 comprising the sequence set forth in SEQ ID NO: 8; or (12) a nucleic acid sequence at least 15 nucleotides in length complementary to an mRNA encoding ZC3H12A comprising the sequence set forth in SEQ ID NO: 11, and wherein the 5′ and 3′ ends of the recombinant nucleic acid(s) comprise nucleotide sequences that are homologous to genomic sequences flanking an insertion site in the genome of the primary cell.

In some embodiments, the nucleic acid sequence is an shRNA complementary to the mRNA encoding CD5 and comprises a sequence set forth in any one of SEQ ID NOs: 47-72. In some embodiments, the nucleic acid sequence is an shRNA complementary to the mRNA encoding CBLB and comprises a sequence set forth in any one of SEQ ID NOs: 23-46. In some embodiments, the nucleic acid sequence is an shRNA complementary to the mRNA encoding CISH and comprises a sequence set forth in any one of SEQ ID NOs: 73-95. In some embodiments, the nucleic acid sequence is an shRNA complementary to the mRNA encoding DGKA and comprises a sequence set forth in any one of SEQ ID NOs: 181-204. In some embodiments, the nucleic acid sequence is an shRNA complementary to the mRNA encoding DNMT3A and comprises a sequence set forth in any one of SEQ ID NOs: 96-122. In some embodiments, the nucleic acid sequence is an shRNA complementary to the mRNA encoding TET2 and comprises a sequence set forth in any one of SEQ ID NOs: 147-175. In some embodiments, the nucleic acid sequence is an shRNA complementary to the mRNA encoding PTPN2 and comprises a sequence set forth in any one of SEQ ID NOs: 123-146. In some embodiments, the nucleic acid sequence is an shRNA complementary to the mRNA encoding ZC3H12A and comprises a sequence set forth in any one of SEQ ID NOs: 176-180 and 205-207.

In some embodiments, the cell further comprises a priming receptor comprising a first extracellular antigen-binding domain that specifically binds to a first antigen and a chimeric antigen receptor (CAR) comprising a second extracellular antigen-binding domain that specifically binds to a second antigen, wherein the first antigen and the second antigen are distinct.

In one aspect, provided herein are population of cells comprising a plurality of immune cells disclosed herein.

In one aspect, provided herein are pharmaceutical compositions comprising the immune cell disclosed herein or the population of cells disclosed herein, and a pharmaceutically acceptable excipient.

In one aspect, provided herein are pharmaceutical compositions comprising the recombinant nucleic acid disclosed herein, or the vector disclosed herein, and a pharmaceutically acceptable excipient.

In one aspect, provided herein are methods of editing an immune cell, comprising:

providing a ribonucleoprotein (RNP) comprising a nuclease domain and a guide RNA, wherein the guide RNA comprises a sequence as set forth in SEQ ID NOs: 12-22; non-virally introducing the RNP into the immune cell, wherein the guide RNA specifically hybridizes to a target region of the genome of the primary immune cell, and wherein the nuclease domain cleaves the target region to create a double stranded break site in the genome of the immune cell.

In one aspect, provided herein are methods of editing an immune cell, comprising: providing a ribonucleoprotein (RNP)-recombinant nucleic acid(s) complex, wherein the RNP comprises a nuclease domain and a guide RNA, wherein the recombinant nucleic acid(s) comprises the recombinant nucleic acid(s) disclosed herein, and wherein the 5′ and 3′ ends of the recombinant nucleic acid(s) comprise nucleotide sequences that are homologous to genomic sequences flanking an insertion site in the genome of the immune cell; non-virally introducing the RNP-recombinant nucleic acid(s) complex into the immune cell, wherein the guide RNA specifically hybridizes to a target region of the genome of the primary immune cell, and wherein the nuclease domain cleaves the target region to create the insertion site in the genome of the immune cell; and editing the immune cell via insertion of the recombinant nucleic acid(s) disclosed herein into the insertion site in the genome of the immune cell.

In some embodiments, non-virally introducing comprises electroporation.

In some embodiments, the nuclease domain comprises a CRISPR-associated endonuclease (Cas), optionally a Cas9 nuclease.

In some embodiments, the target region of the genome of the cell is a T Cell Receptor Alpha Constant (TRAC) locus or a genomic safe harbor (GSH) locus.

In some embodiments, the recombinant nucleic acid(s) is a double-stranded recombinant nucleic acid(s) or a single-stranded recombinant nucleic acid(s).

In some embodiments, the recombinant nucleic acid(s) is a linear recombinant nucleic acid(s) or a circular recombinant nucleic acid(s), optionally wherein the circular recombinant nucleic acid(s) is a plasmid.

In some embodiments, the immune cell is a primary human immune cell.

In some embodiments, the immune cell is an autologous immune cell.

In some embodiments, the immune cell is an allogeneic immune cell.

In some embodiments, the immune cell is a natural killer (NK) cell, a natural killer T (NKT) cell, a T cell, a γδ T cell, a CD8+ T cell, a CD4+ T cell, a primary T cell, a T cell progenitor, or an induced pluripotent stem cell (iPSC).

In some embodiments, the immune cell is a primary T cell.

In some embodiments, the immune cell is a primary human T cell.

In some embodiments, the immune cell is virus-free.

In some embodiments, further comprising obtaining the immune cell from a patient and introducing the recombinant nucleic acid in vitro.

In one aspect, provided herein are methods of treating a disease in a subject comprising administering the immune cell(s) disclosed herein or the pharmaceutical composition disclosed herein to the subject.

In some embodiments, the disease is cancer.

In some embodiments, the cancer is a solid cancer or a liquid cancer.

In some embodiments, the cancer is breast cancer, HER2-positive breast cancer, estrogen-receptor positive breast cancer, progesterone-receptor positive breast cancer, HER2-/estrogen-receptor-/progesterone-receptor-negative breast cancer, triple negative breast cancer, non-small cell lung cancer (NSCLC), lung adenocarcinoma, lung squamous cell carcinoma, lung adenosquamous carcinoma, prostate cancer, castration-resistant prostate cancer, colon cancer, rectal cancer, microsatellite instable (MSI) colon cancer, non-MSI colon cancer, or non-MSI or rectal cancer.

In some embodiments, the administration of the cell(s) enhances an immune response.

In some embodiments, the enhanced immune response is an adaptive immune response.

In some embodiments, the enhanced immune response is increased T cell cytotoxicity.

In some embodiments, the enhanced immune response is increased T cell expansion and/or proliferation.

In some embodiments, the enhanced immune response is an innate immune response.

In one aspect, provided herein are methods of enhancing an immune response in a subject comprising administering the immune cell(s) disclosed herein or the pharmaceutical composition disclosed herein to the subject.

In some embodiments, the enhanced immune response is an adaptive immune response.

In some embodiments, the enhanced immune response is increased T cell cytotoxicity.

In some embodiments, the enhanced immune response is increased T cell expansion and/or proliferation.

In some embodiments, the enhanced immune response is an innate immune response.

In some embodiments, expression of CD5, CBLB, CISH, DGKA, DGKZ, DNMT3A, MAP4K1, NR4A1, PTPN2, TET2, and/or ZC3H12A in the immune cell is reduced by at least 50%, 55%, 60%, 65%, 75%, 80%, 85%, 90%, 95%, or 99% as compared to a control cell that does not comprise the first nucleic acid or RNP complex.

In some embodiments, expression of CD5, CBLB, CISH, DGKA, DGKZ, DNMT3A, MAP4K1, NR4A1, PTPN2, TET2, and/or ZC3H12A in the immune cell is reduced by at least 50%, 55%, 60%, 65%, 75%, 80%, 85%, 90%, 95%, or 99% as compared to a control cell that does not comprise the second nucleic acid or RNP complex.

In some embodiments, expression of CD5, CBLB, CISH, DGKA, DGKZ, DNMT3A, MAP4K1, NR4A1, PTPN2, TET2, and/or ZC3H12A in the immune cell is determined by a nucleic acid assay or a protein assay.

In some embodiments, the nucleic acid assay comprises at least one of polymerase chain reaction (PCR), quantitative PCR (qPCR), RT-qPCR, microarray, gene array, or RNAseq.

In some embodiments, the protein assay comprises at least one of immunoblotting, fluorescence activated cell sorting, flow-cytometry, magnetic-activated cell sorting, or affinity-based cell separation.

In some embodiments, further comprising administering an immunotherapy to the subject concurrently with the immune cell or subsequently to the immune cell.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 shows the target cell killing in a repetitive stimulation assay by integrated circuit T cells with individual or combination gene perturbations in the indicated genes relative to non-targeting control cells. Bars represent median values of 3-4 replicates, error bars represent standard deviation.

FIG. 2A provides the target cell growth after incubation with T cells with gene perturbations in the CD5 gene (single KO) or with CD5 and a second gene as indicated (+CD5 KO). FIG. 2B provides the target cell growth after incubation with T cells with gene perturbations in the CBLB gene (single KO) or with CBLB and a second gene as indicated (+CBLB KO). FIG. 2C provides the target cell growth after incubation with T cells with gene perturbations in the CISH gene (single KO) or with CISH and a second gene as indicated (+CISH KO). FIG. 2D provides the target cell growth after incubation with T cells with gene perturbations in the DNMT3A gene (single KO) or with DNMT3A and a second gene as indicated (+DNMT3A KO). FIG. 2E provides the target cell growth after incubation with T cells with gene perturbations in the DGKA gene (single KO) or with DGKA and a second gene as indicated (+DGKA KO). FIG. 2F provides the target cell growth after incubation with T cells with gene perturbations in the DGKZ gene (single KO) or with DGKZ and a second gene as indicated (+DGKZ KO). FIG. 2G provides the target cell growth after incubation with T cells with gene perturbations in the MAP4K1 gene (single KO) or with MAP4K1 and a second gene as indicated (+MAP4K1 KO). FIG. 2H provides the target cell growth after incubation with T cells with gene perturbations in the NR4A1 gene (single KO) or with NR4A1 and a second gene as indicated (+NR4A1 KO). FIG. 2I provides the target cell growth after incubation with T cells with gene perturbations in the PTPN2 gene (single KO) or with PTPN2 and a second gene as indicated (+PTPN2 KO). FIG. 2J provides the target cell growth after incubation with T cells with gene perturbations in the TET2 gene (single KO) or with TET2 and a second gene as indicated (+ TET2 KO). FIG. 2K provides the target cell growth after incubation with T cells with gene perturbations in the ZC3H12A gene (single KO) or with ZC3H12A and a second gene as indicated (+ZC3H12A KO). Open bars are perturbations of the indicated gene in isolation, filled bars are in combination with an additional gene perturbation as indicated, error bars represent standard deviation. Combinations exhibiting superior killing relative to the addition of both indicated genes with FDRs <0.05, and <0.01, are indicated with *, and ** respectively.

FIG. 3 shows the T cell expansion in a repetitive stimulation assay by integrated circuit T cells with individual or combination gene perturbations in the indicated genes relative to non-targeting control cells. Bars represent median values of 3-4 replicates, error bars represent standard deviation.

FIG. 4A provides the T cell expansion after incubation of target cells with T cells with gene perturbations in the CD5 gene (single KO) or with CD5 and a second gene as indicated (+CD5 KO). FIG. 4B provides the T cell expansion after incubation of target cells with T cells with gene perturbations in the CBLB gene (single KO) or with CBLB and a second gene as indicated (+CBLB KO). FIG. 4C provides the T cell expansion after incubation of target cells with T cells with gene perturbations in the CISH gene (single KO) or with CISH and a second gene as indicated (+CISH KO). FIG. 4D provides the T cell expansion after incubation of target cells with T cells with gene perturbations in the DNMT3A gene (single KO) or with DNMT3A and a second gene as indicated (+DNMT3A KO). FIG. 4E provides the T cell expansion after incubation of target cells with T cells with gene perturbations in the DGKA gene (single KO) or with DGKA and a second gene as indicated (+DGKA KO). FIG. 4F provides the T cell expansion after incubation of target cells with T cells with gene perturbations in the DGKZ gene (single KO) or with DGKZ and a second gene as indicated (+DGKZ KO). FIG. 4G provides the T cell expansion after incubation of target cells with T cells with gene perturbations in the MAP4K1 gene (single KO) or with MAP4K1 and a second gene as indicated (+MAP4K1 KO). FIG. 4H provides the T cell expansion after incubation of target cells with T cells with gene perturbations in the NR4A1 gene (single KO) or with NR4A1 and a second gene as indicated (+NR4A1 KO). FIG. 4I provides the T cell expansion after incubation of target cells with T cells with gene perturbations in the PTPN2 gene (single KO) or with PTPN2 and a second gene as indicated (+PTPN2 KO). FIG. 4J provides the T cell expansion after incubation of target cells with T cells with gene perturbations in the TET2 gene (single KO) or with TET2 and a second gene as indicated (+ TET2 KO). FIG. 4K provides the T cell expansion after incubation of target cells with T cells with gene perturbations in the ZC3H12A gene (single KO) or with ZC3H12A and a second gene as indicated (+ZC3H12A KO). Open bars are perturbations of the indicated gene in isolation, filled bars are in combination with an additional gene perturbation as indicated, error bars represent standard deviation. Combinations exhibiting superior T cell expansion relative to the addition of both indicated genes with FDRs <0.05, and <0.01, are indicated with *, and ** respectively.

FIG. 5 depicts a graph of combined performance (log 10 relative proliferation-log 2 relative tumor growth) of ICT cells having the indicated gene perturbations.

FIGS. 6A-6E depict graphs of tumor volumes in mice engrafted with ALPG/MSLN-expressing tumor cells and treated with logic gate-expressing tumor cells having indicated gene perturbations. FIG. 6A depicts tumor volumes in mice having DNMT3A/CBLB double knockout. FIG. 6B depicts tumor volumes in mice having TET2/PTPN2 double knockout. FIG. 6C depicts tumor volumes in mice having CBLB/PTPN2 double knockout. FIG. 6D depicts tumor volumes in mice having PTPN2/CISH double knockout. FIG. 6E depicts tumor volumes in mice having PTPN2/ZC3H12A double knockout.

FIG. 7A-7H depict graphs of mRNA expression of target genes as measured by qPCR following expression of indicated shRNAs. FIG. 7A depicts expression of CBLB following expression of indicated shRNAs. FIG. 7B depicts expression of CD5 following expression of indicated shRNAs. FIG. 7C depicts expression of CISH following expression of indicated shRNAs.

FIG. 7D depicts expression of DNMT3A following expression of indicated shRNAs. FIG. 7E depicts expression of PTPN2 following expression of indicated shRNAs. FIG. 7F depicts expression of TET2 following expression of indicated shRNAs. FIG. 7G depicts expression of ZC3H12A following expression of indicated shRNAs. FIG. 7H depicts expression of DGKA following expression of indicated shRNAs.

FIGS. 8A-8H depict gene expression analysis by RNAseq of cells expressing an shRNA or a validated sgRNA against ZC3H12A compared to control. Points represent individual genes differentially expressed in at least one of shRNA- or sgRNA-expressing cells compared to control. Horizontal axes represent log fold-change (logFC) of genes in sgRNA-expressing cells compared to control. Vetical axes represent logFC of genes in shRNA-expressing cells compared to control. FIG. 8A depicts gene expression analysis of ZC3H12A shRNA 19-expressing cells and sgRNA-expressing cells compared to control. FIG. 8B depicts gene expression analysis of ZC3H12A shRNA 29-expressing cells and sgRNA-expressing cells compared to control. FIG. 8C depicts gene expression analysis of ZC3H12A shRNA 10-expressing cells and sgRNA-expressing cells compared to control. FIG. 8D depicts gene expression analysis of ZC3H12A shRNA 86-expressing cells and sgRNA-expressing cells compared to control. FIG. 8E depicts gene expression analysis of ZC3H12A shRNA 40-expressing cells and sgRNA-expressing cells compared to control. FIG. 8F depicts gene expression analysis of ZC3H12A shRNA 97-expressing cells and sgRNA-expressing cells compared to control. FIG. 8G depicts gene expression analysis of ZC3H12A shRNA 99-expressing cells and sgRNA-expressing cells compared to control. FIG. 8H depicts gene expression analysis of ZC3H12A shRNA 106-expressing cells and sgRNA-expressing cells compared to control.

FIG. 9 depicts a graph of combined performance (log 10 relative proliferation-log 2 relative tumor growth) of ICT cells expressing the indicated shRNA combinations.

### DETAILED DESCRIPTION

### Definitions

Terms used in the claims and specification are defined as set forth below unless otherwise specified.

As used herein, the term “locus” refers to a specific, fixed physical location on a chromosome where a gene or genetic marker is located.

The term “safe harbor locus” refers to a locus at which genes or genetic elements can be incorporated without disruption to expression or regulation of adjacent genes. These safe harbor loci are also referred to as safe harbor sites (SHS) or genomic safe harbor (GSH) sites. As used herein, a safe harbor locus refers to an “integration site” or “knock-in site” at which a sequence encoding a transgene, as defined herein, can be inserted. In some embodiments the insertion occurs with replacement of a sequence that is located at the integration site. In some embodiments, the insertion occurs without replacement of a sequence at the integration site. Examples of integration sites contemplated are provided in Table D.

As used herein, the term “insert” refers to a nucleotide sequence that is integrated (inserted) at a target locus or safe harbor site. The insert can be used to refer to the genes or genetic elements that are incorporated at the target locus or safe harbor site using, for example, homology-directed repair (HDR) CRISPR/Cas9 genome-editing or other methods for inserting nucleotide sequences into a genomic region known to those of ordinary skill in the art.

The term “inserting” refers to a manipulation of a nucleotide sequence to introduce a non-native sequence. This is done, for example, via the use of restriction enzymes and ligases whereby the DNA sequence of interest, usually encoding the gene of interest, can be incorporated into another nucleic acid molecule by digesting both molecules with appropriate restriction enzymes in order to create compatible overlaps and then using a ligase to join the molecules together. One skilled in the art is very familiar with such manipulations and examples may be found in Sambrook et al. (Sambrook, Fritsch, & Maniatis, “Molecular Cloning: A Laboratory Manual”, 2nd ed., Cold Spring Harbor Laboratory, 1989), which is hereby incorporated by reference in its entirety including any drawings, figures and tables.

The “CRISPR/Cas” system refers to a widespread class of bacterial systems for defense against foreign nucleic acid. CRISPR/Cas systems are found in a wide range of eubacterial and archaeal organisms. CRISPR/Cas systems include type I, II, and III sub-types. Wild-type type II CRISPR/Cas systems utilize an RNA-mediated nuclease, Cas9 in complex with guide and activating RNA to recognize and cleave foreign nucleic acid. Guide RNAs having the activity of both a guide RNA and an activating RNA are also known in the art. In some cases, such dual activity guide RNAs are referred to as a small guide RNA (sgRNA).

Cas9 homologs are found in a wide variety of eubacteria, including, but not limited to bacteria of the following taxonomic groups: Actinobacteria, Aquificae, Bacteroidetes-Chlorobi, Chlamydiae-Verrucomicrobia, Chlroflexi, Cyanobacteria, Firmicutes, Proteobacteria, Spirochaetes, and Thermotogae. An exemplary Cas9 protein is the Streptococcus pyogenes Cas9 protein. Additional Cas9 proteins and homologs thereof are described in, e.g., Chylinksi, et al., RNA Biol. 2013 May 1; 10 (5): 726-737; Nat. Rev. Microbiol. 2011 June; 9 (6): 467-477; Hou, et al., Proc Natl Acad Sci USA. 2013 Sep. 24; 110 (39): 15644-9; Sampson et al., Nature. 2013 May 9; 497 (7448): 254-7; and Jinek, et al., Science. 2012 Aug. 17; 337 (6096): 816-21. The Cas9 nuclease domain can be optimized for efficient activity or enhanced stability in the host cell.

As used herein, the term “Cas9” refers to an RNA-mediated nuclease (e.g., of bacterial or archeal orgin, or derived therefrom). Exemplary RNA-mediated nuclases include the foregoing Cas9 proteins and homologs thereof, and include but are not limited to, CPF1 (See, e.g., Zetsche et al., Cell, Volume 163, Issue 3, p759-771, 22 Oct. 2015). Similarly, as used herein, the term “Cas9 ribonucleoprotein” complex and the like refers to a complex between the Cas9 protein, and a crRNA (e.g., guide RNA or small guide RNA), the Cas9 protein and a trans-activating crRNA (tracrRNA), the Cas9 protein and a small guide RNA, or a combination thereof (e.g., a complex containing the Cas9 protein, a tracrRNA, and a crRNA guide RNA).

As used herein, the phrase “immune cell” is inclusive of all cell types that can give rise to immune cells, including hematopoietic cells such hematopoietic stem cells, pluripotent stem cells, and induced pluripotent stem cells (iPSCs). In some embodiments, the immune cell is a B cell, macrophage, a natural killer (NK) cell, an induced pluripotent stem cell (iPSC), a human pluripotent stem cell (HSPC), a T cell or a T cell progenitor or dendritic cell. In some embodiments, the cell is an innate immune cell.

As used herein, the term “primary” in the context of a primary cell or primary stem cell refers to a cell that has not been transformed or immortalized. Such primary cells can be cultured, sub-cultured, or passaged a limited number of times (e.g., cultured 0, 1, 2, 3, 4, 5, 6, 7, 8, 9, 10, 11, 12, 13, 14, 15, 16, 17, 18, 19, or 20 times). In some cases, the primary cells are adapted to in vitro culture conditions. In some cases, the primary cells are isolated from an organism, system, organ, or tissue, optionally sorted, and utilized, e.g., directly without culturing or sub-culturing. In some cases, the primary cells are stimulated, activated, or differentiated. For example, primary T cells can be activated by contact with (e.g., culturing in the presence of) CD3, CD28 agonists, IL-2, IFN-γ, or a combination thereof.

As used herein, the terms “T lymphocyte” and “T cell” are used interchangeably and refer to cells that have completed maturation in the thymus, and identify certain foreign antigens in the body. The terms also refer to the major leukocyte types that have various roles in the immune system, including activation and deactivation of other immune cells. The T cell can be any T cell such as a cultured T cell, e.g., a primary T cell, or a T cell derived from a cultured T cell line, e.g., a Jurkat, SupT1, etc., or a T cell obtained from a mammal. T cells include, but are not limited to, naïve T cells, stimulated T cells, primary T cells (e.g., uncultured), cultured T cells, immortalized T cells, helper T cells, cytotoxic T cells, memory T cells, regulatory T cells, natural killer T cells, combinations thereof, or sub-populations thereof. The T cell can be a CD3+ cell. T cells can be CD4⁺, CD8⁺, or CD4⁺ and CD8⁺. The T cell can be any type of T cell, CD4+/CD8+double positive T cells, CD4+ helper T cells (e.g. Th1 and Th2 cells), CD8+ T cells (e.g. cytotoxic T cells), peripheral Including but not limited to blood mononuclear cells (PBMC), peripheral blood leukocytes (PBL), tumor infiltrating lymphocytes (TIL), memory T cells, naive T cells, regulatory T cells, γδ T cells, etc. It can be any T cell at any stage of development. Additional types of helper T cells include Th3 (Treg) cells, Th17 cells, Th9 cells, or Tfh cells. Additional types of memory T cells include cells such as central memory T cells (Tcm cells), effector memory T cells (Tem cells and TEMRA cells). A T cell can also refer to a genetically modified T cell, such as a T cell that has been modified to express a T cell receptor (TCR), a chimeric antigen receptor (CAR), or a priming receptor (primeR). T cells can also be differentiated from stem cells or progenitor cells.

“CD4+ T cells” refers to a subset of T cells that express CD4 on their surface and are associated with a cellular immune response. CD4+ T cells are characterized by a post-stimulation secretion profile that can include secretion of cytokines such as IFN-γ, TNF-α, IL-2, IL-4 and IL-10. “CD4” is a 55 kD glycoprotein originally defined as a differentiation antigen on T lymphocytes, but was also found on other cells including monocytes/macrophages. The CD4 antigen is a member of the immunoglobulin superfamily and has been implicated as an associative recognition element in MHC (major histocompatibility complex) class II restricted immune responses. On T lymphocytes, the CD4 antigen defines a helper/inducer subset.

“CD8+ T cells” refers to a subset of T cells that express CD8 on their surface, are MHC class I restricted, and function as cytotoxic T cells. The “CD8” molecule is a differentiation antigen present on thymocytes, as well as on cytotoxic and suppressor T lymphocytes. The CD8 antigen is a member of the immunoglobulin superfamily and is an associative recognition element in major histocompatibility complex class I restriction interactions.

As used herein, the phrase “hematopoietic stem cell” refers to a type of stem cell that can give rise to a blood cell. Hematopoietic stem cells can give rise to cells of the myeloid or lymphoid lineages, or a combination thereof. Hematopoietic stem cells are predominantly found in the bone marrow, although they can be isolated from peripheral blood, or a fraction thereof. Various cell surface markers can be used to identify, sort, or purify hematopoietic stem cells. In some cases, hematopoietic stem cells are identified as c-kit⁺ and lin. In some cases, human hematopoietic stem cells are identified as CD34⁺, CD59⁺, Thy1/CD90⁺, CD38^{lo/-}, C-kit/CD117⁺, lin. In some cases, human hematopoietic stem cells are identified as CD34⁻, CD59⁺, Thy1/CD90⁺, CD38^{lo/-}, C-kit/CD117⁺, lin⁻. In some cases, human hematopoietic stem cells are identified as CD133⁺, CD59⁺, Thy1/CD90⁺, CD38^{lo/-}, C-kit/CD117⁺, lin⁻. In some cases, mouse hematopoietic stem cells are identified as CD34^{lo/-}, SCA-1⁺, Thy1^{+/lo}, CD38⁺, C-kit⁺, lin⁻. In some cases, the hematopoietic stem cells are CD150⁺CD48⁻CD244⁻.

As used herein, the phrase “hematopoietic cell” refers to a cell derived from a hematopoietic stem cell. The hematopoietic cell may be obtained or provided by isolation from an organism, system, organ, or tissue (e.g., blood, or a fraction thereof). Alternatively, an hematopoietic stem cell can be isolated and the hematopoietic cell obtained or provided by differentiating the stem cell. Hematopoietic cells include cells with limited potential to differentiate into further cell types. Such hematopoietic cells include, but are not limited to, multipotent progenitor cells, lineage-restricted progenitor cells, common myeloid progenitor cells, granulocyte-macrophage progenitor cells, or megakaryocyte-erythroid progenitor cells. Hematopoietic cells include cells of the lymphoid and myeloid lineages, such as lymphocytes, erythrocytes, granulocytes, monocytes, and thrombocytes.

As used herein, the term “construct” refers to a complex of molecules, including macromolecules or polynucleotides.

As used herein, the term “integration” refers to the process of stably inserting one or more nucleotides of a construct into the cell genome, i.e., covalently linking to a nucleic acid sequence in the chromosomal DNA of the cell. It may also refer to nucleotide deletions at a site of integration. Where there is a deletion at the insertion site, “integration” may further include substitution of the endogenous sequence or nucleotide deleted with one or more inserted nucleotides.

The term “deletion,” perturb,” or “perturbation” in reference to a gene refers to full, partial, or functional deletion of a target gene.

The term “exogenous” refers to a molecule or activity that has been introduced into a host cell and is not native to that cell. The molecule can be introduced, for example, by introduction of the encoding nucleic acid into host genetic material, such as by integration into a host chromosome, or as non-chromosomal genetic material, such as a plasmid. Thus, the term, when used in connection with expression of an encoding nucleic acid, refers to the introduction of the encoding nucleic acid into a cell in an expressible form. The term “endogenous” refers to a molecule or activity that is present in a host cell under natural, unedited conditions. Similarly, the term, when used in connection with expression of the encoding nucleic acid, refers to expression of the encoding nucleic acid that is contained within the cell and not introduced exogenously.

The term “heterologous” refers to a nucleic acid or polypeptide sequence or domain which is not native to a flanking sequence, e.g., wherein the heterologous sequence is not found in nature coupled to the nucleic acid or polypeptide sequences occurring at one or both ends.

The term “homologous” refers to a nucleic acid or polypeptide sequence or domain which is native to a flanking sequence, e.g., wherein the homologous sequence is found in nature coupled to the nucleic acid or polypeptide sequences occurring at one or both ends.

As used herein, a “polynucleotide donor construct” refers to a nucleotide sequence (e.g. DNA sequence) that is genetically inserted into a polynucleotide and is exogenous to that polynucleotide. The polynucleotide donor construct is transcribed into RNA and optionally translated into a polypeptide. The polynucleotide donor construct can include prokaryotic sequences, cDNA from eukaryotic mRNA, genomic DNA sequences from eukaryotic (e.g., mammalian) DNA, and synthetic DNA sequences. For example, the polynucleotide donor construct can be a miRNA, shRNA, natural polypeptide (i.e., a naturally occurring polypeptide) or fragment thereof or a variant polypeptide (e.g. a natural polypeptide having less than 100% sequence identity with the natural polypeptide) or fragments thereof.

As used herein, the term “complementary” or “complementarity” refers to specific base pairing between nucleotides or nucleic acids. Complementary nucleotides are, generally, A and T (or A and U), and G and C. The guide RNAs described herein can comprise sequences, for example, DNA targeting sequence that are perfectly complementary or substantially complementary (e.g., having 1-4 mismatches) to a genomic sequence in a cell.

As used herein, the term “transgene” refers to a polynucleotide that has been transferred naturally, or by any of a number of genetic engineering techniques from one organism to another. It is optionally translated into a polypeptide. It is optionally translated into a recombinant protein. A “recombinant protein” is a protein encoded by a gene—recombinant DNA—that has been cloned in a system that supports expression of the gene and translation of messenger RNA (see expression system). The recombinant protein can be a therapeutic agent, e.g. a protein that treats a disease or disorder disclosed herein. As used, transgene can refer to a polynucleotide that encodes a polypeptide.

The terms “protein,” “polypeptide,” and “peptide” are used herein interchangeably.

As used herein, the term “operably linked” or “operatively linked” refers to the binding of a nucleic acid sequence to a single nucleic acid fragment such that one function is affected by the other. For example, if a promoter is capable of affecting the expression of a coding sequence or functional RNA (i.e., the coding sequence or functional RNA is under transcriptional control by the promoter), the promoter is operably linked thereto. Coding sequences can be operably linked to control sequences in both sense and antisense orientation.

As used herein, the term “developmental cell states” refers to, for example, states when the cell is inactive, actively expressing, differentiating, senescent, etc. developmental cell state may also refer to a cell in a precursor state (e.g., a T cell precursor).

As used, the term “encoding” refers to a sequence of nucleic acids which codes for a protein or polypeptide of interest. The nucleic acid sequence may be either a molecule of DNA or RNA. In preferred embodiments, the molecule is a DNA molecule. In other preferred embodiments, the molecule is a RNA molecule. When present as a RNA molecule, it will comprise sequences which direct the ribosomes of the host cell to start translation (e.g., a start codon, ATG) and direct the ribosomes to end translation (e.g., a stop codon). Between the start codon and stop codon is an open reading frame (ORF). Such terms are known to one of ordinary skill in the art.

As used herein, the term “subject” refers to a mammalian subject. Exemplary subjects include humans, monkeys, dogs, cats, mice, rats, cows, horses, camels, goats, rabbits, pigs and sheep. In certain embodiments, the subject is a human. In some embodiments the subject has a disease or condition that can be treated with an engineered cell provided herein or population thereof. In some aspects, the disease or condition is a cancer.

As used herein, the term “promoter” refers to a nucleotide sequence (e.g. DNA sequence) capable of controlling the expression of a coding sequence or functional RNA. The promoter sequence consists of proximal and more distal upstream elements, the latter elements often referred to as enhancers. A promoter can be derived from natural genes in its entirety, can be composed of different elements from different promoters found in nature, and/or may comprise synthetic DNA segments. A promoter, as contemplated herein, can be endogenous to the cell of interest or exogenous to the cell of interest. It is appreciated by those skilled in the art that different promoters can induce gene expression in different tissue or cell types, or at different developmental stages, or in response to different environmental conditions. As is known in the art, a promoter can be selected according to the strength of the promoter and/or the conditions under which the promoter is active, e.g., constitutive promoter, strong promoter, weak promoter, inducible/repressible promoter, tissue specific Or developmentally regulated promoters, cell cycle-dependent promoters, and the like.

A promoter can be an inducible promoter (e.g., a heat shock promoter, tetracycline-regulated promoter, steroid-regulated promoter, metal-regulated promoter, estrogen receptor-regulated promoter, etc.). The promoter can be a constitutive promoter (e.g., CMV promoter, UBC promoter). In some embodiments, the promoter can be a spatially restricted and/or temporally restricted promoter (e.g., a tissue specific promoter, a cell type specific promoter, etc.). See for example US Publication 20180127786, the disclosure of which is herein incorporated by reference in its entirety.

Gene editing, as contemplated herein, may involve a gene (or nucleotide sequence) knock-in or knock-out. As used herein, the term “knock-in” refers to an addition of a DNA sequence, or fragment thereof into a genome. Such DNA sequences to be knocked-in may include an entire gene or genes, may include regulatory sequences associated with a gene or any portion or fragment of the foregoing. For example, a polynucleotide donor construct encoding a recombinant protein may be inserted into the genome of a cell carrying a mutant gene. In some embodiments, a knock-in strategy involves substitution of an existing sequence with the provided sequence, e.g., substitution of a mutant allele with a wild-type copy. On the other hand, the term “knock-out” refers to the elimination of a gene or the expression of a gene. For example, a gene can be knocked out by either a deletion or an addition of a nucleotide sequence that leads to a disruption of the reading frame. As another example, a gene may be knocked out by replacing a part of the gene with an irrelevant (.e.g., non-coding) sequence.

As used herein, the term “non-homologous end joining” or NHEJ refers to a cellular process in which cut or nicked ends of a DNA strand are directly ligated without the need for a homologous template nucleic acid. NHEJ can lead to the addition, the deletion, substitution, or a combination thereof, of one or more nucleotides at the repair site.

As used herein, the term “homology directed repair” or HDR refers to a cellular process in which cut or nicked ends of a DNA strand are repaired by polymerization from a homologous template nucleic acid. Thus, the original sequence is replaced with the sequence of the template. The homologous template nucleic acid can be provided by homologous sequences elsewhere in the genome (sister chromatids, homologous chromosomes, or repeated regions on the same or different chromosomes). Alternatively, an exogenous template nucleic acid can be introduced to obtain a specific HDR-induced change of the sequence at the target site. In this way, specific mutations can be introduced at the cut site.

As used herein, a single-stranded DNA template or a double-stranded DNA template refers to a DNA oligonucleotide that can be used by a cell as a template for HDR. Generally, the single-stranded DNA template or a double-stranded DNA template has at least one region of homology to a target site. In some cases, the single-stranded DNA template or double-stranded DNA template has two homologous regions flanking a region that contains a heterologous sequence to be inserted at a target cut site.

The terms “vector” and “plasmid” are used interchangeably and as used herein refer to polynucleotide vehicles useful to introduce genetic material into a cell. Vectors can be linear or circular. Vectors can integrate into a target genome of a host cell or replicate independently in a host cell. Vectors can comprise, for example, an origin of replication, a multicloning site, and/or a selectable marker. An expression vector typically comprises an expression cassette. Vectors and plasmids include, but are not limited to, integrating vectors, prokaryotic plasmids, eukaryotic plasmids, plant synthetic chromosomes, episomes, cosmids, and artificial chromosomes.

As used herein, the phrase “introducing” in the context of introducing a nucleic acid or a complex comprising a nucleic acid, for example, an RNP-DNA template complex, refers to the translocation of the nucleic acid sequence or the RNP-DNA template complex from outside a cell to inside the cell. In some cases, introducing refers to translocation of the nucleic acid or the complex from outside the cell to inside the nucleus of the cell. Various methods of such translocation are contemplated, including but not limited to, electroporation, contact with nanowires or nanotubes, receptor mediated internalization, translocation via cell penetrating peptides, liposome mediated translocation, and the like.

As used herein the term “expression cassette” is a polynucleotide construct, generated recombinantly or synthetically, comprising regulatory sequences operably linked to a selected polynucleotide to facilitate expression of the selected polynucleotide in a host cell. For example, the regulatory sequences can facilitate transcription of the selected polynucleotide in a host cell, or transcription and translation of the selected polynucleotide in a host cell. An expression cassette can, for example, be integrated in the genome of a host cell or be present in an expression vector.

As used herein, the phrase “subject in need thereof” refers to a subject that exhibits and/or is diagnosed with one or more symptoms or signs of a disease or disorder as described herein.

A “chemotherapeutic agent” refers to a chemical compound useful in the treatment of cancer. Chemotherapeutic agents include “anti-hormonal agents” or “endocrine therapeutics” which act to regulate, reduce, block, or inhibit the effects of hormones that can promote the growth of cancer.

The term “composition” refers to a mixture that contains, e.g., an engineered cell or nucleic acid contemplated herein. In some embodiments, the composition may contain additional components, such as adjuvants, stabilizers, excipients, and the like. The term “composition” or “pharmaceutical composition” refers to a preparation which is in such form as to permit the biological activity of an active ingredient contained therein to be effective in treating a subject, and which contains no additional components which are unacceptably toxic to the subject in the amounts provided in the pharmaceutical composition.

The term “in situ” refers to processes that occur in a living cell growing separate from a living organism, e.g., growing in tissue culture.

The term “in vivo” refers to processes that occur in a living organism.

As used herein, the term “ex vivo” generally includes experiments or measurements made in or on living tissue, preferably in an artificial environment outside the organism, preferably with minimal differences from natural conditions.

The term “mammal” as used herein includes both humans and non-humans and include but is not limited to humans, non-human primates, canines, felines, murines, bovines, equines, and porcines.

The term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

The term “sufficient amount” means an amount sufficient to produce a desired effect, e.g., an amount sufficient to modulate protein aggregation in a cell.

The term “therapeutically effective amount” is an amount that is effective to ameliorate a symptom of a disease.

The term “ameliorating” refers to any therapeutically beneficial result in the treatment of a disease state, e.g., a cancer disease state, lessening in the severity or progression, remission, or cure thereof.

As used herein, the term “effective amount” refers to the amount of a compound (e.g., a compositions described herein, cells described herein) sufficient to effect beneficial or desired results. An effective amount can be administered in one or more administrations, applications or dosages and is not intended to be limited to a particular formulation or administration route.

As used herein, the term “treating” includes any effect, e.g., lessening, reducing, modulating, ameliorating or eliminating, that results in the improvement of the condition, disease, disorder, and the like, or ameliorating a symptom thereof.

The terms “modulate” and “modulation” refer to reducing or inhibiting or, alternatively, activating or increasing, a recited variable.

The terms “increase” and “activate” refer to an increase of 10%, 20%, 30%, 40%, 50%, 60%, 70%, 75%, 80%, 85%, 90%, 95%, 100%, 2-fold, 3-fold, 4-fold, 5-fold, 10-fold, 20-fold, 50-fold, 100-fold, or greater in a recited variable.

The terms “reduce” and “inhibit” refer to a decrease of 10%, 20%, 30%, 40%, 50%, 60%, 70%, 75%, 80%, 85%, 90%, 95%, 2-fold, 3-fold, 4-fold, 5-fold, 10-fold, 20-fold, 50-fold, 100-fold, or greater in a recited variable.

It must be noted that, as used in the specification and the appended claims, the singular forms “a,” “an” and “the” include plural referents unless the context clearly dictates otherwise.

### Recombinant Nucleic Acid Compositions

CD5 Antigen (CD5, HGNC: 1685, NCBI Entrez Gene: 921) is a member of the scavenger receptor cysteine-rich (SRCR) superfamily.

Cbl Proto-Oncogene B (CBLB, HGNC: 1542, NCBI Entrez Gene: 868) is an E3 ubiquitin-protein ligase.

Cytokine Inducible SH2 Containing Protein (CISH, HGNC: 1984, NCBI Entrez Gene: 1154) is a member of the cytokine-induced STAT inhibitor (CIS) protein family.

Diacylglycerol Kinase Alpha (DGKA, HGNC: 2849, NCBI Entrez Gene: 1606) acts as a modulator that competes with protein kinase C for the second messenger diacylglycerol in intracellular signaling pathways.

Diacylglycerol Kinase Zeta (DGKZ, HGNC: 2857, NCBI Entrez Gene: 8525) is another diacylglycerol kinase family member, like DGKA.

DNA Methyltransferase 3 Alpha (DNMT3A, HGNC: 2978, NCBI Entrez Gene: 1788) is a DNA methyltransferase.

Mitogen-Activated Protein Kinase Kinase Kinase Kinase 1 (MAP4K1, HGNC: 6863, NCBI Entrez Gene: 11184) is involved in ATP binding activity and MAP kinase kinase kinase kinase activity.

Nuclear Receptor Subfamily 4 Group A Member 1 (NR4A1, HGNC: 7980, NCBI Entrez Gene: 3164) is a member of the steroid-thyroid hormone-retinoid receptor superfamily.

Protein Tyrosine Phosphatase Non-Receptor Type 2 (PTPN2, HGNC: 9650, NCBI Entrez Gene: 5771) is a member of the protein tyrosine phosphatase (PTP) family.

Tet Methylcytosine Dioxygenase 2 (TET2, HGNC: 25941; NCBI Entrez Gene: 54790) is involved in myelopoiesis, and defects in this gene have been associated with several myeloproliferative disorders.

Zinc Finger CCCH-Type Containing 12A (ZC3H12A, HGNC: 26259, NCBI Entrez Gene: 80149) is a transcriptional activator and causes cell death of cardiomyocytes.

As used herein, “target gene” refers to a nucleic acid sequence in a cell, wherein the expression of the sequence may be specifically and effectively modulated using the recombinant nucleic acid molecules and methods described herein. In certain embodiments, the target gene may be implicated in the growth (proliferation), maintenance (survival), and/or immune behavior of an individual's immune cells.

In some embodiments, the target gene is PTPN2. In some embodiments, the target gene is CD5. In some embodiments, the target gene is CBLB. In some embodiments, the target gene is CISH. In some embodiments, the target gene is DGKA. In some embodiments, the target gene is DGKZ. In some embodiments, the target gene is DNMT3A. In some embodiments, the target gene is MAP4K1. In some embodiments, the target gene is NR4A1. In some embodiments, the target gene is ZC3H12A.

In some embodiments, more than one target gene is deleted or modulated using a recombinant nucleic acid molecule and methods described herein. In some embodiments, at least two target gene are deleted or modulated using the recombinant nucleic acid molecules and methods described herein. In some embodiments, the recombinant nucleic acid molecule(s) is an shRNA. In some embodiments, the recombinant nucleic acid molecule(s) is a guide RNA.

In some embodiments, the one or more recombinant nucleic acids comprising at least one sequence. In some embodiments, the one or more recombinant nucleic acids comprising at least two sequences. In some embodiments, the one or more recombinant nucleic acids comprising at least three sequences. In some embodiments, the one or more recombinant nucleic acids comprising at least four sequences. In some embodiments, the one or more recombinant nucleic acids comprising at least five sequences. In some embodiments, the one or more recombinant nucleic acids are encoded on one polynucleotide. In some embodiments, the one or more recombinant nucleic acids are encoded on two or more polynucleotides. In some embodiments, the one or more recombinant nucleic acids are encoded on three or more polynucleotides. In some embodiments, the one or more recombinant nucleic acids are encoded on four or more polynucleotides. In some embodiments, the one or more recombinant nucleic acids are encoded on five or more polynucleotides.

In some embodiments, the nucleic acid comprises a first nucleic acid and a second nucleic acid, wherein the first and second nucleic acids are distinct. In some embodiments, the first nucleic acid comprises a sequence set forth in SEQ ID NO: 12 and the second nucleic acid comprises a sequence set forth in SEQ ID NO: 13. In some embodiments, the first nucleic acid comprises a sequence set forth in SEQ ID NO: 21 and the second nucleic acid comprises a sequence set forth in SEQ ID NO: 13. In some embodiments, the first nucleic acid comprises a sequence set forth in SEQ ID NO: 21 and the second nucleic acid comprises a sequence set forth in SEQ ID NO: 12. In some embodiments, the first nucleic acid comprises a sequence set forth in SEQ ID NO: 12 and the second nucleic acid comprises a sequence set forth in SEQ ID NO: 14. In some embodiments, the first nucleic acid comprises a sequence set forth in SEQ ID NO: 13 and the second nucleic acid comprises a sequence set forth in SEQ ID NO: 20. In some embodiments, the first nucleic acid comprises a sequence set forth in SEQ ID NO: 21 and the second nucleic acid comprises a sequence set forth in SEQ ID NO: 20. In some embodiments, the first nucleic acid comprises a sequence set forth in SEQ ID NO: 20 and the second nucleic acid comprises a sequence set forth in SEQ ID NO: 22. In some embodiments, the first nucleic acid comprises a sequence set forth in SEQ ID NO: 17 and the second nucleic acid comprises a sequence set forth in SEQ ID NO: 13. In some embodiments, the first nucleic acid comprises a sequence set forth in SEQ ID NO: 20 and the second nucleic acid comprises a sequence set forth in SEQ ID NO: 14. In some embodiments, the first nucleic acid comprises a sequence set forth in SEQ ID NO: 21 and the second nucleic acid comprises a sequence set forth in SEQ ID NO: 22

In some embodiments, the nucleic acid sequence is at least 16, 17, 18, 19, 20, 21, or 22 nucleotides in length.

In some embodiments, the nucleic acid is a an RNA interference (RNAi) molecule. Exemplary RNAi molecules include short hairpin RNA (shRNA), a small interfering RNA (siRNA), a double stranded RNA (dsRNA), or an antisense oligonucleotide. In some embodiments, the nucleic acid is a short hairpin RNA (shRNA), a small interfering RNA (siRNA), a double stranded RNA (dsRNA), or an antisense oligonucleotide. In some embodiments, the nucleic acid is an shRNA.

Single-stranded hairpin ribonucleic acids (shRNAs) are short duplexes where the sense and antisense strands are linked by a hairpin loop. They consist of a stem-loop structure that can be transcribed in cells from an RNA polymerase II or RNA polymerase III promoter on a plasmid construct. Once expressed, shRNAs are processed into RNAi species. Expression of shRNA from a plasmid is known to be relatively stable, thereby providing strong advantages over, for example, the use of synthetic siRNAs. shRNA expression units may be incorporated into a variety of plasmids, liposomes, viral vectors, and other vehicles for delivery and integration into a target cell. Expression of shRNA from a plasmid can be stably integrated for constitutive expression. shRNAs are synthesized in the nucleus of cells, further processed and transported to the cytoplasm, and then incorporated into the RNA-induced silencing complex (RISC) for activity. The shRNAs are converted into active siRNA molecules (which are capable of binding to, sequestering, and/or preventing the translation of mRNA transcripts encoded by target genes).

The Argonaute family of proteins is the major component of RISC. Within the Argonaute family of proteins, only Ago2 contains endonuclease activity that is capable of cleaving and releasing the passenger strand from the stem portion of the shRNA molecule. The remaining three members of Argonaute family, Ago1, Ago3 and Ago4, which do not have identifiable endonuclease activity, are also assembled into RISC and are believed to function through a cleavage-independent manner. Thus, RISC can be characterized as having cleavage-dependent and cleavage-independent pathways.

RNAi (e.g., antisense RNA, siRNA, microRNA, shRNA, etc.) are described in International Publication Nos. WO2018232356A1, WO2019084552A1, WO2019226998A1, WO2020014235A1, WO2020123871A1, and WO2020186219A1, each of which is herein incorporated by reference for all purposes.

Antisense oligonucleotide structure and chemical modifications are described in International PCT Publication No. WO20/132521, which is hereby incorporated by reference.

dsRNA and shRNA molecules and methods of use and production are described in U.S. Pat. Nos. 8,829,264; 9,556,431; and 8,252,526, each of which are hereby incorporated by reference.

In some embodiments, the one or more recombinant nucleic acids comprise an shRNA comprising a nucleic acid sequence set forth in any one of SEQ ID NOs: 23-207. In some embodiments, the one or more recombinant nucleic acids comprise an shRNA complementary to an mRNA encoding CBLB and comprising a nucleic acid sequence set forth in any one of SEQ ID NOS: 23-46. In some embodiments, the one or more recombinant nucleic acids comprise an shRNA complementary to an mRNA encoding CD5 and comprising a nucleic acid sequence set forth in any one of SEQ ID NOs: 47-72. In some embodiments, the one or more recombinant nucleic acids comprise an shRNA complementary to an mRNA encoding CISH and comprising a nucleic acid sequence set forth in any one of SEQ ID NOs: 73-95. In some embodiments, the one or more recombinant nucleic acids comprise an shRNA complementary to an mRNA encoding DNMT3A and comprising a nucleic acid sequence set forth in any one of SEQ ID NOs: 96-122. In some embodiments, the one or more recombinant nucleic acids comprise an shRNA complementary to an mRNA encoding PTPN2 and comprising a nucleic acid sequence set forth in any one of SEQ ID NOs: 123-146. In some embodiments, the one or more recombinant nucleic acids comprise an shRNA complementary to an mRNA encoding TET2 and comprising a nucleic acid sequence set forth in any one of SEQ ID NOs: 147-175. In some embodiments, the one or more recombinant nucleic acids comprise an shRNA complementary to an mRNA encoding ZC3H12A and comprising a nucleic acid sequence set forth in any one of SEQ ID NOs: 176-180 and 205-207. In some embodiments, the one or more recombinant nucleic acids comprise an shRNA complementary to an mRNA encoding DGKA and comprising a nucleic acid sequence set forth in any one of SEQ ID NOs: 181-204.

In some embodiments, the one or more recombinant nucleic acids comprise first nucleic acid and a second nucleic acid, wherein the first and second nucleic acids are different. In some embodiments, the first and second nucleic acids are each an shRNA.

In some embodiments, the first nucleic acid is an shRNA complementary to an mRNA encoding DNMT3A, and the second nucleic acid is an shRNA complementary to an mRNA encoding CBLB. In some embodiments, the first nucleic acid is an shRNA complementary to an mRNA encoding PTPN2, and the second nucleic acid is an shRNA complementary to an mRNA encoding CBLB. In some embodiments, the first nucleic acid is an shRNA complementary to an mRNA encoding TET2, and the second nucleic acid is an shRNA complementary to an mRNA encoding CBLB. In some embodiments, the first nucleic acid is an shRNA complementary to an mRNA encoding CD5, and the second nucleic acid is an shRNA complementary to an mRNA encoding CISH. In some embodiments, the first nucleic acid is an shRNA complementary to an mRNA encoding PTPN2, and the second nucleic acid is an shRNA complementary to an mRNA encoding CISH. In some embodiments, the first nucleic acid is an shRNA complementary to an mRNA encoding PTPN2, and the second nucleic acid is an shRNA complementary to an mRNA encoding TET2. In some embodiments, the first nucleic acid is an shRNA complementary to an mRNA encoding PTPN2, and the second nucleic acid is an shRNA complementary to an mRNA encoding ZC3H12A. In some embodiments, the first nucleic acid is an shRNA complementary to an mRNA encoding TET2, and the second nucleic acid is an shRNA complementary to an mRNA encoding ZC3H12A.

In some embodiments, the first nucleic acid comprises a sequence set forth in SEQ ID NO: 37 and the second nucleic acid comprises a sequence set forth in SEQ ID NO: 120. In some embodiments, the first nucleic acid comprises a sequence set forth in SEQ ID NO: 45 and the second nucleic acid comprises a sequence set forth in SEQ ID NO: 120. In some embodiments, the first nucleic acid comprises a sequence set forth in SEQ ID NO: 120 and the second nucleic acid comprises a sequence set forth in SEQ ID NO: 29. In some embodiments, the first nucleic acid comprises a sequence set forth in SEQ ID NO: 45 and the second nucleic acid comprises a sequence set forth in SEQ ID NO: 122. In some embodiments, the first nucleic acid comprises a sequence set forth in SEQ ID NO: 29 and the second nucleic acid comprises a sequence set forth in SEQ ID NO: 122. In some embodiments, the first nucleic acid comprises a sequence set forth in SEQ ID NO: 37 and the second nucleic acid comprises a sequence set forth in SEQ ID NO: 111. In some embodiments, the first nucleic acid comprises a sequence set forth in SEQ ID NO: 122 and the second nucleic acid comprises a sequence set forth in SEQ ID NO: 37. In some embodiments, the first nucleic acid comprises a sequence set forth in SEQ ID NO: 46 and the second nucleic acid comprises a sequence set forth in SEQ ID NO: 122. In some embodiments, the first nucleic acid comprises a sequence set forth in SEQ ID NO: 46 and the second nucleic acid comprises a sequence set forth in SEQ ID NO: 120. In some embodiments, the first nucleic acid comprises a sequence set forth in SEQ ID NO: 37 and the second nucleic acid comprises a sequence set forth in SEQ ID NO: 141. In some embodiments, the first nucleic acid comprises a sequence set forth in SEQ ID NO: 141 and the second nucleic acid comprises a sequence set forth in SEQ ID NO: 44. In some embodiments, the first nucleic acid comprises a sequence set forth in SEQ ID NO: 143 and the second nucleic acid comprises a sequence set forth in SEQ ID NO: 29. In some embodiments, the first nucleic acid comprises a sequence set forth in SEQ ID NO: 46 and the second nucleic acid comprises a sequence set forth in SEQ ID NO: 141. In some embodiments, the first nucleic acid comprises a sequence set forth in SEQ ID NO: 29 and the second nucleic acid comprises a sequence set forth in SEQ ID NO: 141. In some embodiments, the first nucleic acid comprises a sequence set forth in SEQ ID NO: 37 and the second nucleic acid comprises a sequence set forth in SEQ ID NO: 143. In some embodiments, the first nucleic acid comprises a sequence set forth in SEQ ID NO: 45 and the second nucleic acid comprises a sequence set forth in SEQ ID NO: 146. In some embodiments, the first nucleic acid comprises a sequence set forth in SEQ ID NO: 45 and the second nucleic acid comprises a sequence set forth in SEQ ID NO: 141. In some embodiments, the first nucleic acid comprises a sequence set forth in SEQ ID NO: 29 and the second nucleic acid comprises a sequence set forth in SEQ ID NO: 146. In some embodiments, the first nucleic acid comprises a sequence set forth in SEQ ID NO: 37 and the second nucleic acid comprises a sequence set forth in SEQ ID NO: 174. In some embodiments, the first nucleic acid comprises a sequence set forth in SEQ ID NO: 46 and the second nucleic acid comprises a sequence set forth in SEQ ID NO: 170. In some embodiments, the first nucleic acid comprises a sequence set forth in SEQ ID NO: 170 and the second nucleic acid comprises a sequence set forth in SEQ ID NO: 29. In some embodiments, the first nucleic acid comprises a sequence set forth in SEQ ID NO: 46 and the second nucleic acid comprises a sequence set forth in SEQ ID NO: 174. In some embodiments, the first nucleic acid comprises a sequence set forth in SEQ ID NO: 44 and the second nucleic acid comprises a sequence set forth in SEQ ID NO: 170. In some embodiments, the first nucleic acid comprises a sequence set forth in SEQ ID NO: 29 and the second nucleic acid comprises a sequence set forth in SEQ ID NO: 174. In some embodiments, the first nucleic acid comprises a sequence set forth in SEQ ID NO: 72 and the second nucleic acid comprises a sequence set forth in SEQ ID NO: 93. In some embodiments, the first nucleic acid comprises a sequence set forth in SEQ ID NO: 93 and the second nucleic acid comprises a sequence set forth in SEQ ID NO: 69. In some embodiments, the first nucleic acid comprises a sequence set forth in SEQ ID NO: 72 and the second nucleic acid comprises a sequence set forth in SEQ ID NO: 94. In some embodiments, the first nucleic acid comprises a sequence set forth in SEQ ID NO: 71 and the second nucleic acid comprises a sequence set forth in SEQ ID NO: 95. In some embodiments, the first nucleic acid comprises a sequence set forth in SEQ ID NO: 95 and the second nucleic acid comprises a sequence set forth in SEQ ID NO: 69. In some embodiments, the first nucleic acid comprises a sequence set forth in SEQ ID NO: 141 and the second nucleic acid comprises a sequence set forth in SEQ ID NO: 94. In some embodiments, the first nucleic acid comprises a sequence set forth in SEQ ID NO: 94 and the second nucleic acid comprises a sequence set forth in SEQ ID NO: 146. In some embodiments, the first nucleic acid comprises a sequence set forth in SEQ ID NO: 94 and the second nucleic acid comprises a sequence set forth in SEQ ID NO: 143. In some embodiments, the first nucleic acid comprises a sequence set forth in SEQ ID NO: 93 and the second nucleic acid comprises a sequence set forth in SEQ ID NO: 146. In some embodiments, the first nucleic acid comprises a sequence set forth in SEQ ID NO: 95 and the second nucleic acid comprises a sequence set forth in SEQ ID NO: 146. In some embodiments, the first nucleic acid comprises a sequence set forth in SEQ ID NO: 141 and the second nucleic acid comprises a sequence set forth in SEQ ID NO: 95. In some embodiments, the first nucleic acid comprises a sequence set forth in SEQ ID NO: 174 and the second nucleic acid comprises a sequence set forth in SEQ ID NO: 141. In some embodiments, the first nucleic acid comprises a sequence set forth in SEQ ID NO: 143 and the second nucleic acid comprises a sequence set forth in SEQ ID NO: 174. In some embodiments, the first nucleic acid comprises a sequence set forth in SEQ ID NO: 146 and the second nucleic acid comprises a sequence set forth in SEQ ID NO: 170. In some embodiments, the first nucleic acid comprises a sequence set forth in SEQ ID NO: 146 and the second nucleic acid comprises a sequence set forth in SEQ ID NO: 178. In some embodiments, the first nucleic acid comprises a sequence set forth in SEQ ID NO: 146 and the second nucleic acid comprises a sequence set forth in SEQ ID NO: 177. In some embodiments, the first nucleic acid comprises a sequence set forth in SEQ ID NO: 143 and the second nucleic acid comprises a sequence set forth in SEQ ID NO: 176. In some embodiments, the first nucleic acid comprises a sequence set forth in SEQ ID NO: 146 and the second nucleic acid comprises a sequence set forth in SEQ ID NO: 176. In some embodiments, the first nucleic acid comprises a sequence set forth in SEQ ID NO: 141 and the second nucleic acid comprises a sequence set forth in SEQ ID NO: 178. In some embodiments, the first nucleic acid comprises a sequence set forth in SEQ ID NO: 143 and the second nucleic acid comprises a sequence set forth in SEQ ID NO: 178. In some embodiments, the first nucleic acid comprises a sequence set forth in SEQ ID NO: 141 and the second nucleic acid comprises a sequence set forth in SEQ ID NO: 177. In some embodiments, the first nucleic acid comprises a sequence set forth in SEQ ID NO: 176 and the second nucleic acid comprises a sequence set forth in SEQ ID NO: 141. In some embodiments, the first nucleic acid comprises a sequence set forth in SEQ ID NO: 174 and the second nucleic acid comprises a sequence set forth in SEQ ID NO: 176. In some embodiments, the first nucleic acid comprises a sequence set forth in SEQ ID NO: 174 and the second nucleic acid comprises a sequence set forth in SEQ ID NO: 177. In some embodiments, the first nucleic acid comprises a sequence set forth in SEQ ID NO: 170 and the second nucleic acid comprises a sequence set forth in SEQ ID NO: 176. In some embodiments, the first nucleic acid comprises a sequence set forth in SEQ ID NO: 170 and the second nucleic acid comprises a sequence set forth in SEQ ID NO: 177.

siRNA molecules and methods of use and production are described in U.S. Pat. No. 7,361,752 and US Patent Publication No. US2005/0048647, both of which are hereby incorporated by reference.

Additional methods and compositions for RNA interference such as shRNA, siRNA, dsRNA, and antisense oligonucleotides are generally known in the art, and are further described in U.S. Pat. Nos. 7,361,752; 8,829,264; 9,556,431; 8,252,526, International PCT Publication No. WO00/44895; International PCT Publication No. WO01/36646; International PCT Publication No. WO99/32619; International PCT Publication No. WO00/01846; International PCT Publication No. WO01/29058; and International PCT Publication No. WO00/44914; International PCT Publication No. WO04/030634; each of which are hereby incorporated by reference.

The nucleic acid sequences (or constructs) that may be used to encode the RNAi molecules, such as an shRNA described herein, may comprise a promoter, which is operably linked (or connected), directly or indirectly, to a sequence encoding the RNAi molecules. Such promoters may be selected based on the host cell and the effect sought. Non-limiting examples of suitable promoters include constitutive and inducible promoters, such as inducible RNA polymerase II (pol II)-based promoters. Non-limiting examples of suitable promoters further include the tetracycline inducible or repressible promoter, EF1a, RNA polymerase I or III-based promoters, the pol II dependent viral promoters, such as the CMV-IE promoter, and the pol III U6 and H1 promoters. The bacteriophage T7 promoter may also be used (in which case it will be appreciated that the T7 polymerase must also be present). The nucleic acid sequences need not be restricted to the use of any single promoter, especially since the nucleic acid sequences may comprise two or more shRNAs (i.e., a combination of effectors), including but not limited to incorporated shRNA molecules. Each incorporated promoter may control one, or any combination of, the shRNA molecule components.

In certain embodiments, the promoter may be preferentially active in the targeted cells, e.g., it may be desirable to preferentially express at least one recombinant nucleic acid in immune cells using an immune cell-specific promoter. Introduction of such constructs into host cells may be effected under conditions whereby the two or more recombinant nucleic acids that are contained within the recombinant nucleic acid precursor transcript initially reside within a single primary transcript, such that the separate RNA molecules (for example, shRNA each comprising its own stem-loop structure) are subsequently excised from such precursor transcript by an endogenous ribonuclease. The resulting mature recombinant nucleic acids (e.g., shRNAs) may then induce degradation, and/or translation repression, of target gene mRNA transcripts produced in the cell. Alternatively, each of the precursor stem-loop structures may be produced as part of a separate transcript, in which case each recombinant nucleic acid sequence will preferably include its own promoter and transcription terminator sequences. Additionally, the multiple recombinant nucleic acid precursor transcripts may reside within a single primary transcript.

The stem-loop structures of the shRNA recombinant nucleic acids described herein may be about 40 to 100 nucleotides long or, preferably, about 50 to 75 nucleotides long. The stem region may be about 15-45 nucleotides in length (or more), or about 20-30 nucleotides in length. In some embodiments, the stem region is 22 nucleotides in length. In some embodiments, the stem region is 15, 16, 17, 18, 19, 20, 21, 22, 23, 24, 25, 26, 27 28 29, 30, 31, 32, 33, 34, 35, 36, 37, 38, 39, 40, 41, 42, 43, 44, or 45 nucleotides in length.

The stem may comprise a perfectly complementary duplex (but for any 3′ tail), however, bulges or interior loops may be present on either arm of the stem. The number of such bulges and asymmetric interior loops are preferably few in number (e.g., 1, 2 or 3) and are about 3 nucleotides or less in size. The terminal loop portion may comprise about 4 or more nucleotides, but preferably not more than about 25. The loop portion will preferably be 6-15 nucleotides in size.

As described herein, the stem regions of the shRNAs comprise passenger strands and guide strands, whereby the guide strands contain sequences complementary to the target mRNA transcript encoded by the target gene(s). Preferably, the G-C content and matching of guide strand and passenger strand is carefully designed for thermodynamically-favorable strand unwind activity with or without endonuclease cleavage. Furthermore, the specificity of the guide strand is preferably confirmed via a BLAST search (www.ncbi.nim.nih.gov/BLAST).

The invention provides that the expression level of multiple target genes may be modulated using the methods and recombinant nucleic acids described herein. For example, the invention provides that a first set of recombinant nucleic acids may be designed to include a sequence (a guide strand) that is designed to reduce the expression level of a first target gene, whereas a second set of recombinant nucleic acids may be designed to include a sequence (a guide strand) that is designed to reduce the expression level of a second target gene. The different sets of recombinant nucleic acids may be expressed and reside within the same, or separate, preliminary transcripts. In certain embodiments, such multiplex approach, i.e., the use of the recombinant nucleic acids described herein to modulate the expression level of two or more target genes, may have an enhanced therapeutic effect on a patient. For example, if a patient is provided with cells expressing the recombinant nucleic acid molecules described herein to treat, prevent, or ameliorate the effects of cancer, it may be desirable to provide the patient with two or more types of recombinant nucleic acid molecules, which are designed to reduce the expression level of multiple genes that are implicated in activation or repression of immune cells.

The recombinant nucleic acid molecule(s) described herein may be capable of reducing target gene expression in a cell by at least more than about 50% as compared to a control cell that does not comprise the recombinant nucleic acid molecule(s). For example, the recombinant nucleic acid molecule(s) (e.g., shRNA) can be capable of reducing expression of a target gene selected from the group consisting of CD5, CBLB, CISH, DGKA, DGKZ, DNMT3A, MAP4K1, NR4A1, PTPN2, TET2, and/or ZC3H12A in the cell by at least 50%, 55%, 60%, 65%, 75%, 80%, 85%, 90%, 95%, 97%, 98%, 99%, or more as compared to a control cell that does not comprise the recombinant nucleic acid molecule(s). The recombinant nucleic acid molecule(s) can be capable of reducing expression of a target gene selected from the group consisting of CD5, CBLB, CISH, DGKA, DGKZ, DNMT3A, MAP4K1, NR4A1, PTPN2, TET2, and/or ZC3H12A in the cell by at least between about 50-100%, 50-99%, 50-95%, 50-90%, 50-85%, 50-80%, 50-75%, 50-70%, 50-65%, 50-60%, 50-55%, or as compared to a control cell that does not comprise the recombinant nucleic acid molecule(s).

The recombinant nucleic acid molecule(s) may be chemically synthesized, or in vitro transcribed, and may further include one or more modifications to phosphate-sugar backbone or nucleosides residues.

Other methods known in the art for introducing nucleic acids to cells may be used, such as lipid-mediated carrier transport, chemical mediated transport, such as calcium phosphate, and the like. Thus, the recombinant nucleic acid molecule(s) construct may be introduced along with components that perform one or more of the following activities: enhance RNA uptake by the cell, promote annealing of the duplex strands for shRNA, stabilize the annealed shRNA strands, or otherwise increase inhibition of the target gene.

In some embodiments, the one or more recombinant nucleic acid(s) further comprises a 5′ homology directed repair arm and/or a 3′ homology directed repair arm complementary to an insertion site in a host cell chromosome. In some embodiments, the one or more recombinant nucleic acid(s) comprises the 5′ homology directed repair arm and the 3′ homology directed repair arm. In some embodiments, the one or more recombinant nucleic acid(s) is incorporated into an expression cassette or an expression vector. In some embodiments, the expression cassette or the expression vector further comprises a constitutive promoter upstream of the one or more recombinant nucleic acid(s).

In some embodiments, the one or more recombinant nucleic acid(s) comprises at least a first nucleic acid and at least a second nucleic acid. The first and second nucleic acids can be RNAi molecules, such as shRNA. In some embodiments, the first nucleic acid and the second nucleic acid are incorporated into a single expression cassette or a single expression vector. In some embodiments, the expression cassette or the expression vector further comprises a constitutive promoter upstream of the first nucleic acid and/or upstream of the second nucleic acid. In some embodiments, the expression vector is a non-viral vector.

### Recombinant Cells

Also provided herein is a recombinant cell comprising a deletion or perturbation of at least a first target gene selected from the group consisting of CD5, CBLB, CISH, DGKA, DGKZ, DNMT3A, MAP4K1, NR4A1, PTPN2, TET2, and/or ZC3H12A. In some embodiments, the cell further comprises deletion or perturbation of at least a second target gene selected from the group consisting of CD5, CBLB, CISH, DGKA, DGKZ, DNMT3A, MAP4K1, NR4A1, PTPN2, TET2, and/or ZC3H12A, and wherein the first target gene and the second target gene are distinct.

In some embodiments, the at least first or second target gene(s) are deleted or perturbed via CRISPR-Cas9 gene editing. In some embodiments, expression of the first or second target gene in the immune cell is reduced by at least 50%, 55%, 60%, 65%, 75%, 80%, 85%, 90%, 95%, or 99% as compared to a control cell that does not comprise the deletion of the first or second target gene.

Also provided herein are cells comprising a first guide RNA, wherein the first guide RNA comprises a sequence set forth in SEQ ID NOs: 12-22. In some embodiments, the cell further comprises a second guide RNA comprising a sequence set forth in SEQ ID NOs: 12-22.

In some embodiments, the cell further comprises a protein comprising a nuclease domain, wherein the nucleic acid and protein form a ribonucleoprotein (RNP) complex. In some embodiments, the nuclease domain comprises a CRISPR-associated endonuclease (Cas), optionally a Cas9 nuclease.

In some embodiments, the first or second nucleic acid reduces expression of CD5, CBLB, CISH, DGKA, DGKZ, DNMT3A, MAP4K1, NR4A1, PTPN2, TET2, and/or ZC3H12A in the immune cell by at least 50%, 55%, 60%, 65%, 75%, 80%, 85%, 90%, 95%, or 99% as compared to a control cell that does not comprise the first nucleic acid. In some embodiments, expression of CD5, CBLB, CISH, DGKA, DGKZ, DNMT3A, MAP4K1, NR4A1, PTPN2, TET2, and/or ZC3H12A in the immune cell is reduced by at least 50%, 55%, 60%, 65%, 75%, 80%, 85%, 90%, 95%, or 99% as compared to a control cell that does not comprise the first or second nucleic acid.

In some embodiments, expression of CD5, CBLB, CISH, DGKA, DGKZ, DNMT3A, MAP4K1, NR4A1, PTPN2, TET2, and/or ZC3H12A is determined by a nucleic acid assay or a protein assay.

Also provided herein is a recombinant cell comprising at least one recombinant nucleic acid(s) non-virally inserted into a target region of the genome of the cell. In some embodiments, the immune cell comprises a first nucleic acid sequence at least 15 nucleotides in length, wherein the first nucleic acid sequence is (1) a nucleic acid sequence at least 15 nucleotides in length complementary to an mRNA encoding human CD5 comprising the sequence set forth in SEQ ID NO: 1; (2) a nucleic acid sequence at least 15 nucleotides in length complementary to an mRNA encoding human CBLB comprising the sequence set forth in SEQ ID NO: 2; (3) a nucleic acid sequence at least 15 nucleotides in length complementary to an mRNA encoding human CISH comprising the sequence set forth in SEQ ID NO: 3; (4) a nucleic acid sequence at least 15 nucleotides in length complementary to an mRNA encoding DGKA comprising the sequence set forth in SEQ ID NO: 4; (5) a nucleic acid sequence at least 15 nucleotides in length complementary to an mRNA encoding DGKZ comprising the sequence set forth in SEQ ID NO: 5; (6) a nucleic acid sequence at least 15 nucleotides in length complementary to an mRNA encoding DNMT3A comprising the sequence set forth in SEQ ID NO: 6; (7) a nucleic acid sequence at least 15 nucleotides in length complementary to an mRNA encoding MAP4K1 comprising the sequence set forth in SEQ ID NO: 7; (9) a nucleic acid sequence at least 15 nucleotides in length complementary to an mRNA encoding TET2 comprising the sequence set forth in SEQ ID NO: 10; (10) a nucleic acid sequence at least 15 nucleotides in length complementary to an mRNA encoding human Protein Tyrosine Phosphatase Non-Receptor Type 2 (PTPN2) comprising the sequence set forth in SEQ ID NO: 9; (11) a nucleic acid sequence at least 15 nucleotides in length complementary to an mRNA encoding NR4A1 comprising the sequence set forth in SEQ ID NO: 8; or (12) a nucleic acid sequence at least 15 nucleotides in length complementary to an mRNA encoding ZC3H12A comprising the sequence set forth in SEQ ID NO: 11.

In some embodiments, the cell is an immune cell. In some embodiments, the immune cell is a primary human immune cell. The primary immune cell is a natural killer (NK) cell, a natural killer T (NKT) cell, a T cell, a γδ T cell, a CD8+ T cell, a CD4+ T cell, a primary T cell, a T cell progenitor, or an induced pluripotent stem cell (iPSC). In some embodiments, the primary immune cell is a primary T cell. In some embodiments, the primary immune cell is a primary human T cell. In some embodiments, the immune cell is virus-free. In some embodiments, the immune cell is a viable, virus-free, primary cell. In some embodiments, the immune cell is an autologous immune cell. In some embodiments, the immune cell is an allogeneic immune cell.

In some embodiments, the expression of the gene targeted (e.g., CD5, CBLB, CISH, DGKA, DGKZ, DNMT3A, MAP4K1, NR4A1, PTPN2, TET2, and/or ZC3H12A) by the recombinant nucleic acid molecule(s) is reduced or decreased in the target cell. The target gene expression can be reduced by at least about 50%, 55%, 60%, 65%, 70%, 75%, 80%, 85%, 90%, 95%, 99%, or more. The target gene expression can be reduced by between about 50-100%, 50-99%, 50-95%, 50-90%, 50-85%, 50-80%, 50-75%, 50-70%, 50-65%, 50-60%, 50-55%, or as compared to a control cell that does not comprise the recombinant nucleic acid molecule(s).

A cell comprising a recombinant nucleic acid molecule(s) insert at a target locus or safe harbor site as described in the present disclosure can be referred to as an engineered cell. In some embodiments, the immune cell is any cell that can give rise to a pluripotent immune cell. In some embodiments, the immune cell can be an induced pluripotent stem cell (iPSC) or a human pluripotent stem cell (HSPC). In some embodiments, the immune cell comprises primary hematopoietic cells or primary hematopoietic stem cells. In some embodiments, that engineered cell is a stem cell, a human cell, a primary cell, an hematopoietic cell, an adaptive immune cell, an innate immune cell, a natural killer (NK) cell, a T cell, a CD8+ cell, a CD4+ cell, or a T cell progenitor. In some embodiments, the immune cells are T cells. In some embodiments, the T cells are regulatory T cells, effector T cells, or naïve T cells. In some embodiments, the T cells are CD8⁺ T cells. In some embodiments, the T cells are CD4⁺ T cells. In some embodiments, the T cells are CD4⁺CD8⁺ T cells.

In some embodiments, the engineered cell is a stem cell, a human cell, a primary cell, an hematopoietic cell, an adaptive immune cell, an innate immune cell, a T cell or a T cell progenitor. Non-limiting examples of immune cells that are contemplated in the present disclosure include T cell, B cell, natural killer (NK) cell, NKT/INKT cell, macrophage, myeloid cell, and dendritic cells. Non-limiting examples of stem cells that are contemplated in the present disclosure include pluripotent stem cells (PSCs), embryonic stem cells (ESCs), induced pluripotent stem cells (iPSCs), embryo-derived embryonic stem cells obtained by nuclear transfer (ntES; nuclear transfer ES), male germline stem cells (GS cells), embryonic germ cells (EG cells), hematopoietic stem/progenitor stem cells (HSPCs), somatic stem cells (adult stem cells), hemangioblasts, neural stem cells, mesenchymal stem cells and stem cells of other cells (including osteocyte, chondrocyte, myocyte, cardiac myocyte, neuron, tendon cell, adipocyte, pancreocyte, hepatocyte, nephrocyte and follicle cells and so on). In some embodiments, the engineered cells is a T cell, NK cells, iPSC, and HSPC. In some embodiments, the engineered cells used in the present disclosure are human cell lines grown in vitro (e.g. deliberately immortalized cell lines, cancer cell lines, etc.).

In some embodiments, the immune cell is an autologous immune cell. In some embodiments, the immune cell is an allogeneic immune cell.

Also provided herein are populations of cells comprising a plurality of the primary immune cell. In some embodiments, the genome of at least 20%, 30%, 40%, 50%, 60%, 70%, 80%, 90%, 95%, 99% or greater of the cells comprises at least one a recombinant nucleic acid molecule(s). In some embodiments, the genome of at least 20%, 30%, 40%, 50%, 60%, 70%, 80%, 90%, 95%, 99% or greater of the cells comprises at least two shRNA molecules. In some embodiments, the genome of at least 20%, 30%, 40%, 50%, 60%, 70%, 80%, 90%, 95%, 99% or greater of the cells comprises at least three, four, five, six, seven, eight, nine, ten or more a recombinant nucleic acid molecule(s).

Also provided herein are populations of cells comprising the recombinant nucleic acid(s).

The cell can further comprise chimeric proteins such as T cell receptors, (TCR), chimeric antigen receptors (CAR) or priming receptors. In some embodiments, the cell comprises at least one T cell receptor (TCR). In some embodiments, the cell comprises at least one chimeric antigen receptor. In some embodiments, the cell comprises at least one priming receptor. In some embodiments, the cell comprises at least one chimeric antigen receptor and at least one priming receptor. The at least one recombinant nucleic acid molecule(s) encoding at least one RNAi molecule can encoded on the same DNA template or nucleic acid fragment as the at least one RNAi molecule(s) or on a different DNA template or nucleic acid fragment as the RNAi molecule(s).

In the case that the TCR and RNAi recombinant nucleic acid molecule(s) are encoded on the same DNA template or nucleic acid fragment, the various components can be placed in any order on the DNA template. For example, the DNA template may comprise, in a 5′ to 3′ direction: the TCR and the at least one RNAi recombinant nucleic acid. Alternatively, the DNA template may comprise, in a 5′ to 3′ direction: the at least one RNAi recombinant nucleic acid and the TCR.

In the case that the CAR, priming receptor, and RNAi recombinant nucleic acid molecule(s) are encoded on the same DNA template or nucleic acid fragment, the various components can be placed in any order on the DNA template. For example, the DNA template may comprise, in a 5′ to 3′ direction: the CAR, the at least one RNAi recombinant nucleic acid, and the priming receptor. Alternatively, the DNA template may comprise, in a 5′ to 3′ direction: i) the priming receptor, the at least one RNAi recombinant nucleic acid, and the CAR; ii) the at least one RNAi recombinant nucleic acid, the priming receptor, and the CAR; iii) the at least one RNAi recombinant nucleic acid, the CAR, and the priming receptor; iv) the priming receptor, the CAR, and the at least one RNAi recombinant nucleic acid; v) the CAR, the priming receptor, and the at least one RNAi recombinant nucleic acid; vi) the at least one RNAi recombinant nucleic acid, the priming receptor, the CAR; vii) the at least one RNAi recombinant nucleic acid, the CAR, and the priming receptor. In some embodiments, the at least one RNAi recombinant nucleic acid comprises two recombinant nucleic acids. In some embodiments, the recombinant nucleic acid comprises a nucleic acid that is complementary to one or more of CD5, CBLB, CISH, DGKA, DGKZ, DNMT3A, MAP4K1, NR4A1, PTPN2, TET2, and/or ZC3H12A.

In some embodiments, the priming receptor comprises a first extracellular antigen-binding domain that specifically binds to a first antigen and the chimeric antigen receptor (CAR) comprises a second extracellular antigen-binding domain that specifically binds to a second antigen.

### Methods of Reducing Gene Expression

Another aspect of the invention provides a method for attenuating expression of a target gene in mammalian cells, comprising introducing into the mammalian cells at least a first recombinant nucleic acid complementary to the target gene mRNA, such as a guide RNA, and a ribonucleoprotein (RNP) comprising a nuclease domain. In some embodiments, the guide RNA specifically hybridizes to at least a first target gene of the primary immune cell, and wherein the nuclease domain cleaves the target region to create a double stranded break site in the genome of the immune cell. In some embodiments, the nuclease domain comprises a CRISPR-associated endonuclease (Cas), optionally a Cas9 nuclease. In some embodiments, the at least a first target gene is one or more of CD5, CBLB, CISH, DGKA, DGKZ, DNMT3A, MAP4K1, NR4A1, PTPN2, TET2, and/or ZC3H12A. In some embodiments, the target gene is CD5. In some embodiments, the at least a first target gene is CBLB. In some embodiments, the at least a first target gene is CISH. In some embodiments, the at least a first target gene is DGKA. In some embodiments, the at least a first target gene is DGKZ. In some embodiments, the at least a first target gene is DNMT3A. In some embodiments, the at least a first target gene is FAS. In some embodiments, the at least a first target gene is MAP4K1. In some embodiments, the at least a first target gene is NR4A1. In some embodiments, the at least a first target gene is PTPN2. In some embodiments, the at least a first target gene is TET2. In some embodiments, the at least a first target gene is TOX. In some embodiments, the at least a first target gene is ZC3H12A.

In some embodiments, the method comprises introducing into the mammalian cells at least a second recombinant nucleic acid complementary to at least a second target gene mRNA, such as a guide RNA, and a ribonucleoprotein (RNP) comprising a nuclease domain. In some embodiments, the at least a second target gene is one or more of CD5, CBLB, CISH, DGKA, DGKZ, DNMT3A, MAP4K1, NR4A1, PTPN2, TET2, and/or ZC3H12A. In some embodiments, the at least a second target gene is CD5. In some embodiments, the target gene is CBLB. In some embodiments, the at least a second target gene is CISH. In some embodiments, the at least a second target gene is DGKA. In some embodiments, the at least a second target gene is DGKZ. In some embodiments, the at least a second target gene is DNMT3A. In some embodiments, the at least a second target gene is FAS. In some embodiments, the at least a second target gene is MAP4K1. In some embodiments, the at least a second target gene is NR4A1. In some embodiments, the at least a second target gene is PTPN2. In some embodiments, the at least a second target gene is TET2. In some embodiments, the at least a second target gene is TOX. In some embodiments, the at least a second target gene is ZC3H12A.

Another aspect of the invention provides a method for attenuating expression of a target gene in mammalian cells, comprising introducing into the mammalian cells a recombinant nucleic acid complementary to the target gene mRNA, such as a single-stranded hairpin ribonucleic acid (shRNA), siRNA, dsRNA, or antisense oligonucleotide. In some embodiments, the recombinant nucleic acid complementary to the target gene mRNA is an shRNA. In some embodiments, the shRNA comprises self-complementary sequences of 19 to 100 nucleotides that form a duplex region, which self-complementary sequences hybridize under intracellular conditions to a target gene mRNA transcript. In some embodiments, the shRNA comprises self-complementary sequences of 22 nt. In some embodiments, the shRNA: (i) is a substrate for cleavage by a RNaseIII enzyme to produce a double-stranded RNA product, (ii) does not produce a general sequence-independent killing of the mammalian cells, and (iii) reduces expression of said target gene in a manner dependent on the sequence of said complementary regions. In some embodiments, the target gene is one or more of CD5, CBLB, CISH, DGKA, DGKZ, DNMT3A, MAP4K1, NR4A1, PTPN2, TET2, and/or ZC3H12A.

The immune cell comprising the recombinant nucleic acid can have reduced or decreased expression of a target gene selected from the group consisting one or more of CD5, CBLB, CISH, DGKA, DGKZ, DNMT3A, MAP4K1, NR4A1, PTPN2, TET2, and/or ZC3H12A. In some embodiments, the immune cell has reduced one or more of CD5, CBLB, CISH, DGKA, DGKZ, DNMT3A, MAP4K1, NR4A1, PTPN2, TET2, and/or ZC3H12A expression of between about 50-100%, 50-99%, 50-95%, 50-90%, 50-85%, 50-80%, 50-75%, 50-70%, 50-65%, 50-60%, 50-55%, as compared to a control cell that does not comprise the recombinant nucleic acid molecule(s).

In some embodiments, expression of one or more of CD5, CBLB, CISH, DGKA, DGKZ, DNMT3A, MAP4K1, NR4A1, PTPN2, TET2, and/or ZC3H12A in the immune cell is reduced by at least 50%, 55%, 60%, 65%, 75%, 80%, 85%, 90%, 95%, or 99% as compared to a control cell that does not comprise the recombinant nucleic acid(s), first nucleic acid, or second nucleic acid.

Another aspect of the invention provides a method for attenuating expression of a target gene in mammalian cells, comprising introducing into the mammalian cells a recombinant nucleic acid complementary to the target gene mRNA. In some embodiments, the recombinant nucleic acid is a guide RNA. In some embodiments, the recombinant nucleic acid further comprises a protein comprising a nuclease domain, wherein the nucleic acid and protein form a ribonucleoprotein (RNP) complex. In some embodiments, the nuclease domain comprises a CRISPR-associated endonuclease (Cas), optionally a Cas9 nuclease. In some embodiments, the ribonucleoprotein (RNP) complex reduces expression of one or more of CD5, CBLB, CISH, DGKA, DGKZ, DNMT3A, MAP4K1, NR4A1, PTPN2, TET2, and/or ZC3H12A in a cell by at least 50%, 55%, 60%, 65%, 75%, 80%, 85%, 90%, 95%, or 99% as compared to a control cell that does not comprise the RNP complex.

In some embodiments, the one or more recombinant nucleic acids comprises a sequence set forth in SEQ ID NO: 12 and a sequence set forth in SEQ ID NO:13. In some embodiments, the one or more recombinant nucleic acids comprises a sequence set forth in SEQ ID NO: 21 and a sequence set forth in SEQ ID NO: 13. In some embodiments, the one or more recombinant nucleic acids comprises a sequence set forth in SEQ ID NO: 21 and a sequence set forth in SEQ ID NO: 12. In some embodiments, the one or more recombinant nucleic acids comprises a sequence set forth in SEQ ID NO: 12 and a sequence set forth in SEQ ID NO: 14.

In some embodiments, expression of one or more of CD5, CBLB, CISH, DGKA, DGKZ, DNMT3A, MAP4K1, NR4A1, PTPN2, TET2, and/or ZC3H12A is determined by a nucleic acid assay or a protein assay. In some embodiments, the nucleic acid assay comprises at least one of polymerase chain reaction (PCR), quantitative PCR (qPCR), RT-qPCR, microarray, gene array, or RNAseq.

### Method of Treating Cancer

In another aspect, the invention provides methods of treating an immune-related condition (e.g., cancer) in an individual comprising administering to the individual an effective amount of a composition comprising a cell comprising a deletion or perturbation in at least one gene selected from the group consisting of CD5, CBLB, CISH, DGKA, DGKZ, DNMT3A, MAP4K1, NR4A1, PTPN2, TET2, and ZC3H12A.

In another aspect, the invention provides methods of treating an immune-related condition (e.g., cancer) in an individual comprising administering to the individual an effective amount of a composition comprising a cell comprising at least one sequence as set forth in SEQ ID NOs: 12-22.

In another aspect, the invention provides methods of treating an immune-related condition (e.g., cancer) in an individual comprising administering to the individual an effective amount of a composition comprising a cell comprising at least one recombinant nucleic acid that comprises a nucleic acid sequence at least 15 nucleotides in length complementary to a target selected from the group consisting of one or more of CD5, CBLB, CISH, DGKA, DGKZ, DNMT3A, MAP4K1, NR4A1, PTPN2, TET2, and/or ZC3H12A. In some embodiments, the recombinant nucleic acid is an shRNA molecule. In some embodiments, the shRNA is selected from the group consisting of a CD5, CBLB, CISH, DGKA, DGKZ, DNMT3A, MAP4K1, NR4A1, PTPN2, TET2, and/or ZC3H12A shRNA molecule. In some embodiments, the cell comprises at least a CD5, CBLB, CISH, DGKA, DGKZ, DNMT3A, MAP4K1, NR4A1, PTPN2, TET2, and/or ZC3H12A shRNA molecule.

In another aspect, the invention provides methods of enhancing an immune response in an individual comprising administering to the individual an effective amount of a composition comprising a cell comprising at least one shRNA molecule, wherein the shRNA molecule is complementary to an mRNA encoding a protein selected from the group consisting of one or more CD5, CBLB, CISH, DGKA, DGKZ, DNMT3A, MAP4K1, NR4A1, PTPN2, TET2, and ZC3H12A shRNA molecules. In some embodiments, the cell comprises at least one shRNA molecule comprising a nucleotide sequence set forth in any one of SEQ ID NOs: 23-207. In some embodiments, the cell comprises an shRNA complementary to an mRNA encoding CD5 and comprising a nucleotide sequence set forth in any one of SEQ ID NOs: 47-72. In some embodiments, the cell comprises an shRNA complementary to an mRNA encoding CBLB and comprising a nucleotide sequence set forth in any one of SEQ ID NOs: 23-46. In some embodiments, the cell comprises an shRNA complementary to an mRNA encoding CISH and comprising a nucleotide sequence set forth in any one of SEQ ID NOs: 73-95. In some embodiments, the cell comprises an shRNA complementary to an mRNA encoding DGKA and comprising a nucleotide sequence set forth in any one of SEQ ID NOs: 181-204. In some embodiments, the cell comprises an shRNA complementary to an mRNA encoding DNMT3A and comprising a nucleotide sequence set forth in any one of SEQ ID NOs: 96-122. In some embodiments, the cell comprises an shRNA complementary to an mRNA encoding TET2 and comprising a nucleotide sequence set forth in any one of SEQ ID NOs: 147-175. In some embodiments, the cell comprises an shRNA complementary to an mRNA encoding PTPN2 and comprising a nucleotide sequence set forth in any one of SEQ ID NOs: 123-146. In some embodiments, the cell comprises an shRNA complementary to an mRNA encoding ZC3H12A and comprising a nucleotide sequence set forth in any one of SEQ ID NOs: 176-180 and 205-207.

In some embodiments, the methods provided herein are useful for the treatment of an immune-related condition in an individual. In one embodiment, the individual is a human.

In some embodiments, the methods provided herein (such as methods of enhancing an immune response) are useful for the treatment of cancer and as such an individual receiving the system described herein has cancer. In some embodiments, the cancer is a solid cancer. In some embodiments, the cancer is a liquid cancer. In some embodiments, the cancer is immunoevasive. In some embodiments, the cancer is immunoresponsive. In particular embodiments, the breast cancer, HER2-positive breast cancer, estrogen-receptor positive breast cancer, progesterone-receptor positive breast cancer, HER2-/estrogen-receptor-/progesterone-receptor-negative breast cancer, triple negative breast cancer, non-small cell lung cancer (NSCLC), lung adenocarcinoma, lung squamous cell carcinoma, lung adenosquamous carcinoma, prostate cancer, castration-resistant prostate cancer, colon cancer, rectal cancer, microsatellite instable (MSI) colon cancer, non-MSI colon cancer, or non-MSI or rectal cancer.

In some embodiments, the treatment results in a decrease in the cancer volume or size. In some embodiments, the treatment is effective at reducing a cancer volume as compared to the cancer volume prior to administration of the recombinant nucleic acid or recombinant cell. In some embodiments, the treatment results in a decrease in the cancer growth rate. In some embodiments, the treatment is effective at reducing a cancer growth rate as compared to the cancer growth rate prior to administration of the or recombinant cell. In some embodiments, the treatment is effective at eliminating the cancer.

### Method of Immune Modulation

Methods of administration of a cell comprising a deletion or perturbation in at least one gene selected from the group consisting of CD5, CBLB, CISH, DGKA, DGKZ, DNMT3A, MAP4K1, NR4A1, PTPN2, TET2, and ZC3H12A can result in modulation of an immune response. Modulation can be an increase or decrease in an immune response. In some embodiments, modulation is an increase in an immune response.

Methods of administration of a cell comprising at least one sequence as set forth in SEQ ID NOs: 12-22 can result in modulation of an immune response. Modulation can be an increase or decrease in an immune response. In some embodiments, modulation is an increase in an immune response.

Methods of administration of a cell comprising a recombinant nucleic acid comprising a nucleic acid sequence at least 15 nucleotides in length complementary to CD5, CBLB, CISH, DGKA, DGKZ, DNMT3A, MAP4K1, NR4A1, PTPN2, TET2, and ZC3H12A can result in modulation of an immune response. In some embodiments, the cell comprises at least one shRNA molecule comprising a nucleotide sequence set forth in any one of SEQ ID NOs: 23-207. In some embodiments, the cell comprises an shRNA complementary to an mRNA encoding CD5 and comprising a nucleotide sequence set forth in any one of SEQ ID NOs: 47-72. In some embodiments, the cell comprises an shRNA complementary to an mRNA encoding CBLB and comprising a nucleotide sequence set forth in any one of SEQ ID NOs: 23-46. In some embodiments, the cell comprises an shRNA complementary to an mRNA encoding CISH and comprising a nucleotide sequence set forth in any one of SEQ ID NOs: 73-95. In some embodiments, the cell comprises an shRNA complementary to an mRNA encoding DGKA and comprising a nucleotide sequence set forth in any one of SEQ ID NOs: 181-204. In some embodiments, the cell comprises an shRNA complementary to an mRNA encoding DNMT3A and comprising a nucleotide sequence set forth in any one of SEQ ID NOs: 96-122. In some embodiments, the cell comprises an shRNA complementary to an mRNA encoding TET2 and comprising a nucleotide sequence set forth in any one of SEQ ID NOs: 147-175. In some embodiments, the cell comprises an shRNA complementary to an mRNA encoding PTPN2 and comprising a nucleotide sequence set forth in any one of SEQ ID NOs: 123-146. In some embodiments, the cell comprises an shRNA complementary to an mRNA encoding ZC3H12A and comprising a nucleotide sequence set forth in any one of SEQ ID NOs: 176-180 and 205-207. Modulation can be an increase or decrease in an immune response. In some embodiments, modulation is an increase in an immune response.

In one aspect, administration of a cell as described herein can result in induction of pro-inflammatory molecules, such as cytokines or chemokines. In some embodiments, the cytokine is IFNg. Generally, induced pro-inflammatory molecules are present at levels greater than that achieved with isotype control. Such pro-inflammatory molecules in turn result in activation of anti-tumor immunity, including, but not limited to, T cell activation, T cell proliferation, T cell differentiation, M1-like macrophage activation, and NK cell activation. Thus, the administration of a system comprising a recombinant nucleic acid as disclosed herein or a deletion or perturbation of a target gene as described herein can induce multiple anti-tumor immune mechanisms that lead to tumor destruction.

In another aspect, provided herein are methods of increasing an immune response in an individual comprising administering to the individual an effective amount of a cell comprising at least one sequence as set forth in SEQ ID NOs: 12-22.

In another aspect, provided herein are methods of increasing an immune response in an individual comprising administering to the individual an effective amount of a cell comprising a deletion of at least a first target gene selected from the group consisting of CD5, CBLB, CISH, DGKA, DGKZ, DNMT3A, MAP4K1, NR4A1, PTPN2, TET2, and ZC3H12A. In some embodiments, the cell further comprises deletion of at least a second target gene selected from the group consisting of CD5, CBLB, CISH, DGKA, DGKZ, DNMT3A, MAP4K1, NR4A1, PTPN2, TET2, and ZC3H12A, and wherein the first target gene and the second target gene are distinct.

In another aspect, provided herein are methods of increasing an immune response in an individual comprising administering to the individual an effective amount of a cell comprising a recombinant nucleic acid comprising a first nucleic acid sequence at least 15 nucleotides in length complementary to CD5, CBLB, CISH, DGKA, DGKZ, DNMT3A, MAP4K1, NR4A1, PTPN2, TET2, and ZC3H12A. In some embodiments, the method of increasing an immune response in a subject comprises administering to the subject a cell comprising a recombinant nucleic acid comprising a second nucleic acid sequence at least 15 nucleotides in length complementary to CD5, CBLB, CISH, DGKA, DGKZ, DNMT3A, MAP4K1, NR4A1, PTPN2, TET2, and ZC3H12A.

In some embodiments, the cell is present in a pharmaceutical composition further comprising a pharmaceutically acceptable excipient.

In any and all aspects of increasing an immune response as described herein, any increase or decrease or alteration of an aspect of characteristic(s) or function(s) is as compared to a cell not comprising a recombinant nucleic acid comprising a nucleic acid sequence at least 15 nucleotides in length complementary to CD5, CBLB, CISH, DGKA, DGKZ, DNMT3A, MAP4K1, NR4A1, PTPN2, TET2, and ZC3H12A or a cell not comprising a deletion in the target gene (e.g, comprises a functional target gene).

Increasing an immune response can be both enhancing an immune response or inducing an immune response. For instance, increasing an immune response encompasses both the start or initiation of an immune response, or ramping up or amplifying an on-going or existing immune response. In some embodiments, the treatment induces an immune response. In some embodiments, the induced immune response is an adaptive immune response. In some embodiments, the induced immune response is an innate immune response. In some embodiments, the treatment enhances an immune response. In some embodiments, the enhanced immune response is an adaptive immune response. In some embodiments, the enhanced immune response is an innate immune response. In some embodiments, the treatment increases an immune response. In some embodiments, the increased immune response is an adaptive immune response. In some embodiments, the increased immune response is an innate immune response. In some embodiments, the immune response is started or initiated by administration of a cell comprising a recombinant nucleic acid comprising at least one nucleic acid sequence at least 15 nucleotides in length complementary to CD5, CBLB, CISH, DGKA, DGKZ, DNMT3A, MAP4K1, NR4A1, PTPN2, TET2, and ZC3H12A. In some embodiments, the immune response is enhanced by administration of cell comprising at least one recombinant nucleic acid comprising a nucleic acid sequence at least 15 nucleotides in length complementary to CD5, CBLB, CISH, DGKA, DGKZ, DNMT3A, MAP4K1, NR4A1, PTPN2, TET2, and ZC3H12A. In some embodiments, the immune response is started or initiated by administration of a cell comprising a deletion of at least a second target gene selected from the group consisting of CD5, CBLB, CISH, DGKA, DGKZ, DNMT3A, MAP4K1, NR4A1, PTPN2, TET2, and ZC3H12A. In some embodiments, the immune response is started or initiated by administration of a cell comprising at least one sequence as set forth in SEQ ID NOs: 12-22 and a protein comprising a nuclease domain, wherein the nucleic acid and protein form a ribonucleoprotein (RNP) complex.

In another aspect, the present application provides methods of genetically editing a cell with a recombinant nucleic acid comprising at least one nucleic acid sequence at least 15 nucleotides in length complementary to an mRNA encoding a protein selected from the group consisting of CD5, CBLB, CISH, DGKA, DGKZ, DNMT3A, MAP4K1, NR4A1, PTPN2, TET2, and ZC3H12A, which results in the modulation of the immune function of the cell. The modulation can be increasing an immune response. In some embodiments, the modulation is an increase in immune function. In some embodiments, the modulation of function leads to the activation of a cell comprising the recombinant nucleic acid comprising at least one nucleic acid sequence at least 15 nucleotides in length complementary to an mRNA encoding a protein selected from the group consisting of CD5, CBLB, CISH, DGKA, DGKZ, DNMT3A, MAP4K1, NR4A1, PTPN2, TET2, and ZC3H12A. In some embodiments, the cell comprises at least one shRNA molecule comprising a nucleotide sequence set forth in any one of SEQ ID NOs: 23-207. In some embodiments, the cell comprises an shRNA complementary to an mRNA encoding CD5 and comprising a nucleotide sequence set forth in any one of SEQ ID NOs: 47-72. In some embodiments, the cell comprises an shRNA complementary to an mRNA encoding CBLB and comprising a nucleotide sequence set forth in any one of SEQ ID NOs: 23-46. In some embodiments, the cell comprises an shRNA complementary to an mRNA encoding CISH and comprising a nucleotide sequence set forth in any one of SEQ ID NOs: 73-95. In some embodiments, the cell comprises an shRNA complementary to an mRNA encoding DGKA and comprising a nucleotide sequence set forth in any one of SEQ ID NOs: 181-204. In some embodiments, the cell comprises an shRNA complementary to an mRNA encoding DNMT3A and comprising a nucleotide sequence set forth in any one of SEQ ID NOs: 96-122. In some embodiments, the cell comprises an shRNA complementary to an mRNA encoding TET2 and comprising a nucleotide sequence set forth in any one of SEQ ID NOs: 147-175. In some embodiments, the cell comprises an shRNA complementary to an mRNA encoding PTPN2 and comprising a nucleotide sequence set forth in any one of SEQ ID NOs: 123-146. In some embodiments, the cell comprises an shRNA complementary to an mRNA encoding ZC3H12A and comprising a nucleotide sequence set forth in any one of SEQ ID NOs: 176-180 and 205-207.

In another aspect, the present application provides methods of genetically editing a cell with a ribonucleoprotein (RNP) comprising a nuclease domain and a guide RNA, wherein the guide RNA comprises a sequence as set forth in SEQ ID NOs: 12-22 which results in the modulation of the immune function of the cell. The modulation can be increasing an immune response. In some embodiments, the modulation is an increase in immune function. In some embodiments, the modulation of function leads to the activation of a cell comprising a deletion or perturbation in at least one of CD5, CBLB, CISH, DGKA, DGKZ, DNMT3A, MAP4K1, NR4A1, PTPN2, TET2, and ZC3H12A.

In some embodiments, the cell is a natural killer (NK) cell, a T cell, a CD8+ T cell, a CD4+ T cell, a primary T cell, or a T cell progenitor.

In some embodiments, the modulation of function of the cells comprising the recombinant nucleic acid(s) as described herein leads to an increase in the cells' abilities to stimulate both native and activated T-cells, for example, by increasing cytokine or chemokine secretion by the cells expressing the recombinant nucleic acid(s). In some embodiments, the modulation of function enhances or increases the cells' ability to produce cytokines, chemokines, CARs, or costimulatory or activating receptors. In some embodiments, the modulation increases the T-cell stimulatory function of the cells expressing the recombinant nucleic acid(s), including, for example, the cells' abilities to trigger T-cell receptor (TCR) signaling, T-cell proliferation, or T-cell cytokine production.

In some embodiments, the increased immune response is secretion of cytokines and chemokines. In some embodiments, the recombinant nucleic acid(s) induces increased expression of at least one cytokine or chemokine in a cell as compared to an isotype control cell.

In some embodiments, the enhanced immune response is anti-tumor immune cell recruitment and activation.

In some embodiments, the cell expressing the recombinant nucleic acid(s) induces a memory immune response as compared to an isotype control cell. In general, a memory immune response is a protective immune response upon a subsequent exposure to pathogens or antigens that the immune system encountered previously. Exemplary memory immune responses include the immune response after infection or vaccination with an antigen. In general, memory immune responses are mediated by lymphocytes such as T cells or B cells. In some embodiments, the memory immune response is a protective immune response to cancer, including cancer cell growth, proliferation, or metastasis. In some embodiments, the memory immune response inhibits, prevents, or reduces cancer cell growth, proliferation, or metastasis.

### Methods of Editing Cells

The terms “gene editing” or “genome editing”, as used herein, refer to a type of genetic manipulation in which DNA is inserted, replaced, or removed from the genome using artificially manipulated nucleases or “molecular scissors”. It is a useful tool for elucidating the function and effect of sequence-specific genes or proteins or altering cell behavior (e.g. for therapeutic purposes).

Currently available genome editing tools include zinc finger nucleases (ZFN) and transcription activator-like effector nucleases (TALENs) to incorporate genes at safe harbor loci (.e.g. the adeno-associated virus integration site 1 (AAVS1) safe harbor locus). The DICE (dual integrase cassette exchange) system utilizing phiC31 integrase and Bxb1 integrase is a tool for target integration. Additionally, clustered regularly interspaced short palindromic repeat/Cas9 (CRISPR/Cas9) techniques can be used for targeted gene insertion.

Site specific gene editing approaches can include homology dependent mechanisms or homology independent mechanisms.

All methods known in the art for targeted insertion of gene sequences are contemplated in the methods described herein to insert constructs at gene targets or safe harbor loci.

Provided herein are methods of inserting one or more recombinant RNAi nucleic acids, in the absence of a viral vector. In some embodiments, the one or more recombinant nucleic acids can be inserted into the genome of a primary immune cell, in the absence of a viral vector

Described herein are methods and compositions for achieving integration of a nucleotide sequence encoding one or more recombinant nucleic acids into the genome of a cell. In some methods the efficiency of integration is increased, off-target effects are reduced and/or loss of cell viability is reduced.

A plasmid encoding one or more recombinant nucleic acids is introduced into an immune cell with a nuclease, such as a CRISPR-associated system (Cas). The nuclease can be introduced in a ribonucleoprotein format with a guide RNA (gRNA) that targets a specific site on the genome of the immune cell. The nuclease cuts the genomic DNA at this specific site. The specific site may be a portion of the genome that encodes an endogenous immune cell receptor. Thus, cutting the genome at this site will cause the immune cell to no longer express an endogenous immune cell receptor.

The plasmid may include 5′ and 3′ homology-directed repair arms complementary to sequences at a specific site on the genome of the immune cell. The complementary sequences are on either side of the site cut by the nuclease, which allows the plasmid to be incorporated at a specified insertion site on the immune cell's genome. Once the plasmid is incorporated, the cell will express the shRNA.

Initially, an immune cell, such as a T cell, is activated. The immune cell may be obtained from a patient. Thus, the present disclosure provides methods in which immune cells, such as T cells, are harvested from a patient. Then, the plasmid that encodes the one or more recombinant nucleic acids is introduced into a T cell. Advantageously, the plasmids of the present disclosure can be introduced using electroporation. When introducing the plasmid via electroporation, the nuclease may also be introduced. By using electroporation, methods of the present disclosure avoid the use of viral vectors for introducing transgenes, which is a known bottleneck in immune cell engineering. The immune cells are then expanded and co-cultured to create a sufficient quantity of engineered immune cells to be used as a therapeutic treatment.

Methods for editing the genome of a cell can include a) providing a Cas9 ribonucleoprotein complex (RNP)-DNA template complex comprising: (i) the RNP, wherein the RNP comprises a Cas9 nuclease domain and a guide RNA, wherein the guide RNA specifically hybridizes to a target region of the genome of the cell, and wherein the Cas9 nuclease domain cleaves the target region to create an insertion site in the genome of the cell; and (ii) a double-stranded or single-stranded DNA template, wherein the 5′ and 3′ ends of the DNA template comprise nucleotide sequences that are homologous to genomic sequences flanking the insertion site, and wherein the molar ratio of RNP to DNA template in the complex is from about 3:1 to about 100:1; and b) introducing the RNP-DNA template complex into the cell.

In some embodiments, the methods described herein provide an efficiency of delivery of the RNP-DNA template complex of at least about 20%, 25%, 30%, 35%, 40%, 45%, 50%, 55%, 60%, 65%, 70%, 75%, 80%, 85%, 90%, 95%, 97.5%, 99%, 99.5%, 99%, or higher. In some cases, the efficiency is determined with respect to cells that are viable after introducing the RNP-DNA template into the cell. In some cases, the efficiency is determined with respect to the total number of cells (viable or non-viable) in which the RNP-DNA template is introduced into the cell.

As another example, the efficiency of delivery can be determined by quantifying the number of genome edited cells in a population of cells (as compared to total cells or total viable cells obtained after the introducing step). Various methods for quantifying genome editing can be utilized. These methods include, but are not limited to, the use of a mismatch-specific nuclease, such as T7 endonuclease I; sequencing of one or more target loci (e.g., by sanger sequencing of cloned target locus amplification fragments); and high-throughput deep sequencing.

In some embodiments, loss of cell viability is reduced as compared to loss of cell viability after introduction of naked DNA into a cell or introduction of DNA into a cell using a viral vector. The reduction can be a reduction of at least 10%, 20%, 30%, 40%, 50%, 60%, 70%, 80%, 90%, 100% or any percentage in between these percentages. In some embodiments, off-target effects of integration are reduced as compared to off-target integration after introduction of naked DNA into a cell or introduction of DNA into a cell using a viral vector. The reduction can be a reduction of at least 10%, 20%, 30%, 40%, 50%, 60%, 70%, 80%, 90%, 100% or any percentage in between these percentages.

In some cases, the methods described herein provide for high cell viability of cells to which the RNP-DNA template has been introduced. In some cases, the viability of the cells to which the RNP-DNA template has been introduced is at least about 20%, 25%, 30%, 35%, 40%, 45%, 50%, 55%, 60%, 65%, 70%, 75%, 80%, 85%, 90%, 95%, 97.5%, 99%, 99.5%, 99%, or higher. In some cases, the viability of the cells to which the RNP-DNA template has been introduced is from about 20% to about 99%, from about 30% to about 90%, from about 35% to about 85% or 90% or higher, from about 40% to about 85% or 90% or higher, from about 50% to about 85% or 90% or higher, from about 50% to about 85% or 90% or higher, from about 60% to about 85% or 90% or higher, or from about 70% to about 85% or 90% or higher.

In the methods provided herein, the molar ratio of RNP to DNA template can be from about 3:1 to about 100:1. For example, the molar ratio can be from about 5:1 to 10:1, from about 5:1 to about 15:1, 5:1 to about 20:1; 5:1 to about 25:1; from about 8:1 to about 12:1; from about 8:1 to about 15:1, from about 8:1 to about 20:1, or from about 8:1 to about 25:1.

In some embodiments, the DNA template is at a concentration of about 2.5 pM to about 25 pM. For example, the concentration of DNA template can be about 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25 pM or any concentration in between these concentrations.

In some embodiments, the amount of DNA template is about 1 μg to about 10 μg. For example, the amount of DNA template can be about 1 μg to about 2 μg, about 1 μg to about 3 μg, about 1 μg to about 4 μg, about 1 μg to about 5 μg, about 1 μg to about 6 μg, about 1 μg to about 7 μg, about 1 μg to about 8 μg, about 1 μg to about 9 μg, about 1 μg to about 10 μg. In some embodiments the amount of DNA template is about 2 μg to about 3 μg, about 2 μg to about 4 μg, about 2 μg to about 5 μg, about 2 μg to about 6 μg, about 2 μg to about 7 μg, about 2 μg to about 8 μg, about 2 μg to about 9 μg, or 2 μg to about 10 μg. In some embodiments the amount of DNA template is about 3 μg to about 4 μg, about 3 μg to about 5 μg, about 3 μg to about 6 μg, about 3 μg to about 7 μg, about 3 μg to about 8 μg, about 3 μg to about 9 μg, or about 3 μg to about 10 μg. In some embodiments, the amount of DNA template is about 4 μg to about 5 μg, about 4 μg to about 6 μg, about 4 μg to about 7 μg, about 4 μg to about 8 μg, about 4 μg to about 9 μg, or about 4 μg to about 10 μg. In some embodiments, the amount of DNA template is about 5 μg to about 6 μg, about 5 μg to about 7 μg, about 5 μg to about 8 μg, about 5 μg to about 9 μg, or about 5 μg to about 10 μg. In some embodiments, the amount of DNA template is about 6 μg to about 7 μg, about 6 μg to about 8 μg, about 6 μg to about 9 μg, or about 6 μg to about 10 μg. In some embodiments, the amount of DNA template is about 7 μg to about 8 μg, about 7 μg to about 9 μg, or about 7 μg to about 10 μg. In some embodiments, the amount of DNA template is about 8 μg to about 9 μg, or about 8 μg to about 10 μg. In some embodiments, the amount of DNA template is about 9 μg to about 10 μg.

In some embodiments, the DNA template encodes an shRNA molecule or a fragment thereof. In some embodiments, the DNA template encodes at least one shRNA molecule. In some embodiments, the DNA template encodes at least two shRNA molecules. In some embodiments, the DNA template encodes one, two, three, four, five, six, seven, eight, nine, ten, or more shRNA molecules.

In some embodiments, the DNA template includes regulatory sequences, for example, a promoter sequence and/or an enhancer sequence to regulate expression of the heterologous protein or fragment thereof after insertion into the genome of a cell.

In some cases, the DNA template is a linear DNA template. In some cases, the DNA template is a single-stranded DNA template. In some cases, the single-stranded DNA template is a pure single-stranded DNA template. As used herein, by “pure single-stranded DNA” is meant single-stranded DNA that substantially lacks the other or opposite strand of DNA. By “substantially lacks” is meant that the pure single-stranded DNA lacks at least 100-fold more of one strand than another strand of DNA.

In some cases, the RNP-DNA template complex is formed by incubating the RNP with the DNA template for less than about one minute to about thirty minutes, at a temperature of about 20° C. to about 25° C. For example, the RNP can be incubated with the DNA template for about 5 seconds, 10 seconds, 15 seconds, 20 seconds, 25 seconds, 30 seconds, 35 seconds, 40 seconds, 45 seconds, 50 seconds, 55 seconds, 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes or 30 minutes or any amount of time in between these times, at a temperature of about 20° C., 21° C., 22° C., 23° C., 24° C., or 25° C. In another example, the RNP can be incubated with the DNA template for less than about one minute to about one minute, for less than about one minute to about 5 minutes, for less than about 1 minute to about 10 minutes, for about 5 minutes to 10 minutes, for about 5 minutes to 15 minutes, for about 10 to about 15 minutes, for about 10 minutes to about 20 minutes, or for about 10 minutes to about 30 minutes, at a temperature of about 20° C. to about 25° C. In some embodiments, the RNP-DNA template complex and the cell are mixed prior to introducing the RNP-DNA template complex into the cell.

In some embodiments introducing the RNP-DNA template complex comprises electroporation. Methods, compositions, and devices for electroporating cells to introduce a RNP-DNA template complex can include those described in the examples herein. Additional or alternative methods, compositions, and devices for electroporating cells to introduce a RNP-DNA template complex can include those described in WO/2006/001614 or Kim, J. A. et al. Biosens. Bioelectron. 23, 1353-1360 (2008). Additional or alternative methods, compositions, and devices for electroporating cells to introduce a RNP-DNA template complex can include those described in U.S. Patent Appl. Pub. Nos. 2006/0094095; 2005/0064596; or 2006/0087522. Additional or alternative methods, compositions, and devices for electroporating cells to introduce a RNP-DNA template complex can include those described in Li, L. H. et al. Cancer Res. Treat. 1, 341-350 (2002); U.S. Pat. Nos. 6,773,669; 7,186,559; 7,771,984; 7,991,559; 6,485,961; 7,029,916; and U.S. Patent Appl. Pub. Nos: 2014/0017213; and 2012/0088842, all of which are hereby incorporated by reference. Additional or alternative methods, compositions, and devices for electroporating cells to introduce a RNP-DNA template complex can include those described in Geng, T. et al. J. Control Release 144, 91-100 (2010); and Wang, J., et al. Lab. Chip 10, 2057-2061 (2010), all of which are hereby incorporated by reference.

In some embodiments, the Cas9 protein can be in an active endonuclease form, such that when bound to target nucleic acid as part of a complex with a guide RNA or part of a complex with a DNA template, a double strand break is introduced into the target nucleic acid. The double strand break can be repaired by NHEJ to introduce random mutations, or HDR to introduce specific mutations. Various Cas9 nucleases can be utilized in the methods described herein. For example, a Cas9 nuclease that requires an NGG protospacer adjacent motif (PAM) immediately 3′ of the region targeted by the guide RNA can be utilized. Such Cas9 nucleases can be targeted to any region of a genome that contains an NGG sequence. As another example, Cas9 proteins with orthogonal PAM motif requirements can be utilized to target sequences that do not have an adjacent NGG PAM sequence. Exemplary Cas9 proteins with orthogonal PAM sequence specificities include, but are not limited to, CFP1, those described in Nature Methods 10, 1116-1121 (2013), and those described in Zetsche et al., Cell, Volume 163, Issue 3, p759-771, 22 Oct. 2015, both of which are hereby incorporated by reference.

In some cases, the Cas9 protein is a nickase, such that when bound to target nucleic acid as part of a complex with a guide RNA, a single strand break or nick is introduced into the target nucleic acid. A pair of Cas9 nickases, each bound to a structurally different guide RNA, can be targeted to two proximal sites of a target genomic region and thus introduce a pair of proximal single stranded breaks into the target genomic region. Nickase pairs can provide enhanced specificity because off-target effects are likely to result in single nicks, which are generally repaired without lesion by base-excision repair mechanisms. Exemplary Cas9 nickases include Cas9 nucleases having a D10A or H840A mutation.

In some embodiments, the RNP comprises a Cas9 nuclease. In some embodiments, the RNP comprises a Cas9 nickase. In some embodiments, the RNP-DNA template complex comprises at least two structurally different RNP complexes. In some embodiments, the at least two structurally different RNP complexes contain structurally different Cas9 nuclease domains In some embodiments, the at least two structurally different RNP complexes contain structurally different guide RNAs. In some embodiments, wherein the at least two structurally different RNP complexes contain structurally different guide RNAs, each of the structurally different RNP complexes comprises a Cas9 nickase, and the structurally different guide RNAs hybridize to opposite strands of the target region.

In some cases, a plurality of RNP-DNA templates comprising structurally different ribonucleoprotein complexes is introduced into the cell. For example a Cas9 protein can be complexed with a plurality (e.g., 2, 3, 4, 5, or more, e.g., 2-10, 5-100, 20-100) of structurally different guide RNAs to target insertion of a DNA template at a plurality of structurally different target genomic regions.

In the methods and compositions provided herein, cells include, but are not limited to, eukaryotic cells, prokaryotic cells, animal cells, plant cells, fungal cells and the like. Optionally, the cell is a mammalian cell, for example, a human cell. The cell can be in vitro, ex vivo or in vivo. The cell can also be a primary cell, a germ cell, a stem cell or a precursor cell. The precursor cell can be, for example, a pluripotent stem cell, or a hematopoietic stem cell. In some embodiments, the cell is a primary hematopoietic cell or a primary hematopoietic stem cell. In some embodiments, the primary hematopoietic cell is an immune cell. In some embodiments, the immune cell is a T cell. In some embodiments, the T cell is a regulatory T cell, an effector T cell, or a naïve T cell. In some embodiments, the T cell is a CD4⁺ T cell. In some embodiments, the T cell is a CD8⁺ T cell. In some embodiments, the T cell is a CD4⁺CD8⁺ T cell. In some embodiments, the T cell is a CD4⁻CD8⁻ T cell. Populations of any of the cells modified by any of the methods described herein are also provided. In some embodiments, the methods further comprise expanding the population of modified cells.

In some cases, the cells are removed from a subject, modified using any of the methods described herein and administered to the patient. In other cases, any of the constructs described herein is delivered to the patient in vivo. See, for example, U.S. Pat. No. 9,737,604 and Zhang et al. “Lipid nanoparticle-mediated efficient delivery of CRISPR/Cas9 for tumor therapy,” NPG Asia Materials Volume 9, page e441 (2017), both of which are hereby incorporated by reference.

In some embodiments, the RNP-DNA template complex is introduced into about 1×10⁵ to about 2×10⁶ cells. For example, the RNP-DNA template complex can be introduced into about 1×10⁵ to about 5×10⁵ cells, about 1×10⁵ to about 1×10⁶, 1×10⁵ to about 1.5×10⁶, 1×10⁵ to about 2×10⁶, about 1×10⁶ to about 1.5×10⁶ cells or about 1×10⁶ to about 2×10⁶.

In some cases, the methods and compositions described herein can be used for generation, modification, use, or control of recombinant immune cells, such as chimeric antigen receptor T cells (CAR T cells), or T cells expressing priming receptors (primeR) or recombinant T cell receptors (TCR). Such CAR T cells can be used to treat or prevent cancer, an infectious disease, or autoimmune disease in a subject. For example, in some embodiments, one or more gene products are inserted or knocked-in to a T cell to express a heterologous protein (e.g., a chimeric antigen receptor (CAR), a priming receptor, or a T cell receptor (TCR)).

### Insertion Sites

Methods for editing the genome of an immune cell include a method of editing the genome of a human T cell comprise inserting a nucleic acid sequence or construct into a target region in exon 1 of the TCR-α subunit (TRAC) gene in the human immune cell. In some embodiments, the target region is in exon 1 of the constant domain of TRAC gene. In other embodiments, the target region is in exon 1, exon 2 or exon 3, prior to the start of the sequence encoding the TCR-α transmembrane domain.

Methods for editing the genome of an immune cell also include a method of editing the genome of a human immune T cell comprise inserting a nucleic acid sequence or construct into a target region in exon 1 of a TCR-β subunit (TRBC) gene in the human T cell. In some embodiments, the target region is in exon 1 of the TRBC1 or TRBC2 gene.

Methods for editing the genome of an immune cell, specifically, include a method of editing the genome of a human immune cell comprise inserting a nucleic acid sequence or construct into a target region of a genomic safe harbor (GSH).

Methods for editing the genome of a T cell also include a method of editing the genome of a human T cell comprise inserting a nucleic acid sequence or construct into a GS94 target region (locus chr11: 128340000-128350000).

In some embodiments, the target region is the GS94 locus.

Gene editing therapies include, for example, vector integration and site specific integration. Site-specific integration is a promising alternative to random integration of viral vectors, as it mitigates the risks of insertional mutagenesis or insertional oncogenesis (Kolb et al. Trends Biotechnol. 2005 23:399-406; Porteus et al. Nat Biotechnol. 2005 23:967-973; Paques et al. Curr Gen Ther. 2007 7:49-66). However, site specific integration continues to face challenges such as poor knock-in efficiency, risk of insertional oncogenesis, unstable and/or anomalous expression of adjacent genes or the transgene, low accessibility (e.g. within 20 kB of adjacent genes), etc. These challenges can be addressed, in part, through the identification and use of safe harbor loci or safe harbor sites (SHS), which are sites in which genes or genetic elements can be incorporated without disruption to expression or regulation of adjacent genes.

The most widely used of the putative human safe harbor sites is the AAVS1 site on chromosome 19q, which was initially identified as a site for recurrent adenoassociated virus insertion. Other potential SHS have been identified on the basis of homology, with sites first identified in other species (e.g., the human homolog of the permissive murine Rosa26 locus) or among the growing number of human genes that appear non-essential under some circumstances. One putative SHS of this type is the CCR5 chemokine receptor gene, which, when disrupted, confers resistance to human immunodeficiency virus infection. Additional potential genomic SHS have been identified in human and other cell types on the basis of viral integration site mapping or gene-trap analyses, as was the original murine Rosa26 locus. The three top SHS, AAVS1, CCR5, and Rosa26, are in close proximity to many protein coding genes and regulatory elements. (See Sadelain, M., et al. (2012). Safe harbours for the integration of new DNA in the human genome. Nature reviews Cancer, 12 (1), 51-58, the relevant disclosures of which are herein incorporated by reference in their entirety).

The AAVS1 (also known as the PPP1R12C locus) on human chromosome 19 is a known SHS for hosting transgenes (e.g. DNA transgenes) with expected function. It is at position 19q13.42. It has an open chromatin structure and is transcription-competent. The canonical SHS locus for AAVS1 is chr19: 55,625,241-55,629,351. See Pellenz et al. “New Human Chromosomal Sites with “Safe Harbor” Potential for Targeted Transgene Insertion.” Human gene therapy vol. 30, 7 (2019): 814-828, the relevant disclosures of which are herein incorporated by reference. An exemplary AAVS1 target gRNA and target sequence are provided below:

AAVS1-gRNA sequence:
ggggccactagggacaggatGTTTTAGAGCTAGAAATAGCAAGTTAAAA
TAAGGCTAGTCCGTTATCAACTTGAAAAAGTGGCACCGAGTCGGTGCTT
TTTTT
AAVS1 target sequence:
ggggccactagggacaggat

CCR5, which is located on chromosome 3 at position 3p21.31, encodes the major co-receptor for HIV-1. Disruption at this site in the CCR5 gene has been beneficial in HIV/AIDS therapy and prompted the development of zinc-finger nucleases that target its third exon. The canonical SHS locus for CCR5 is chr3: 46,414,443-46,414,942. See Pellenz et al. “New Human Chromosomal Sites with “Safe Harbor” Potential for Targeted Transgene Insertion.” Human gene therapy vol. 30, 7 (2019): 814-828, the relevant disclosures of which are herein incorporated by reference.

The mouse Rosa26 locus is particularly useful for genetic modification as it can be targeted with high efficiency and is expressed in most cell types tested. Irion et al. 2007 (“Identification and targeting of the ROSA26 locus in human embryonic stem cells.” Nature biotechnology 25.12 (2007): 1477-1482, the relevant disclosure of which are herein incorporated by reference) identified the human homolog, human ROSA26, in chromosome 3 (position 3p25.3). The canonical SHS locus for human Rosa26 (hRosa26) is chr3: 9,415,082-9,414,043. See Pellenz et al. “New Human Chromosomal Sites with “Safe Harbor” Potential for Targeted Transgene Insertion.” Human gene therapy vol. 30, 7 (2019): 814-828, the relevant disclosures of which are herein incorporated by reference.

Additional examples of safe harbor sites are provided in Pellenz et al. “New Human Chromosomal Sites with “Safe Harbor” Potential for Targeted Transgene Insertion.” Human gene therapy vol. 30, 7 (2019): 814-828, the relevant disclosures of which are herein incorporated by reference. Examples of additional integration sites are provided in Table D.

In some embodiments, the safe harbor sites allow for high transgene expression (sufficient to allow for transgene functionality or treatment of a disease of interest) and stable expression of the transgene over several days, weeks or months. In some embodiments, knockout of the gene at the safe harbor locus confers benefit to the function of the cell, or the gene at the safe harbor locus has no known function within the cell. In some embodiments the safe harbor locus results in stable transgene expression in vitro with or without CD3/CD28 stimulation, negligible off-target cleavage as detected by iGuide-Seq or CRISPR-Seq, less off-target cleavage relative to other loci as detected by iGuide-Seq or CRISPR-Seq, negligible transgene-independent cytotoxicity, negligible transgene-independent cytokine expression, negligible transgene-independent chimeric antigen receptor expression, negligible deregulation or silencing of nearby genes, and positioned outside of a cancer-related gene.

As used, a “nearby gene” can refer to a gene that is within about 100 kB, about 125 kB, about 150 kB, about 175 kB, about 200 kB, about 225 kB, about 250 kB, about 275 kB, about 300 kB, about 325 kB, about 350 kB, about 375 kB, about 400 kB, about 425 kB, about 450 kB, about 475 kB, about 500 KB, about 525 kB, about 550 kB away from the safe harbor locus (integration site).

In some embodiments, the present disclosure contemplates nucleic acid inserts that comprise one or more recombinant RNAi nucleic acids, such as at least one shRNA molecule. The integration of the one or more recombinant RNAi nucleic acids can result in, for example, enhanced therapeutic properties. These enhanced therapeutic properties, as used herein, refer to an enhanced therapeutic property of a cell when compared to a typical immune cell of the same normal cell type. For example, an NK cell having “enhanced therapeutic properties” has an enhanced, improved, and/or increased treatment outcome when compared to a typical, unmodified and/or naturally occurring NK cell. The therapeutic properties of immune cells can include, but are not limited to, cell transplantation, transport, homing, viability, self-renewal, persistence, immune response control and regulation, survival, and cytotoxicity. The therapeutic properties of immune cells are also manifested by: antigen-targeted receptor expression; HLA presentation or lack thereof; tolerance to the intratumoral microenvironment; induction of bystander immune cells and immune regulation; improved target specificity with reduction; resistance to treatments such as chemotherapy.

As used herein, the term “insert size” refers to the length of the nucleotide sequence being integrated (inserted) at the target locus or safe harbor site.

The inserts of the present disclosure refer to nucleic acid molecules or polynucleotide inserted at a target locus or safe harbor site. In some embodiments, the nucleotide sequence is a DNA molecule, e.g., genomic DNA, or comprises deoxy-ribonucleotides. In some embodiments, the insert comprises a smaller fragment of DNA, such as a plastid DNA, mitochondrial DNA, or DNA isolated in the form of a plasmid, a fosmid, a cosmid, a bacterial artificial chromosome (BAC), a yeast artificial chromosome (YAC), and/or any other sub-genome segment of DNA. The nucleotides in the insert are contemplated as naturally occurring nucleotides, non-naturally occurring, and modified nucleotides. Nucleotides may be modified chemically or biochemically, or may contain non-natural or derivatized nucleotide bases, as will be readily appreciated by those of skill in the art. Such modifications include, for example, labels, methylation, substitution of one or more of the naturally occurring nucleotides with an analog, internucleotide modifications. The polynucleotides can be in any topological conformation, including single-stranded, double-stranded, partially duplexed, triplexed, hairpinned, circular conformations, and other three-dimension conformations contemplated in the art.

The inserts can have coding and/or non-coding regions. The insert can comprises a non-coding sequence (e.g., control elements, e.g., a promoter sequence). In some embodiments, the insert encodes one or more recombinant RNAi nucleic acids.

In some embodiments, the nucleic acid sequence is inserted into the genome of the immune cell via non-viral delivery. In non-viral delivery methods, the nucleic acid can be naked DNA, or in a non-viral plasmid or vector. Non-viral delivery techniques can be site-specific integration techniques, as described herein or known to those of ordinary skill in the art. Examples of site-specific techniques for integration into the safe harbor loci include, without limitation, homology-dependent engineering using nucleases and homology independent targeted insertion using Cas9 or other CRISPR endonucleases.

In some embodiments, the insert is integrated at a safe harbor site by introducing into the engineered cell, (a) a targeted nuclease that cleaves a target region in the safe harbor site to create the insertion site; and (b) the nucleic acid sequence (insert), wherein the insert is incorporated at the insertion site by, e.g., HDR. Examples of non-viral delivery techniques that can be used in the methods of the present disclosure are provided in U.S. application Ser. Nos. 16/568,116 and 16/622,843, the relevant disclosures of which are herein incorporated by reference in their entirety.

Examples of safe harbor integration sites contemplated are provided in Table D.

TABLE D
safe harbor sgRNA sequences

| | | | | | Median (% |
|---|---|---|---|---|---|
| | | | | | Modified), |
| | | | | | summarized |
| | | | sgRNA | | from 2 |
| sgRNA | | sgRNA start | Target | Integration | donors, 2 |
| ID | sgRNA Sequence | coor GRCH38 | Loci | Site | primersets |
| sgRNA_ | GCACCTGAATACCACGC | chr16:88811818 | APRT | APRT | 79.28 |
| 1 | CTG | | | | |
| sgRNA_ | CGCCTGCGATGTAGTCG | chr16:88811551 | APRT | APRT | 78.60 |
| 2 | ATG | | | | |
| sgRNA_ | CAGGACGGGCGAGATGT | chr16:88811640 | APRT | APRT | 85.25 |
| 3 | CCC | | | | |
| sgRNA_ | CTGAATCTTTGGAGTAC | chr15:44715425 | B2M | B2M | 78.51 |
| 4 | CTG | | | | |
| sgRNA_ | GGCCACGGAGCGAGACA | chr15:44711550 | B2M | B2M | 94.75 |
| 5 | TCT | | | | |
| sgRNA_ | AAGTCAACTTCAATGTC | chr15:44715515 | B2M | B2M | 70.97 |
| 6 | GGA | | | | |
| sgRNA_ | GCTTGGAGGCCTGATCA | chr19:36141111 | CAPNS1 | CAPNS1 | 89.34 |
| 7 | GCG | | | | |
| sgRNA_ | CTTATCTCTTCGCAGCGA | chr19:36142301 | CAPNS1 | CAPNS1 | 91.09 |
| 8 | GG | | | | |
| sgRNA_ | CACACATTACTCCAACA | chr19:36142676 | CAPNS1 | CAPNS1 | 71.98 |
| 9 | TTG | | | | |
| sgRNA_ | TTCCGCAAAATAGAGCC | chr3:105746019 | CBLB | CBLB | 91.55 |
| 10 | CCA | | | | |
| sgRNA_ | TGCACAGAACTATCGTA | chr3:105751622 | CBLB | CBLB | 91.43 |
| 11 | CCA | | | | |
| sgRNA_ | GCAATAAGACTCTTTAA | chr3:105853470 | CBLB | CBLB | 76.18 |
| 12 | AGA | | | | |
| sgRNA_ | CAAAGAGATTACGAATG | chr1:116754658 | CD2 | CD2 | 89.80 |
| 13 | CCT | | | | |
| sgRNA_ | CAAGGCACCCCAGGTTT | chr1:116754663 | CD2 | CD2 | 92.70 |
| 14 | CCA | | | | |
| sgRNA_ | TTACGAATGCCTTGGAA | chr1:116754666 | CD2 | CD2 | 92.82 |
| 15 | ACC | | | | |
| sgRNA_ | CAGAGACGCATCTGACC | chr11:118315540 | CD3E | CD3E | 90.96 |
| 16 | CTC | | | | |
| sgRNA_ | CATGCAGTTCTCACACA | chr11:118313715 | CD3E | CD3E | 87.47 |
| 17 | CTG | | | | |
| sgRNA_ | GTGTGAGAACTGCATGG | chr11:118313715 | CD3E | CD3E | 86.65 |
| 18 | AGA | | | | |
| sgRNA_ | TCTCATTTCAGGAAACC | chr11:118349748 | CD3G | CD3G | 87.24 |
| 19 | ACT | | | | |
| sgRNA_ | AGTCATACACCTTAACC | chr11:118349754 | CD3G | CD3G | 87.99 |
| 20 | AAG | | | | |
| sgRNA_ | TTCAAGGAAACCAGTTG | chr11:118352458 | CD3G | CD3G | 86.55 |
| 21 | AGG | | | | |
| sgRNA_ | GAGCCTTGCCTGGAAAT | chr11:61118177 | CD5 | CD5 | 84.03 |
| 22 | CTG | | | | |
| sgRNA_ | AAGCGTCAAAAGTCTGC | chr11:61118324 | CD5 | CD5 | 89.19 |
| 23 | CAG | | | | |
| sgRNA_ | CGTTCCAACTCGAAGTG | chr11:61118121 | CD5 | CD5 | 83.11 |
| 24 | CCA | | | | |
| sgRNA_ | GAGCGACTGGGACACGG | chr9:136866246 | EDF1 | EDF1 | 88.84 |
| 25 | TGA | | | | |
| sgRNA_ | GCTGCGCAAGAAGGGCC | chr9:136866211 | EDF1 | EDF1 | 91.04 |
| 26 | CTA | | | | |
| sgRNA_ | TTGTTCTGGCCAGCAGC | chr9:136863433 | EDF1 | EDF1 | 85.98 |
| 27 | CCC | | | | |
| sgRNA_ | CTTCCAGAGCCACATCA | chr19:48965791 | FTL | FTL | 93.10 |
| 28 | TCG | | | | |
| sgRNA_ | GGGACTCACCAGAGAGA | chr19:48965601 | FTL | FTL | 88.86 |
| 29 | GGT | | | | |
| sgRNA_ | CGGTCGAAATAGAAGCC | chr19:48965770 | FTL | FTL | 93.14 |
| 30 | CTA | | | | |
| sgRNA_ | AAAAGGATATTGTGCAA | chr10:87933015 | PTEN | PTEN | 92.37 |
| 31 | CTG | | | | |
| sgRNA_ | TGTGCATATTTATTACAT | chr10:87933183 | PTEN | PTEN | 90.64 |
| 32 | CG | | | | |
| sgRNA_ | TTTGTGAAGATCTTGAC | chr10:87933087 | PTEN | PTEN | 85.36 |
| 33 | CAA | | | | |
| sgRNA_ | TGTCATGCTGAACCGCA | chr18:12830972 | PTPN2 | PTPN2 | 87.94 |
| 34 | TTG | | | | |
| sgRNA_ | CCACTCTATGAGGATAG | chr18:12859219 | PTPN2 | PTPN2 | 92.45 |
| 35 | TCA | | | | |
| sgRNA_ | TTGACATAGAAGAGGCA | chr18:12836828 | PTPN2 | PTPN2 | 93.96 |
| 36 | CAA | | | | |
| sgRNA_ | GAGTACTACACTCAGCA | chr12:6952098 | PTPN6 | PTPN6 | 89.61 |
| 37 | GCA | | | | |
| sgRNA_ | TCACGCACAAGAAACGT | chr12:6954872 | PTPN6 | PTPN6 | 82.74 |
| 38 | CCA | | | | |
| sgRNA_ | AGGTCTCGGTGAAACCA | chr12:6951610 | PTPN6 | PTPN6 | 91.27 |
| 39 | CCT | | | | |
| sgRNA_ | AGCATTATCCAAAGAGT | chr1:198696873 | PTPRC | PTPRC | 88.88 |
| 40 | CCG | | | | |
| sgRNA_ | ATATTAATTCTTACCAGT | chr1:198692370 | PTPRC | PTPRC | 88.95 |
| 41 | GG | | | | |
| sgRNA_ | AGCTTTAAATCAAGGTT | chr1:198756176 | PTPRC | PTPRC | 96.89 |
| 42 | CAT | | | | |
| sgRNA_ | ATCCCGAGCCCTAAGGT | chr11:67436325 | PTPRCAP | PTPRCAP | 84.08 |
| 43 | GCA | | | | |
| sgRNA_ | GGCAGCGCGGAGGACA | chr11:67436285 | PTPRCAP | PTPRCAP | 97.74 |
| 44 | GCGT | | | | |
| sgRNA_ | CTCAGGGGGCTACTACC | chr11:67436170 | PTPRCAP | PTPRCAP | 91.50 |
| 45 | ACC | | | | |
| sgRNA_ | GTCACCGACGAGACCAG | chr5:82277810 | RPS23 | RPS23 | 79.40 |
| 46 | AAG | | | | |
| sgRNA_ | GTCGTGGACTTCGTACT | chr5:82277843 | RPS23 | RPS23 | 83.07 |
| 47 | GCT | | | | |
| sgRNA_ | TAATTTTTAGGCAAGTG | chr5:82277860 | RPS23 | RPS23 | 61.94 |
| 48 | TCG | | | | |
| sgRNA_ | TTAGCTGTTAGACTTGA | chr14:51993810 | RTRAF | RTRAF | 85.50 |
| 49 | ATA | | | | |
| sgRNA_ | CGAGAGCCGTCAACTTG | chr14:51989652 | RTRAF | RTRAF | 85.64 |
| 50 | CGT | | | | |
| sgRNA_ | CGGCTTCAACTGCAAAG | chr14:51989700 | RTRAF | RTRAF | 88.77 |
| 51 | GTG | | | | |
| sgRNA_ | TATGAAAAAGCAGAGCG | chr15:43793025 | SERF2 | SERF2 | 89.61 |
| 52 | ACT | | | | |
| sgRNA_ | TCTGGCGGGCGAGCTCA | chr15:43792989 | SERF2 | SERF2 | 86.73 |
| 53 | CGC | | | | |
| sgRNA_ | CTCACGCTGGTTACCGC | chr15:43792977 | SERF2 | SERF2 | 80.57 |
| 54 | CTA | | | | |
| sgRNA_ | AAAGATTACGAACTTCC | chr12:46207559 | SLC38A1 | SLC38A1 | 92.24 |
| 55 | CTG | | | | |
| sgRNA_ | GTTAAAAACAGACATGC | chr12:46229232 | SLC38A1 | SLC38A1 | 91.51 |
| 56 | CTA | | | | |
| sgRNA_ | ATGCCTAAGGAGGTTGT | chr12:46229246 | SLC38A1 | SLC38A1 | 79.48 |
| 57 | ACC | | | | |
| sgRNA_ | CTCCAGGTATCCCATCG | chr18:47869418 | SMAD2 | SMAD2 | 79.53 |
| 58 | AAA | | | | |
| sgRNA_ | CACCAAATACGATAGAT | chr18:47870532 | SMAD2 | SMAD2 | 86.61 |
| 59 | CAG | | | | |
| sgRNA_ | TGGCGGCGTGAATGGCA | chr18:47896729 | SMAD2 | SMAD2 | 82.91 |
| 60 | AGA | | | | |
| sgRNA_ | TAGGATGGTAGCACACA | chr16:11255478 | SOCS1 | SOCS1 | 92.25 |
| 61 | ACC | | | | |
| sgRNA_ | CAGCAGCAGAGCCCCGA | chr16:11255432 | SOCS1 | SOCS1 | 83.79 |
| 62 | CGG | | | | |
| sgRNA_ | CGGCGTGCGAACGGAAT | chr16:11255296 | SOCS1 | SOCS1 | 84.24 |
| 63 | GTG | | | | |
| sgRNA_ | TATAGACGCTGCCCGAC | chr15:40038895 | SRP14 | SRP14 | 95.12 |
| 64 | GTC | | | | |
| sgRNA_ | TCCAAAGAAGGGTACTG | chr15:40038368 | SRP14 | SRP14 | 92.14 |
| 65 | TGG | | | | |
| sgRNA_ | ACAGTACCCTTCTTTGG | chr15:40038358 | SRP14 | SRP14 | 65.82 |
| 66 | AAT | | | | |
| sgRNA_ | GCGACGGGCGCATCTAC | chr12:120469572 | SRSF9 | SRSF9 | 83.68 |
| 67 | GTG | | | | |
| sgRNA_ | CCCGACCTCCATAAGTC | chr12:120465700 | SRSF9 | SRSF9 | 92.56 |
| 68 | CTG | | | | |
| sgRNA_ | GGGGTCCTCGAAGCGCA | chr12:120469426 | SRSF9 | SRSF9 | 89.94 |
| 69 | CGA | | | | |
| sgRNA_ | TGCTCTGTTTAGAAGAT | chr5:32591641 | SUB1 | SUB1 | 79.36 |
| 70 | GAC | | | | |
| sgRNA_ | ATATTCTTTTCTAGTTAA | chr5:32591566 | SUB1 | SUB1 | 70.93 |
| 71 | AG | | | | |
| sgRNA_ | CCTGTAAAGAAACAAAA | chr5:32591614 | SUB1 | SUB1 | 93.66 |
| 72 | GAC | | | | |
| sgRNA_ | TGGAGAAAGACGTAACT | chr4:105234315 | TET2 | TET2 | 83.53 |
| 73 | TCG | | | | |
| sgRNA_ | TCTGCCCTGAGGTATGC | chr4:105234747 | TET2 | TET2 | 90.97 |
| 74 | GAT | | | | |
| sgRNA_ | ATTCCGCTTGGTGAAAA | chr4:105235656 | TET2 | TET2 | 89.62 |
| 75 | CGA | | | | |
| sgRNA_ | CAGGCACAATAGAAACA | chr3:114295571 | TIGIT | TIGIT | 92.65 |
| 76 | ACG | | | | |
| sgRNA_ | CCATTTGTAATGCTGACT | chr3:114295700 | TIGIT | TIGIT | 60.75 |
| 77 | TG | | | | |
| sgRNA_ | CTGGGTCACTTGTGCCG | chr3:114295634 | TIGIT | TIGIT | 87.99 |
| 78 | TGG | | | | |
| sgRNA_ | GTCAGGGTTCTGGATAT | chr14:22547508 | TRAC | TRAC | 98.20 |
| 79 | CTG | | | | |
| sgRNA_ | TGGATTTAGAGTCTCTC | chr14:22547541 | TRAC | TRAC | 88.15 |
| 80 | AGC | | | | |
| sgRNA_ | CTGCGGCTGTGGTCCAG | chr14:22550661 | TRAC | TRAC | 94.77 |
| 81 | CTG | | | | |
| sgRNA_ | ACAAAACTGTGCTAGAC | chr14:22547658 | TRAC | TRAC | 87.86 |
| 82 | ATG | | | | |
| sgRNA_ | TTCTTCCCCAGCCCAGGT | chr14:22547778 | TRAC | TRAC | 89.85 |
| 83 | AA | | | | |
| sgRNA_ | CGTCATGAGCAGATTAA | chr14:22550625 | TRAC | TRAC | 95.81 |
| 84 | ACC | | | | |
| sgRNA_ | GAGAGCGCCTGCGACCC | chr19:58544980 | TRIM28 | TRIM28 | 89.44 |
| 85 | GAG | | | | |
| sgRNA_ | CCAGCGGGTGAAGTACA | chr19:58544869 | TRIM28 | TRIM28 | 94.79 |
| 86 | CCA | | | | |
| sgRNA_ | GGAGCGCTTTTCGCCGC | chr19:58544839 | TRIM28 | TRIM28 | 91.81 |
| 87 | CAG | | | | |
| sgRNA_ | TGAGGCCTGGACCTTAT | chr10:33134193 | chr10: | desert_1 | 69.44 |
| 88 | GCA | | 33130000- | (GS88) | |
| | | | 33140000 | | |
| sgRNA_ | CCTGGTGGAGTGAACCA | chr10:33132917 | chr10: | desert_1 | 95.25 |
| 89 | TGA | | 33130000- | (GS89) | |
| | | | 33140000 | | |
| sgRNA_ | CAAGCACTTAGGTTCCC | chr10:33134633 | chr10: | desert_1 | 91.13 |
| 90 | CTG | | 33130000- | (GS90) | |
| | | | 33140000 | | |
| sgRNA_ | GGTCTCCCTACAATTCA | chr10:72294568 | chr10: | desert_2 | 92.02 |
| 91 | GCG | | 72290000- | (GS91) | |
| | | | 72300000 | | |
| sgRNA_ | CACAGCGCGTGACTGCA | chr10:72298268 | chr10: | desert_2 | 90.22 |
| 92 | ATG | | 72290000- | (GS92) | |
| | | | 72300000 | | |
| sgRNA_ | TCTGGGGCACCAATTCT | chr10:72292786 | chr10: | desert_2 | 86.35 |
| 93 | AGG | | 72290000- | (GS93) | |
| | | | 72300000 | | |
| sgRNA_ | GAGCCATGCTTGGCTTA | chr11:128342576 | chr11: | desert_3 | 91.24 |
| 94 | CGA | | 128340000- | (GS94) | |
| | | | 128350000 | | |
| sgRNA_ | GTACAAGTACTTATCTC | chr11:128343592 | chr11: | desert_3 | 89.02 |
| 95 | ATG | | 128340000- | (GS95) | |
| | | | 128350000 | | |
| sgRNA_ | GAGATAACAACATAACA | chr11:128347170 | chr11: | desert_3 | 96.47 |
| 96 | ACA | | 128340000- | (GS96) | |
| | | | 128350000 | | |
| sgRNA_ | CATATTCCATAGTCTTTG | chr11:65425000 | chr11: | desert_4 | 88.54 |
| 97 | GG | | 65425000- | (GS97) | |
| | | | 65427000 | | |
| | | | (NEAT1) | | |
| sgRNA_ | CTGCCCCTTAGCAACTT | chr11:65425507 | chr11: | desert_4 | 92.76 |
| 98 | AGG | | 65425000- | (GS98) | |
| | | | 65427000 | | |
| | | | (NEAT1) | | |
| sgRNA_ | TGTTTAAAAATATGTTG | chr11:65426264 | chr11: | desert_4 | 90.76 |
| 99 | ACA | | 65425000- | (GS99) | |
| | | | 65427000 | | |
| | | | (NEAT1) | | |
| sgRNA_ | CCAGGAATGGAAACTCA | chr15:92830315 | chr15: | desert_5 | 87.84 |
| 100 | CGC | | 92830000- | (GS100) | |
| | | | 92840000 | | |
| sgRNA_ | GAGGCCGCTGAATTAAC | chr15:92831850 | chr15: | desert_5 | 85.32 |
| 101 | CCG | | 92830000- | (GS101) | |
| | | | 92840000 | | |
| sgRNA_ | ATACACGCACACTTGCA | chr15:92831131 | chr15: | desert_5 | 99.92 |
| 102 | GAA | | 92830000- | (GS102) | |
| | | | 92840000 | | |
| sgRNA_ | GAGCAGACAGAAACCCA | chr16:11225670 | chr16: | desert_6 | 87.92 |
| 103 | GGG | | 11220000- | (GS103) | |
| | | | 11230000 | | |
| sgRNA_ | TGAGTCTCCAAACAGAA | chr16:11226284 | chr16: | desert_6 | 88.53 |
| 104 | CAG | | 11220000- | (GS104) | |
| | | | 11230000 | | |
| sgRNA_ | TAATATCACTGACTTCA | chr16:11225029 | chr16: | desert_6 | 87.65 |
| 105 | CGG | | 11220000- | (GS105) | |
| | | | 11230000 | | |
| sgRNA_ | TACACACAATGTAAGCA | chr2:87467461 | chr2: | desert_7 | 71.79 |
| 106 | GCA | | 87460000- | (GS106) | |
| | | | 87470000 | | |
| sgRNA_ | GGGAGCTCAATTCGAAA | chr2:87468809 | chr2: | desert_7 | 65.89 |
| 107 | CCA | | 87460000- | (GS107) | |
| | | | 87470000 | | |
| sgRNA_ | TTGGACAGGTGAGACAG | chr2:87467001 | chr2: | desert_7 | 72.64 |
| 108 | TCG | | 87460000- | (GS108) | |
| | | | 87470000 | | |
| sgRNA_ | AAGCTCACTCAGATAGT | chr3:186511316 | chr3: | desert_8 | 76.89 |
| 109 | GTG | | 186510000- | (GS109) | |
| | | | 186520000 | | |
| sgRNA_ | CAGGAGAACCACCTTAC | chr3:186515260 | chr3: | desert_8 | 86.31 |
| 110 | ACG | | 186510000- | (GS110) | |
| | | | 186520000 | | |
| sgRNA_ | GGACAGACCCTGATTCA | chr3:186519655 | chr3: | desert_8 | 85.47 |
| 111 | CAA | | 186510000- | (GS111) | |
| | | | 186520000 | | |
| sgRNA_ | ACATGGCAGTCTATGAA | chr3:59451154 | chr3: | desert_9 | 87.77 |
| 112 | CAG | | 59450000- | (GS112) | |
| | | | 59460000 | | |
| sgRNA_ | CCTATAGAGAGTACTAC | chr3:59456416 | chr3: | desert_9 | 79.33 |
| 113 | TTG | | 59450000- | (GS113) | |
| | | | 59460000 | | |
| sgRNA_ | CCAACCGGGTCTTCATT | chr3:59457029 | chr3: | desert_9 | 92.21 |
| 114 | ACG | | 59450000- | (GS114) | |
| | | | 59460000 | | |
| sgRNA_ | TCAAGCGTAGAGTTCCG | chr8:127993006 | chr8: | desert_10 | 93.07 |
| 115 | AGT | | 127980000- | (GS115) | |
| | | | 128000000 | | |
| sgRNA_ | TCATGCAATTATGGACC | chr8:127994663 | chr8: | desert_10 | 89.40 |
| 116 | CAG | | 127980000- | (GS116) | |
| | | | 128000000 | | |
| sgRNA_ | CGGGAAAGTGACTGGCC | chr8:127996766 | chr8: | desert_10 | 87.45 |
| 117 | ATG | | 127980000- | (GS117) | |
| | | | 128000000 | | |
| sgRNA_ | TGAGATTGAAATCAAAT | chr9:7974159 | chr9: | desert_11 | 84.84 |
| 118 | CGG | | 7970000- | (GS118) | |
| | | | 7980000 | | |
| sgRNA_ | TATGCAATATTCATCAC | chr9:7977914 | chr9: | desert_11 | 85.44 |
| 119 | GCG | | 7970000- | (GS119) | |
| | | | 7980000 | | |
| sgRNA_ | AATGTGTTAAATCAAAT | chr9:7976895 | chr9: | desert_11 | 83.48 |
| 120 | GCA | | 7970000- | (GS120) | |
| | | | 7980000 | | |

### CRISPR-Cas Editing

One effective example of gene editing is the CRISPR-Cas approach (e.g. CRISPR-Cas9). This approach incorporates the use of a guide polynucleotide (e.g. guide ribonucleic acid or gRNA) and a cas endonuclease (e.g. Cas9 endonuclease).

As used herein, a polypeptide referred to as a “Cas endonuclease” or having “Cas endonuclease activity” refers to a CRISPR-related (Cas) polypeptide encoded by a Cas gene, wherein a Cas polypeptide is a target DNA sequence that can be cleaved when operably linked to one or more guide polynucleotides (see, e.g., U.S. Pat. No. 8,697,359). Also included in this definition are variants of Cas endonuclease that retain guide polynucleotide-dependent endonuclease activity. The Cas endonuclease used in the donor DNA insertion method detailed herein is an endonuclease that introduces double-strand breaks into DNA at the target site (e.g., within the target locus or at the safe harbor site).

As used herein, the term “guide polynucleotide” relates to a polynucleotide sequence capable of complexing with a Cas endonuclease and allowing the Cas endonuclease to recognize and cleave a DNA target site. The guide polynucleotide can be a single molecule or a double molecule. The guide polynucleotide sequence can be an RNA sequence, a DNA sequence, or a combination thereof (RNA-DNA combination sequence). A guide polynucleotide comprising only ribonucleic acid is also referred to as “guide RNA”. In some embodiments, a polynucleotide donor construct is inserted at a safe harbor locus using a guide RNA (gRNA) in combination with a cas endonuclease (e.g. Cas9 endonuclease).

The guide polynucleotide includes a first nucleotide sequence domain (also referred to as a variable targeting domain or VT domain) that is complementary to a nucleotide sequence in the target DNA, and a second nucleotide that interacts with a Cas endonuclease polypeptide. It can be a double molecule (also referred to as a double-stranded guide polynucleotide) comprising a sequence domain (referred to as a Cas endonuclease recognition domain or CER domain). The CER domain of this double molecule guide polynucleotide comprises two separate molecules that hybridize along the complementary region. The two separate molecules can be RNA sequences, DNA sequences and/or RNA-DNA combination sequences.

Genome editing using CRISPR-Cas approaches relies on the repair of site-specific DNA double-strand breaks (DSBs) induced by the RNA-guided Cas endonuclease (e.g. Cas 9 endonuclease). Homology-directed repair (HDR) of these DSBs enables precise editing of the genome by introducing defined genomic changes, including base substitutions, sequence insertions, and deletions. Conventional HDR-based CRISPR/Cas9 genome-editing involves transfecting cells with Cas9, gRNA and donor DNA containing homologous arms matching the genomic locus of interest.

HITI (homology independent targeted insertion) uses a non-homologous end joining (NHEJ)-based homology-independent strategy and the method can be more efficient than HDR. Guide RNAs (gRNAs) target the insertion site. For HITI, donor plasmids lack homology arms and DSB repair does not occur through the HDR pathway. The donor polynucleotide construct can be engineered to include Cas9 cleavage site(s) flanking the gene or sequence to be inserted. This results in Cas9 cleavage at both the donor plasmid and the genomic target sequence. Both target and donor have blunt ends and the linearized donor DNA plasmid is used by the NHEJ pathway resulting integration into the genomic DSB site. (See, for example, Suzuki, K., et al. (2016). In vivo genome editing via CRISPR/Cas9 mediated homology-independent targeted integration. Nature, 540 (7631), 144-149, the relevant disclosures of which are herein incorporated in their entirety).

Methods for conducing gene editing using CRISPR-Cas approaches are known to those of ordinary skill in the art. (See, for example, US Application Nos. U.S. Ser. No. 16/312,676, U.S. Ser. No. 15/303,722, and U.S. Ser. No. 15/628,533, the disclosures of which are herein incorporated by reference in their entirety). Additionally, uses of endonucleases for inserting transgenes into safe harbor loci are described, for example, in U.S. application Ser. No. 13/036,343, the disclosures of which are herein incorporated by reference in their entirety.

The guide RNAs and/or mRNA (or DNA) encoding an endonuclease can be chemically linked to one or more moieties or conjugates that enhance the activity, cellular distribution, or cellular uptake of the oligonucleotide. Non-limiting examples of such moieties include lipid moieties such as a cholesterol moiety, cholic acid, a thioether, a thiocholesterol, an aliphatic chain (e.g., dodecandiol or undecyl residues), a phospholipid, e.g., di-hexadecyl-rac-glycerol or triethylammonium 1,2-di-O-hexadecyl-rac-glycero-3-H-phosphonate, a polyamine or a polyethylene glycol chain, adamantane acetic acid, a palmityl moiety and an octadecylamine or hexylamino-carbonyl-t oxycholesterol moiety. See for example US Patent Publication No. 20180127786, the disclosure of which is herein incorporated by reference in its entirety.

### Therapeutic Applications

For therapeutic applications, the engineered cells, populations thereof, or compositions thereof are administered to a subject, generally a mammal, generally a human, in an effective amount.

The engineered cells may be administered to a subject by infusion (e.g., continuous infusion over a period of time) or other modes of administration known to those of ordinary skill in the art.

The engineered cells provided herein not only find use in gene therapy but also in non-pharmaceutical uses such as, e.g., production of animal models and production of recombinant cell lines expressing a recombinant nucleic acid of interest.

The engineered cells of the present disclosure can be any cell, generally a mammalian cell, generally a human cell that has been modified by integrating a transgene at a safe harbor locus described herein. Exemplary cells are provided in the Recombinant Cells section.

The engineered cells, compositions and methods of the present disclosure are useful for therapeutic applications such as immune or T cell therapy. In some embodiments, the insertion of a sequence encoding an shRNA molecule within a safe harbor locus maintains the TCR expression relative to instances when there is no insertion and enables transgene expression while maintaining TCR function.

In some embodiments, the present disclosure provides methods of treating a subject in need of treatment by administering to the subject a composition comprising any of the engineered cells described herein. In some embodiments, administration of the engineered cell composition results in a desired pharmacological and/or physiological effect. That effect can be partial or complete cure of the disease and/or adverse effects resulting from the disease. In some embodiments, treatment encompasses any treatment of a disease in a subject (e.g., mammal, e.g., human). Further, treatment may stabilize or reduce undesirable clinical symptoms in subjects (e.g., patients). The cells provided herein populations thereof, or compositions thereof may be administered during or after the occurrence of the disease.

In certain embodiments, the subject has a disease, condition, and/or injury that can be treated and/or ameliorated by cell therapy. In some embodiments, the subject in need of cell therapy is a subject having an injury, disease, or condition, thereby causing cell therapy (e.g., therapy in which cellular material is administered to the subject). However, it is contemplated that it is possible to treat, ameliorate and/or reduce the severity of at least one symptom associated with the injury, disease or condition.

### Method of Administration

An effective amount of the immune cell comprising the system may be administered for the treatment of cancer. The appropriate dosage of the immune cell comprising the system may be determined based on the type of cancer to be treated, the type of the immune cell comprising the system, the severity and course of the cancer, the clinical condition of the individual, the individual's clinical history and response to the treatment, and the discretion of the attending physician.

### Pharmaceutical Compositions

The engineered recombinant cells or recombinant nucleic acids provided herein can be administered as part of a pharmaceutical compositions. These compositions can comprise, in addition to one or more of the recombinant cells, a pharmaceutically acceptable excipient, carrier, buffer, stabiliser or other materials well known to those skilled in the art. Such materials should be non-toxic and should not interfere with the efficacy of the active ingredient. The precise nature of the carrier or other material can depend on the route of administration, e.g. oral, intravenous, cutaneous or subcutaneous, nasal, intramuscular, intraperitoneal routes. The pharmaceutical composition may comprise one or more pharmaceutical excipients. Any suitable pharmaceutical excipient may be used, and one of ordinary skill in the art is capable of selecting suitable pharmaceutical excipients. Accordingly, the pharmaceutical excipients provided below are intended to be illustrative, and not limiting. Additional pharmaceutical excipients include, for example, those described in the Handbook of Pharmaceutical Excipients, Rowe et al. (Eds.) 6th Ed. (2009), incorporated by reference in its entirety.

Various modes of administering the additional therapeutic agents are contemplated herein. In some embodiments, the additional therapeutic agent is administered by any suitable mode of administration.

A composition can be administered alone or in combination with other treatments, either simultaneously or sequentially dependent upon the condition to be treated.

### Kits and Articles of Manufacture

The present application provides kits comprising any one or more of the system or cell compositions described herein along with instructions for use. The instructions for use can be present in the kits as a package insert, in the labeling of the container of the kit or components thereof, or can be in digital form (e.g. on a CD-ROM, via a link on the internet). A kit can include one or more of a genome-targeting nucleic acid, a polynucleotide encoding a genome-targeting nucleic acid, a site-directed polypeptide, and/or a polynucleotide encoding a site-directed polypeptide. Additional components within the kits are also contemplated, for example, buffer (such as reconstituting buffer, stabilizing buffer, diluting buffer), and/or one or more control vectors.

In some embodiments, the kits further contain a component selected from any of secondary antibodies, reagents for immunohistochemistry analysis, pharmaceutically acceptable excipient and instruction manual and any combination thereof. In one specific embodiment, the kit comprises a pharmaceutical composition comprising any one or more of the antibody compositions described herein, with one or more pharmaceutically acceptable excipients.

The present application also provides articles of manufacture comprising any one of the antibody compositions or kits described herein. Examples of an article of manufacture include vials (including sealed vials).

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2^{nd} Edition, 1989); Methods In Enzymology (S. Colowick and N. Kaplan eds., Academic Press, Inc.); Remington's Pharmaceutical Sciences, 18^{th} Edition (Easton, Pennsylvania: Mack Publishing Company, 1990); Carey and Sundberg Advanced Organic Chemistry 3^{rd} Ed. (Plenum Press) Vols A and B (1992).

### Example 1: Identification and Characterization of Combination Gene Perturbations Influencing T Cell Killing or Expansion In Vitro

### Materials

### T Cell Editing and In Vitro Cell-Based Repetitive Stimulation Assessment

Engineered T cells were generated using CITE non-viral gene delivery. Briefly, pan-T cells were isolated from healthy human donors using the Miltenyi StraightFrom® Leukopak® CD4/CD8 MicroBead Kit. Isolated T cells were stimulated with anti-CD3/anti-CD28 beads. Two day after stimulation, cells were resuspended in a solution containing S. pyogenes Cas9 complexed with GS94 guide RNA and donor DNA template encoding transgene of interest. To evaluate effect of various gene knockouts, cells were treated with Cas9 RNP+sgRNA targeting DNMT3A, TET2, CD5, DGKA, DGKZ, MAP4K1, CBLB, FAS, PTPN2, NR4A1, ZC3H12A, or CISH or singly or in combination as indicated. Effective sgRNA sequences are summarized in Table 1. Cells were subsequently electroporated using the Lonza 4-D Nucleofector and recovered in fresh media supplemented with IL-7 and IL-15. Cells were counted and fresh media added every 2-3 days following electroporation. All constructs tested encoded a logic gate expressing a PrimeR receptor to ALPG (SEQ ID NO: 213) and a CAR targeting MSLN (SEQ ID NO: 212). Such cells are termed Integrated Circuit T cell (ICT).

On day 4 post-electroporation, edited cells were enriched via bead-based positive selection for Myc+ cells (a Myc tag was expressed on the priming receptor). T cells were co-cultured with K562 tumor cells engineered to express ALPG and MSLN at a 2:1 effector:target (E:T) ratio. T cells and tumors were quantified via flow cytometry every 2-3 days and at each timepoint T cells were normalized to a defined concentration and restimulated at a 2:1 E:T ratio. Six total stimulations were conducted over a 14-day period.

### Computational Integration of RSA Data

T cell and target tumor cell expansion over the course of the RSA was determined by a computational workflow wherein total cell count per well were imputed with the following formula:

$$Volume = ( BeadCount + 1 ) \div ( BeadConcentration \times uLBeads )$$

$$CellsPer μ L = TagCounts \div Volume$$

$$CellsPermL = ( 1000 \div CellVolume ) \times CellsPer μ L$$

$$TotalCells = ( WellVolume \div 1000 ) \times CellsPermL$$

The primary tags used for workflow were CD3 (T cells) and GFP (target tumor cells). The total T cells per well at day 0 was assigned as the total T cells seeded, which was typically 85,000. The total target tumor cells at day 0 was calculated based on the E:T ratio. For example, in the case of 85,000 T cells seeded and an ET ratio of 2:1, the target tumor cell count at day 0 would be 42,500.

Quality control was run on each well and wells with any of the following criteria were excluded: any wells that reduce to 0 T cell counts and subsequently increase; any wells with less than 100 beads; any wells with less than 20 live cells.

Cells were re-normalized at each restimulation time point with TcellsPerStim typically equal to 85,000, and maintain the 2:1 E:T ratio when possible to ensure the T cells are consistently challenged. T cells were diluted if there are more than TcellsPerStim in each well. Otherwise, no dilution was executed and no additional T cells were added. This was reflected in the computational workflow by normalizing the total T cells observed at each time point by a dilution factor defined as follows where TotalTCells is TotalCells calculated as described above for CD3 tags:

$$Dilution = TcellsPerStim / TotalTCells ifTotalCells \geq TcellsPerStim 1 if TotalCells < TcellsPerStim TotalTcellsRenorm = TotalTCells \times Dilution$$

In the experimental protocol, additional target cells were added at each stimulation if there were fewer than the number of desired target cells based on TcellsPerStim and the E:T ratio. For example, if TcellsPerStim=85,000 and the E:T ratio is 2:1, the target cells were re-normalized to 42,500 cells. On the other hand, target cells were not removed if the number of target cells exceeds this amount, except when dilution occurs based on the T cell count as described above. This is reflected in the computational workflow as follows, where TotalTargetCells and TotalTCells are TotalCells calculated as described above for GFP and CD3 tags respectively:

$$TargetDiluted = TotalTargetCells \times Dilution$$

$$TargetByETratio = TotalTCells \times Dilution \div ETratio$$

$$TotalTargetCellsRenorm = max ( TargetDiluted , TargetByETratio )$$

T cell and target cell expansion values were calculated at each time point as the ratio of total cells observed vs. the amount at the previous re-normalization, and summarized cumulatively as follows where n=total measurements:

$$TCellExpansion = ( TotalTCells + 1 ) \div ( TotalTCellsRenorm + 1 )$$

$$TCellCumulativeExpansion = {{\prod }^{n}}_{i = 0} {TCellExpansion}_{i}$$

TCellExpansion values were capped at 0.05 and 999 to prevent extreme values from dominating the cumulative summary metric. Expansion values for target tumor cells were calculated similarly. T cell and target cell expansion metrics were subsequently normalized to a control sample for downstream analysis.

### Statistical Analysis

A false discovery rate (FDR) statistical approach was used to identify gene perturbations with significant improvement to either T cell mediated target killing or T cell expansion as pairs relative to their individual component genes. Briefly, log values were normalized to non-targeting control (NTC) per plate per donor such that the NTC median across replicates=1; log 2 for T cell expansion (CD3) and log₁₀ for target expansion (GFP). Two tests were performed for both the ICT expansion and target cell killing functional data: 1) one-tailed t-test of the paired perturbation combinations vs. each single perturbation, excluding NTC control and 2) one-tailed t-test of the combination perturbations compared to a synthetic sum of single perturbation values to test for super-additivity. For the single gene perturbations, the statistics used in the t-test were defined as follows: mean was defined as the mean across replicates of gene perturbation A+mean across replicates of gene perturbation B. Standard deviation was defined as sqrt (variance of gene perturbation A+variance of gene perturbation B). n was defined as the number of gene perturbation A samples+number of gene perturbation B samples. The Benjamini-Hochberg FDR correction was implemented as follows: One-tailed t-test of the gene perturbation combinations vs. each individual gene perturbation was corrected across all combinations. One-tailed t-tests of the combination gene perturbation compared to. The synthetic sum of individual component gene perturbations were corrected across all tests. Fold change (i.e., log-scale difference) was calculated as the combination gene perturbation compared to the synthetic sum: (combination gene perturbation mean log value normalized to non-targeting control)-sum (individual gene perturbation mean log values normalized to NTC). Combination gene perturbations compared to each single perturbation: (combination gene perturbation mean log value normalized to NTC)-(single gene perturbation mean log value normalized to NTC). To compile the tables, A relax FDR cutoff of 0.05 was employed.

### Results

To identify genes that provide additional benefit when perturbed as a pair relative to either individual component gene, a pairwise CRISPR screen was executed, wherein DNMT3A, TET2, CD5, DGKA, DGKZ, MAP4K1, CBLB, FAS, PTPN2, NR4A1, ZC3H12A, or CISH were ablated individually in T cells via CRISPR/Cas9 or in combination with each other and subjected to a repetitive cell-based killing assay to induce a high-stress, exhaustion prone setting.

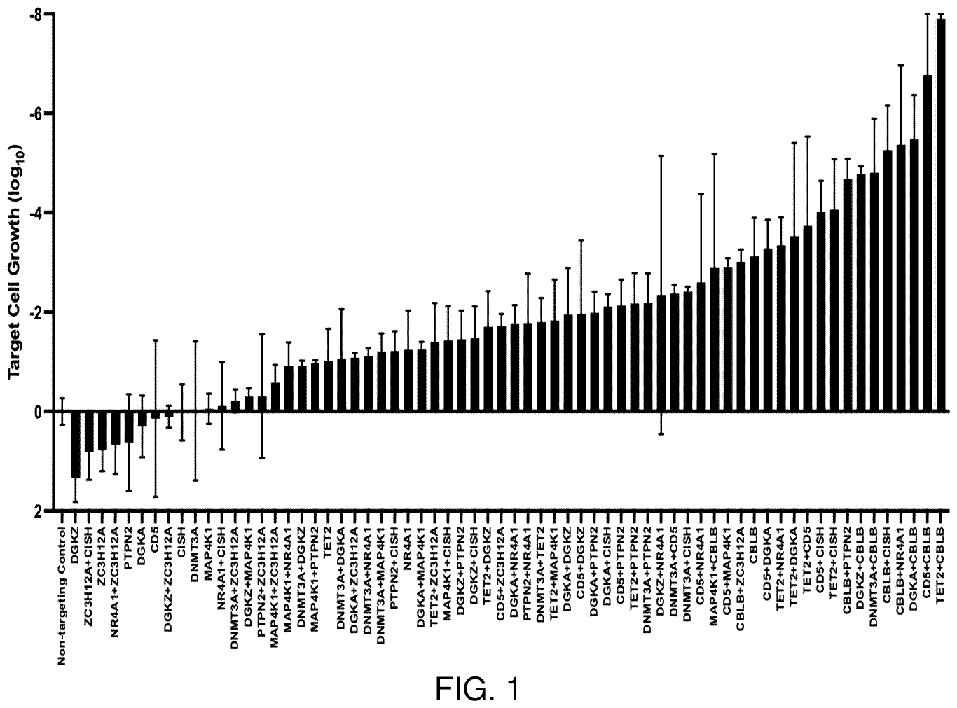

As shown in FIG. 1, T cell killing activity was observed among the single- and double-gene perturbations, ranging from a modest decrease in killing in T-cells harboring perturbations to DGKZ, to over 7-log₁₀ fold increase in killing observed with the combined perturbation of TET2 and CBLB. Combined perturbations with CBLB exhibited pronounced improvements in T cell killing, with 8 of the top ten combination perturbations including CBLB as one of the component genes. A number of gene perturbation combinations were identified which exhibited statistically significant superior T cell killing relative to either individual component gene, summarized in Table 1, indicating that combined perturbation offers a benefit relative to individual perturbation. Further, many gene combinations exhibited statistically significant superior T cell killing relative to the synthetic sum of individual combinations, summarized in Table 2 indicating that these combinations drive super-additive killing activity.

Table 1 provides gene knockout combinations that conferred superior T cell cytotoxicity to either individual component gene.

TABLE 1

| | | | Fold | | Fold | | | |
|---|---|---|---|---|---|---|---|---|
| | | | change: | FDR: | change: | FDR: | Fold- | |
| | | | pair vs | pair vs | pair vs | pair vs | Max | Max |
| Gene Pair | Gene A | Gene B | Gene A | Gene A | Gene B | Gene B | FDR | FDR |
| CBLB + CISH | CBLB | CISH | −2.410 | 0.020 | −5.220 | 0.004 | −2.410 | 0.020 |
| CBLB + NR4A1 | CBLB | NR4A1 | −2.690 | 0.047 | −3.900 | 0.026 | −2.690 | 0.047 |
| CBLB + PTPN2 | CBLB | PTPN2 | −1.680 | 0.029 | −5.110 | 0.008 | −1.680 | 0.029 |
| CD5 + CBLB | CD5 | CBLB | −6.970 | 0.008 | −4.170 | 0.017 | −4.170 | 0.017 |
| CD5 + CISH | CD5 | CISH | −4.250 | 0.022 | −4.260 | 0.008 | −4.250 | 0.022 |
| CD5 + DGKA | CD5 | DGKA | −3.280 | 0.034 | −3.540 | 0.013 | −3.280 | 0.034 |
| CD5 + MAP4K1 | CD5 | MAP4K1 | −2.820 | 0.047 | −3.000 | 0.009 | −2.820 | 0.047 |
| DGKA + CBLB | DGKA | CBLB | −5.930 | 0.011 | −2.870 | 0.026 | −2.870 | 0.026 |
| DGKA + CISH | DGKA | CISH | −2.380 | 0.028 | −2.130 | 0.013 | −2.380 | 0.028 |
| DGKA + DGKZ | DGKA | DGKZ | −2.260 | 0.045 | −3.500 | 0.026 | −2.260 | 0.045 |
| DGKA + PTPN2 | DGKA | PTPN2 | −2.020 | 0.028 | −2.390 | 0.026 | −2.020 | 0.028 |
| DGKZ + CBLB | DGKZ | CBLB | −6.190 | 0.010 | −1.880 | 0.029 | −1.880 | 0.029 |
| DGKZ + CISH | DGKZ | CISH | −2.970 | 0.009 | −1.470 | 0.028 | −1.470 | 0.028 |
| DGKZ + PTPN2 | DGKZ | PTPN2 | −2.890 | 0.016 | −2.010 | 0.034 | −2.010 | 0.034 |
| DGKZ + ZC3H12A | DGKZ | ZC3H12A | −1.440 | 0.035 | −1.020 | 0.046 | −1.020 | 0.046 |
| DNMT3A + CBLB | DNMT3A | CBLB | −5.490 | 0.019 | −2.350 | 0.029 | −2.350 | 0.029 |
| MAP4K1 + CISH | MAP4K1 | CISH | −1.570 | 0.028 | −1.410 | 0.034 | −1.410 | 0.034 |
| PTPN2 + CISH | PTPN2 | CISH | −1.820 | 0.040 | −1.210 | 0.035 | −1.820 | 0.040 |
| TET2 + CBLB | TET2 | CBLB | −6.930 | 0.002 | −4.910 | 0.004 | −4.910 | 0.004 |
| TET2 + CD5 | TET2 | CD5 | −3.550 | 0.036 | −4.330 | 0.026 | −3.550 | 0.036 |
| TET2 + CISH | TET2 | CISH | −2.870 | 0.019 | −3.660 | 0.013 | −2.870 | 0.019 |
| TET2 + DGKA | TET2 | DGKA | −3.420 | 0.043 | −4.470 | 0.028 | −3.420 | 0.043 |
| TET2 + PTPN2 | TET2 | PTPN2 | −1.260 | 0.043 | −2.670 | 0.019 | −1.260 | 0.043 |

Table 2 provides gene knockout combinations that conferred super-additive T cell killing relative to either individual component gene.

TABLE 2

| | | | Fold | |
|---|---|---|---|---|
| | | | change: | FDR: |
| | | | pair vs | pair vs |
| | | | Gene A + | Gene A + |
| Pair | Gene A | Gene B | Gene B | Gene B |
| CBLB + CISH | CBLB | CISH | −2.34 | 0.015 |
| CBLB + PTPN2 | CBLB | PTPN2 | −2.23 | 0.0072 |
| CBLB + ZC3H12A | CBLB | ZC3H12A | −1.14 | 0.027 |
| CD5 + CBLB | CD5 | CBLB | −4.08 | 0.011 |
| CD5 + CISH | CD5 | CISH | −4.18 | 0.0026 |
| CD5 + DGKA | CD5 | DGKA | −3.46 | 0.0076 |
| CD5 + DGKZ | CD5 | DGKZ | −4 | 0.033 |
| CD5 + MAP4K1 | CD5 | MAP4K1 | −2.92 | 0.011 |
| CD5 + PTPN2 | CD5 | PTPN2 | −2.63 | 0.015 |
| CD5 + ZC3H12A | CD5 | ZC3H12A | −2.61 | 0.015 |
| DGKA + CBLB | DGKA | CBLB | −3.05 | 0.018 |
| DGKA + CISH | DGKA | CISH | −2.31 | 0.0026 |
| DGKA + DGKZ | DGKA | DGKZ | −3.69 | 0.017 |
| DGKA + MAP4K1 | DGKA | MAP4K1 | −1.54 | 0.010 |
| DGKA + PTPN2 | DGKA | PTPN2 | −2.57 | 0.0049 |
| DGKA + ZC3H12A | DGKA | ZC3H12A | −2.23 | 0.0049 |
| DGKZ + CBLB | DGKZ | CBLB | −3.31 | 0.0013 |
| DGKZ + CISH | DGKZ | CISH | −2.9 | 0.0026 |
| DGKZ + MAP4K1 | DGKZ | MAP4K1 | −1.84 | 0.0029 |
| DGKZ + PTPN2 | DGKZ | PTPN2 | −3.43 | 0.0029 |
| DGKZ + ZC3H12A | DGKZ | ZC3H12A | −2.45 | 0.0018 |
| DNMT3A + CBLB | DNMT3A | CBLB | −2.61 | 0.024 |
| DNMT3A + CD5 | DNMT3A | CD5 | −2.51 | 0.036 |
| DNMT3A + CISH | DNMT3A | CISH | −2.63 | 0.014 |
| DNMT3A + DGKZ | DNMT3A | DGKZ | −2.61 | 0.018 |
| DNMT3A + PTPN2 | DNMT3A | PTPN2 | −2.9 | 0.014 |
| MAP4K1 + CISH | MAP4K1 | CISH | −1.51 | 0.024 |
| MAP4K1 + PTPN2 | MAP4K1 | PTPN2 | −1.63 | 0.016 |
| MAP4K1 + ZC3H12A | MAP4K1 | ZC3H12A | −1.55 | 0.010 |
| PTPN2 + CISH | PTPN2 | CISH | −1.75 | 0.015 |
| TET2 + CBLB | TET2 | CBLB | −4.05 | 0.0029 |
| TET2 + CD5 | TET2 | CD5 | −3.47 | 0.036 |
| TET2 + CISH | TET2 | CISH | −2.8 | 0.015 |
| TET2 + DGKA | TET2 | DGKA | −3.61 | 0.043 |
| TET2 + DGKZ | TET2 | DGKZ | −2.46 | 0.018 |
| TET2 + PTPN2 | TET2 | PTPN2 | −1.8 | 0.016 |

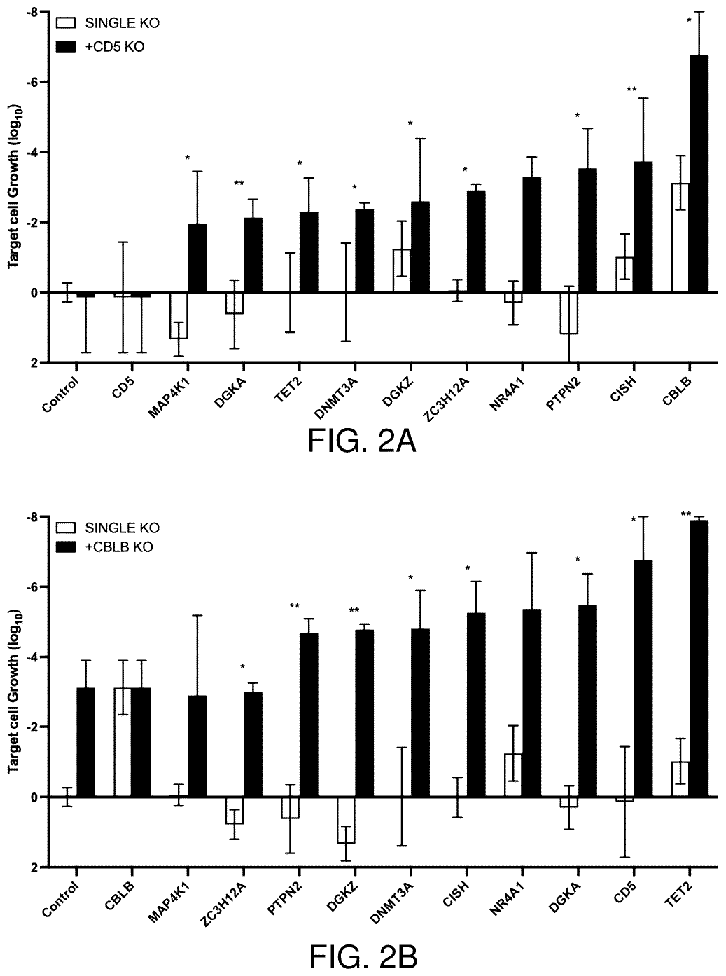

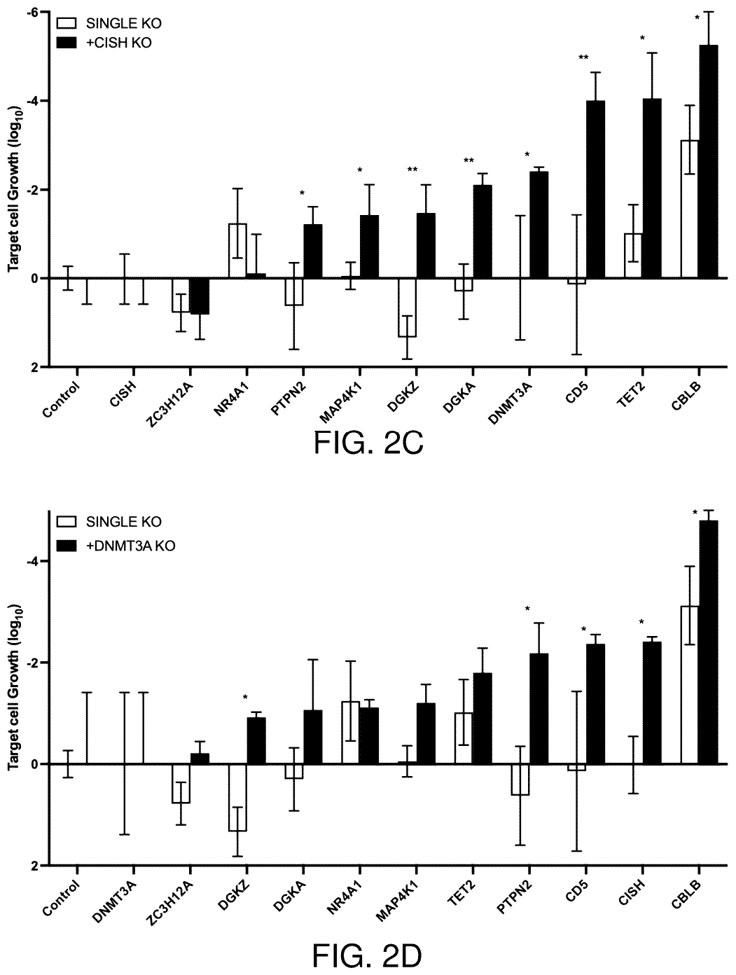

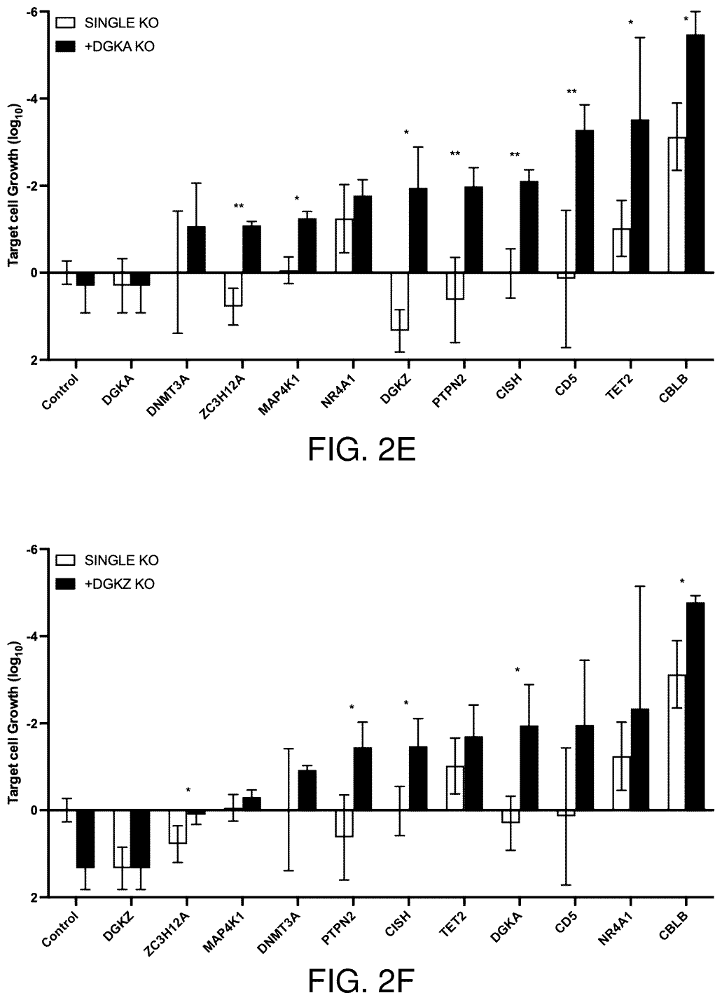

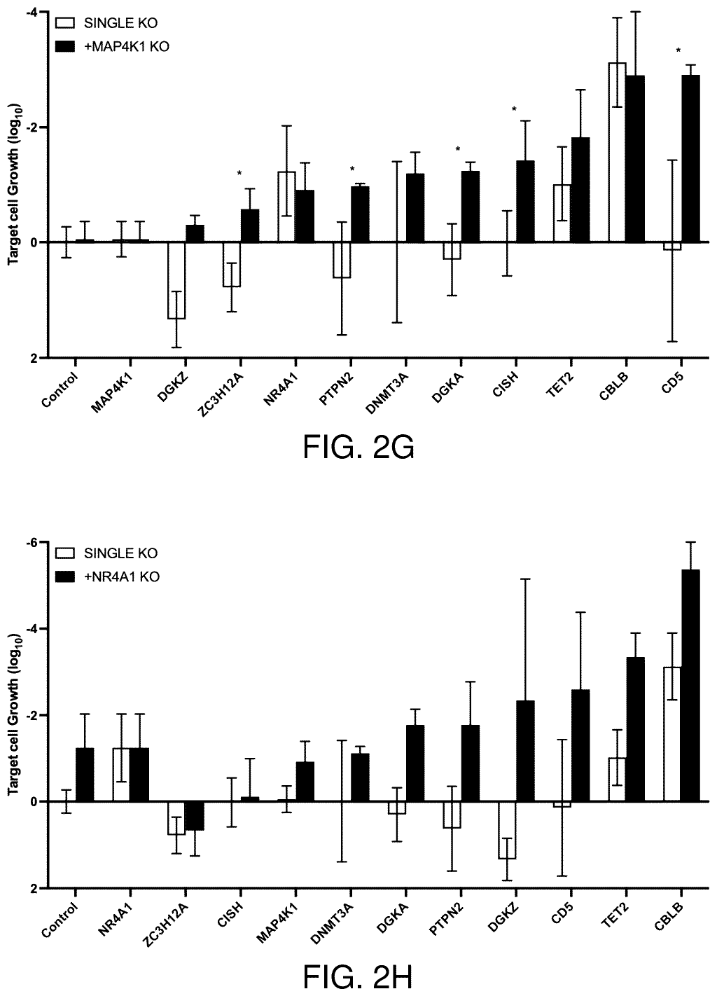

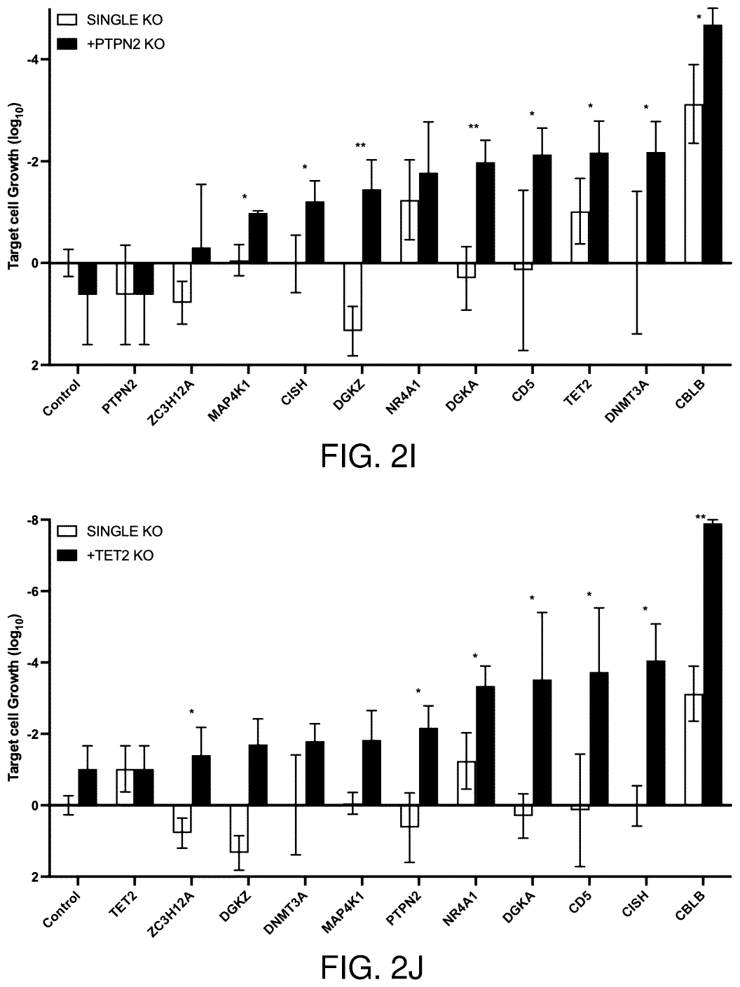

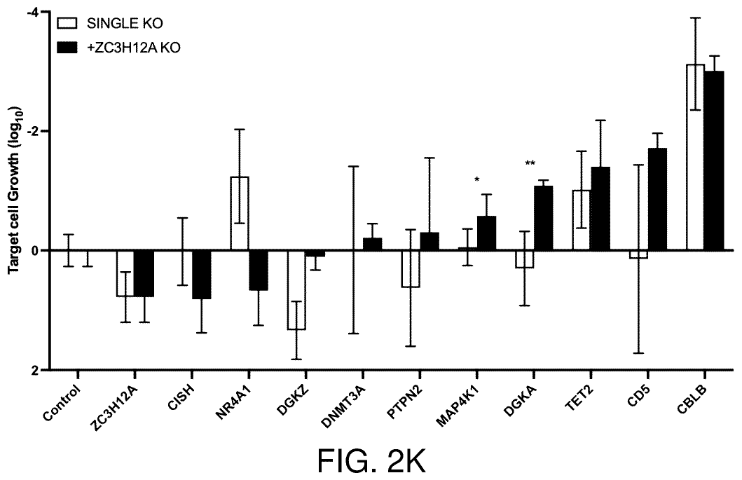

CRISPR-mediated CD5 perturbation did not impart significant improvements in T cell killing as a single gene perturbation relative to a non-targeting control, but pairwise CD5 perturbation with other genes which similarly did not impart improvements to killing when evaluated as single perturbations, including ZC3H12A, DGKZ, PTPN2, DNMT3A, MAP4K1, DGKA, and CISH (FIG. 2A). Combined CD5 perturbation with genes that did impart improved killing as single perturbations, including TET2, and CBLB also exhibited superior killing than would be anticipated by additive activity. The combined perturbation of CD5 with NR4A1 did not significantly differ from either individual perturbation, offering an example that not all combination perturbations with CD5 produce a super-additive effect.

CRISPR-mediated perturbation of CBLB significantly improved T cell killing, exhibiting a 3 log₁₀-fold increase in killing relative to a non-targeting control (FIG. 2B). Pairwise CBLB perturbation with other genes which did not impart improvements to killing when evaluated as single perturbations, including ZC3H12A, PTPN2, DGKZ, DNMT3A, CISH, and DGKA resulted in significant improvements in killing beyond that observed with CBLB perturbation alone. Combined CBLB perturbation with genes that did impart improved killing as single perturbations, including TET2 also exhibited superior killing than would be anticipated by additive activity. The combined perturbation of CBLB with NR4A1 or MAP4K1 did not significantly differ from the singular perturbation of CBLB or the other paired gene in isolation, offering examples of combination perturbations with CBLB that do not produce a super-additive effect.

CRISPR-mediated perturbation of CISH did not significantly alter T cell killing relative to a non-targeting control (FIG. 2C). Pairwise CISH perturbation with other genes which did not impart improvements to killing when evaluated as single perturbations resulted in significant improvements in killing beyond that observed with CISH perturbation alone, including pairings with PTPN2, MAPK41, DGKZ, DGKA, and DNMT3A. Combined CISH perturbation with genes that did impart improved killing as single perturbations, including TET2 and CBLB, also exhibited superior killing than would be anticipated by additive activity. The combined perturbation of CISH with ZC3H12A or NR4A1 did not significantly differ from the singular perturbation of CISH or the other paired gene in isolation, offering examples of combination perturbations with CISH that do not produce a super-additive effect.

CRISPR-mediated perturbation of DNMT3A did not significantly impact T cell killing relative to a non-targeting control (FIG. 2D). Pairwise DNMT3A perturbation with other genes which did not impart improvements to killing when evaluated as single perturbations resulted in significant improvements in killing beyond that observed with DNMT3A perturbation alone, including pairings with DGKZ, PTPN2, CD5, and CISH. Combined DNMT3A perturbation with genes that did impart improved killing as single perturbations, including CBLB, also exhibited superior killing than would be anticipated by additive activity. The combined perturbation of DNMT3A with ZC3H12A, DGKA, NR4A1, MAP4K1, or TET2 did not significantly differ from the singular perturbation of DNMT3A or the other paired gene in isolation, offering examples of combination perturbations with DNMT3A that do not produce a super-additive effect.

CRISPR-mediated perturbation of DGKA did not significantly alter T cell killing relative to a non-targeting control (FIG. 2E). Pairwise DGKA perturbation with other genes which did not impart improvements to killing when evaluated as single perturbations resulted in significant improvements in killing beyond that observed with DGKA perturbation alone, including pairings with ZC3H12A, MAP4K1, DGKZ, PTPN2, CISH, and CD5. Combined DGKA perturbation with genes that did impart improved killing as single perturbations, including TET2 and CBLB, also exhibited superior killing than would be anticipated by additive activity. The combined perturbation of DGKA with DNMT3A or NR4A1 did not significantly differ from the singular perturbation of DGKA or the other paired gene in isolation, offering examples of combination perturbations with DGKA that did not produce a super-additive effect.

CRISPR-mediated perturbation of DGKZ led to a modest decrement in T cell killing relative to a non-targeting control (FIG. 2F). Pairwise DGKZ perturbation with other genes which did not impart improvements to killing when evaluated as single perturbations resulted in significant improvements in killing beyond that observed with DGKZ perturbation alone, including pairings with PTPN2, CISH, and DGKA. Combined DGKZ perturbation with genes that did impart improved killing as single perturbations, including CBLB, also exhibited superior killing than would be anticipated by additive activity. The combined perturbation of DGKZ with MAP4K1, DNMT3A, TET2, CD5, or NR4A1 did not significantly differ from the singular perturbation of DGKZ or the other paired gene in isolation, offering examples of combination perturbations with DGKZ that do not produce a super-additive effect.

CRISPR-mediated perturbation of MAP4K1 did not significantly impact T cell killing relative to a non-targeting control (FIG. 2G). Pairwise MAP4K1 perturbation with other genes which did not impart improvements to killing when evaluated as single perturbations resulted in significant improvements in killing beyond that observed with MAP4K1 perturbation alone, including pairings with ZC3H12A, PTPN2, DGKA, and CISH. Combined MAP4K1 perturbation with genes that did impart improved killing as single perturbations, including TET2 and CBLB did not further improve killing activity. The combined perturbation of MAP4K1 with NR4A1, TET2, or CBLB did not significantly differ from the singular perturbation of MAP4K1 or the other paired gene in isolation, offering examples of combination perturbations with MAP4K1 that do not produce a super-additive effect.

CRISPR-mediated perturbation of NR4A1 resulted in a modest improvement in T cell killing relative to a non-targeting control (FIG. 2H). Pairwise NR4A1 perturbation with other genes failed to result in improvements to killing beyond which would be expected by NR4A1 or the paired gene in isolation. These findings underscore that it is unpredictable which genes have the capacity to complement each other as additive or super-additive contributors to T cell killing when perturbed as pairs.

CRISPR-mediated perturbation of PTPN2 did not significantly impact T cell killing relative to a non-targeting control (FIG. 2I). Pairwise PTPN2 perturbation with other genes which did not impart improvements to killing when evaluated as single perturbations resulted in significant improvements in killing beyond that observed with PTPN2 perturbation alone, including pairings with MAP4K1, CISH, DGKZ, DGKA, CD5, and DNMT3A. Combined PTPN2 perturbation with genes that did impart improved killing as single perturbations, including TET2 or CBLB, also exhibited superior killing than would be anticipated by additive activity. The combined perturbation of PTPN2 with ZC3H12A or NR4A1 did not significantly differ from the singular perturbation of PTPN2 or the other paired gene in isolation, offering examples of combination perturbations with PTPN2 that do not produce a super-additive effect.

CRISPR-mediated perturbation of TET2 led to a modest improvement in T cell killing relative to a non-targeting control (FIG. 2J). Pairwise TET2 perturbation with other genes which did not impart improvements to killing when evaluated as single perturbations resulted in significant improvements in killing beyond that observed with TET2 perturbation alone, including pairings with ZC3H12A, PTPN2, DGKA, CD5, and CISH. Combined TET2 perturbation with genes that did impart improved killing as single perturbations, including NR4A1 or CBLB, also exhibited superior killing than would be anticipated by additive activity. The combined perturbation of TET2 with DGKZ, DNMT3A, or MAP4K1 did not significantly differ from the singular perturbation of TET2 or the other paired gene in isolation, offering examples of combination perturbations with TET2 that do not produce a super-additive effect.

CRISPR-mediated perturbation of ZC3H12A led to a modest decrement in T cell killing relative to a non-targeting control (FIG. 2K). Pairwise ZC3H12A perturbation with other genes which did not impart improvements to killing when evaluated as single perturbations resulted in significant improvements in killing beyond that observed with ZC3H12A perturbation alone, including pairings with MAP4K1 and DGKA. Combined ZC3H12A perturbation with genes that did impart improved killing as single perturbations, result in improvements to killing beyond which would be expected by either of the paired genes in isolation. The combined perturbation of ZC3H12A with CISH, NR4A1, DGKZ, DNMT3A, PTPN2, TET2, CD5, or CBLB did not significantly differ from the singular perturbation of ZC3H12A or the other paired gene in isolation, offering examples of combination perturbations with ZC3H12A that do not produce a super-additive effect.

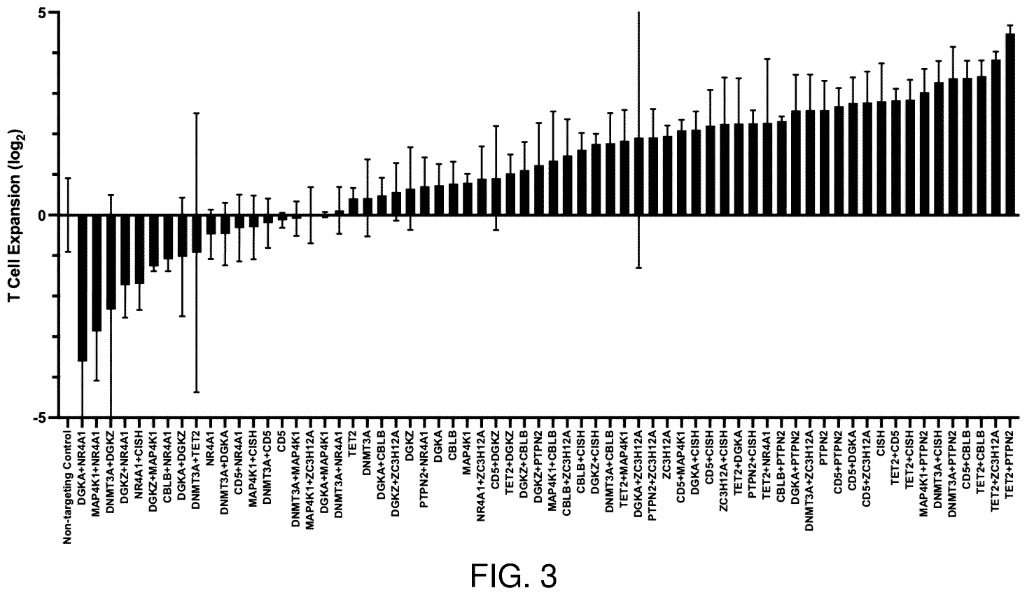

In addition to the varied impact on T cell mediated killing of target cells, a wide range of T cell expansion was observed among the single- and double-gene perturbations, summarized in FIG. 3, ranging from a modest decrease in expansion of T-cells harboring perturbations to DGKA+NR4A1, to over 4-log₁₀ fold increase in expansion observed with the combined perturbation of TET2 and PTPN2 relative to a non-targeting control. Combined perturbations with TET2 exhibited pronounced improvements in T cell killing, with five of the top ten combination perturbations including TET2 as one of the component genes. A number of gene perturbation combinations were identified which exhibited statistically significant superior T cell expansion relative to either individual component gene, summarized in Table 3, indicating that combined perturbation of these genes offers a benefit relative to individual gene perturbation. Further, many gene perturbation combinations exhibited statistically significant superior T cell expansion relative to the synthetic sum of individual combinations, summarized in Table 4 indicating that these combinations drive super-additive killing activity.

Table 3 provides gene knockout combinations that conferred superior T cell expansion to either individual component gene.

TABLE 3

| | | | Fold | | Fold | | | |
|---|---|---|---|---|---|---|---|---|
| | | | change: | FDR: | change: | FDR: | Fold- | |
| | | | pair vs | pair vs | pair vs | pair vs | Max | Max |
| Gene Pair | Gene A | Gene B | Gene A | Gene A | Gene B | Gene B | FDR | FDR |
| CD5 + CBLB | CD5 | CBLB | 3.38 | 0.0316 | 2.54 | 0.0472 | 2.54 | 0.0472 |
| CD5 + MAP4K1 | CD5 | MAP4K1 | 2.22 | 0.0221 | 1.38 | 0.0393 | 1.38 | 0.0393 |
| TET2 + CBLB | TET2 | CBLB | 2.86 | 0.0245 | 2.62 | 0.0472 | 2.62 | 0.0472 |
| TET2 + CD5 | TET2 | CD5 | 2.19 | 0.0221 | 2.79 | 0.0221 | 2.19 | 0.0221 |
| TET2 + ZC3H12A | TET2 | ZC3H12A | 3.22 | 0.0117 | 1.86 | 0.0245 | 1.86 | 0.0245 |

Table 4 provides gene knockout combinations that conferred super-additive T cell expansion relative to either individual component gene.

TABLE 4

| | | | Fold | |
|---|---|---|---|---|
| | | | change: | FDR: |
| | | | pair vs | pair vs |
| | | | Gene A + | Gene A + |
| Gene Pair | Gene A | Gene B | Gene B | Gene B |
| CD5 + CBLB | CD5 | CBLB | 2.6 | 0.0079 |
| CD5 + MAP4K1 | CD5 | MAP4K1 | 1.44 | 0.012 |
| TET2 + CBLB | TET2 | CBLB | 2.08 | 0.011 |
| TET2 + CD5 | TET2 | CD5 | 2.25 | 0.0075 |
| TET2 + PTPN2 | TET2 | PTPN2 | 1.27 | 0.045 |
| TET2 + ZC3H12A | TET2 | ZC3H12A | 1.32 | 0.0075 |

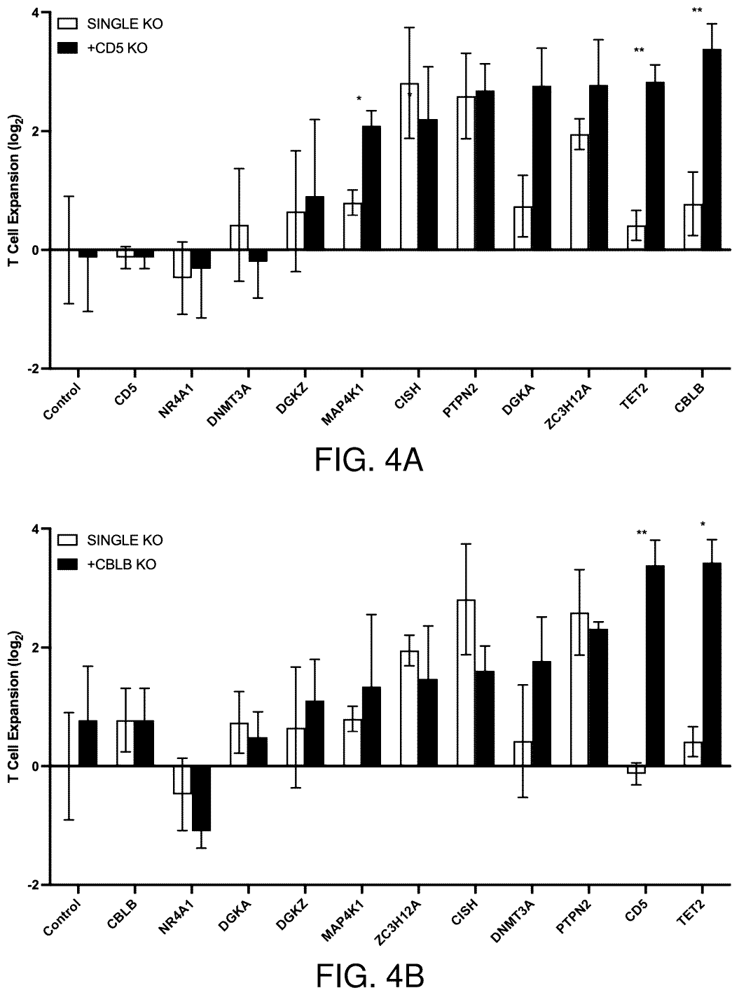

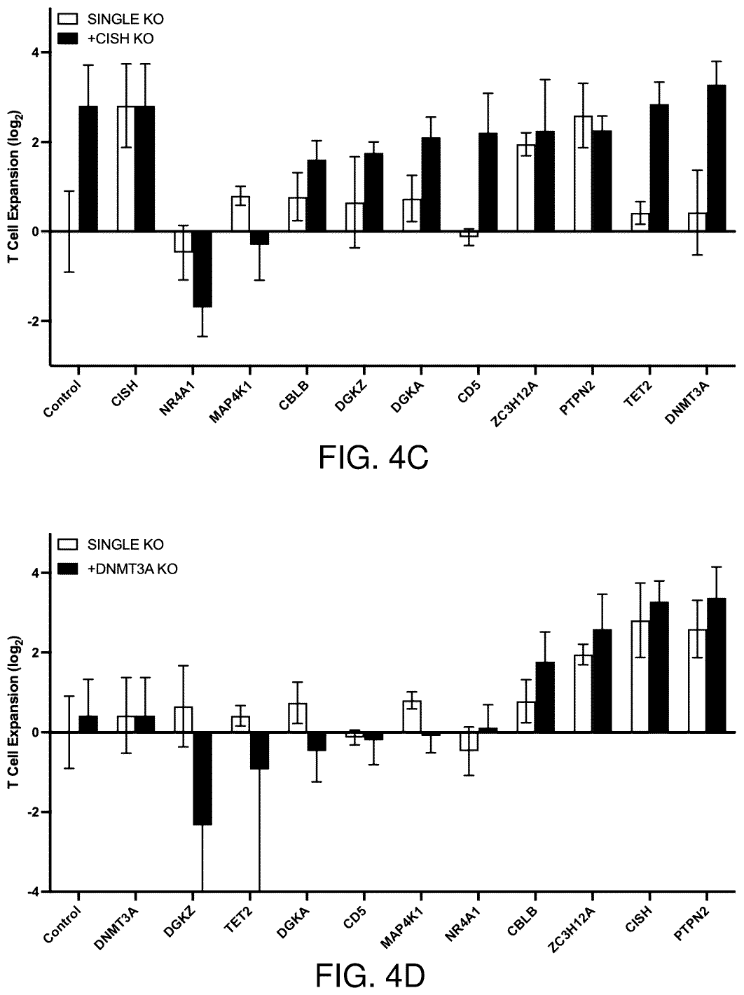

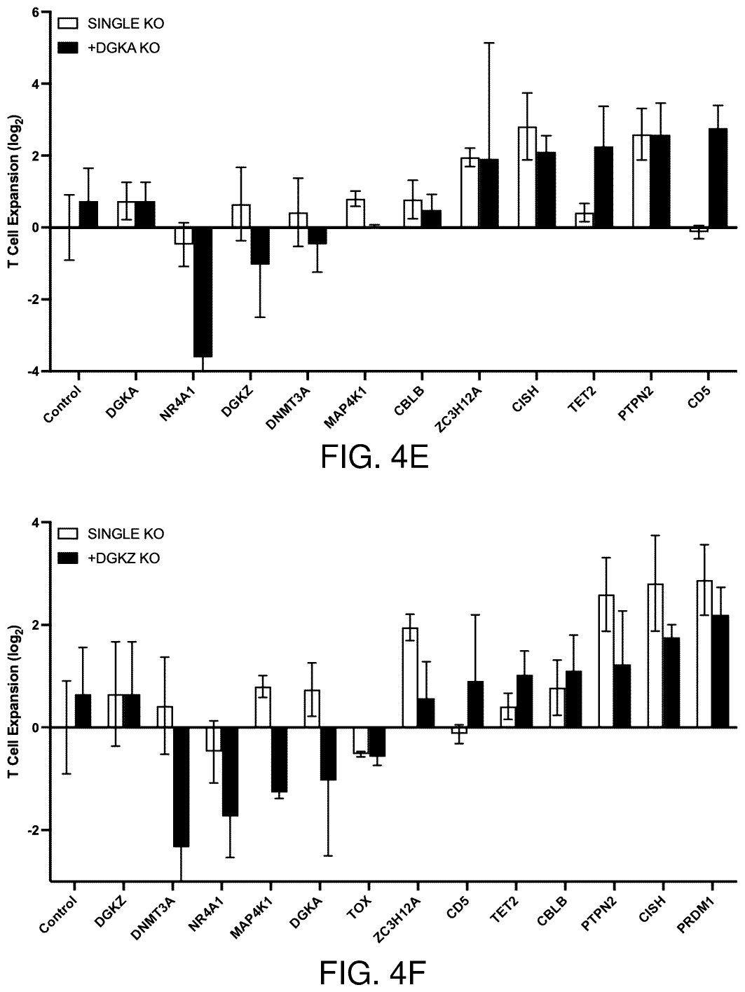

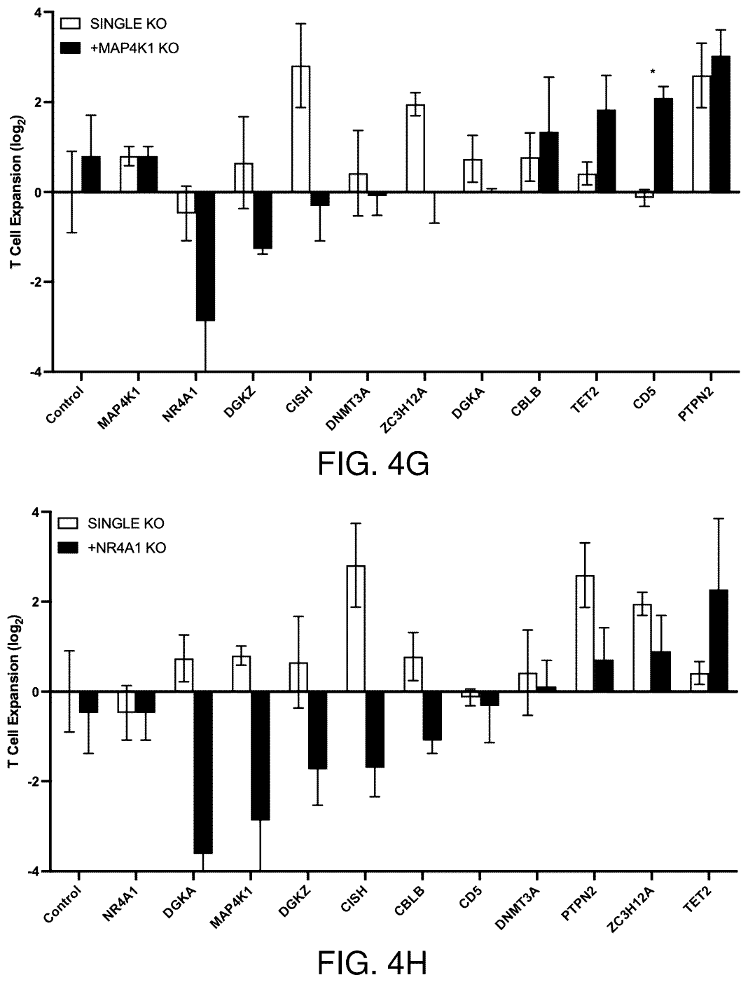

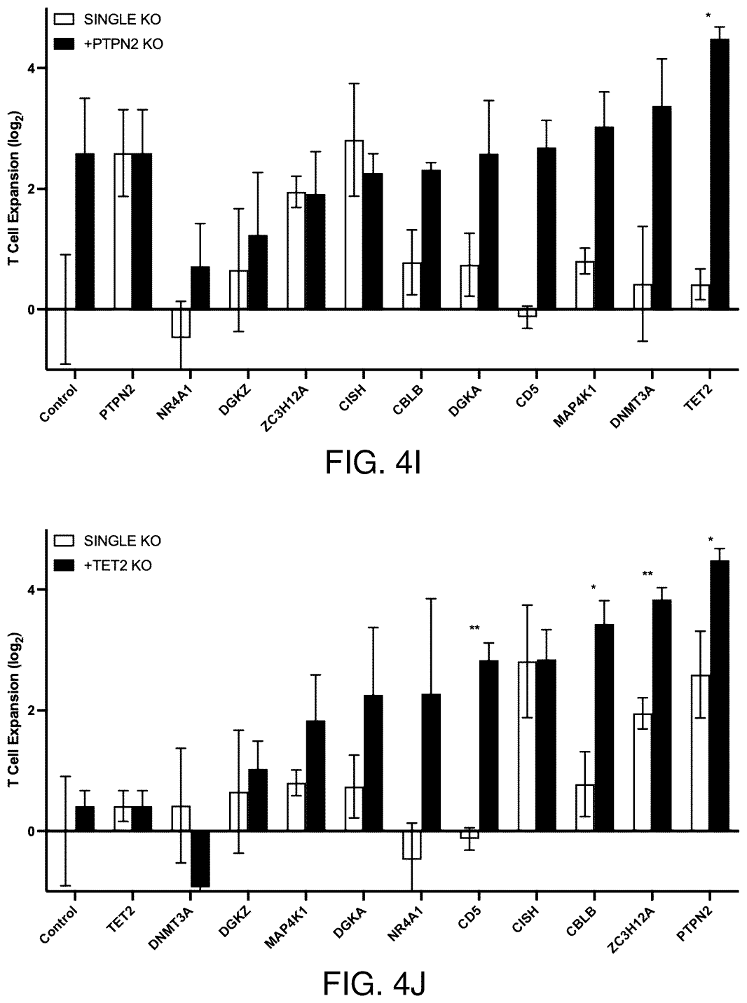

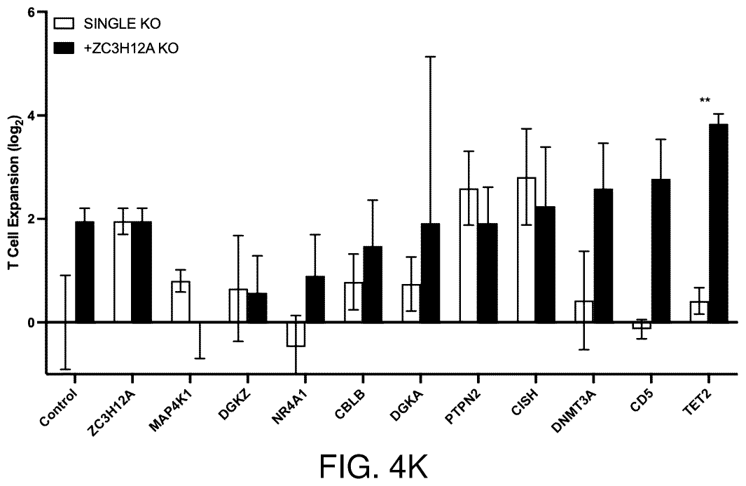

CRISPR-mediated perturbation of CD5 did not significantly impact T cell expansion relative to a non-targeting control (FIG. 4A). Pairwise CD5 perturbation with MAP4K1, TET2, and CBLB improved expansion compared to either component gene or the synthetic sum of these genes, indicating that coincident perturbation of these genes produces a super additive effect. Coincident perturbation of CD5 with NR4A1, DNMT3A, DGKZ, CISH, PTPN2, DGKA, or ZC3H12A did not produce a super-additive effect, although significant impacts were observed with the combinations of CD5 with DGKA or MAP4K1 relative to perturbations of these genes in isolation.

CRISPR-mediated perturbation of CBLB improved T cell expansion relative to a non-targeting control (FIG. 4B). Pairwise CBLB perturbation with CD5 and TET2 improved expansion compared to either component gene or the synthetic sum of these genes, indicating that coincident perturbation of these genes produces a super additive effect. Coincident perturbation of CBLB with NR4A1, DGKA, DGKZ, MPA4K1, ZC3H12A, CISH, DNMT3A, or PTPN2 did not produce a super-additive effect, although significant impacts were observed with the combinations of CBLB with DGKA or MAP4K1 relative to perturbations of these genes in isolation.

CRISPR-mediated perturbation of CISH increased T cell expansion relative to a non-targeting control by roughly 3 logs (FIG. 4C). Pairwise CISH perturbation with other genes did not significantly improve expansion in any pairing beyond the impact of either individual component gene perturbation.

CRISPR-mediated perturbation of DNMT3A did not significantly impact T cell expansion relative to a non-targeting control (FIG. 4D). Pairwise DNMT3A perturbation with other genes did not significantly improve expansion in any pairing beyond the impact of either individual component gene perturbation.

CRISPR-mediated perturbation of DGKA increased T cell expansion relative to a non-targeting control (FIG. 4E). Pairwise DGKA perturbation with other genes did not significantly improve expansion in any pairing beyond the impact of either individual component gene perturbation.

CRISPR-mediated perturbation of DGKZ did not significantly impact T cell expansion relative to a non-targeting control (FIG. 4F). Pairwise DGKZ perturbation with other genes did not significantly improve expansion in any pairing beyond the impact of either individual component gene perturbation.

CRISPR-mediated perturbation of MAP4K1 improved T cell expansion relative to a non-targeting control (FIG. 4G). Pairwise MAP4K1 perturbation with CD5 improved expansion compared to either component gene or the synthetic sum of these genes, indicating that coincident perturbation of these genes produces a super additive effect. Coincident perturbation of MAP4K1 with NR4A1, DGKZ, CISH, DNMT3A, ZC3H12A, DGKA, CBLB, TET2, or PTPN2 did not produce a super-additive effect, although significant impacts were observed with the combinations of MAP4K1 with TET2 relative to either gene perturbation in isolation.

CRISPR-mediated perturbation of NR4A1 did not significantly impact T cell expansion relative to a non-targeting control (FIG. 4H). Pairwise NR4A1 perturbation with other genes did not significantly improve expansion in any pairing beyond the impact of either individual component gene perturbation.

CRISPR-mediated perturbation of PTPN2 increased T cell expansion relative to a non-targeting control by roughly 2 logs (FIG. 4I). Pairwise PTPN2 perturbation with TET2 improved expansion compared to either component gene or the synthetic sum of these genes, indicating that coincident perturbation of these genes produces a super additive effect. Coincident perturbation of MAP4K1 with NR4A1, DGKZ, ZC3H12A, CISH, CBLB, DGKA, CD5, MAP4K1, or DNMT3A did not produce a super-additive effect.

CRISPR-mediated perturbation of TET2 modestly improved T cell expansion relative to a non-targeting control (FIG. 4J). Pairwise TET2 perturbation with CD5, CBLB, ZC3H12A, or PTPN2 improved expansion compared to either component gene or the synthetic sum of these genes, indicating that coincident perturbation of these genes produces a super additive effect. Coincident perturbation of TET2 with DNMT3A, DGKZ, MAP4K1, DGKA, NR4A1, or CISH did not produce a super-additive effect.

CRISPR-mediated perturbation of ZC3H12A improved T cell expansion relative to a non-targeting control (FIG. 4K). Pairwise ZC3H12A perturbation with TET2 improved expansion compared to either component gene or the synthetic sum of these genes, indicating that coincident perturbation of these genes produces a super additive effect. Coincident perturbation of TET2 with MAP4K1, DGKZ, NR4A1, CBLB, DGKA, PTPN2, CISH, DNMT3A, or CD5 did not produce a super-additive effect.

### Example 2: Characterization of Combination Gene Perturbations In Vitro and In Vivo

### Materials

### Logic Gated CAR T Generation

T cells from donors were isolated from Leukopacks® and activated (Day 0). 48 hours post-activation, T cells were engineered (Day 2). To engineer the T cells, sgRNA targeting the designated site or against CD5 (SEQ ID NO: 12), CBLB (SEQ ID NO: 13), CISH (SEQ ID NO: 14), DGKA (SEQ ID NO: 15), DNMT3A (SEQ ID NO: 17), PTPN2, TET2, FAS (SEQ ID NO: 207) and/or ZC3H12A or a dual shRNA construct targeting FAS (SEQ ID NO: 208) and PTPN2 (SEQ ID NO: 141) or a dual shRNA control targeting luciferase (SEQ ID NOs: 208 and 209) were complexed with sNLS-SpCas9-sNLS Nuclease at room temperature for 10 minutes, forming the ribonucleoprotein mix. Specific sgRNA combinations are provided in Table 5. Plasmids containing the logic gate CAR (SEQ ID NO: 212) and PrimeR (SEQ ID NO: 213) and ThermoFisher™ Gene Editing Buffer were then added to the ribonucleoprotein and mixed. The mix was added to activated T cells and electroporated with the Xenon Electroporator and Singleshot system. After electroporation, the engineered T cells were recovered using fresh media supplemented with 12.5 ng/ml of IL-7 and IL-15. The engineered T cells were replenished with fresh media supplemented with 12.5 ng/mL of IL-7 and IL-15 on Day 3 and 5. 6 days post-engineering (Day 8), the cells were assessed for logic gate insertion and cryopreserved.

### Repeat Stimulation Assay In Vitro Evaluation of Logic Gated CAR T

ALPG/MSLN logic gate plus genetic knockouts or shRNA knockdowns were compared in a repeat stimulation assay with H1975 target cells. Engineered ALPG/MSLN CAR were enriched for CAR+ cells via a Myc surface protein tag. H1975 target cells were seeded at 10,000 cells/well in a 96 well plate and 10,000 Myc+CAR T were added for a 1:1 target to CAR ratio. Target cell killing was monitored via a fluorescent tag in target cells and the Incucyte® system for real time imaging. Every 3 days ½ of T cells were removed and seeded on 10,000 fresh target cells for 4 total target cell stimulations. Between reseeding ¼ of T cells were collected for flow analysis for CAR expansion. Performance was quantified as fold improvement over CAR alone in both proliferation and target cell killing.

### Tumor Model In Vivo Evaluation of Logic Gated CAR T

To evaluate the efficacy of T cells engineered with the ALPG/MSLN logic gate, a subcutaneous model of lung cancer H1975 was utilized. Logic gate plus genetic knockouts against combinations of CBLB, CISH, DGKA, DNMT3A, PTPN2, TET2, and/or ZC3H12A genes were compared against non-edited control T cells. NSG MHC I/II KO mice were injected with Hi975 cells overexpressing hALPG and hMSLN. H1975 cells were injected in 100 uL 1:1 suspension of phosphate-buffered saline (PBS) and into the right flank of mice. Animals were randomized into treatment groups according to tumor volumes and were injected into the tail vein with T cells at 0.1×10⁶ dose. Tumor volumes were measured before and twice per week after treatment was started and calculated as V=(length×width2)/2.

### Results

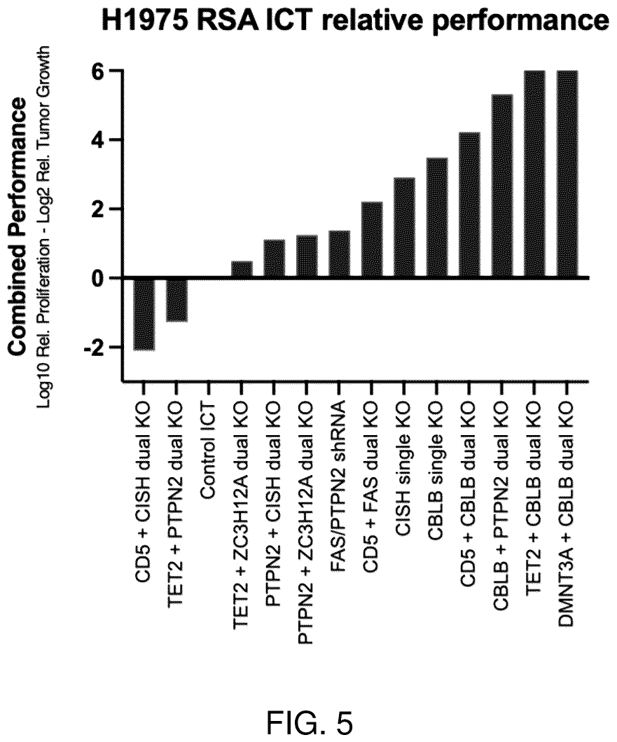

To assess the effects of gene perturbations on the performance of Logic Gate-expressing CAR-T cells, a repetitive stimulation assay was carried out using H1975 cells expressing an exemplary ALPG/MSLN logic gate with CRISPR-mediated perturbations or shRNA-mediated knockdown of selected gene combinations. As shown in FIG. 5, several tested combinations of gene perturbation(s) or knockdown(s) enhanced combined performance of logic gate-expressing T Cells, with two combinations decreasing combined performance. A summary of combinations that enhanced performance of logic gate T cells is provided in Table 5.

TABLE 5
provides gene knockout or knockdown combinations that
conferred enhanced performance relative to control
logic gate T cells.

| | | | Combined |
|---|---|---|---|
| | | | Performance |
| Gene Pair | Gene A | Gene B | Metric |
| TET2 + ZC3H12A | TET2 | ZC3H12A | 0.50 |
| PTPN2 + CISH | PTPN2 | CISH | 1.12 |
| PTPN2 + ZC3H12A | PTPN2 | ZC3H12A | 1.25 |
| Fas + PTPN2 (shRNA) | FAS | PTPN2 | 1.38 |
| CD5 + FAS | CD5 | FAS | 2.22 |
| CISH (single) | CISH | n/a | 2.91 |
| CBLB (single) | CBLB | n/a | 3.49 |
| CD5 + CBLB | CD5 | CBLB | 4.22 |
| CBLB + PTPN2 | CBLB | PTPN2 | 5.32 |
| TET2 + CBLB | TET2 | CBLB | 6.12 |
| DNMT3A + CBLB | DNMT3A | CBLB | 6.34 |

To assess the effects of combined gene knockdown on in vivo anti-tumor activity of logic gate T cells, H1975 cells expressing an exemplary ALPG/MSLN logic gate with CRISPR-mediated perturbations of selected gene combinations were tested in a subcutaneous NSG lung cancer model.

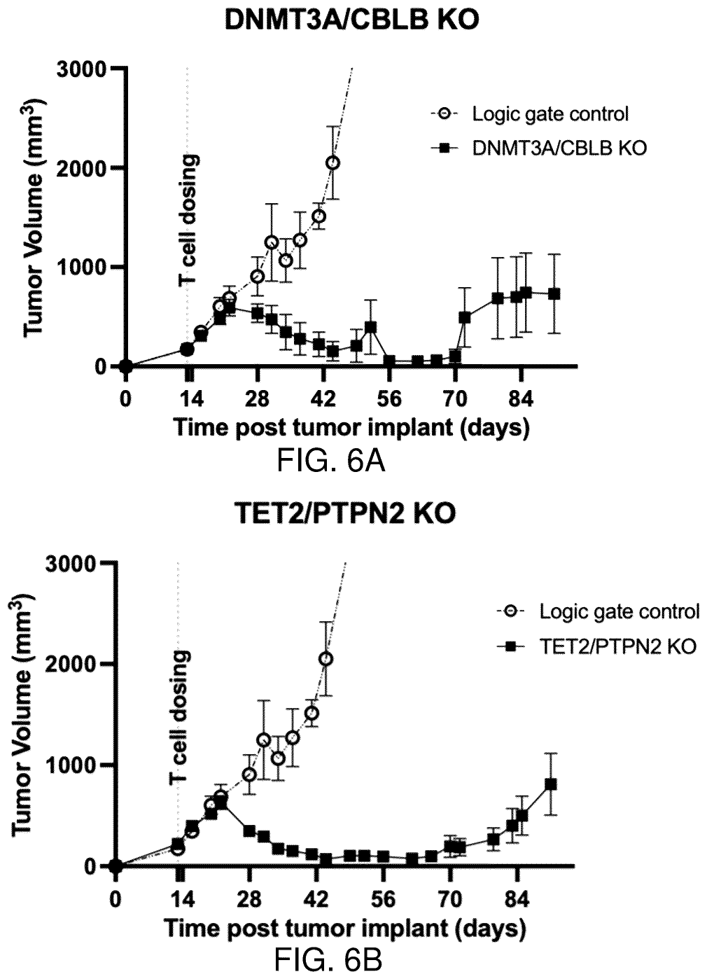

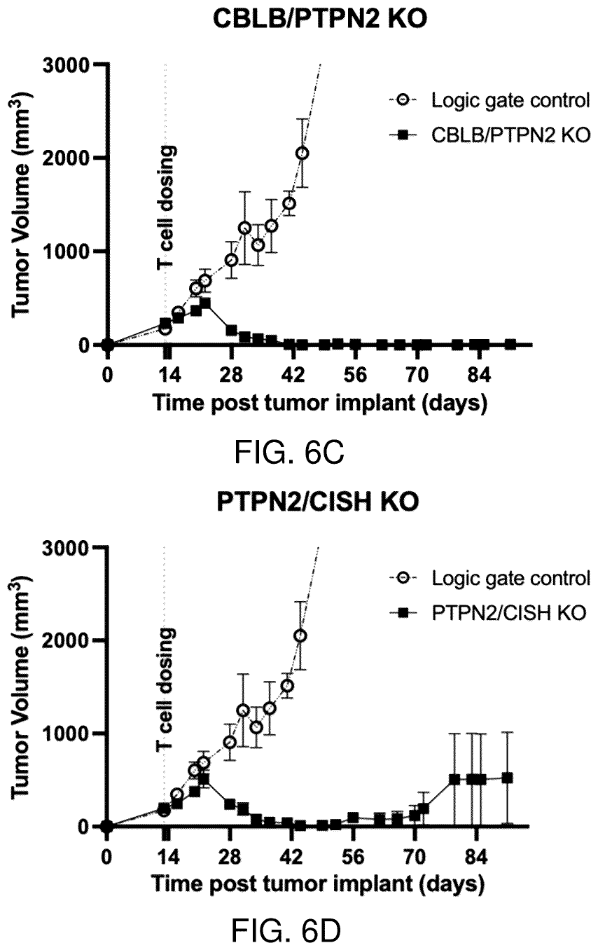

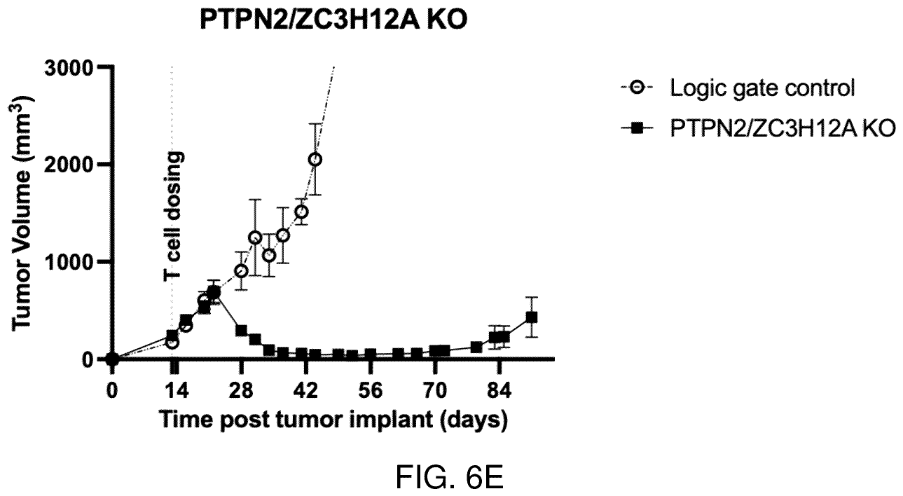

Combined knockout of DNMT3A and CBLB led to an initial decrease in tumor volume with a subsequent expansion beginning after 70 days post-tumor engraftment (FIG. 6A). Combined knockout of TET2 and PTPN2 led to an initial decrease in tumor volume with a subsequent expansion beginning around 70 days post-tumor engraftment (FIG. 6B). Combined knockout of CBLB and PTPN2 led to a sustained decrease in tumor volume over the duration of the experiment (FIG. 6C). Combined knockout of PTPN2 and CISH led to an initial decrease in tumor volume with a subsequent expansion beginning around 70 days post-tumor engraftment (FIG. 6D). Combined knockout of PTPN2 and ZC3H12A led to an initial decrease in tumor volume with a subsequent expansion beginning around 80 days post-tumor engraftment (FIG. 6E).

### Example 3: Validation of shRNAs Against Target Genes In Vitro

### Methods

shRNA-Mediated Gene Knock-Down

T cells from at least 3 donors were engineered to express shRNA modules containing sequences against luciferase control (SEQ ID NOs: 205 and 206), or against CBLB (SEQ ID NOs: 23-46), CISH (SEQ ID NOs: 73-95), DGKA (SEQ ID NOs: 181-204), DNMT3A (SEQ ID NOs: 96-122), PTPN2 (SEQ ID NOs: 123-146), TET2 (SEQ ID NOs: 147-175), or ZC3H12A (SEQ ID NOS: 176-180). Six days post-engineering, magnetic enrichment was performed using Dynabeads MyOne Streptavidin T1 and a biotinylated anti-Myc antibody. The highly pure populations of edited T cells (i.e., >80%) by measurement with flow cytometry using anti-Myc PE were then lysed and mRNA was extracted using the Dynabeads mRNA Direct Purification Kit. Once extracted, the mRNA was quantified using the Quant-it RiboGreen RNA Assay Kit, and used to synthesize cDNA with the SuperScript IV First-Strand Synthesis kit. The cDNA was then used to perform real-time Quantitative Reverse Transcription PCR (qPCR) with the TaqMan Fast Advanced Master Mix and RPL13A, CBLB, CISH, DGKA, DNMT3A, PTPN2, TET2, or ZC3H12A TaqMan assays. Raw data was opened and exported using Design and Analysis software by ThermoFisher Scientific.

For assessment of CD5 knockdown, T cells from at least 3 donors were engineered to express shRNA modules containing sequences against luciferase (control) or against CD5 (SEQ ID NOs: 47-72). Six days post-editing, T cells were stained for Myc and CD5 expression using anti-Myc AF647 and anti-CD5 PE, respectively, and analyzed by flow cytometry on an Attune NxT flow cytometer. Relative CD5 expression was quantified by taking the ratio of the gMFI of CD5 for Myc+ cells divided by Myc-cells. This value was then normalized to the relative CD5 expression of the control group to calculate knockdown.

For simultaneous assessment of on-target and off-target effects of ZC3H12A shRNAs (SEQ ID NOs 176-180 and 205-207), T cells were engineered with an ICT and single shRNAs against ZC3H12A. As a positive control for on-target, indirect effects, cells were separately engineered with an exemplary MSLN/APLG logic gate and a single, validated sgRNA against ZC3H12A. Six days after engineering, logic gate-expressing cells were enriched by positive selection using an anti-Myc antibody. Enriched cell pellets were lysed and RNA was extracted.

Enriched cell pellets were resuspended in lysis buffer and transferred into a 384-well plate. Barcoded oligoDT primers with UMIs were dispensed into individual cell lysate and samples were incubated for primer annealing. Reverse transcription master mix with diluted ERCC synthetic control was dispensed to each sample and the plate was incubated for reverse transcription. Reverse transcribed samples were pooled into one reaction and purified with Agencourt RNAClean XP beads. Purified sample was digested with Exonuclease I and then amplified. Post cDNA amplification, cDNA was purified with Agencourt SPRISelect XP beads. Purified cDNA was tagmented with Illumina transposase and indexed. Libraries were quantified with Tapestation before sequencing on NovaSeq (Illumina). A minimum of two million reads per cell pellet were targeted for sequencing

RNA-seq reads were aligned to the GRCh38 genome using STAR (v2.7.7a) and the STARsolo mode to deduplicate UMIs and assign reads to samples via HT-RNA sample barcodes. Expression was quantified also by STAR using the quantmode GeneCounts option and the Ensembl GRCh38 genome annotation. Differential expression analysis was performed using edgeR (v3.34).

Differentially expressed genes were selected based on comparison between control and shRNA or control and sgRNA samples (defined as having an FDR less than 0.05). From this filtered gene list, a Pearson correlation test was performed between the log 2 fold-change between control vs. shRNA and control vs. sgRNA samples to determine the correlation coefficient and the associated p-value.

### Logic Gated CAR T Generation

T cells from donors were isolated from Leukopacks® and frozen for later use. On day of use, T cells from donors were thawed, and activated (Day 0). 48 hours post-activation, T cells were engineered (Day 2). To engineer the T cells, sgRNA targeting the designated site was complexed with sNLS-SpCas9-sNLS Nuclease at room temperature for 10 minutes, forming the ribonucleoprotein mix. Plasmids containing the shRNA modules containing sequences against luciferase (control) or against combinations of CBLB, CISH, DGKA, DNMT3A, PTPN2, TET2, and/or ZC3H12A outlined in Table 6, plus the logic gate CAR (SEQ ID NO: 212) and primeR (SEQ ID NO: 213) and supplemented Primary P3 Solution were then added to the ribonucleoprotein and mixed. The mix was added to activated T cells and electroporated with the Lonza 96-well Shuttle System. After electroporation, the engineered T cells were recovered using fresh media supplemented with 12.5 ng/ml of IL-7 and IL-15. The engineered T cells were replenished with fresh media supplemented with 12.5 ng/ml of IL-7 and IL-15 on Day 3 and 5. 6 days post-engineering (Day 8), the cells were processed for downstream experiments.

TABLE 6
details tested shRNA combinations

| | | Gene 1 shRNA | | Gene 2 shRNA |
|---|---|---|---|---|
| Combination | Gene 1 | SEQ ID NOS | Gene 2 | SEQ ID NOS |
| DNMT3A + CBLB | DNMT3A | 111, 120, and 122 | CBLB | 29, 37, 45, and 46 |
| PTPN2 + CBLB | PTPN2 | 141, 143, and 146 | CBLB | 29, 37, 44, 45, and 46 |
| TET2 + CBLB | TET2 | 170 and 174 | CBLB | 29, 37, 44, and 46 |
| CD5 + CISH | CD5 | 69, 71, and 72 | CISH | 93, 94, and 95 |
| PTPN2 + CISH | PTPN2 | 141, 143, and 146 | CISH | 93, 94, and 95 |
| PTPN2 + TET2 | PTPN2 | 141, 143, and 146 | TET2 | 170 and 174 |
| PTPN2 + ZC3H12A | ZC3H12A | 141, 143, and 146 | ZC3H12A | 176, 177, and 178 |
| TET2 + ZCH312A | TET2 | 170 and 174 | ZCH312A | 176 and 177 |

### Repeat Stimulation Assay In Vitro Evaluation of Logic Gated CAR T

ALPG/MSLN CAR plus genetic knockdown via dual shRNA modules were compared in a repeat stimulation assay with H1975 target cells. Engineered ALPG/MSLN logic gate-expressing T cells were enriched for CAR+ cells via a Myc surface protein tag

H1975 target cells were seeded at 10,000 cells/well in a 96 well plate and 10,000 Myc+CAR T were added for a 1:1 target to CAR ratio. Target cell killing was monitored via a fluorescent tag in target cells and the Incucyte® system for real time imaging. Every 3 days ½ of T cells were removed and seeded on 10,000 fresh target cells for 4 total target cell stimulations. Between reseeding ¼ of T cells were collected for flow analysis for CAR expansion. Performance was quantified as fold improvement over CAR alone in both proliferation and target cell killing.

### Results

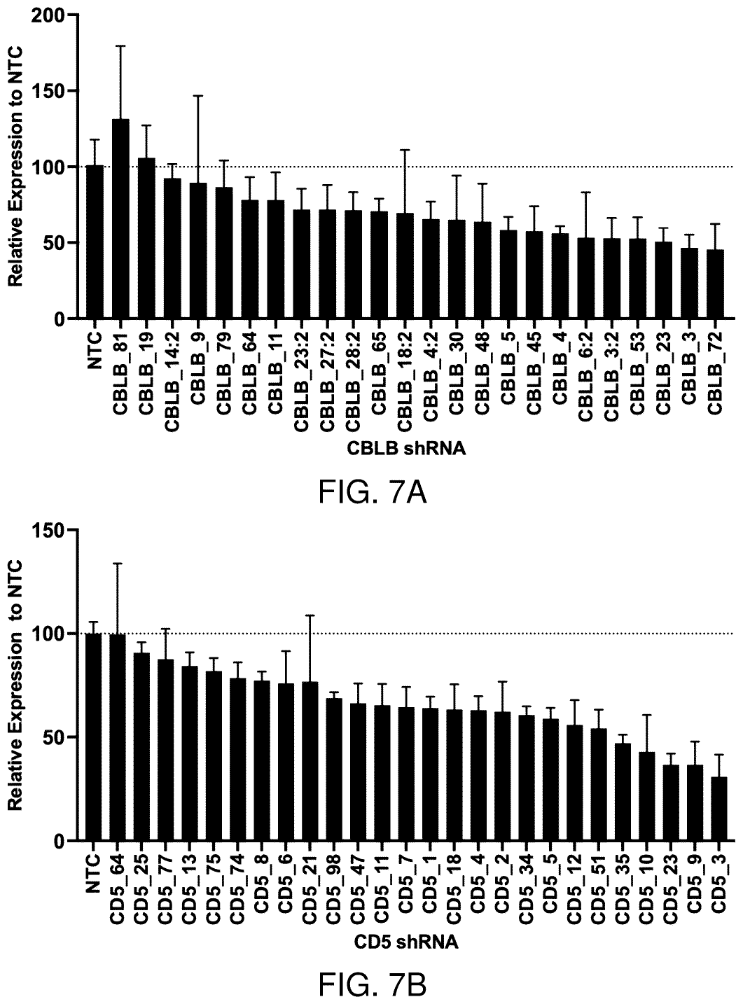

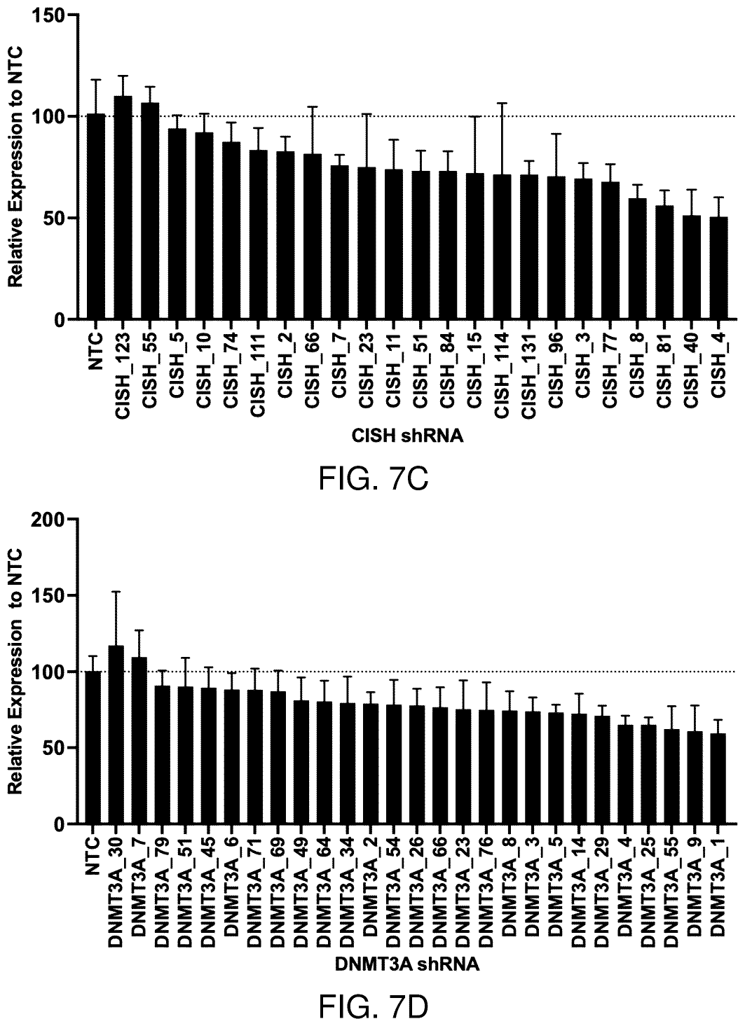

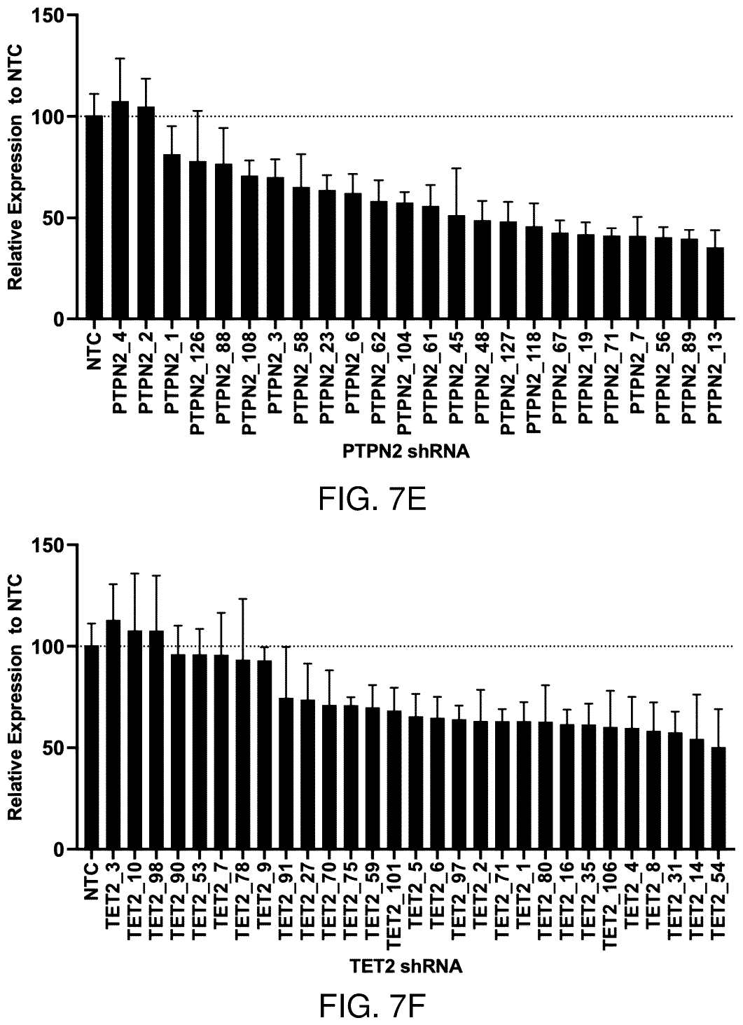

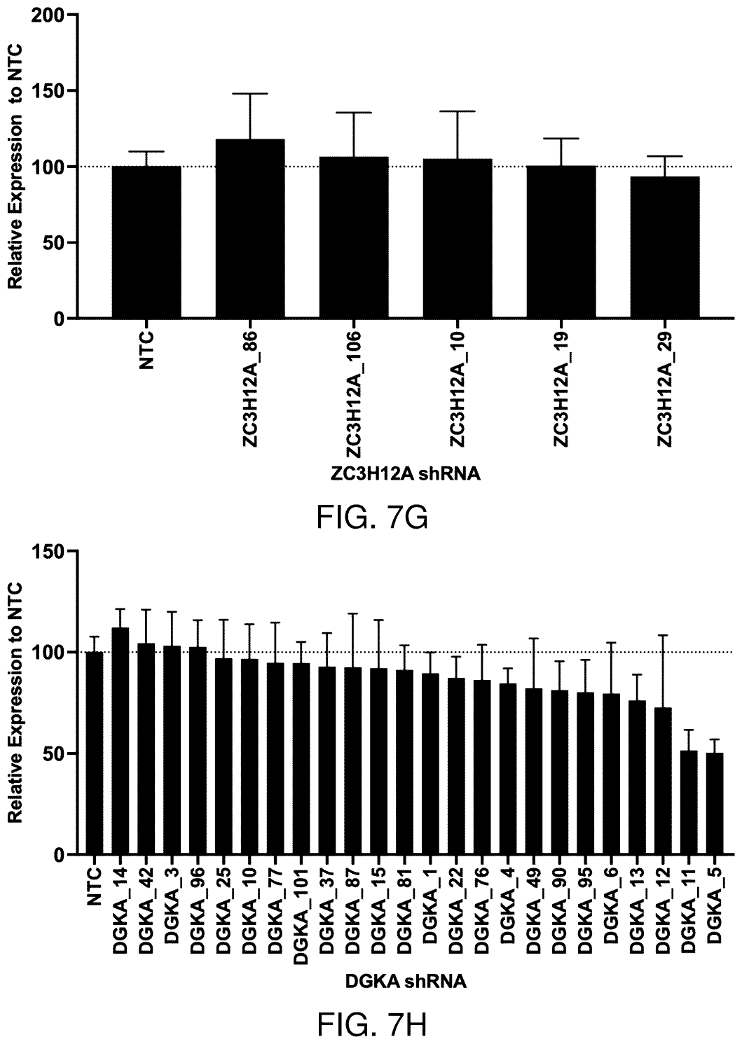

To assess the efficiency of shRNA in suppressing expression of target genes, a qPCR screen for mRNA levels (CBLB, CISH, DNMT3, PTPN2, TET2, ZC3H12A, and DGKA) and a flow cytometry screen for protein levels (CD5) were performed. Results of shRNA screens are shown in FIGS. 7A-7H, and quantification of gene expression for top-performing shRNAs for each gene are listed in Table 7.

TABLE 7
provides quantification top-performing shRNAs for each target gene

| | | Relative | Relative | | | |
|---|---|---|---|---|---|---|
| Target | | Expression | Expression | | | FDR |
| Gene | shRNA | (mean) | (std dev) | P-value | N | (Bonferoni) |
| CBLB | CBLB_11 | 0.7811882902 | 0.1817449062 | 8.40E−03 | 24 | 2.02E−01 |
| CBLB | CBLB_23 | 0.5064121824 | 0.09030256776 | 4.79E−03 | 24 | 1.15E−01 |
| CBLB | CBLB_3 | 0.4653553889 | 0.086001324 | 2.48E−03 | 24 | 5.94E−02 |
| CBLB | CBLB_48 | 0.6400791195 | 0.2490622559 | 1.84E−02 | 24 | 4.42E−01 |
| CBLB | CBLB_72 | 0.455072833 | 0.1699008204 | 7.80E−08 | 24 | 1.87E−06 |
| CD5 | CD5_10 | 0.3388466766 | 0.05749316079 | 5.79E−10 | 13 | 7.53E−09 |
| CD5 | CD5_3 | 0.3009034067 | 0.1194872431 | 4.73E−12 | 13 | 6.15E−11 |
| CD5 | CD5_9 | 0.3522695686 | 0.1236178504 | 9.74E−12 | 13 | 1.27E−10 |
| CISH | CISH_4 | 0.5027719393 | 0.1036409325 | 2.29E−10 | 22 | 5.03E−09 |
| CISH | CISH_40 | 0.4946740586 | 0.1272717367 | 1.76E−07 | 22 | 3.87E−06 |
| CISH | CISH_81 | 0.5622487676 | 0.07461933061 | 6.89E−03 | 22 | 1.52E−01 |
| DGKA | DGKA_11 | 0.5155510818 | 0.1016915177 | 9.51E−03 | 24 | 2.28E−01 |
| DGKA | DGKA_5 | 0.5029489289 | 0.06581258898 | 4.90E−04 | 24 | 1.18E−02 |
| DNMT3A | DNMT3A_1 | 0.5970636409 | 0.08779952857 | 5.73E−05 | 27 | 1.55E−03 |
| DNMT3A | DNMT3A_23 | 0.7551843186 | 0.1879517037 | 2.64E−02 | 27 | 7.14E−01 |
| DNMT3A | DNMT3A_55 | 0.6244501177 | 0.1499761015 | 5.05E−02 | 27 | 1.36E+00 |
| DNMT3A | DNMT3A_9 | 0.6105194531 | 0.1681719158 | 6.90E−02 | 27 | 1.86E+00 |
| PTPN2 | PTPN2_13 | 0.3520551368 | 0.0842121105 | 6.76E−03 | 24 | 1.62E−01 |
| PTPN2 | PTPN2_19 | 0.4095567925 | 0.06622379553 | 1.25E−09 | 24 | 2.99E−08 |
| PTPN2 | PTPN2_7 | 0.3828885454 | 0.1008643433 | 2.75E−05 | 24 | 6.61E−04 |
| TET2 | TET2_106 | 0.6030034673 | 0.1775375391 | 1.63E−04 | 29 | 4.72E−03 |
| TET2 | TET2_14 | 0.5445736972 | 0.2174730229 | 2.27E−01 | 29 | 6.58E+00 |
| TET2 | TET2_54 | 0.503674663 | 0.1867663012 | 7.11E−04 | 29 | 2.06E−02 |
| ZC3H12A | ZC3H12A_10 | 1.05319672 | 0.3104389546 | 7.16E−01 | 5 | 3.58E+00 |
| ZC3H12A | ZC3H12A_106 | 1.067203569 | 0.287356382 | 6.28E−01 | 5 | 3.14E+00 |
| ZC3H12A | ZC3H12A_86 | 1.181297584 | 0.2988798341 | 2.22E−01 | 5 | 1.11E+00 |

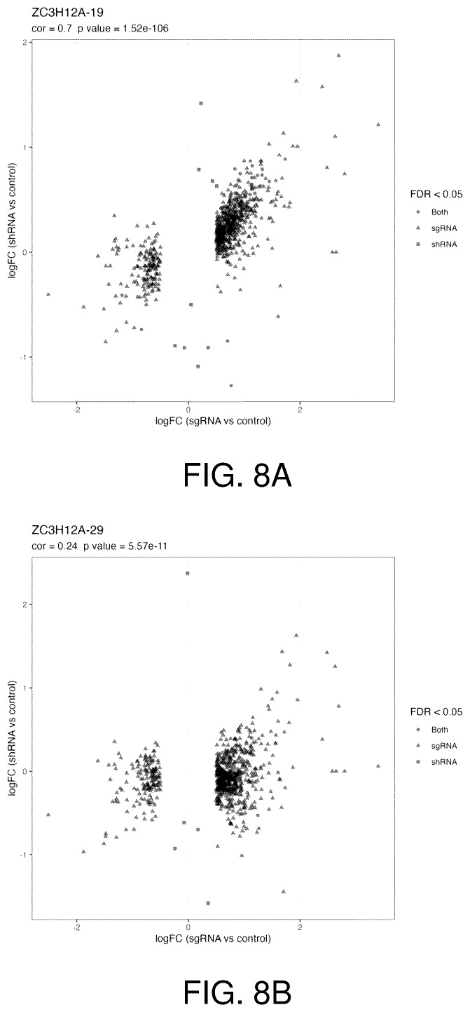

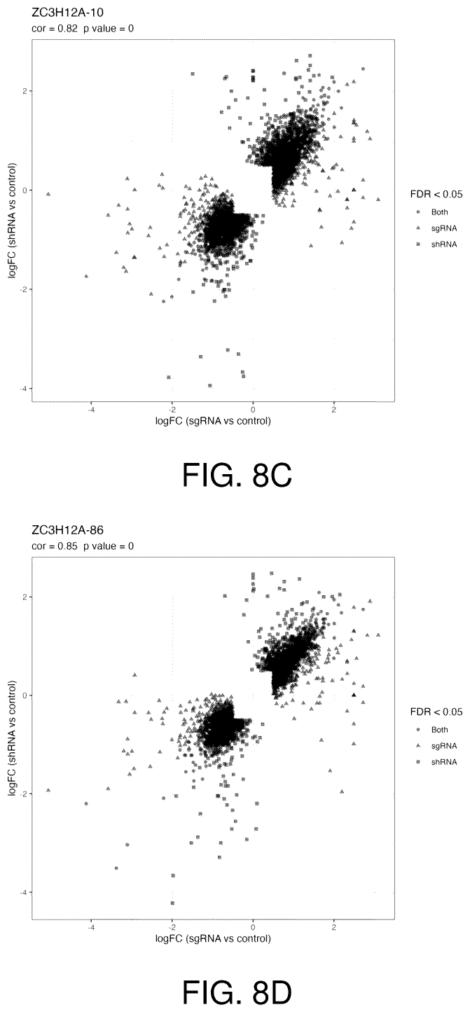

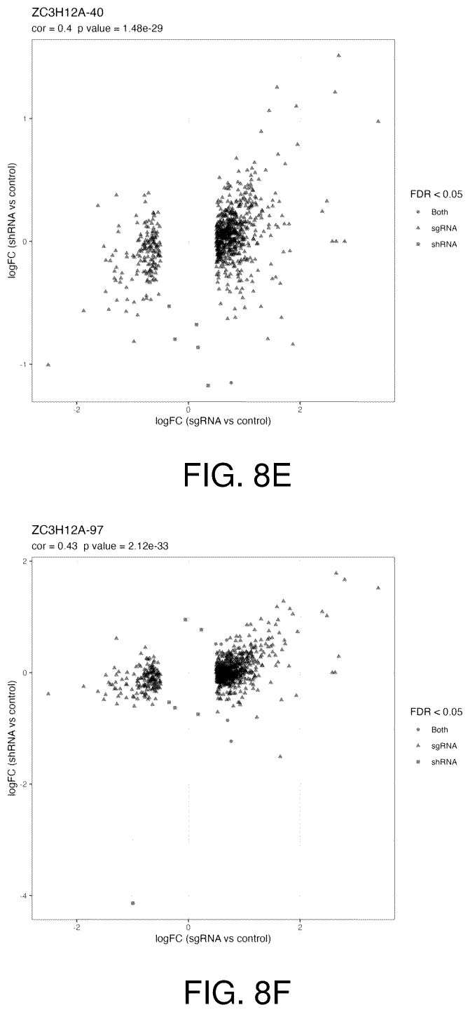

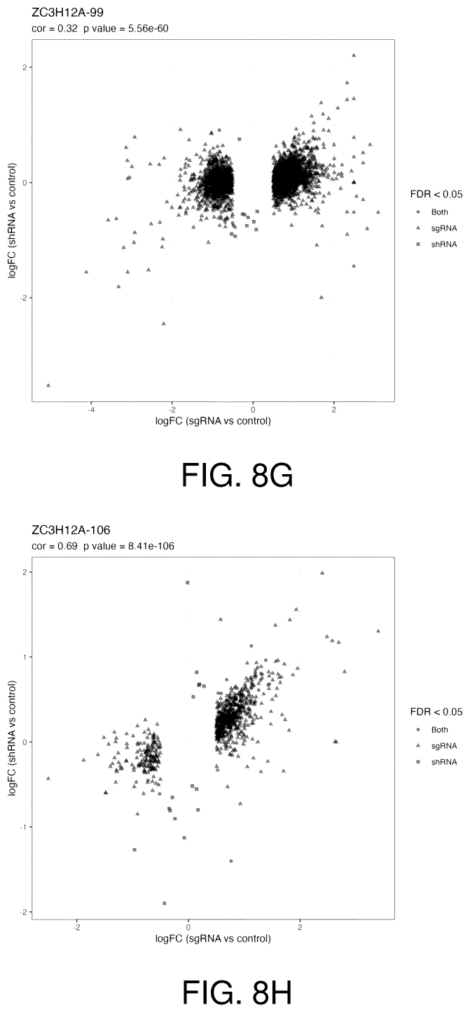

RNAseq was performed to further assess on- and off-target effects of ZC3H12A shRNAs. A significant positive correlation among genes differentially expressed between shRNA or sgRNA vs. control was observed (FIGS. 8A-8H). Low resolution of detection direct knockdown of ZC3H12A 5 was observed using RNAseq or qRT-PCR, shRNAs targeting ZC3H12A were therefore selected for further analysis based on correlation analysis.

A repeat stimulation assay was performed to assess the effects of combined gene targeting on the performance of exemplary ALPG/MSLN logic gate-expressing T cells. Selected shRNAs were tested for the gene combinations outlined in Table 6.

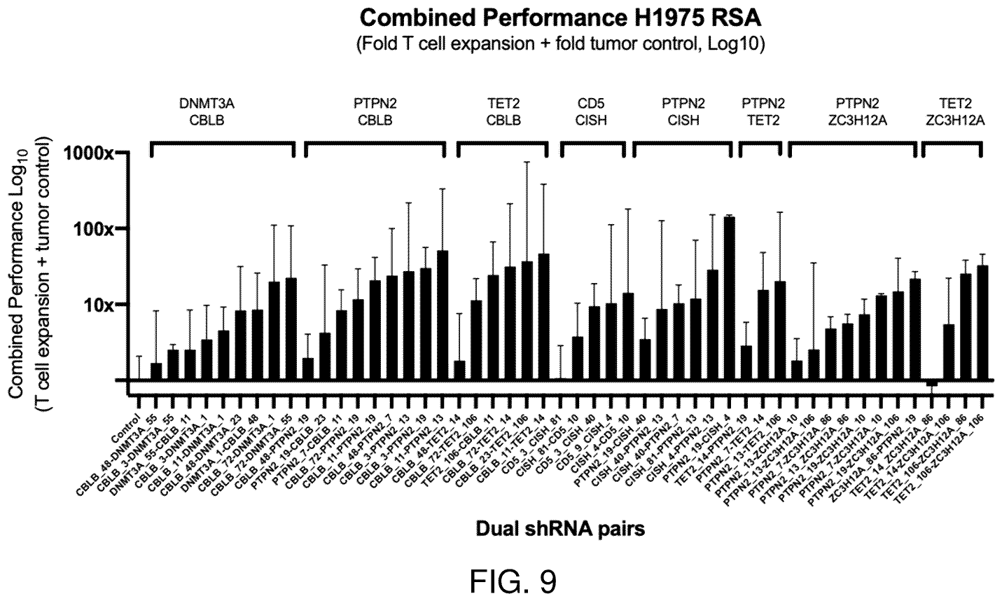

Dual shRNAs with significant enhancement of logic gate-expressing T cells were identified for each gene combination (FIG. 9). Top-performing dual shRNAs for each gene pairing are summarized in Table 8.

TABLE 8
provides shRNA combinations that conferred enhanced
performance relative to control logic gate T cells.

| | | | Combined |
|---|---|---|---|
| | | | Performance |
| Gene Pair | Gene A | Gene B | (log₁₀) |
| CBLB × DNMT3A | CBLB_48 | DNMT3A_55 | 0.23 |
| CBLB × DNMT3A | CBLB_3 | DNMT3A_55 | 0.40 |
| CBLB × DNMT3A | DNMT3A_55 | CBLB_11 | 0.41 |
| CBLB × DNMT3A | CBLB_3 | DNMT3A | 0.54 |
| CBLB × DNMT3A | CBLB_11 | DNMT3A_1 | 0.66 |
| CBLB × DNMT3A | CBLB_48 | DNMT3A_23 | 0.92 |
| CBLB × DNMT3A | DNMT3A 1 | CBLB_48 | 0.93 |
| CBLB × DNMT3A | CBLB_72 | DNMT3A_1 | 1.30 |
| CBLB × DNMT3A | CBLB_72 | DNMT3A_55 | 1.35 |
| CBLB × PTPN2 | CBLB_48 | PTPN2_19 | 0.29 |
| CBLB × PTPN2 | PTPN2_19 | CBLB_23 | 0.63 |
| CBLB × PTPN2 | PTPN2_7 | CBLB_11 | 0.92 |
| CBLB × PTPN2 | CBLB_72 | PTPN2_19 | 1.07 |
| CBLB × PTPN2 | CBLB_11 | PTPN2_19 | 1.32 |
| CBLB × PTPN2 | CBLB_48 | PTPN2_7 | 1.38 |
| CBLB × PTPN2 | CBLB_3 | PTPN2_13 | 1.44 |
| CBLB × PTPN2 | CBLB_3 | PTPN2_19 | 1.48 |
| CBLB × PTPN2 | CBLB_11 | PTPN2_13 | 1.71 |
| CBLB × TET22 | CBLB_48 | TET2_14 | 0.26 |
| CBLB × TET22 | CBLB_72 | TET2_106 | 1.05 |
| CBLB × TET22 | TET2_106 | CBLB_11 | 1.39 |
| CBLB × TET22 | CBLB_72 | TET2_14 | 1.50 |
| CBLB × TET22 | CBLB_23 | TET2_106 | 1.57 |
| CBLB × TET22 | CBLB_11 | TET2_14 | 1.67 |
| CD5 × CISH | CD5_3 | CISH_81 | 0.03 |
| CD5 × CISH | CISH_81 | CD5_10 | 0.58 |
| CD5 × CISH | CD5_3 | CISH_4 | 0.98 |
| CD5 × CISH | CD5_9 | CISH_4 | 1.02 |
| CD5 × CISH | CISH_4 | CD5_10 | 1.15 |
| CISH × PTPN2 | PTPN2_19 | CISH_40 | 0.54 |
| CISH × PTPN2 | CISH_40 | PTPN2_13 | 0.94 |
| CISH × PTPN2 | CISH_40 | PTPN2_7 | 1.02 |
| CISH × PTPN2 | CISH_81 | PTPN2_13 | 1.08 |
| CISH × PTPN2 | CISH_4 | PTPN2_13 | 1.46 |
| CISH × PTPN2 | PTPN2_19 | CISH_4 | 2.15 |
| PTPN2 × TET2 | TET2_14 | PTPN2_19 | 0.46 |
| PTPN2 × TET2 | PTPN2_7 | TET2_14 | 1.19 |
| PTPN2 × TET2 | PTPN2_13 | TET2_106 | 1.31 |
| PTPN2 × ZC3H12A | PTPN2_13 | ZCH12A 14 | 0.26 |
| PTPN2 × ZC3H12A | PTPN2_13 | ZC3H12A_106 | 0.41 |
| PTPN2 × ZC3H12A | PTPN2_7 | ZC3H12A_86 | 0.68 |
| PTPN2 × ZC3H12A | PTPN2_13 | ZC3H12A_86 | 0.75 |
| PTPN2 × ZC3H12A | PTPN2_19 | ZC3H12A 10 | 0.87 |
| PTPN2 × ZC3H12A | PTPN2_7 | ZC3H12A 10 | 1.12 |
| PTPN2 × ZC3H12A | PTPN2_19 | ZC3H12A_106 | 1.17 |
| PTPN2 × ZC3H12A | ZC3H12A_86 | PTPN2_19 | 1.34 |
| TET2 × ZC3H12A | TET2_14 | ZC3H12A_86 | −0.08 |
| TET2 × ZC3H12A | TET2 14 | ZC3H12A_106 | 0.74 |
| TET2 × ZC3H12A | TET2_106 | ZC3H12A_86 | 1.41 |
| TET2 × ZC3H12A | TET2_106 | ZC3H12A_106 | 1.52 |

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

INFORMAL SEQUENCE LISTING

| SEQ ID NO | Name | Sequence |
|---|---|---|
| 1 | CD5 | GAGATACCCGGCCAGACACCCTCACCTGCGGTGCCCAGCTGCCCAGGCTGAGG |
| | mRNA | CAAGAGAAGGCCAGAAACCATGCCCATGGGGTCTCTGCAACCGCTGGCCACCT |
| | NCBI | TGTACCTGCTGGGGATGCTGGTCGCTTCCTGCCTCGGACGGCTCAGCTGGTATG |
| | NM_014207 | ACCCAGATTTCCAGGCAAGGCTCACCCGTTCCAACTCGAAGTGCCAGGGCCAGC |
| | | TGGAGGTCTACCTCAAGGACGGATGGCACATGGTTTGCAGCCAGAGCTGGGGC |
| | | CGGAGCTCCAAGCAGTGGGAGGACCCCAGTCAAGCGTCAAAAGTCTGCCAGCG |
| | | GCTGAACTGTGGGGTGCCCTTAAGCCTTGGCCCCTTCCTTGTCACCTACACACCT |
| | | CAGAGCTCAATCATCTGCTACGGACAACTGGGCTCCTTCTCCAACTGCAGCCAC |
| | | AGCAGAAATGACATGTGTCACTCTCTGGGCCTGACCTGCTTAGAACCCCAGAAG |
| | | ACAACACCTCCAACGACAAGGCCCCCGCCCACCACAACTCCAGAGCCCACAGC |
| | | TCCTCCCAGGCTGCAGCTGGTGGCACAGTCTGGCGGCCAGCACTGTGCCGGCGT |
| | | GGTGGAGTTCTACAGCGGCAGCCTGGGGGGTACCATCAGCTATGAGGCCCAGG |
| | | ACAAGACCCAGGACCTGGAGAACTTCCTCTGCAACAACCTCCAGTGTGGCTCCT |
| | | TCTTGAAGCATCTGCCAGAGACTGAGGCAGGCAGAGCCCAAGACCCAGGGGAG |
| | | CCACGGGAACACCAGCCCTTGCCAATCCAATGGAAGATCCAGAACTCAAGCTG |
| | | TACCTCCCTGGAGCATTGCTTCAGGAAAATCAAGCCCCAGAAAAGTGGCCGAG |
| | | TTCTTGCCCTCCTTTGCTCAGGTTTCCAGCCCAAGGTGCAGAGCCGTCTGGTGGG |
| | | GGGCAGCAGCATCTGTGAAGGCACCGTGGAGGTGCGCCAGGGGGCTCAGTGGG |
| | | CAGCCCTGTGTGACAGCTCTTCAGCCAGGAGCTCGCTGCGGTGGGAGGAGGTGT |
| | | GCCGGGAGCAGCAGTGTGGCAGCGTCAACTCCTATCGAGTGCTGGACGCTGGT |
| | | GACCCAACATCCCGGGGGCTCTTCTGTCCCCATCAGAAGCTGTCCCAGTGCCAC |
| | | GAACTTTGGGAGAGAAATTCCTACTGCAAGAAGGTGTTTGTCACATGCCAGGAT |
| | | CCAAACCCCGCAGGCCTGGCCGCAGGCACGGTGGCAAGCATCATCCTGGCCCT |
| | | GGTGCTCCTGGTGGTGCTGCTGGTCGTGTGCGGCCCCCTTGCCTACAAGAAGCT |
| | | AGTGAAGAAATTCCGCCAGAAGAAGCAGCGCCAGTGGATTGGCCCAACGGGAA |
| | | TGAACCAAAACATGTCTTTCCATCGCAACCACACGGCAACCGTCCGATCCCATG |
| | | CTGAGAACCCCACAGCCTCCCACGTGGATAACGAATACAGCCAACCTCCCAGG |
| | | AACTCCCACCTGTCAGCTTATCCAGCTCTGGAAGGGGCTCTGCATCGCTCCTCC |
| | | ATGCAGCCTGACAACTCCTCCGACAGTGACTATGATCTGCATGGGGCTCAGAGG |
| | | CTGTAAAGAACTGGGATCCATGAGCAAAAAGCCGAGAGCCAGACCTGTTTGTC |
| | | CTGAGAAAACTGTCCGCTCTTCACTTGAAATCATGTCCCTATTTCTACCCCGGCC |
| | | AGAACATGGACAGAGGCCAGAAGCCTTCCGGACAGGCGCTGCTGCCCCGAGTG |
| | | GCAGGCCAGCTCACACTCTGCTGCACAACAGCTCGGCCGCCCCTCCACTTGTGG |
| | | AAGCTGTGGTGGGCAGAGCCCCAAAACAAGCAGCCTTCCAACTAGAGACTCGG |
| | | GGGTGTCTGAAGGGGGCCCCCTTTCCCTGCCCGCTGGGGAGCGGCGTCTCAGTG |
| | | AAATCGGCTTTCTCCTCAGACTCTGTCCCTGGTAAGGAGTGACAAGGAAGCTCA |
| | | CAGCTGGGCGAGTGCATTTTGAATAGTTTTTTGTAAGTAGTGCTTTTCCTCCTTC |
| | | CTGACAAATCGAGCGCTTTGGCCTCTTCTGTGCAGCATCCACCCCTGCGGATCC |
| | | CTCTGGGGAGGACAGGAAGGGGACTCCCGGAGACCTCTGCAGCCGTGGTGGTC |
| | | AGAGGCTGCTCACCTGAGCACAAAGACAGCTCTGCACATTCACCGCAGCTGCC |
| | | AGCCAGGGGTCTGGGTGGGCACCACCCTGACCCACAGCGTCACCCCACTCCCTC |
| | | TGTCTTATGACTCCCCTCCCCAACCCCCTCATCTAAAGACACCTTCCTTTCCACT |
| | | GGCTGTCAAGCCCACAGGGCACCAGTGCCACCCAGGGCCCGGCACAAAGGGGC |
| | | GCCTAGTAAACCTTAACCAACTTGGTTTTTTGCTTCACCCAGCAATTAAAAGTCC |
| | | CAAGCTGAGGTAGTTTCAGTCCATCACAGTTCATCTTCTAACCCAAGAGTCAGA |
| | | GATGGGGCTGGTCATGTTCCTTTGGTTTGAATAACTCCCTTGACGAAAACAGAC |
| | | TCCTCTAGTACTTGGAGATCTTGGACGTACACCTAATCCCATGGGGCCTCGGCT |
| | | TCCTTAACTGCAAGTGAGAAGAGGAGGTCTACCCAGGAGCCTCGGGTCTGATC |
| | | AAGGGAGAGGCCAGGCGCAGCTCACTGCGGCGGCTCCCTAAGAAGGTGAAGCA |
| | | ACATGGGAACACATCCTAAGACAGGTCCTTTCTCCACGCCATTTGATGCTGTAT |
| | | CTCCTGGGAGCACAGGCATCAATGGTCCAAGCCGCATAATAAGTCTGGAAGAG |
| | | CAAAAGGGAGTTACTAGGATATGGGGTGGGCTGCTCCCAGAATCTGCTCAGCTT |
| | | TCTGCCCCCACCAACACCCTCCAACCAGGCCTTGCCTTCTGAGAGCCCCCGTGG |
| | | CCAAGCCCAGGTCACAGATCTTCCCCCGACCATGCTGGGAATCCAGAAACAGG |
| | | GACCCCATTTGTCTTCCCATATCTGGTGGAGGTGAGGGGGCTCCTCAAAAGGGA |
| | | ACTGAGAGGCTGCTCTTAGGGAGGGCAAAGGTTCGGGGGCAGCCAGTGTCTCC |
| | | CATCAGTGCCTTTTTTAATAAAAGCTCTTTCATCTATAGTTTGGCCACCATACAG |
| | | TGGCCTCAAAGCAACCATGGCCTACTTAAAAACCAAACCAAAAATAAAGAGTT |
| | | TAGTTGAGGAGAAA |
| 2 | CBLB | GGGGCCGGGAAGGGGCCTGAGCAGGGAAGGGACACAGGCGTCCTGAAACAAA |
| | mRNA | GCTTTGGGTTGAGGGCAGAGGCGAGGAGCCAGAGAAGTGAACTCCTCCTTTCC |
| | NM_001321786 | GTGGCCGCTCTTGTCATTTTTCTCTTTTCTCTTTTCAAATGGGCTATTTGTGTGTT |
| | | AATTTCATTTGGTTCTTGGGAATAACGACTCACCGCGTTGATTTAAAGAAAGAA |
| | | CTAAAATTCCAGATGGCAAACTCAATGAATGGCAGAAACCCTGGTGGTCGAGG |
| | | AGGAAATCCCCGAAAAGGTCGAATTTTGGGTATTATTGATGCTATTCAGGATGC |
| | | AGTTGGACCCCCTAAGCAAGCTGCCGCAGATCGCAGGACCGTGGAGAAGACTT |
| | | GGAAGCTCATGGACAAAGTGGTAAGACTGTGCCAAAATCCCAAACTTCAGTTG |
| | | AAAAATAGCCCACCATATATACTTGATATTTTGCCTGATACATATCAGCATTTA |
| | | CGACTTATATTGAGTAAATATGATGACAACCAGAAACTTGCCCAACTCAGTGAG |
| | | AATGAGTACTTTAAAATCTACATTGATAGCCTTATGAAAAAGTCAAAACGGGCA |
| | | ATAAGACTCTTTAAAGAAGGCAAGGAGAGAATGTATGAAGAACAGTCACAGGA |
| | | CAGACGAAATCTCACAAAACTGTCCCTTATCTTCAGTCACATGCTGGCAGAAAT |
| | | CAAAGCAATCTTTCCCAATGGTCAATTCCAGGGAGATAACTTTCGTATCACAAA |
| | | AGCAGATGCTGCTGAATTCTGGAGAAAGTTTTTTGGAGACAAAACTATCGTACC |
| | | ATGGAAAGTATTCAGACAGTGCCTTCATGAGGTCCACCAGATTAGCTCTGGCCT |
| | | GGAAGCAATGGCTCTAAAATCAACAATTGATTTAACTTGCAATGATTACATTTC |
| | | AGTTTTTGAATTTGATATTTTTACCAGGCTGTTTCAGCCTTGGGGCTCTATTTTG |
| | | CGGAATTGGAATTTCTTAGCTGTGACACATCCAGGTTACATGGCATTTCTCACA |
| | | TATGATGAAGTTAAAGCACGACTACAGAAATATAGCACCAAACCCGGAAGCTA |
| | | TATTTTCCGGTTAAGTTGCACTCGATTGGGACAGTGGGCCATTGGCTATGTGAC |
| | | TGGGGATGGGAATATCTTACAGACCATACCTCATAACAAGCCCTTATTTCAAGC |
| | | CCTGATTGATGGCAGCAGGGAAGGATTTTATCTTTATCCTGATGGGAGGAGTTA |
| | | TAATCCTGATTTAACTGGATTATGTGAACCTACACCTCATGACCATATAAAAGT |
| | | TACACAGGAACAATATGAATTATATTGTGAAATGGGCTCCACTTTTCAGCTCTG |
| | | TAAGATTTGTGCAGAGAATGACAAAGATGTCAAGATTGAGCCTTGTGGGCATTT |
| | | GATGTGCACCTCTTGCCTTACGGCATGGCAGGAGTCGGATGGTCAGGGCTGCCC |
| | | TTTCTGTCGTTGTGAAATAAAAGGAACTGAGCCCATAATCGTGGACCCCTTTGA |
| | | TCCAAGAGATGAAGGCTCCAGGTGTTGCAGCATCATTGACCCCTTTGGCATGCC |
| | | GATGCTAGACTTGGACGACGATGATGATCGTGAGGAGTCCTTGATGATGAATCG |
| | | GTTGGCAAACGTCCGAAAGTGCACTGACAGGCAGAACTCACCAGTCACATCAC |
| | | CAGGATCCTCTCCCCTTGCCCAGAGAAGAAAGCCACAGCCTGACCCACTCCAGA |
| | | TCCCACATCTAAGCCTGCCACCCGTGCCTCCTCGCCTGGATCTAATTCAGAAAG |
| | | GCATAGTTAGATCTCCCTGTGGCAGCCCAACGGGTTCACCAAAGTCTTCTCCTT |
| | | GCATGGTGAGAAAACAAGATAAACCACTCCCAGCACCACCTCCTCCCTTAAGA |
| | | GATCCTCCTCCACCGCCACCTGAAAGACCTCCACCAATCCCACCAGACAATAGA |
| | | CTGAGTAGACACATCCATCATGTGGAAAGCGTGCCTTCCAGAGACCCGCCAATG |
| | | CCTCTTGAAGCATGGTGCCCTCGGGATGTGTTTGGGACTAATCAGCTTGTGGGA |
| | | TGTCGACTCCTAGGGGAGGGCTCTCCAAAACCTGGAATCACAGCGAGTTCAAAT |
| | | GTCAATGGAAGGCACAGTAGAGTGGGCTCTGACCCAGTGCTTATGCGGAAACA |
| | | CAGACGCCATGATTTGCCTTTAGAAGGAGCTAAGGTCTTTTCCAATGGTCACCT |
| | | TGGAAGTGAAGAATATGATGTTCCTCCCCGGCTTTCTCCTCCTCCTCCAGTTACC |
| | | ACCCTCCTCCCTAGCATAAAGTGTACTGGTCCGTTAGCAAATTCTCTTTCAGAG |
| | | AAAACAAGAGACCCAGTAGAGGAAGATGATGATGAATACAAGATTCCTTCATC |
| | | CCACCCTGTTTCCCTGAATTCACAACCATCTCATTGTCATAATGTAAAACCTCCT |
| | | GTTCGGTCTTGTGATAATGGTCACTGTATGCTGAATGGAACACATGGTCCATCT |
| | | TCAGAGAAGAAATCAAACATCCCTGACTTAAGCATATATTTAAAGGGAGATGTT |
| | | TTTGATTCAGCCTCTGATCCCGTGCCATTACCACCTGCCAGGCCTCCAACTCGGG |
| | | ACAATCCAAAGCATGGTTCTTCACTCAACAGGACGCCCTCTGATTATGATCTTC |
| | | TCATCCCTCCATTAGGTGAAGATGCTTTTGATGCCCTCCCTCCATCTCTCCCACC |
| | | TCCCCCACCTCCTGCAAGGCATAGTCTCATTGAACATTCAAAACCTCCTGGCTC |
| | | CAGTAGCCGGCCATCCTCAGGACAGGATCTTTTTCTTCTTCCTTCAGATCCCTTT |
| | | GTTGATCTAGCAAGTGGCCAAGTTCCTTTGCCTCCTGCTAGAAGGTTACCAGGT |
| | | GAAAATGTCAAAACTAACAGAACATCACAGGACTATGATCAGCTTCCTTCATGT |
| | | TCAGATGGTTCACAGGCACCAGCCAGACCCCCTAAACCACGACCGCGCAGGAC |
| | | TGCACCAGAAATTCACCACAGAAAACCCCATGGGCCTGAGGCGGCATTGGAAA |
| | | ATGTCGATGCAAAAATTGCAAAACTCATGGGAGAGGGTTATGCCTTTGAAGAG |
| | | GTGAAGAGAGCCTTAGAGATAGCCCAGAATAATGTCGAAGTTGCCCGGAGCAT |
| | | CCTCCGAGAATTTGCCTTCCCTCCTCCAGTATCCCCACGTCTAAATCTATAGCAG |
| | | CCAGAACTGTAGACACCAAAATGGAAAGCAATCGATGTATTCCAAGAGTGTGG |
| | | AAATAAAGAGAACTGAGATGGAATTCAAGAGAGAAGTGTCTCCTCCTCGTGTA |
| | | GCAGCTTGAGAAGAGGCTTGGGAGTGCAGCTTCTCAAAGGAGACCGATGCTTG |
| | | CTCAGGATGTCGACAGCTGTGGCTTCCTTGTTTTTGCTAGCCATATTTTTAAATC |
| | | AGGGTTGAACTGACAAAAATAATTTAAAGACGTTTACTTCCCTTGAACTTTGAA |
| | | CCTGTGAAATGCTTTACCTTGTTTACAGTTTGGCAAAGTTGCAGTTTGTTCTTGT |
| | | TTTTAGTTTAGTTTTGTTTTGGTGTTTTGATACCTGTACTGTGTTCTTCACAGACC |
| | | CTTTGTAGCGTGGTCAGGTCTGCTGTAACATTTCCCACCAACTCTCTTGCTGTCC |
| | | ACATCAACAGCTAAATCATTTATTCATATGGATCTCTACCATCCCCATGCCTTGC |
| | | CCAGGTCCAGTTCCATTTCTCTCATTCACAAGATGCTTTGAAGGTTCTGATTTTC |
| | | AACTGATCAAACTAATGCAAAAAAAAAAAAGTATGTATTCTTCACTACTGAGTT |
| | | TCTTCTTTGGAAACCATCACTATTGAGAGATGGGAAAAACCTGAATGTATAAAG |
| | | CATTTATTTGTCAATAAACTGCCTTTTGTAAGGGGTTTTCACATAACATAGAGG |
| | | AGCTTCCCTTTTTTGTTTAAGTTTTGTAACCTTTAATCCTCCATATTCTCATGTCT |
| | | GTCATCCCAGGGGTGTCACAACTGTAAAAATCTACAATATTAGAAAGCAGCTAC |
| | | TACAGATGTGGAAGAGAGAACACTTGTATAAGACAATGCTGTACTGAAGTTAT |
| | | ATAACAAGGTCCCCTGATACTTATGCCTGCATTACTTTGAGGGATGCTGAATGA |
| | | GAGGACCATCTCCCTGAAATATATTAATAATTTTCAGATAAAATTATGAACATA |
| | | ACATACCTTTTCTCAAGTGGAATTAGAGTTCATTTCCCATTTAAGTGATTACACT |
| | | CTATCTCCTAAGAGCTGTGGGAACCAGTTCATATTGCAGTGGAAACATTAACTC |
| | | TTCGTTGCACTGTTGAGTGTCAAGGCTACTGTGGCAATGTTTTTTGCAAAGTTCT |
| | | CAGTAATTTTCTCTCTGCAATCAAATTAGAGTTCATATTTAATTTGCAGATTCTC |
| | | TCATTCATTGCTCCACACTGCTTTATCAAATCAGGTGAAAGAAATTAATGTAGT |
| | | TTTGCCTATTTTACAAAATGGTGGTTGGTTCTTTAAACACCATGTTATTACTCTT |
| | | AATAAATCTACAAATATTTATTGAGCATCTATGATATGCTTGGTGCTGTTAGGC |
| | | ACTGTGTTTTTAAAGCACAAACTCTCAAAAGCTGATTCCAGCGTAATGTTTTATT |
| | | TTTGTTTTTCTGTGTTTTTTATGACAAAGTCTGTCTGATAGACAAAGCCTGGAAG |
| | | GTTTCCCCCAGGGCTTTAGAATTTAGAATTAGCTGGTTTTCATTTCAAATGGGAA |
| | | ATTAATGGAGAAATGAGATAAAATCATCTCTGTGTAAATGCTTGAACCCACAGA |
| | | AAGTTTTCTAAATATGGACTAATTGTGGGTTTGTGCATATTTCTGTGTCTCCTCT |
| | | TTTCACTTCACACCCACCATGATATAAAAAGATGCATCAGAGAGTGAGCCTGCC |
| | | TTCAGGACCTGAAAGGACATGACTGAACATCTAGTAGATCTGGTGGAAATTAAT |
| | | GTTATACAAAAGACACGTAGACTAAGTTGACCCTGGGAGAGGAGTTAATACAT |
| | | TCCTTTGCAAAGTGACATGTGCCCTTTTTTTGACAGTCTGGCTTTCATGGTATGA |
| | | GATGAGGACTCACAGGCCTTGGGTAATTTCTGCTTTTGTATGAATTTTTTCAGAT |
| | | GGTTCTGAGCTGGTTATGTCCCTGGAGGGGAATTTTTCATTTGCCTTAGATGTTC |
| | | TTCCCATGAGTAGGTACATGAGTGAAGTTTCTTACACTTTTAAAATTGCATTGGC |
| | | AAATTTTCTTTTGCTTTATGAACCCCTGTTTTGTCATTATGATTTTTCACTTGTTT |
| | | TAACAGTTTCAGATGTCACCTTGGAATAGCACTGTTTATTTTGGCCTTTTTATGA |
| | | TTGCTGATGATAGGTACAGTCTAAAGGCCAAAAGTTGCCACTTCATTGTGATAA |
| | | CATTGTTGCAACATTCCTAATATATTAGAGAATGATGTAAAATAAGAATGTTAG |
| | | AGAACATAGCACATTCTCATTCAGTTTATAAACAATATTTGAAAATTTCCAGTG |
| | | AGAATTAAAAAAGGACTAGTGATGATATAGCAAACCTGGACCTGAAATTTGGG |
| | | GGTGGGGGGTGGGGGGATATCTGTTTTGCAGAGAAAATTTACCAGCCTCCAGC |
| | | AAAATCTCTGCATCTTGTACTCAATAGCAACCATTAGTATTGTCATTTAAAAAG |
| | | AATGAATCAAAAATTCATACGTAATGGAATGAAAAGAGGAAAACTTTCTATTA |
| | | AACACTGTCAAGAAAGACCCATTGCTATCTATCACCTATGAAATTAAACCTAAA |
| | | AAAATTTTTTTTTATTGTAAAAGCGACTGCTCGTTGATGTCTGGAAGTGATCCAG |
| | | ACTTTGTTTCACTGTGATCCAAAAAGTTAGACATTCCTTCCATTTCTCAAGATTT |
| | | GGTAACAATTTTTTTTTAAGTCAGCATTTCATATAATACATAAAATTAAGACAA |
| | | ATTAACTTACTGATATGTGGCCAATATCAACCCTTCTCACCCAACCCAAACATG |
| | | TTCTGGAGATAGAAGGACTTATCCTCAATTATATTCCAGTAAATTTATCTCCATA |
| | | ATCTGTTTTATCCCCTAATGTATGATATATCTCACCAAATTTTTCTAGAATTGAA |
| | | TAAAAGCAGTTTTTGCTTAACTTGAATTTCCTTCCACAAATACAACCTTCAGCTC |
| | | TTCCCTCTCTGTTTCCCTTTTGCCCACATTGCAAATAGTCTTCATAATAGAACCC |
| | | TCACTGCAACTTAAAAGGGTCCCATTTCTTAACTCAGCATTTCATATACCAAGA |
| | | ACACTTGCAAAGATTTCTTCATAATGTGATTTTTTAATTTTTCAGTTCCAGAAGC |
| | | ATTTCCTACAGTTCGAACGTTATTTTTTCTCTCAGATTTTCAACTTGAAGCTTTG |
| | | ATTTGCACTGAATTAACTTGGATAAAATTTTAGTTTCTCTCTTCAGCTAACAGAT |
| | | TTGGAAGCAAATTACAGTATACTTGAATAATTCAGGTACTACCTGTTGACCTGG |
| | | AATTTTAGTGATTCTGGGAAAGTTTTCCAGACAAAGACACCAATAGAGTGAAG |
| | | GGTTTTTTCCCCAATTACTATTTTATTGTTTTTTAAAATGCCTTTTTAAAACTTGT |
| | | GTGATGTGGCAAAATAGTCATATGTTCAATTAATTTATATTTTATTCATTCCTTT |
| | | TCAATTCTGGTTATTATGTAACCTCAAGTACTTTATCTGTTCTTTTAAAGTATTTT |
| | | ATAAAATGAACGTTAACTAAA |
| 3 | CISH | AGTCCCTGTCCTGCGCCCGCGCGCCCCGGGAGCCTACCCAGCACGCGCTCCGCG |
| | mRNA | CCCACTGGTTCCCTCCAGCCGCCGCCGTCCAGCCGAGTCCCCACTCCGGAGTCG |
| | NCBI | CCGCTGCCGCGGGGACATGGTCCTCTGCGTTCAGGGGTGAGCACCCCCTTGTAA |
| | NM_013324 | GCTCAGGGCTACTGTTGGGTGTCAGGGAACAAAGTTTTAGACTGCTGCGCTCCA |
| | | AAGCGGGCACACACATGTACCTAGAACACACCAGCCACTGTCCCCACCATGAT |
| | | GATGACACAGCCATGGACACACCCCTGCCCAGACCTCGTCCTTTGCTGGCTGTG |
| | | GAGCGGACTGGGCAGCGGCCCCTGTGGGCCCCGTCCCTGGAACTGCCCAAGCC |
| | | AGTCATGCAGCCCTTGCCTGCTGGGGCCTTCCTCGAGGAGGTGGCAGAGGGTAC |
| | | CCCAGCCCAGACAGAGAGTGAGCCAAAGGTGCTGGACCCAGAGGAGGATCTGC |
| | | TGTGCATAGCCAAGACCTTCTCCTACCTTCGGGAATCTGGCTGGTATTGGGGTT |
| | | CCATTACGGCCAGCGAGGCCCGACAACACCTGCAGAAGATGCCAGAAGGCACG |
| | | TTCTTAGTACGTGACAGCACGCACCCCAGCTACCTGTTCACGCTGTCAGTGAAA |
| | | ACCACTCGTGGCCCCACCAATGTACGCATTGAGTATGCCGACTCCAGCTTCCGT |
| | | CTGGACTCCAACTGCTTGTCCAGGCCACGCATCCTGGCCTTTCCGGATGTGGTC |
| | | AGCCTTGTGCAGCACTATGTGGCCTCCTGCACTGCTGATACCCGAAGCGACAGC |
| | | CCCGATCCTGCTCCCACCCCGGCCCTGCCTATGCCTAAGGAGGATGCGCCTAGT |
| | | GACCCAGCACTGCCTGCTCCTCCACCAGCCACTGCTGTACACCTAAAACTGGTG |
| | | CAGCCCTTTGTACGCAGAAGCAGTGCCCGCAGCCTGCAACACCTGTGCCGCCTT |
| | | GTCATCAACCGTCTGGTGGCCGACGTGGACTGCCTGCCACTGCCCCGGCGCATG |
| | | GCCGACTACCTCCGACAGTACCCCTTCCAGCTCTGACTGTACGGGGCAATCTGC |
| | | CCACCCTCACCCAGTCGCACCCTGGAGGGGACATCAGCCCCAGCTGGACTTGGG |
| | | CCCCCACTGTCCCTCCTCCAGGCATCCTGGTGCCTGCATACCTCTGGCAGCTGGC |
| | | CCAGGAAGAGCCAGCAAGAGCAAGGCATGGGAGAGGGGAGGTGTCACACAAC |
| | | TTGGAGGTAAATGCCCCCAGGCCGCATGTGGCTTCATTATACTGAGCCATGTGT |
| | | CAGAGGATGGGGAGACAGGCAGGACCTTGTCTCACCTGTGGGCTGGGCCCAGA |
| | | CCTCCACTCGCTTGCCTGCCCTGGCCACCTGAACTGTATGGGCACTCTCAGCCCT |
| | | GGTTTTTCAATCCCCAGGGTCGGGTAGGACCCCTACTGGCAGCCAGCCTCTGTT |
| | | TCTGGGAGGATGACATGCAGAGGAACTGAGATCGACAGTGACTAGTGACCCCT |
| | | TGTTGAGGGGTAAGCCAGGCTAGGGGACTGCACAATTATACACTATTTATTTAT |
| | | TTATTCTCCTTGGGGTTGGTGTCAGGGGCGAGCCAACCCCACCTCTATGCCCTG |
| | | AGCCCTGGTAGTCCAGAGACCCCAACTCTGCCCTGGCTTCTCTGGTTCTTCCCTG |
| | | TGGAAAGCCCATCCTGAGACATCTTGCTGGAACCAAGGCAATCCTGGATGTCCT |
| | | GGTACTGACCCACCCGTCTGTGAATGTGTCCACTCTCTTCTGCCCCCAGCCATAT |
| | | TTGGGGAGGATGGACAACTACAATAGGTAAGAAAATGCAGCCGGAGCCTCAGT |
| | | CCCCAGCAGAGCCTGTGTCTCACCCCCTCACAGGACAGAGCTGTATCTGCATAG |
| | | AGCTGGTCTCACTGTGGCGCAGGCCCCGGGGGGAGTGCCTGTGCTGTCAGGAA |
| | | GAGGGGGTGCTGGTTTGAGGGCCGCCACTGCAGTTCTGCTAGGTCTGCTTCCTG |
| | | CCCAGGAAGGTGCCTGCACATGAGAGGAGAGAAATACACGTCTGATAAGACTT |
| | | CATGAAATAATAATTATAGCAAAGAACAGTTTGGTGGTCTTTTCTCTTCCACTG |
| | | ATTTTTCTGTAATGACATTATACCTTTATTACCTCTTTATTTTATTACCTCTATAA |
| | | TAAAATGATACCTTTCATGTA |
| 4 | DGKA | AGCTCGGGCTCCAGCTCCAGCGCCGGCGCTTCAGCTGCGACCGCGAGCCCTCTC |
| | mRNA | AAGCAAGGTAGAGACCCCGCGAGCCCCCCAGCGCCCCACCCTCCCCCATCGGA |
| | NCBI | AAAGGACAGGGGTAGGGAAGCCGAGGACCCACGGGCTGCGTTCGTGCTGCTGG |
| | NM_201444 | GTCGGGAAGGAGGAAGCGTGACAGCTGGAGCGGGCCTACCCTCTGAAGAGGTC |
| | | CAAGCAACGGAAGTACTACTACGAAGCTGCCTTTCTGGCCATCCTTGAGAAAAA |
| | | TAGACAGATGGCCAAGGAGAGGGGCCTAATAAGCCCCAGTGATTTTGCCCAGC |
| | | TGCAAAAATACATGGAATACTCCACCAAAAAGGTCAGTGATGTCCTAAAGCTCT |
| | | TCGAGGATGGCGAGATGGCTAAATATGTCCAAGGAGATGCCATTGGGTACGAG |
| | | GGATTCCAGCAATTCCTGAAAATCTATCTCGAAGTGGATAATGTTCCCAGACAC |
| | | CTAAGCCTGGCACTGTTTCAATCCTTTGAGACTGGTCACTGCTTAAATGAGACA |
| | | AATGTGACAAAAGATGTGGTGTGTCTCAATGATGTTTCCTGCTACTTTTCCCTTC |
| | | TGGAGGGTGGTCGGCCAGAAGACAAGTTAGAATTCACCTTCAAGCTGTACGAC |
| | | ACGGACAGAAATGGGATCCTGGACAGCTCAGAAGTGGACAAAATTATCCTACA |
| | | GATGATGCGAGTGGCTGAATACCTGGATTGGGATGTGTCTGAGCTGAGGCCGAT |
| | | TCTTCAGGAGATGATGAAAGAGATTGACTATGATGGCAGTGGCTCTGTCTCTCA |
| | | AGCTGAGTGGGTCCGGGCTGGGGCCACCACCGTGCCACTGCTAGTGCTGCTGGG |
| | | TCTGGAGATGACTCTGAAGGACGACGGACAGCACATGTGGAGGCCCAAGAGGT |
| | | TCCCCAGACCAGTCTACTGCAATCTGTGCGAGTCAAGCATTGGTCTTGGCAAAC |
| | | AGGGACTGAGCTGTAACCTCTGTAAGTACACTGTTCACGACCAGTGTGCCATGA |
| | | AAGCCCTGCCTTGTGAAGTCAGCACCTATGCCAAGTCTCGGAAGGACATTGGTG |
| | | TCCAATCACATGTGTGGGTGCGAGGAGGCTGTGAGTCCGGGCGCTGCGACCGCT |
| | | GTCAGAAAAAGATCCGGATCTACCACAGTCTGACCGGGCTGCATTGTGTATGGT |
| | | GCCACCTAGAGATCCACGATGACTGCCTGCAAGCGGTGGGCCATGAGTGTGAC |
| | | TGTGGGCTGCTCCGGGATCACATCCTGCCTCCATCTTCCATCTATCCCAGTGTCC |
| | | TGGCCTCTGGACCGGATCGTAAAAATAGCAAAACAAGCCAGAAGACCATGGAT |
| | | GATTTAAATTTGAGCACCTCTGAGGCTCTGCGGATTGACCCTGTTCCTAACACC |
| | | CACCCACTTCTCGTCTTTGTCAATCCTAAGAGTGGCGGGAAGCAGGGGCAAAGG |
| | | GTGCTCTGGAAGTTCCAGTATATATTAAACCCTCGACAGGTGTTCAACCTCCTA |
| | | AAGGATGGTCCTGAGATAGGGCTCCGATTATTCAAGGATGTTCCTGATAGCCGG |
| | | ATTTTGGTGTGTGGTGGAGACGGCACAGTAGGCTGGATTCTAGAGACCATTGAC |
| | | AAAGCTAACTTGCCAGTTTTGCCTCCTGTTGCTGTGTTGCCCCTGGGTACTGGAA |
| | | ATGATCTGGCTCGATGCCTAAGATGGGGAGGAGGTTATGAAGGACAGAATCTG |
| | | GCAAAGATCCTCAAGGATTTAGAGATGAGTAAAGTGGTACATATGGATCGATG |
| | | GTCTGTGGAGGTGATACCTCAACAAACTGAAGAAAAAAGTGACCCAGTCCCCT |
| | | TTCAAATCATCAATAACTACTTCTCTATTGGCGTGGATGCCTCTATTGCTCATCG |
| | | ATTCCACATCATGCGAGAGAAATATCCGGAGAAGTTCAACAGCAGAATGAAGA |
| | | ACAAGCTATGGTACTTCGAATTTGCCACATCTGAATCCATCTTCTCAACATGCA |
| | | AAAAGCTGGAGGAGTCTTTGACAGTTGAGATCTGTGGGAAACCGCTGGATCTG |
| | | AGCAACCTGTCCCTAGAAGGCATCGCAGTGCTAAACATCCCTAGCATGCATGGT |
| | | GGCTCCAACCTCTGGGGTGATACCAGGAGACCCCATGGGGATATCTATGGGATC |
| | | AACCAGGCCTTAGGTGCTACAGCTAAAGTCATCACCGACCCTGATATCCTGAAA |
| | | ACCTGTGTACCAGACCTAAGTGACAAGAGACTGGAAGTGGTTGGGCTGGAGGG |
| | | TGCAATTGAGATGGGCCAAATCTATACCAAGCTCAAGAATGCTGGACGTCGGCT |
| | | GGCCAAGTGCTCTGAGATCACCTTCCACACCACAAAAACCCTTCCCATGCAAAT |
| | | TGACGGAGAACCCTGGATGCAGACGCCCTGTACAATCAAGATCACCCACAAGA |
| | | ACCAGATGCCCATGCTCATGGGCCCACCCCCCCGCTCCACCAATTTCTTTGGCTT |
| | | CTTGAGCTAAGGGGGACACCCTTGGCCTCCAAGCCAGCCTTGAACCCACCTCCC |
| | | TGTCCCTGGACTCTACTCCCGAGGCTCTGTACATTGCTGCCACATACTCCTGCCA |
| | | GCTTGGGGGAGTGTTCCTTCACCCTCACAGTATTTATTATCCTGCACCACCTCAC |
| | | TGTTCCCCATGCGCACACACATACACACACCCCAAAACACATACATTGAAAGTG |
| | | CCTCATCTGAATAAAATGACTTGTGTTTCCCCTTTGGGATCTGCTAA |
| 5 | DGKZ | CAAACTAGGACTTGCTCAGCAGAGGCCGCCAGCCCGGAGCTGGATCCAGAGCC |
| | mRNA | CGGCCTTGGGGACCCCAGCTCCCACCTGCGCCCTGCCTTCCAGATCAGCCAACC |
| | NCBI | GCCTGCCATGGAGACTTTCTTTAGGAGACATTTCCGGGGGAAGGTGCCAGGCCC |
| | NM_001105540 | TGGAGAGGGGCAGCAGCGGCCCAGCAGCGTGGGGCTGCCCACAGGCAAGGCCC |
| | | GGCGTCGCTCCCCCGCTGGGCAGGCCTCCTCCTCACTGGCACAGCGGCGCCGCT |
| | | CCAGCGCCCAGCTCCAGGGCTGCCTCCTGAGTTGCGGGGTGAGGGCCCAGGGTT |
| | | CCAGCCGCCGGCGCTCCAGCACTGTGCCCCCTTCCTGCAACCCCCGCTTCATCG |
| | | TGGATAAGGTGCTCACTCCACAGCCTACCACCGTGGGGGCCCAGCTTCTGGGTG |
| | | CACCCCTGCTGTTGACCGGGCTTGTGGGCATGAATGAGGAGGAGGGTGTCCAG |
| | | GAGGATGTGGTAGCCGAGGCATCGAGCGCCATCCAGCCAGGCACCAAGACACC |
| | | AGGGCCACCCCCACCTCGGGGCGCCCAGCCGCTGTTGCCCCTACCCCGCTACCT |
| | | GCGCCGAGCCTCCTCCCACCTGCTCCCCGCGGATGCCGTATATGACCACGCTCT |
| | | CTGGGGCCTGCACGGCTACTATCGGCGCCTCAGCCAGCGGCGGCCCTCAGGCCA |
| | | GCACCCTGGCCCTGGGGGCCGAAGAGCCTCAGGCACCACCGCCGGCACCATGC |
| | | TGCCCACCCGTGTGCGCCCACTGTCCCGCAGGCGCCAGGTAGCCCTACGGCGCA |
| | | AGGCGGCCGGACCCCAGGCCTGGAGCGCCCTGCTCGCGAAAGCCATCACCAAG |
| | | TCGGGCCTCCAGCACCTGGCCCCCCCTCCGCCCACCCCTGGGGCCCCGTGCAGC |
| | | GAGTCAGAGCGGCAGATCCGGAGTACAGTGGACTGGAGCGAGTCAGCGACATA |
| | | TGGGGAGCACATCTGGTTCGAGACCAACGTGTCCGGGGACTTCTGCTACGTTGG |
| | | GGAGCAGTACTGTGTAGCCAGGATGCTGAAGTCAGTGTCTCGAAGAAAGTGCG |
| | | CAGCCTGCAAGATTGTGGTGCACACGCCCTGCATCGAGCAGCTGGAGAAGATA |
| | | AATTTCCGCTGTAAGCCGTCCTTCCGTGAATCAGGCTCCAGGAATGTCCGCGAG |
| | | CCAACCTTTGTACGGCACCACTGGGTACACAGACGACGCCAGGACGGCAAGTG |
| | | TCGGCACTGTGGGAAGGGATTCCAGCAGAAGTTCACCTTCCACAGCAAGGAGA |
| | | TTGTGGCCATCAGCTGCTCGTGGTGCAAGCAGGCATACCACAGCAAGGTGTCCT |
| | | GCTTCATGCTGCAGCAGATCGAGGAGCCGTGCTCGCTGGGGGTCCACGCAGCC |
| | | GTGGTCATCCCGCCCACCTGGATCCTCCGCGCCCGGAGGCCCCAGAATACTCTG |
| | | AAAGCAAGCAAGAAGAAGAAGAGGGCATCCTTCAAGAGGAAGTCCAGCAAGA |
| | | AAGGGCCTGAGGAGGGCCGCTGGAGACCCTTCATCATCAGGCCCACCCCCTCCC |
| | | CGCTCATGAAGCCCCTGCTGGTGTTTGTGAACCCCAAGAGTGGGGGCAACCAG |
| | | GGTGCAAAGATCATCCAGTCTTTCCTCTGGTATCTCAATCCCCGACAAGTCTTCG |
| | | ACCTGAGCCAGGGAGGGCCCAAGGAGGCGCTGGAGATGTACCGCAAAGTGCAC |
| | | AACCTGCGGATCCTGGCGTGCGGGGGCGACGGCACGGTGGGCTGGATCCTCTC |
| | | CACCCTGGACCAGCTACGCCTGAAGCCGCCACCCCCTGTTGCCATCCTGCCCCT |
| | | GGGTACTGGCAACGACTTGGCCCGAACCCTCAACTGGGGTGGGGGCTACACAG |
| | | ATGAGCCTGTGTCCAAGATCCTCTCCCACGTGGAGGAGGGGAACGTGGTACAG |
| | | CTGGACCGCTGGGACCTCCACGCTGAGCCCAACCCCGAGGCAGGGCCTGAGGA |
| | | CCGAGATGAAGGCGCCACCGACCGGTTGCCCCTGGATGTCTTCAACAACTACTT |
| | | CAGCCTGGGCTTTGACGCCCACGTCACCCTGGAGTTCCACGAGTCTCGAGAGGC |
| | | CAACCCAGAGAAATTCAACAGCCGCTTTCGGAATAAGATGTTCTACGCCGGGA |
| | | CAGCTTTCTCTGACTTCCTGATGGGCAGCTCCAAGGACCTGGCCAAGCACATCC |
| | | GAGTGGTGTGTGATGGAATGGACTTGACTCCCAAGATCCAGGACCTGAAACCC |
| | | CAGTGTGTTGTTTTCCTGAACATCCCCAGGTACTGTGCGGGCACCATGCCCTGG |
| | | GGCCACCCTGGGGAGCACCACGACTTTGAGCCCCAGCGGCATGACGACGGCTA |
| | | CCTCGAGGTCATTGGCTTCACCATGACGTCGTTGGCCGCGCTGCAGGTGGGCGG |
| | | ACACGGCGAGCGGCTGACGCAGTGTCGCGAGGTGGTGCTCACCACATCCAAGG |
| | | CCATCCCGGTGCAGGTGGATGGCGAGCCCTGCAAGCTTGCAGCCTCACGCATCC |
| | | GCATCGCCCTGCGCAACCAGGCCACCATGGTGCAGAAGGCCAAGCGGCGGAGC |
| | | GCCGCCCCCCTGCACAGCGACCAGCAGCCGGTGCCAGAGCAGTTGCGCATCCA |
| | | GGTGAGTCGCGTCAGCATGCACGACTATGAGGCCCTGCACTACGACAAGGAGC |
| | | AGCTCAAGGAGGCCTCTGTGCCGCTGGGCACTGTGGTGGTCCCAGGAGACAGT |
| | | GACCTAGAGCTCTGCCGTGCCCACATTGAGAGACTCCAGCAGGAGCCCGATGG |
| | | TGCTGGAGCCAAGTCCCCGACATGCCAGAAACTGTCCCCCAAGTGGTGCTTCCT |
| | | GGACGCCACCACTGCCAGCCGCTTCTACAGGATCGACCGAGCCCAGGAGCACC |
| | | TCAACTATGTGACTGAGATCGCACAGGATGAGATTTATATCCTGGACCCTGAGC |
| | | TGCTGGGGGCATCGGCCCGGCCTGACCTCCCAACCCCCACTTCCCCTCTCCCCA |
| | | CCTCACCCTGCTCACCCACGCCCCGGTCACTGCAAGGGGATGCTGCACCCCCTC |
| | | AAGGTGAAGAGCTGATTGAGGCTGCCAAGAGGAACGACTTCTGTAAGCTCCAG |
| | | GAGCTGCACCGAGCTGGGGGCGACCTCATGCACCGAGACGAGCAGAGTCGCAC |
| | | GCTCCTGCACCACGCAGTCAGCACTGGCAGCAAGGATGTGGTCCGCTACCTGCT |
| | | GGACCACGCCCCCCCAGAGATCCTTGATGCGGTGGAGGAAAACGGGGAGACCT |
| | | GTTTGCACCAAGCAGCGGCCCTGGGCCAGCGCACCATCTGCCACTACATCGTGG |
| | | AGGCCGGGGCCTCGCTCATGAAGACAGACCAGCAGGGCGACACTCCCCGGCAG |
| | | CGGGCTGAGAAGGCTCAGGACACCGAGCTGGCCGCCTACCTGGAGAACCGGCA |
| | | GCACTACCAGATGATCCAGCGGGAGGACCAGGAGACGGCTGTGTAGCGGGCCG |
| | | CCCACGGGCAGCAGGAGGGACAATGCGGCCAGGGGACGAGCGCCTTCCTTGCC |
| | | CACCTCACTGCCACATTCCAGTGGGACGGCCACGGGGGGACCTAGGCCCCAGG |
| | | GAAAGAGCCCCATGCCGCCCCCTAAGGAGCCGCCCAGACCTAGGGCTGGACTC |
| | | AGGAGCTGGGGGGGCCTCACCTGTTCCCCTGAGGACCCCGCCGGACCCGGAGG |
| | | CTCACAGGGAACAAGACACGGCTGGGTTGGATATGCCTTTGCCGGGGTTCTGGG |
| | | GCAGGGCGCTCCCTGGCCGCAGCAGATGCCCTCCCAGGAGTGGAGGGGCTGGA |
| | | GAGGGGGAGGCCTTCGGGAAGAGGCTTCCTGGGCCCCCTGGTCTTCGGCCGGG |
| | | TCCCCAGCCCCCGCTCCTGCCCCACCCCACCTCCTCCGGGCTTCCTCCCGGAAAC |
| | | TCAGCGCCTGCTGCACTTGCCTGCCCTGCCTTGCTTGGCACCCGCTCCGGCGACC |
| | | CTCCCCGCTCCCCTGTCATTTCATCGCGGACTGTGCGGCCTGGGGGTGGGGGGC |
| | | GGGACTCTCACGGTGACATGTTTACAGCTGGGTGTGACTCAGTAAAGTGGATTT |
| | | TTTTTTC |
| 6 | DNMT3A | GCGCGGGCGGCGGCGGCGGCGAGAGCAGAGGACGAGCCGGGACGCGGCGCCG |
| | mRNA | CGGCACCAGGGCGCGCAGCCGGGCCGGCCCGACCCCACCGGCCATACGGTGGA |
| | NM_022552 | GCCATCGAAGCCCCCACCCACAGGCTGACAGAGGCACCGTTCACCAGAGGGCT |
| | | CAACACCGGGATCTATGTTTAAGTTTTAACTCTCGCCTCCAAAGACCACGATAA |
| | | TTCCTTCCCCAAAGCCCAGCAGCCCCCCAGCCCCGCGCAGCCCCAGCCTGCCTC |
| | | CCGGCGCCCAGATGCCCGCCATGCCCTCCAGCGGCCCCGGGGACACCAGCAGC |
| | | TCTGCTGCGGAGCGGGAGGAGGACCGAAAGGACGGAGAGGAGCAGGAGGAGC |
| | | CGCGTGGCAAGGAGGAGCGCCAAGAGCCCAGCACCACGGCACGGAAGGTGGG |
| | | GCGGCCTGGGAGGAAGCGCAAGCACCCCCCGGTGGAAAGCGGTGACACGCCAA |
| | | AGGACCCTGCGGTGATCTCCAAGTCCCCATCCATGGCCCAGGACTCAGGCGCCT |
| | | CAGAGCTATTACCCAATGGGGACTTGGAGAAGCGGAGTGAGCCCCAGCCAGAG |
| | | GAGGGGAGCCCTGCTGGGGGGCAGAAGGGCGGGGCCCCAGCAGAGGGAGAGG |
| | | GTGCAGCTGAGACCCTGCCTGAAGCCTCAAGAGCAGTGGAAAATGGCTGCTGC |
| | | ACCCCCAAGGAGGGCCGAGGAGCCCCTGCAGAAGCGGGCAAAGAACAGAAGG |
| | | AGACCAACATCGAATCCATGAAAATGGAGGGCTCCCGGGGCCGGCTGCGGGGT |
| | | GGCTTGGGCTGGGAGTCCAGCCTCCGTCAGCGGCCCATGCCGAGGCTCACCTTC |
| | | CAGGCGGGGGACCCCTACTACATCAGCAAGCGCAAGCGGGACGAGTGGCTGGC |
| | | ACGCTGGAAAAGGGAGGCTGAGAAGAAAGCCAAGGTCATTGCAGGAATGAAT |
| | | GCTGTGGAAGAAAACCAGGGGCCCGGGGAGTCTCAGAAGGTGGAGGAGGCCA |
| | | GCCCTCCTGCTGTGCAGCAGCCCACTGACCCCGCATCCCCCACTGTGGCTACCA |
| | | CGCCTGAGCCCGTGGGGTCCGATGCTGGGGACAAGAATGCCACCAAAGCAGGC |
| | | GATGACGAGCCAGAGTACGAGGACGGCCGGGGCTTTGGCATTGGGGAGCTGGT |
| | | GTGGGGGAAACTGCGGGGCTTCTCCTGGTGGCCAGGCCGCATTGTGTCTTGGTG |
| | | GATGACGGGCCGGAGCCGAGCAGCTGAAGGCACCCGCTGGGTCATGTGGTTCG |
| | | GAGACGGCAAATTCTCAGTGGTGTGTGTTGAGAAGCTGATGCCGCTGAGCTCGT |
| | | TTTGCAGTGCGTTCCACCAGGCCACGTACAACAAGCAGCCCATGTACCGCAAAG |
| | | CCATCTACGAGGTCCTGCAGGTGGCCAGCAGCCGCGCGGGGAAGCTGTTCCCG |
| | | GTGTGCCACGACAGCGATGAGAGTGACACTGCCAAGGCCGTGGAGGTGCAGAA |
| | | CAAGCCCATGATTGAATGGGCCCTGGGGGGCTTCCAGCCTTCTGGCCCTAAGGG |
| | | CCTGGAGCCACCAGAAGAAGAGAAGAATCCCTACAAAGAAGTGTACACGGACA |
| | | TGTGGGTGGAACCTGAGGCAGCTGCCTACGCACCACCTCCACCAGCCAAAAAG |
| | | CCCCGGAAGAGCACAGCGGAGAAGCCCAAGGTCAAGGAGATTATTGATGAGCG |
| | | CACAAGAGAGCGGCTGGTGTACGAGGTGCGGCAGAAGTGCCGGAACATTGAGG |
| | | ACATCTGCATCTCCTGTGGGAGCCTCAATGTTACCCTGGAACACCCCCTCTTCGT |
| | | TGGAGGAATGTGCCAAAACTGCAAGAACTGCTTTCTGGAGTGTGCGTACCAGTA |
| | | CGACGACGACGGCTACCAGTCCTACTGCACCATCTGCTGTGGGGGCCGTGAGGT |
| | | GCTCATGTGCGGAAACAACAACTGCTGCAGGTGCTTTTGCGTGGAGTGTGTGGA |
| | | CCTCTTGGTGGGGCCGGGGGCTGCCCAGGCAGCCATTAAGGAAGACCCCTGGA |
| | | ACTGCTACATGTGCGGGCACAAGGGTACCTACGGGCTGCTGCGGCGGCGAGAG |
| | | GACTGGCCCTCCCGGCTCCAGATGTTCTTCGCTAATAACCACGACCAGGAATTT |
| | | GACCCTCCAAAGGTTTACCCACCTGTCCCAGCTGAGAAGAGGAAGCCCATCCG |
| | | GGTGCTGTCTCTCTTTGATGGAATCGCTACAGGGCTCCTGGTGCTGAAGGACTT |
| | | GGGCATTCAGGTGGACCGCTACATTGCCTCGGAGGTGTGTGAGGACTCCATCAC |
| | | GGTGGGCATGGTGCGGCACCAGGGGAAGATCATGTACGTCGGGGACGTCCGCA |
| | | GCGTCACACAGAAGCATATCCAGGAGTGGGGCCCATTCGATCTGGTGATTGGG |
| | | GGCAGTCCCTGCAATGACCTCTCCATCGTCAACCCTGCTCGCAAGGGCCTCTAC |
| | | GAGGGCACTGGCCGGCTCTTCTTTGAGTTCTACCGCCTCCTGCATGATGCGCGG |
| | | CCCAAGGAGGGAGATGATCGCCCCTTCTTCTGGCTCTTTGAGAATGTGGTGGCC |
| | | ATGGGCGTTAGTGACAAGAGGGACATCTCGCGATTTCTCGAGTCCAACCCTGTG |
| | | ATGATTGATGCCAAAGAAGTGTCAGCTGCACACAGGGCCCGCTACTTCTGGGGT |
| | | AACCTTCCCGGTATGAACAGGCCGTTGGCATCCACTGTGAATGATAAGCTGGAG |
| | | CTGCAGGAGTGTCTGGAGCATGGCAGGATAGCCAAGTTCAGCAAAGTGAGGAC |
| | | CATTACTACGAGGTCAAACTCCATAAAGCAGGGCAAAGACCAGCATTTTCCTGT |
| | | CTTCATGAATGAGAAAGAGGACATCTTATGGTGCACTGAAATGGAAAGGGTAT |
| | | TTGGTTTCCCAGTCCACTATACTGACGTCTCCAACATGAGCCGCTTGGCGAGGC |
| | | AGAGACTGCTGGGCCGGTCATGGAGCGTGCCAGTCATCCGCCACCTCTTCGCTC |
| | | CGCTGAAGGAGTATTTTGCGTGTGTGTAAGGGACATGGGGGCAAACTGAGGTA |
| | | GCGACACAAAGTTAAACAAACAAACAAAAAACACAAAACATAATAAAACACC |
| | | AAGAACATGAGGATGGAGAGAAGTATCAGCACCCAGAAGAGAAAAAGGAATT |
| | | TAAAACAAAAACCACAGAGGCGGAAATACCGGAGGGCTTTGCCTTGCGAAAAG |
| | | GGTTGGACATCATCTCCTGATTTTTCAATGTTATTCTTCAGTCCTATTTAAAAAC |
| | | AAAACCAAGCTCCCTTCCCTTCCTCCCCCTTCCCTTTTTTTTCGGTCAGACCTTTT |
| | | ATTTTCTACTCTTTTCAGAGGGGTTTTCTGTTTGTTTGGGTTTTGTTTCTTGCTGT |
| | | GACTGAAACAAGAAGGTTATTGCAGCAAAAATCAGTAACAAAAAATAGTAACA |
| | | ATACCTTGCAGAGGAAAGGTGGGAGAGAGGAAAAAAGGAAATTCTATAGAAA |
| | | TCTATATATTGGGTTGTTTTTTTTTTTGTTTTTTGTTTTTTTTTTTTGGGTTTTTTTT |
| | | TTTACTATATATCTTTTTTTTGTTGTCTCTAGCCTGATCAGATAGGAGCACAAGC |
| | | AGGGGACGGAAAGAGAGAGACACTCAGGCGGCAGCATTCCCTCCCAGCCACTG |
| | | AGCTGTCGTGCCAGCACCATTCCTGGTCACGCAAAACAGAACCCAGTTAGCAGC |
| | | AGGGAGACGAGAACACCACACAAGACATTTTTCTACAGTATTTCAGGTGCCTAC |
| | | CACACAGGAAACCTTGAAGAAAATCAGTTTCTAGAAGCCGCTGTTACCTCTTGT |
| | | TTACAGTTTATATATATATGATAGATATGAGATATATATATAAAAGGTACTGTT |
| | | AACTACTGTACAACCCGACTTCATAATGGTGCTTTCAAACAGCGAGATGAGTAA |
| | | AAACATCAGCTTCCACGTTGCCTTCTGCGCAAAGGGTTTCACCAAGGATGGAGA |
| | | AAGGGAGACAGCTTGCAGATGGCGCGTTCTCACGGTGGGCTCTTCCCCTTGGTT |
| | | TGTAACGAAGTGAAGGAGGAGAACTTGGGAGCCAGGTTCTCCCTGCCAAAAAG |
| | | GGGGCTAGATGAGGTGGTCGGGCCCGTGGACAGCTGAGAGTGGGATTCATCCA |
| | | GACTCATGCAATAACCCTTTGATTGTTTTCTAAAAGGAGACTCCCTCGGCAAGA |
| | | TGGCAGAGGGTACGGAGTCTTCAGGCCCAGTTTCTCACTTTAGCCAATTCGAGG |
| | | GCTCCTTGTGGTGGGATCAGAACTAATCCAGAGTGTGGGAAAGTGACAGTCAA |
| | | AACCCCACCTGGAGCAAATAAAAAAACATACAAAACGTACTGGTGCTTTCCTGT |
| | | CTAAGTCGCCTTTTGTGTGTTCTTTTATGAGGCCCCCACCATCCACCCCTCTGTG |
| | | CACAAGGCGGCTCCCGGCCCCTGGAATGTGATGTTTTTGGTCATCTCCAAAGGC |
| | | TGCAGTTTTATACTGGGAGGCTGATGACACCTTTTATTATAATTATTCTTATGGT |
| | | TCTGGTTATAATTATGTTTGTTTTAAGATTTTCTTTAAGAAAACAAAAACCCAAC |
| | | ACCCTTCCCTTTAGGTTTCAAACCAAGGTGCGGGAGCAGGGTGCTTCTGAGTTA |
| | | GCGGTGGCTCTGGTGCCCTGGCTCCCACCCCTCGGGCCAGGTGGGCCACTGGGC |
| | | ATGGTGACAGCTGGGCGGCCAGGGAGTCCCCAAGGTCTGCCTTCTGGGCTTGTC |
| | | ACTCTTCTCTTCTACTTCCCCTCCTCTTGAGTCGGGTGTGTCAGGGCTGGAGGGA |
| | | GGACCGGCCGTCTCTCTCCTTGTGTGTGTGATTGGAGTGGTGTGTATTTCTTTTC |
| | | TAGTACTTGGTCTGATGATAGCTGTGCTCTTACCTCGAGTCAGCAGCACCTGGA |
| | | GCCCCAAGTGCTCAACACTTTGTTCTACTTCCTACAAGGCAGGCGGCCTGGCGT |
| | | CAGTAGTAGGAAGGGATGGCATTGTTGGCTCCAGGGTGAGGGCCCGACGGGCA |
| | | GGTGCTTGCCTGGGTAGATGGGGTGTAGATATGTGGCATATAGAGATATATATT |
| | | TTATAATGGGAGGGGGGATGGCACCTCCTGGGACTGGTAGAGCTGGGAGGTGT |
| | | GTCGGCAAGCACATGGCCCCAAGTCCACGTCCCTAGAAAGATGGGGCCTACAG |
| | | TGATAGGCGCTCTATAAATACATAGATAGGGTCATGTTTAAGAAATATTATGGG |
| | | GTGGGGAGTGGGGGAAGCAGGCTTAGTGTGCTGAGCCTCGGTCCCCACTGCAG |
| | | ACCTAAACCCCACACCAATCAGTCAATCTCACACGCATACACAGGAGCATATTT |
| | | CTGTACTGATGCATAATGAATGCACTCACACACACACACACACGCCAGGGGGC |
| | | TCCCAGCACTCTTCTCCCAGGGTTCAGGACTTTGTGGAGTCAGCCCGTGCAGCT |
| | | ATTTACCTCTGTTGCCCTGATTGCAGGTAGACTGTCAATCCCAGAAGTTGCTAG |
| | | GTGCTGAGGCAGGAAAAGGAGGAGGTTTCTTTTAGCAGGTGCTCTCCTCTAGCA |
| | | GGTAGTGTTCTATCTCTTTTGCCCCTGGTGATGTCAAAAGATTCTTCTGTTAGCA |
| | | ACCTGTGATACTGACCTGCAACTCTAGGCACCAGTCAAGTCAGCCCTCAACTCC |
| | | CCCTACCTATTCTCCCCAACTGAAATCAAATTGACATCCAGGTCTGTCTTGGCCA |
| | | GGGGGCTTCCATTGGGGAAAAAAAATGGGACAGTGAACCTCACCTATTGAATA |
| | | AACCAAATTAGTAATTAGTGACAAAACCAAACCAAGGCTTTGGACCCCCTGCTC |
| | | ATTACTCAGGAGTAAATTCACACAGCTGGTGGCCGGTCTCACCCCTTTTTGGGG |
| | | TGCTTGGTCCTGGACCCCAATCTACTGTCTCCTCGACAGATTGGTGTTTAAGATC |
| | | ATGCCCAGTTGCTTCCTGTCACCTCTTGACAGGTGGTGACTGGGCTGCTGTGGG |
| | | GCTGGAGCAGCGAGGAAGGGAGCCTCTGCCCCCTCAGTGGAGTCCCTGTGCTA |
| | | ACTGTGGTGCTCACCCAGCTCCTCCTTGCTCCCGCGGGGCTCTGTCCTTCGGGCC |
| | | TGGAGTCCACCAGCGCCACTCCAGGAGGGCTGGGGGAGGAGGGGCTGCCTCAG |
| | | GCAGTCCAAGGTGCTCCCTCCCCCGCTGGCCAATGCTAAGGTCTTCCCGGCTCC |
| | | CAGGGTCAGGACAGAGTCTTCTAAGGCTCAGGCTCTCCCAAGCCACTGCTTAGT |
| | | GACAGTTAGCCTTTCCCAGCGGCCCAAGCTCCACCGGAACTGGGCCTGGTGCTC |
| | | CAACCAAAGGGTTGGTGTTGGGACTTGGGAAGAGACTTGGTAGCCAAGGGCAT |
| | | GGCAAGCCTGGGGGATTCCTGTGGGCCGGCTTGCTCGAGGAGGGAGTGCTGGG |
| | | CACAGAGGAGCCCTTATGGCTGACAGCTTGAGAGCACTTGTTCCTCCAGATCTA |
| | | GCCACTTGACCAGGCCCTAGAATGGACCAGATGCCCCAGGGGTCATGGCCCCC |
| | | CCCCCCCCCCCCTTGCAGGGACGAGAGTTAACAATCACCAGCTTTTCTTCCGGC |
| | | CCAGCCTGTGTGCTTGGTCTCACAGTAGAATTTAGGAGAGTGTGGGGCTTTGGG |
| | | ATTGTGCCAACTTGGCCAAGCTGAGATATGCTTAATCCACCTCAACGCTGGGGC |
| | | CTCTGGGGACAGGTATTGGGTCACCTTCCAAGGGGTGCTGAACTTCAGCAGGCT |
| | | GTAGATAAGGCAGAGGTTAGAGGCAGTGGAGTGGGGGAACCAAGAAGGCCTA |
| | | GGGAAAATTGCCAGGCATATTCCCTGTGGAATTTTCTTTCCGAAGAGTAGGCCC |
| | | ATTTCCCCCTTAGAAACAAGGGTTCTGGGGGCTGATGACAGCTTCCCCTGCAGG |
| | | TGCCAGGTGGCGCATGATGCCAAAGAGATCCGTGGCTCTTGCCTGGGGGAGGT |
| | | AGCCAGGTGCTCGTGCTGGGGACACAGTCCTTGGCCTCCTGCCTAAGCAGGAAG |
| | | CTAGTTTCCAGCAGGCGGCTGGTACTAACCCCCCGCCCCACGGGAGCGACAGG |
| | | CTGGTGCACCGGCAGACTGGGGGGCCGCAGGCGGGCACTAGCAGGGGTCTGCA |
| | | GTCCATAGATGATGACATGAATCTGTTGCTGTCCTTTTGTCTCTAGGCCCCAGAG |
| | | ACTTCTTGCTATTGAACATCCTATTGGAAAGCAAACTTTCCAAGAGAGCACCCG |
| | | GGACCTGTTAGCTTTTGAAACAGGCCTGGGTGTGATTGACTGGTAGAGATGCCT |
| | | GTGTGATTTTTGGCGCATTGCAGGAGTGTGGATTGGGACAGGCACCCCAGCAGG |
| | | AAGGAAGGCTGACACCAAAGGGGTCTCACAGGGCCCAGCTCCCCTTGCTTCCC |
| | | GATAATTGGCCACCAGGCCATGGCGACTCTGGGGAAGCCTAGATGGGAACACC |
| | | CACCTTCTCCAGCAGTAGAAGGAATATTTCAGCAGAGCCATCTGGGAGATGTTC |
| | | TTATAGGAAAACTGCAGTCATTCATTTCATAGTTTTGCTTATATAAATAGAGTAC |
| | | ATTTGGGAGGCAGTTTTGAATTCCTGTTGCTACTCTGCTTGGTGCGGAGTTGCAG |
| | | TGGCCCCAAGTGTCTTTCAGAGCTGTTGCTTTTAGGATCCCCCTCAAGCTGTGGG |
| | | TTAGGGCCAGGGCAGGGAGGGGGTGCTGCCTGGCACTGTGCCCTGAAGAGGGC |
| | | TGCTTGCTTCCTTTGGTCGGGAGCACGGCAGAGCCAACATTCTGGTGGGAACCG |
| | | AGTTTCTAGCCAGGCCCCTGTTTTGGTCCCGGGAGGTGAGTAATCTGAGTTTGT |
| | | CATTGCGTATTCTCCACGCTGCCTGCTCTCAGTGCTGGAGACTAGAGAGCGACC |
| | | CTGGAGTTGTCCTCTGGGAGTCCTGCCTGTTTGCCAGGGAGCAGCTGAAACCTC |
| | | CCCGCTCCCAATCCCGGGGGTGGAAAAGGTGTAGTGGAGGAATGGTCTGGAGC |
| | | TGGGATGGCCCTTCTGGCCATGCCCTGCTGAGCCAGGATTAAGGAAAGTGTCAG |
| | | CCGCCTCCTTACCAGGCCCTGTGCTGGGGTCAGGAGGACCGGGAGGAGAGGGA |
| | | AGGGGTGAGAGGATGGACTCTGCTGTTGTAGTGTGAGCTCTGGAATCTTTACGT |
| | | TTTGGTAAAGGCAGGCCAGGGTCTTTGAGGATCGAGTTGGGGAACCTTGCCTCT |
| | | GTGTTTCCGAGACCCAGCTAAGGTGGGCCTTTGGTGGGTGACCTGTCTCTGACC |
| | | CGAGGCAGCAGAGTTTTGGGGGAGGGAGAAACAAGCAAGGAGCAGGGAGGAC |
| | | AAGGACACAAGATCCCTTTGCTGGTGGTGACTTACTCACTTCCCCCACCAGAGG |
| | | GCCTGGGACTCCCGAAACTCCCGTAACTGGAAAGCTCCCTGGCTCAGCTCCACT |
| | | GGGGGGTGAGGAGGGCGGCTCCCTCAGCCCCAGCCTAGCGGCCTCCAACCCTG |
| | | TGCCTGAGAACATGAGCAGCGGTCTAAAGCATCCCCTAGTCCCATTTCACTTCA |
| | | CAGACCGATTTGGTTGCACAGCCCTAGAATTCAACAGACAACAAAAATGCTGA |
| | | AATTCCTTAGAATACGCAGAGGGAGGAGGTGACTCACCAAGGTGCTAAAACAT |
| | | TTTATTAAAAATATATATTGTTAAATTAAGTCTGCTGTCTGGACAATGACTGTTT |
| | | GTTTTGTTTTCTTGTAGTGGAGTTTAAAAGAGACTATTATTTTACTCTGATATAT |
| | | TATTATTAAAAAGGCATTTTAACTTTGTACTTGAAAACTAAATGAGCGATTTCA |
| | | TGTGTTTTAGCTGAGGCATTGAAGTAGCGGGTGCTTACTTATTTGTGGGAAATC |
| | | ATGTATTCATACTGAAGAATAAACTCCGTAGCTGATTTGGTAATAACTTTTACT |
| | | GTTACAGACGATTTCGCTTTGACCCCGGTGGCAGAGCACTTTTACTCCACATTCC |
| | | ACGCAGCTAAATGCAGGACTTCCCAGACTCTTCCCTCCCCAGCCCTCTTTCCTTT |
| | | CTCTTAAGTTCCTCTTTTCTCCTTTATTATTATTATTTTTTTTTCCTGGTTCCAGCA |
| | | TCCTTTCACCACTGTATTTTTTTTTTTTTTTTTTTTTTTTTTTTTTTTTTTTTTTTTTT |
| | | TTTTTTTTTAGGGTTGCTTCTCTATTTATTGACAATAATAATGTACATTTCACAGT |
| | | CTGGTTCTAGGACATTCAGGGAAGGGCGAGTGGGGGCCCGAGGGGTCCCTGCC |
| | | CCACCCGCCTGGTGTCCTGTTGTATCTCTGTGTAAGTGATGCTCGTGACATAGCT |
| | | GTTTATGAAATAATTAAAGAGGTCAATGCGTACAACAGATGTAGAAAGTCTGTC |
| | | AGTTCCGCCTTCATCACATTTTTTTTTGTGCCCAGAAGAATATAATAAAGCTCCT |
| | | TTCTAATGTACTTGTGCTGGAGAACACTTGAATAAATGGACTGTTTTTGTGCAA |
| | | AAAGAAAAA |
| 7 | MAP4K1 | AGTTGAGCCCTGAGGCCCTGCTGGCCCCTGGGCGCAGGCCCAGCTCAGGCCCCC |
| | mRNA | AGGGATGGACGTCGTGGACCCTGACATTTTCAATAGAGACCCCCGGGACCACT |
| | NCBI | ATGACCTGCTACAGCGGCTGGGTGGCGGCACGTATGGGGAAGTCTTTAAGGCTC |
| | NM_001042600 | GAGACAAGGTGTCAGGGGACCTGGTGGCACTGAAGATGGTGAAGATGGAGCCT |
| | | GATGATGATGTCTCCACCCTTCAGAAGGAAATCCTCATATTGAAAACTTGCCGG |
| | | CACGCCAACATCGTGGCCTACCATGGGAGTTATCTCTGGTTGCAGAAACTCTGG |
| | | ATCTGCATGGAATTCTGTGGGGCTGGTTCTCTCCAGGACATCTACCAAGTGACA |
| | | GGCTCCCTGTCAGAGCTCCAGATTAGCTATGTCTGCCGGGAAGTGCTCCAGGGA |
| | | CTGGCCTATTTGCACTCACAGAAGAAGATACACAGGGACATCAAGGGAGCTAA |
| | | CATCCTCATCAATGATGCTGGGGAGGTCAGATTGGCTGACTTTGGCATCTCGGC |
| | | CCAGATTGGGGCTACACTGGCCAGACGCCTCTCTTTCATTGGGACACCCTACTG |
| | | GATGGCTCCGGAAGTGGCAGCTGTGGCCCTGAAGGGAGGATACAATGAGCTGT |
| | | GTGACATCTGGTCCCTGGGCATCACGGCCATCGAACTGGCCGAGCTACAGCCAC |
| | | CGCTCTTTGATGTGCACCCTCTCAGAGTTCTCTTCCTCATGACCAAGAGTGGCTA |
| | | CCAGCCTCCCCGACTGAAGGAAAAAGGCAAATGGTCGGCTGCCTTCCACAACTT |
| | | CATCAAAGTCACTCTGACTAAGAGTCCCAAGAAACGACCCAGCGCCACCAAGA |
| | | TGCTCAGTCATCAACTGGTATCCCAGCCTGGGCTGAATCGAGGCCTGATCCTGG |
| | | ATCTTCTTGACAAACTGAAGAATCCCGGGAAAGGACCCTCCATTGGGGACATTG |
| | | AGGATGAGGAGCCCGAGCTACCCCCTGCTATCCCTCGGCGGATCAGATCCACCC |
| | | ACCGCTCCAGCTCTCTGGGGATCCCAGATGCAGACTGCTGTCGGCGGCACATGG |
| | | AGTTCAGGAAGCTCCGAGGAATGGAGACCAGACCCCCAGCCAACACCGCTCGC |
| | | CTACAGCCTCCTCGAGACCTCAGGAGCAGCAGCCCCAGGAAGCAACTGTCAGA |
| | | GTCGTCTGACGATGACTATGACGACGTGGACATCCCCACCCCTGCAGAGGACAC |
| | | ACCTCCTCCACTTCCCCCCAAGCCCAAGTTCCGTTCTCCATCAGACGAGGGTCCT |
| | | GGGAGCATGGGGGATGATGGGCAGCTGAGCCCGGGGGTGCTGGTCCGGTGTGC |
| | | CAGTGGGCCCCCACCAAACAGCCCCCGTCCTGGGCCTCCCCCATCCACCAGCAG |
| | | CCCCCACCTCACCGCCCATTCAGAACCCTCACTCTGGAACCCACCCTCCCGGGA |
| | | GCTTGACAAGCCCCCACTTCTGCCCCCCAAGAAGGAAAAGATGAAGAGAAAGG |
| | | GATGTGCCCTTCTCGTAAAGTTGTTCAATGGCTGCCCCCTCCGGATCCACAGCA |
| | | CGGCCGCCTGGACACATCCCTCCACCAAGGACCAGCACCTGCTCCTGGGGGCA |
| | | GAGGAAGGCATCTTCATCCTGAACCGGAATGACCAGGAGGCCACGCTGGAAAT |
| | | GCTCTTTCCTAGCCGGACTACGTGGGTGTACTCCATCAACAACGTTCTCATGTCT |
| | | CTCTCAGGAAAGACCCCCCACCTGTATTCTCATAGCATCCTTGGCCTGCTGGAA |
| | | CGGAAAGAGACCAGAGCAGGAAACCCCATCGCTCACATTAGCCCCCACCGCCT |
| | | ACTGGCAAGGAAGAACATGGTTTCCACCAAGATCCAGGACACCAAAGGCTGCC |
| | | GGGCGTGCTGTGTGGCGGAGGGTGCGAGCTCTGGGGGCCCGTTCCTGTGCGGTG |
| | | CATTGGAGACGTCCGTTGTCCTGCTTCAGTGGTACCAGCCCATGAACAAATTCC |
| | | TGCTTGTCCGGCAGGTGCTGTTCCCACTGCCGACGCCTCTGTCCGTGTTCGCGCT |
| | | GCTGACCGGGCCAGGCTCTGAGCTGCCCGCTGTGTGCATCGGCGTGAGCCCCGG |
| | | GCGGCCGGGGAAGTCGGTGCTCTTCCACACGGTGCGCTTTGGCGCGCTCTCTTG |
| | | CTGGCTGGGCGAGATGAGCACCGAGCACAGGGGACCCGTGCAGGTGACCCAGG |
| | | TAGAGGAAGATATGGTGATGGTGTTGATGGATGGCTCTGTGAAGCTGGTGACCC |
| | | CGGAGGGGTCCCCAGTCCGGGGACTTCGCACACCTGAGATCCCCATGACCGAA |
| | | GCGGTGGAGGCCGTGGCTATGGTTGGAGGTCAGCTTCAGGCCTTCTGGAAGCAT |
| | | GGAGTGCAGGTGTGGGCTCTAGGCTCGGATCAGCTGCTACAGGAGCTGAGAGA |
| | | CCCTACCCTCACTTTCCGTCTGCTTGGCTCCCCCAGGCCTGTAGTGGTGGAGACA |
| | | CGCCCAGTGGATGATCCTACTGCTCCCAGCAACCTCTACATCCAGGAATGAGTC |
| | | CCTAGGGGGGTGTCAGGAACTAGTCCTTGCACCCCCTCCCCCATAGACACACTA |
| | | GTGGTCATGGCATGTCCTCATCTCCCAATAAACATGACTTTAGCCTCTGC |
| 8 | NR4A1 | AGTCCCAGTGGCGGAGGCTACGAAACTTGGGGGAGTGCACAGAAGAACTTCGG |
| | mRNA | GAGCGCACGCGGGACCAGGGACCAGGCTGAGACTCGGGGCGCCAGTCCGGGCA |
| | NCBI | GGGGCAGCGGGAGCCGGCCGGGTAGGGTGCAGCCTGAGGCTTGTTCAGCAGAA |
| | NM_002135 | CAGGTGCAAGCCACATTGTTGCCAAGACCTGCCTGAAGCCGGATTCTCCCCACT |
| | | GCCTCCTTCAACCCCGCCTCTTCCTCCTCCTGTGGGACTGCTCCCCCCTCCTGTG |
| | | AGGCTAGATAGATGCCCTGTATCCAAGCCCAATATGGGACACCAGCACCGAGT |
| | | CCGGGACCCCGTGACCACCTGGCAAGCGACCCCCTGACCCCTGAGTTCATCAAG |
| | | CCCACCATGGACCTGGCCAGCCCCGAGGCAGCCCCCGCTGCCCCCACTGCCCTG |
| | | CCCAGCTTCAGCACCTTCATGGACGGCTACACAGGAGAGTTTGACACCTTCCTC |
| | | TACCAGCTGCCAGGAACAGTCCAGCCATGCTCCTCAGCCTCCTCCTCGGCCTCC |
| | | TCCACATCCTCGTCCTCAGCCACCTCCCCTGCCTCTGCCTCCTTCAAGTTCGAGG |
| | | ACTTCCAGGTGTACGGCTGCTACCCCGGCCCCCTGAGCGGCCCAGTGGATGAGG |
| | | CCCTGTCCTCCAGTGGCTCTGACTACTATGGCAGCCCCTGCTCGGCCCCGTCGCC |
| | | CTCCACGCCCAGCTTCCAGCCGCCCCAGCTCTCTCCCTGGGATGGCTCCTTCGGC |
| | | CACTTCTCGCCCAGCCAGACTTACGAAGGCCTGCGGGCATGGACAGAGCAGCT |
| | | GCCCAAAGCCTCTGGGCCCCCACAGCCTCCAGCCTTCTTTTCCTTCAGTCCTCCC |
| | | ACCGGCCCCAGCCCCAGCCTGGCCCAGAGCCCCCTGAAGTTGTTCCCCTCACAG |
| | | GCCACCCACCAGCTGGGGGAGGGAGAGAGCTATTCCATGCCTACGGCCTTCCC |
| | | AGGTTTGGCACCCACTTCTCCACACCTTGAGGGCTCGGGGATACTGGATACACC |
| | | CGTGACCTCAACCAAGGCCCGGAGCGGGGCCCCAGGTGGAAGTGAAGGCCGCT |
| | | GTGCTGTGTGTGGGGACAACGCTTCATGCCAGCATTATGGTGTCCGCACATGTG |
| | | AGGGCTGCAAGGGCTTCTTCAAGCGCACAGTGCAGAAAAACGCCAAGTACATC |
| | | TGCCTGGCTAACAAGGACTGCCCTGTGGACAAGAGGCGGCGAAACCGCTGCCA |
| | | GTTCTGCCGCTTCCAGAAGTGCCTGGCGGTGGGCATGGTGAAGGAAGTTGTCCG |
| | | AACAGACAGCCTGAAGGGGCGGCGGGGCCGGCTACCTTCAAAACCCAAGCAGC |
| | | CCCCAGATGCCTCCCCTGCCAATCTCCTCACTTCCCTGGTCCGTGCACACCTGGA |
| | | CTCAGGGCCCAGCACTGCCAAACTGGACTACTCCAAGTTCCAGGAGCTGGTGCT |
| | | GCCCCACTTTGGGAAGGAAGATGCTGGGGATGTACAGCAGTTCTACGACCTGCT |
| | | CTCCGGTTCTCTGGAGGTCATCCGCAAGTGGGCGGAGAAGATCCCTGGCTTTGC |
| | | TGAGCTGTCACCGGCTGACCAGGACCTGTTGCTGGAGTCGGCCTTCCTGGAGCT |
| | | CTTCATCCTCCGCCTGGCGTACAGGTCTAAGCCAGGCGAGGGCAAGCTCATCTT |
| | | CTGCTCAGGCCTGGTGCTACACCGGCTGCAGTGTGCCCGTGGCTTCGGGGACTG |
| | | GATTGACAGTATCCTGGCCTTCTCAAGGTCCCTGCACAGCTTGCTTGTCGATGTC |
| | | CCTGCCTTCGCCTGCCTCTCTGCCCTTGTCCTCATCACCGACCGGCATGGGCTGC |
| | | AGGAGCCGCGGCGGGTGGAGGAGCTGCAGAACCGCATCGCCAGCTGCCTGAAG |
| | | GAGCACGTGGCAGCTGTGGCGGGCGAGCCCCAGCCAGCCAGCTGCCTGTCACG |
| | | TCTGTTGGGCAAACTGCCCGAGCTGCGGACCCTGTGCACCCAGGGCCTGCAGCG |
| | | CATCTTCTACCTCAAGCTGGAGGACTTGGTGCCCCCTCCACCCATCATTGACAA |
| | | GATCTTCATGGACACGCTGCCCTTCTGACCCCTGCCTGGGAACACGTGTGCACA |
| | | TGCGCACTCTCATATGCCACCCCATGTGCCTTTAGTCCACGGACCCCCAGAGCA |
| | | CCCCCAAGCCTGGGCTTGAGCTGCAGAATGACTCCACCTTCTCACCTGCTCCAG |
| | | GAGGTTTGCAGGGAGCTCAAGCCCTTGGGGAGGGGGATGCCTTCATGGGGGTG |
| | | ACCCCACGATTTGTCTTATCCCCCCCAGCCTGGCCCCGGCCTTTATGTTTTTTGT |
| | | AAGATAAACCGTTTTTAACACATAGCGCCGTGCTGTAAATAAGCCCAGTGCTGC |
| | | TGTAAATACAGGAAGAAAGAGCTTGAGGTGGGAGCGGGGCTGGGAGGAAGGG |
| | | ATGGGCCCCGCCTTCCTGGGCAGCCTTTCCAGCCTCCTGCTGGCTCTCTCTTCCT |
| | | ACCCTCCTTCCACATGTACATAAACTGTCACTCTAGGAAGAAGACAAATGACAG |
| | | ATTCTGACATTTATATTTGTGTATTTTCCTGGATTTATAGTATGTGACTTTTCTGA |
| | | TTAATATATTTAATATATTGAATAAAAAATAGACATGTAGTTGGAA |
| 9 | PTPN2 | GCATGCGCCGCAGCGCCAGCGCTCTCCCCGGATCGTGCGGGGCCTGAGCCTCTC |
| | mRNA | CGCCGGCGCAGGCTCTGCTCGCGCCAGCTCGCTCCCGCAGCCATGCCCACCACC |
| | NCBI | ATCGAGCGGGAGTTCGAAGAGTTGGATACTCAGCGTCGCTGGCAGCCGCTGTA |
| | NM_002828.4 | CTTGGAAATTCGAAATGAGTCCCATGACTATCCTCATAGAGTGGCCAAGTTTCC |
| | | AGAAAACAGAAATCGAAACAGATACAGAGATGTAAGCCCATATGATCACAGTC |
| | | GTGTTAAACTGCAAAATGCTGAGAATGATTATATTAATGCCAGTTTAGTTGACA |
| | | TAGAAGAGGCACAAAGGAGTTACATCTTAACACAGGGTCCACTTCCTAACACA |
| | | TGCTGCCATTTCTGGCTTATGGTTTGGCAGCAGAAGACCAAAGCAGTTGTCATG |
| | | CTGAACCGCATTGTGGAGAAAGAATCGGTTAAATGTGCACAGTACTGGCCAAC |
| | | AGATGACCAAGAGATGCTGTTTAAAGAAACAGGATTCAGTGTGAAGCTCTTGTC |
| | | AGAAGATGTGAAGTCGTATTATACAGTACATCTACTACAATTAGAAAATATCAA |
| | | TAGTGGTGAAACCAGAACAATATCTCACTTTCATTATACTACCTGGCCAGATTT |
| | | TGGAGTCCCTGAATCACCAGCTTCATTTCTCAATTTCTTGTTTAAAGTGAGAGAA |
| | | TCTGGCTCCTTGAACCCTGACCATGGGCCTGCGGTGATCCACTGTAGTGCAGGC |
| | | ATTGGGCGCTCTGGCACCTTCTCTCTGGTAGACACTTGTCTTGTTTTGATGGAAA |
| | | AAGGAGATGATATTAACATAAAACAAGTGTTACTGAACATGAGAAAATACCGA |
| | | ATGGGTCTTATTCAGACCCCAGATCAACTGAGATTCTCATACATGGCTATAATA |
| | | GAAGGAGCAAAATGTATAAAGGGAGATTCTAGTATACAGAAACGATGGAAAG |
| | | AACTTTCTAAGGAAGACTTATCTCCTGCCTTTGATCATTCACCAAACAAAATAA |
| | | TGACTGAAAAATACAATGGGAACAGAATAGGTCTAGAAGAAGAAAAACTGAC |
| | | AGGTGACCGATGTACAGGACTTTCCTCTAAAATGCAAGATACAATGGAGGAGA |
| | | ACAGTGAGAGTGCTCTACGGAAACGTATTCGAGAGGACAGAAAGGCCACCACA |
| | | GCTCAGAAGGTGCAGCAGATGAAACAGAGGCTAAATGAGAATGAACGAAAAA |
| | | GAAAAAGGTGGTTATATTGGCAACCTATTCTCACTAAGATGGGGTTTATGTCAG |
| | | TCATTTTGGTTGGCGCTTTTGTTGGCTGGACACTGTTTTTTCAGCAAAATGCCCT |
| | | ATAAACAATTAATTTTGCCCAGCAAGCTTCTGCACTAGTAACTGACAGTGCTAC |
| | | ATTAATCATAGGGGTTTGTCTGCAGCAAACGCCTCATATCCCAAAAACGGTGCA |
| | | GTAGAATAGACATCAACCAGATAAGTGATATTTACAGTCACAAGCCCAACATCT |
| | | CAGGACTCTTGACTGCAGGTTCCTCTGAACCCCAAACTGTAAATGGCTGTCTAA |
| | | AATAAAGACATTCATGTTTGTTAAAAACTGGTAAATTTTGCAACTGTATTCATA |
| | | CATGTCAAACACAGTATTTCACCTGACCAACATTGAGATATCCTTTATCACAGG |
| | | ATTTGTTTTTGGAGGCTATCTGGATTTTAACCTGCACTTGATATAAGCAATAAAT |
| | | ATTGTGGTTTTATCTACGTTATTGGAAAGAAAATGACATTTAAATAATGTGTGT |
| | | AATGTATAATGTACTATTGACATGGGCATCAACACTTTTATTCTTAAGCATTTCA |
| | | GGGTAAATATATTTTATAAGTATCTATTTAATCTTTTGTAGTTAACTGTACTTTTT |
| | | AAGAGCTCAATTTGAAAAATCTGTTACTAAAAAAATAAATTGTATGTCGATTGA |
| | | ATTGTACTGGATACATTTTCCATTTTTCTAAAGAGAAGTTTGATATGAGCAGTTA |
| | | GAAGTTGGAATAAGCAATTTCTACTATATATTGCATTTCTTTTATGTTTTACAGT |
| | | TTTCCCCATTTTAAAAAGAAAAGCAAACAAAGAAACAAAAGTTTTTCCTAAAA |
| | | ATATCTTTGAAGGAAAATTCTCCTTACTGGGATAGTCAGGTAAACAGTTGGTCA |
| | | AGACTTTGTAAAGAAATTGGTTTCTGTAAATCCCATTATTGATATGTTTATTTTT |
| | | CATGAAAATTTCAATGTAGTTGGGGTAGATTATGATTTAGGAAGCAAAAGTAA |
| | | GAAGCAGCATTTTATGATTCATAATTTCAGTTTACTAGACTGAAGTTTTGAAGT |
| | | AAACACTTTTCAGTTTCTTTCTACTTCAATAAATAGTATGATTATATGCAAACCT |
| | | TACATTGTCATTTTAACTTAATGAATATTTTTTAAAGCAAACTGTTTAATGAATT |
| | | TAACTGCTCATTTGAATGCTAGCTTTCCTCAGATTTCAACATTCCATTCAGTGTT |
| | | TAATTTGTCTTACTTAAACTTGAAATTGTTGTTACAAATTTAATTGCTAGGAGGC |
| | | ATGGATAGCATACATTATTATGGATAGCATACCTTATTTCAGTGGTTTTCAAACT |
| | | ATGCTCATTGGATGTCCAGGTGGGTCAAGAGGTTACTTTCAACCACAGCATCTC |
| | | TGCCTTGTCTCTTTATATGCCACATAAGATTTCTGCATAAGGCTTAAGTATTTTA |
| | | AAGGGGGCAGTTATCATTTAAAAACAGTTTGGTCGGGCGCGGTGGCTCATGCCT |
| | | GTAATCCCAGCACTTTGGGAGGCTGAAGTGGGCAGATCACCTGAGGTCAGGAG |
| | | TTCAAGACCAGCCTGGCCAACGTGGTGAAACACCATCTCTACTAAAAATGCAA |
| | | AAATTAGCTGGGCATGGTGGAGGGCACCTGTAATCTCAGCTACTCAGGAGGCT |
| | | GAGGTAGGAGAATTGCTTGAACCCAGGAGATGGAGGTTGCAGTGAGCTGAGAT |
| | | CACGTCACTGCACTCCAGCCAGGGCGACAGAGCGAGACTCCATCTCAAAAGAA |
| | | ACAAACAAAAAAAACAGTTTGGGCCGGGTGTGGTGGCTCACGCTTGTAATCCC |
| | | AGCACTTCGGAAGGCCAAGGCGGGCGGATCACGAGGTCAAGAGATGGAGACTG |
| | | TCCTGGCCAACATGGTGAAATCCCTTCTTTACTAAAAATACAAAAATTATCTGG |
| | | GCGTGGTGGTGCATGCCTGTAGTCCCAGCTCCTTGGGAGGCTAAGGCAGGAGA |
| | | ATCACTTGAACCCGGGAGGCAGAGGTTGCAGTGAGCCGAGATTGCACCACTGC |
| | | ACTCCAGCCTGGCAACAGAGCAAGACTTCGTCTCAAAAAAAAAAAAAAAAAAA |
| | | GTTTGAAAACCATTGGTATAGATAGATATTTTGAATTGATTTGCATAGTCTCCTT |
| | | GAATGTGTTAAATTATGTTGAAAGTATGAAAGCAGGATGTAGGTGGTACTACAT |
| | | ATTAAATAAGATTTATATAACA |
| 10 | TET2 | AAACAGAAGGTGGGCCGGGGGGGGGAGAAACAGAACTCGGTCAATTTCCCAGT |
| | mRNA | TTGTCGGGTCTTTAAAAATACAGGCCCCTAAAGCACTAAGGGCATGCCCTCGGT |
| | NCBI | GAAACAGGGGAGCGCTTCTGCTGAATGAGATTAAAGCGACAGAAAAGGGAAA |
| | XM_024454103.2 | GGAGAGCGCGGGCAACGGGATCTAAAGGGAGATAGAGACGCGGGCCTCTGAG |
| | | GGCTGGCAAACATTCAGCAGCACACCCTCTCAAGATTGTTTACTTGCCTTTGCTC |
| | | CTGTTGAGTTACAACGCTTGGAAGCAGGAGATGGGCTCAGCAGCAGCCAATAG |
| | | GACATGATCCAGGAAGAGCAGTAAGGGACTGAGCTGCTGAATTCAACTAGAGG |
| | | GCAGCCTTGTGGATGGCCCCGAAGCAAGCCTGATGGAACAGGATAGAACCAAC |
| | | CATGTTGAGGGCAACAGACTAAGTCCATTCCTGATACCATCACCTCCCATTTGC |
| | | CAGACAGAACCTCTGGCTACAAAGCTCCAGAATGGAAGCCCACTGCCTGAGAG |
| | | AGCTCATCCAGAAGTAAATGGAGACACCAAGTGGCACTCTTTCAAAAGTTATTA |
| | | TGGAATACCCTGTATGAAGGGAAGCCAGAATAGTCGTGTGAGTCCTGACTTTAC |
| | | ACAAGAAAGTAGAGGGTATTCCAAGTGTTTGCAAAATGGAGGAATAAAACGCA |
| | | CAGTTAGTGAACCTTCTCTCTCTGGGCTCCTTCAGATCAAGAAATTGAAACAAG |
| | | ACCAAAAGGCTAATGGAGAAAGACGTAACTTCGGGGTAAGCCAAGAAAGAAA |
| | | TCCAGGTGAAAGCAGTCAACCAAATGTCTCCGATTTGAGTGATAAGAAAGAAT |
| | | CTGTGAGTTCTGTAGCCCAAGAAAATGCAGTTAAAGATTTCACCAGTTTTTCAA |
| | | CACATAACTGCAGTGGGCCTGAAAATCCAGAGCTTCAGATTCTGAATGAGCAG |
| | | GAGGGGAAAAGTGCTAATTACCATGACAAGAACATTGTATTACTTAAAAACAA |
| | | GGCAGTGCTAATGCCTAATGGTGCTACAGTTTCTGCCTCTTCCGTGGAACACAC |
| | | ACATGGTGAACTCCTGGAAAAAACACTGTCTCAATATTATCCAGATTGTGTTTC |
| | | CATTGCGGTGCAGAAAACCACATCTCACATAAATGCCATTAACAGTCAGGCTAC |
| | | TAATGAGTTGTCCTGTGAGATCACTCACCCATCGCATACCTCAGGGCAGATCAA |
| | | TTCCGCACAGACCTCTAACTCTGAGCTGCCTCCAAAGCCAGCTGCAGTGGTGAG |
| | | TGAGGCCTGTGATGCTGATGATGCTGATAATGCCAGTAAACTAGCTGCAATGCT |
| | | AAATACCTGTTCCTTTCAGAAACCAGAACAACTACAACAACAAAAATCAGTTTT |
| | | TGAGATATGCCCATCTCCTGCAGAAAATAACATCCAGGGAACCACAAAGCTAG |
| | | CGTCTGGTGAAGAATTCTGTTCAGGTTCCAGCAGCAATTTGCAAGCTCCTGGTG |
| | | GCAGCTCTGAACGGTATTTAAAACAAAATGAAATGAATGGTGCTTACTTCAAGC |
| | | AAAGCTCAGTGTTCACTAAGGATTCCTTTTCTGCCACTACCACACCACCACCAC |
| | | CATCACAATTGCTTCTTTCTCCCCCTCCTCCTCTTCCACAGGTTCCTCAGCTTCCT |
| | | TCAGAAGGAAAAAGCACTCTGAATGGTGGAGTTTTAGAAGAACACCACCACTA |
| | | CCCCAACCAAAGTAACACAACACTTTTAAGGGAAGTGAAAATAGAGGGTAAAC |
| | | CTGAGGCACCACCTTCCCAGAGTCCTAATCCATCTACACATGTATGCAGCCCTT |
| | | CTCCGATGCTTTCTGAAAGGCCTCAGAATAATTGTGTGAACAGGAATGACATAC |
| | | AGACTGCAGGGACAATGACTGTTCCATTGTGTTCTGAGAAAACAAGACCAATGT |
| | | CAGAACACCTCAAGCATAACCCACCAATTTTTGGTAGCAGTGGAGAGCTACAG |
| | | GACAACTGCCAGCAGTTGATGAGAAACAAAGAGCAAGAGATTCTGAAGGGTCG |
| | | AGACAAGGAGCAAACACGAGATCTTGTGCCCCCAACACAGCACTATCTGAAAC |
| | | CAGGATGGATTGAATTGAAGGCCCCTCGTTTTCACCAAGCGGAATCCCATCTAA |
| | | AACGTAATGAGGCATCACTGCCATCAATTCTTCAGTATCAACCCAATCTCTCCA |
| | | ATCAAATGACCTCCAAACAATACACTGGAAATTCCAACATGCCTGGGGGGCTCC |
| | | CAAGGCAAGCTTACACCCAGAAAACAACACAGCTGGAGCACAAGTCACAAATG |
| | | TACCAAGTTGAAATGAATCAAGGGCAGTCCCAAGGTACAGTGGACCAACATCT |
| | | CCAGTTCCAAAAACCCTCACACCAGGTGCACTTCTCCAAAACAGACCATTTACC |
| | | AAAAGCTCATGTGCAGTCACTGTGTGGCACTAGATTTCATTTTCAACAAAGAGC |
| | | AGATTCCCAAACTGAAAAACTTATGTCCCCAGTGTTGAAACAGCACTTGAATCA |
| | | ACAGGCTTCAGAGACTGAGCCATTTTCAAACTCACACCTTTTGCAACATAAGCC |
| | | TCATAAACAGGCAGCACAAACACAACCATCCCAGAGTTCACATCTCCCTCAAA |
| | | ACCAGCAACAGCAGCAAAAATTACAAATAAAGAATAAAGAGGAAATACTCCA |
| | | GACTTTTCCTCACCCCCAAAGCAACAATGATCAGCAAAGAGAAGGATCATTCTT |
| | | TGGCCAGACTAAAGTGGAAGAATGTTTTCATGGTGAAAATCAGTATTCAAAATC |
| | | AAGCGAGTTCGAGACTCATAATGTCCAAATGGGACTGGAGGAAGTACAGAATA |
| | | TAAATCGTAGAAATTCCCCTTATAGTCAGACCATGAAATCAAGTGCATGCAAAA |
| | | TACAGGTTTCTTGTTCAAACAATACACACCTAGTTTCAGAGAATAAAGAACAGA |
| | | CTACACATCCTGAACTTTTTGCAGGAAACAAGACCCAAAACTTGCATCACATGC |
| | | AATATTTTCCAAATAATGTGATCCCAAAGCAAGATCTTCTTCACAGGTGCTTTC |
| | | AAGAACAGGAGCAGAAGTCACAACAAGCTTCAGTTCTACAGGGATATAAAAAT |
| | | AGAAACCAAGATATGTCTGGTCAACAAGCTGCGCAACTTGCTCAGCAAAGGTA |
| | | CTTGATACATAACCATGCAAATGTTTTTCCTGTGCCTGACCAGGGAGGAAGTCA |
| | | CACTCAGACCCCTCCCCAGAAGGACACTCAAAAGCATGCTGCTCTAAGGTGGC |
| | | ATCTCTTACAGAAGCAAGAACAGCAGCAAACACAGCAACCCCAAACTGAGTCT |
| | | TGCCATAGTCAGATGCACAGGCCAATTAAGGTGGAACCTGGATGCAAGCCACA |
| | | TGCCTGTATGCACACAGCACCACCAGAAAACAAAACATGGAAAAAGGTAACTA |
| | | AGCAAGAGAATCCACCTGCAAGCTGTGATAATGTGCAGCAAAAGAGCATCATT |
| | | GAGACCATGGAGCAGCATCTGAAGCAGTTTCACGCCAAGTCGTTATTTGACCAT |
| | | AAGGCTCTTACTCTCAAATCACAGAAGCAAGTAAAAGTTGAAATGTCAGGGCC |
| | | AGTCACAGTTTTGACTAGACAAACCACTGCTGCAGAACTTGATAGCCACACCCC |
| | | AGCTTTAGAGCAGCAAACAACTTCTTCAGAAAAGACACCAACCAAAAGAACAG |
| | | CTGCTTCTGTTCTCAATAATTTTATAGAGTCACCTTCCAAATTACTAGATACTCC |
| | | TATAAAAAATTTATTGGATACACCTGTCAAGACTCAATATGATTTCCCATCTTGC |
| | | AGATGTGTAGAGCAAATTATTGAAAAAGATGAAGGTCCTTTTTATACCCATCTA |
| | | GGAGCAGGTCCTAATGTGGCAGCTATTAGAGAAATCATGGAAGAAAGGTTTGG |
| | | ACAGAAGGGTAAAGCTATTAGGATTGAAAGAGTCATCTATACTGGTAAAGAAG |
| | | GCAAAAGTTCTCAGGGATGTCCTATTGCTAAGTGGGTGGTTCGCAGAAGCAGCA |
| | | GTGAAGAGAAGCTACTGTGTTTGGTGCGGGAGCGAGCTGGCCACACCTGTGAG |
| | | GCTGCAGTGATTGTGATTCTCATCCTGGTGTGGGAAGGAATCCCGCTGTCTCTG |
| | | GCTGACAAACTCTACTCGGAGCTTACCGAGACGCTGAGGAAATACGGCACGCT |
| | | CACCAATCGCCGGTGTGCCTTGAATGAAGAGAGAACTTGCGCCTGTCAGGGGCT |
| | | GGATCCAGAAACCTGTGGTGCCTCCTTCTCTTTTGGTTGTTCATGGAGCATGTAC |
| | | TACAATGGATGTAAGTTTGCCAGAAGCAAGATCCCAAGGAAGTTTAAGCTGCTT |
| | | GGGGATGACCCAAAAGAGGAAGAGAAACTGGAGTCTCATTTGCAAAACCTGTC |
| | | CACTCTTATGGCACCAACATATAAGAAACTTGCACCTGATGCATATAATAATCA |
| | | GATTGAATATGAACACAGAGCACCAGAGTGCCGTCTGGGTCTGAAGGAAGGCC |
| | | GTCCATTCTCAGGGGTCACTGCATGTTTGGACTTCTGTGCTCATGCCCACAGAG |
| | | ACTTGCACAACATGCAGAATGGCAGCACATTGGTATGCACTCTCACTAGAGAA |
| | | GACAATCGAGAATTTGGAGGAAAACCTGAGGATGAGCAGCTTCACGTTCTGCC |
| | | TTTATACAAAGTCTCTGACGTGGATGAGTTTGGGAGTGTGGAAGCTCAGGAGGA |
| | | GAAAAAACGGAGTGGTGCCATTCAGGTACTGAGTTCTTTTCGGCGAAAAGTCA |
| | | GGATGTTAGCAGAGCCAGTCAAGACTTGCCGACAAAGGAAACTAGAAGCCAAG |
| | | AAAGCTGCAGCTGAAAAGCTTTCCTCCCTGGAGAACAGCTCAAATAAAAATGA |
| | | AAAGGAAAAGTCAGCCCCATCACGTACAAAACAAACTGAAAACGCAAGCCAG |
| | | GCTAAACAGTTGGCAGAACTTTTGCGACTTTCAGGACCAGTCATGCAGCAGTCC |
| | | CAGCAGCCCCAGCCTCTACAGAAGCAGCCACCACAGCCCCAGCAGCAGCAGAG |
| | | ACCCCAGCAGCAGCAGCCACATCACCCTCAGACAGAGTCTGTCAACTCTTATTC |
| | | TGCTTCTGGATCCACCAATCCATACATGAGACGGCCCAATCCAGTTAGTCCTTA |
| | | TCCAAACTCTTCACACACTTCAGATATCTATGGAAGCACCAGCCCTATGAACTT |
| | | CTATTCCACCTCATCTCAAGCTGCAGGTTCATATTTGAATTCTTCTAATCCCATG |
| | | AACCCTTACCCTGGGCTTTTGAATCAGAATACCCAATATCCATCATATCAATGC |
| | | AATGGAAACCTATCAGTGGACAACTGCTCCCCATATCTGGGTTCCTATTCTCCC |
| | | CAGTCTCAGCCGATGGATCTGTATAGGTATCCAAGCCAAGACCCTCTGTCTAAG |
| | | CTCAGTCTACCACCCATCCATACACTTTACCAGCCAAGGTTTGGAAATAGCCAG |
| | | AGTTTTACATCTAAATACTTAGGTTATGGAAACCAAAATATGCAGGGAGATGGT |
| | | TTCAGCAGTTGTACCATTAGACCAAATGTACATCATGTAGGGAAATTGCCTCCT |
| | | TATCCCACTCATGAGATGGATGGCCACTTCATGGGAGCCACCTCTAGATTACCA |
| | | CCCAATCTGAGCAATCCAAACATGGACTATAAAAATGGTGAACATCATTCACCT |
| | | TCTCACATAATCCATAACTACAGTGCAGCTCCGGGCATGTTCAACAGCTCTCTT |
| | | CATGCCCTGCATCTCCAAAACAAGGAGAATGACATGCTTTCCCACACAGCTAAT |
| | | GGGTTATCAAAGATGCTTCCAGCTCTTAACCATGATAGAACTGCTTGTGTCCAA |
| | | GGAGGCTTACACAAATTAAGTGATGCTAATGGTCAGGAAAAGCAGCCATTGGC |
| | | ACTAGTCCAGGGTGTGGCTTCTGGTGCAGAGGACAACGATGAGGTCTGGTCAG |
| | | ACAGCGAGCAGAGCTTTCTGGATCCTGACATTGGGGGAGTGGCCGTGGCTCCA |
| | | ACTCATGGGTCAATTCTCATTGAGTGTGCAAAGCGTGAGCTGCATGCCACAACC |
| | | CCTTTAAAGAATCCCAATAGGAATCACCCCACCAGGATCTCCCTCGTCTTTTAC |
| | | CAGCATAAGAGCATGAATGAGCCAAAACATGGCTTGGCTCTTTGGGAAGCCAA |
| | | AATGGCTGAAAAAGCCCGTGAGAAAGAGGAAGAGTGTGAAAAGTATGGCCCA |
| | | GACTATGTGCCTCAGAAATCCCATGGCAAAAAAGTGAAACGGGAGCCTGCTGA |
| | | GCCACATGAAACTTCAGAGCCCACTTACCTGCGTTTCATCAAGTCTCTTGCCGA |
| | | AAGGACCATGTCCGTGACCACAGACTCCACAGTAACTACATCTCCATATGCCTT |
| | | CACTCGGGTCACAGGGCCTTACAACAGATATATATGATATCACCCCCTTTTGTT |
| | | GGTTACCTCACTTGAAAAGACCACAACCAACCTGTCAGTAGTATAGTTCTCATG |
| | | ACGTGGGCAGTGGGGAAAGGTCACAGTATTCATGACAAATGTGGTGGGAAAAA |
| | | CCTCAGCTCACCAGCAACAAAAGAGGTTATCTTACCATAGCACTTAATTTTCAC |
| | | TGGCTCCCAAGTGGTCACAGATGGCATCTAGGAAAAGACCAAAGCATTCTATG |
| | | CAAAAAGAAGGTGGGGAAGAAAGTGTTCCGCAATTTACATTTTTAAACACTGG |
| | | TTCTATTATTGGACGAGATGATATGTAAATGTGATCCCCCCCCCCCGCTTACAA |
| | | CTCTACACATCTGTGACCACTTTTAATAATATCAAGTTTGCATAGTCATGGAAC |
| | | ACAAATCAAACAAGTACTGTAGTATTACAGTGACAGGAATCTTAAAATACCATC |
| | | TGGTGCTGAATATATGATGTACTGAAATACTGGAATTATGGCTTTTTGAAATGC |
| | | AGTTTTTACTGTAATCTTAACTTTTATTTATCAAAATAGCTACAGGAAACATGAA |
| | | TAGCAGGAAAACACTGAATTTGTTTGGATGTTCTAAGAAATGGTGCTAAGAAA |
| | | ATGGTGTCTTTAATAGCTAAAAATTTAATGCCTTTATATCATCAAGATGCTATCA |
| | | GTGTACTCCAGTGCCCTTGAATAATAGGGGTACCTTTTCATTCAAGTTTTTATCA |
| | | TAATTACCTATTCTTACACAAGCTTAGTTTTTAAAATGTGGACATTTTAAAGGCC |
| | | TCTGGATTTTGCTCATCCAGTGAAGTCCTTGTAGGACAATAAACGTATATATGT |
| | | ACATATATACACAAACATGTATATGTGCACACACATGTATATGTATAAATATTT |
| | | TAAATGGTGTTTTAGAAGCACTTTGTCTACCTAAGCTTTGACAACTTGAACAAT |
| | | GCTAAGGTACTGAGATGTTTAAAAAACAAGTTTACTTTCATTTTAGAATGCAAA |
| | | GTTGATTTTTTTAAGGAAACAAAGAAAGCTTTTAAAATATTTTTGCTTTTAGCCA |
| | | TGCATCTGCTGATGAGCAATTGTGTCCATTTTTAACACAGCCAGTTAAATCCAC |
| | | CATGGGGCTTACTGGATTCAAGGGAATACGTTAGTCCACAAAACATGTTTTCTG |
| | | GTGCTCATCTCACATGCTATACTGTAAAACAGTTTTATACAAAATTGTATGACA |
| | | AGTTCATTGCTCAAAAATGTACAGTTTTAAGAATTTTCTATTAACTGCAGGTAAT |
| | | AATTAGCTGCATGCTGCAGACTCAACAAAGCTAGTTCACTGAAGCCTATGCTAT |
| | | TTTATGGATCATAGGCTCTTCAGAGAACTGAATGGCAGTCTGCCTTTGTGTTGAT |
| | | AATTATGTACATTGTGACGTTGTCATTTCTTAGCTTAAGTGTCCTCTTTAACAAG |
| | | AGGATTGAGCAGACTGATGCCTGCATAAGATGAATAAACAGGGTTAGTTCCAT |
| | | GTGAATCTGTCAGTTAAAAAGAAACAAAAACAGGCAGCTGGTTTGCTGTGGTG |
| | | GTTTTAAATCATTAATTTGTATAAAGAAGTGAAAGAGTTGTATAGTAAATTAAA |
| | | TTGTAAACAAAACTTTTTTAATGCAATGCTTTAGTATTTTAGTACTGTAAAAAAA |
| | | TTAAATATATACATATATATATATATATATATATATATATATATGAGTTTGAAGC |
| | | AGAATTCACATCATGATGGTGCTACTCAGCCTGCTACAAATATATCATAATGTG |
| | | AGCTAAGAATTCATTAAATGTTTGAGTGATGTTCCTACTTGTCATATACCTCAAC |
| | | ACTAGTTTGGCAATAGGATATTGAACTGAGAGTGAAAGCATTGTGTACCATCAT |
| | | TTTTTTCCAAGTCCTTTTTTTTATTGTTAAAAAAAAAAGCATACCTTTTTTCAATA |
| | | CTTGATTTCTTAGCAAGTATAACTTGAACTTCAACCTTTTTGTTCTAAAAATTCA |
| | | GGGATATTTCAGCTCATGCTCTCCCTATGCCAACATGTCACCTGTGTTTATGTAA |
| | | AATTGTTGTAGGTTAATAAATATATTCTTTGTCAGGGATTTAACCCTTTTATTTT |
| | | GAATCCCTTCTATTTTACTTGTACATGTGCTGATGTAACTAAAACTAATTTTGTA |
| | | AATCTGTTGGCTCTTTTTATTGTAAAGAAAAGCATTTTAAAAGTTTGAGGAATCT |
| | | TTTGACTGTTTCAAGCAGGAAAAAAAAATTACATGAAAATAGAATGCACTGAG |
| | | TTGATAAAGGGAAAAATTGTAAGGCAGGAGTTTGGCAAGTGGCTGTTGGCCAG |
| | | AGACTTACTTGTAACTCTCTAAATGAAGTTTTTTTGATCCTGTAATCACTGAAGG |
| | | TACATACTCCATGTGGACTTCCCTTAAACAGGCAAACACCTACAGGTATGGTGT |
| | | GCAACAGATTGTACAATTACATTTTGGCCTAAATACATTTTTGCTTACTAGTATT |
| | | TAAAATAAATTCTTAATCAGAGGAGGCCTTTGGGTTTTATTGGTCAAATCTTTGT |
| | | AAGCTGGCTTTTGTCTTTTTAAAAAATTTCTTGAATTTGTGGTTGTGTCCAATTT |
| | | GCAAACATTTCCAAAAATGTTTGCTTTGCTTACAAACCACATGATTTTAATGTTT |
| | | TTTGTATACCATAATATCTAGCCCCAAACATTTGATTACTACATGTGCATTGGTG |
| | | ATTTTGATCATCCATTCTTAATATTTGATTTCTGTGTCACCTACTGTCATTTGTTA |
| | | AACTGCTGGCCAACAAGAACAGGAAGTATAGTTTGGGGGGTTGGGGAGAGTTT |
| | | ACATAAGGAAGAGAAGAAATTGAGTGGCATATTGTAAATATCAGATCTATAAT |
| | | TGTAAATATAAAACCTGCCTCAGTTAGAATGAATGGAAAGCAGATCTACAATTT |
| | | GCTAATATAGGAATATCAGGTTGACTATATAGCCATACTTGAAAATGCTTCTGA |
| | | GTGGTGTCAACTTTACTTGAATGAATTTTTCATCTTGATTGACGCACAGTGATGT |
| | | ACAGTTCACTTCTGAAGCTAGTGGTTAACTTGTGTAGGAAACTTTTGCAGTTTG |
| | | ACACTAAGATAACTTCTGTGTGCATTTTTCTATGCTTTTTTAAAAACTAGTTTCA |
| | | TTTCATTTTCATGAGATGTTTGGTTTATAAGATCTGAGGATGGTTATAAATACTG |
| | | TAAGTATTGTAATGTTATGAATGCAGGTTATTTGAAAGCTGTTTATTATTATATC |
| | | ATTCCTGATAATGCTATGTGAGTGTTTTTAATAAAATTTATATTTATTTAATGCA |
| | | CTCTAA |
| 11 | ZC3H12A | AGGACTCGGCCCCATGGAGACGCCGCCGGCCGCCGCTGGCGCATGGCGGGTAG |
| | mRNA | GAGCTGTGGCGCGGGGCCTTCCAGGAGTCTGAGCTATGAGTGGCCCCTGTGGA |
| | NCBI | GAGAAGCCTGTCCTGGAAGCCAGCCCCACCATGAGTCTGTGGGAATTTGAGGA |
| | XM_011542198 | CAGCCACAGCCGTCAGGGCACCCCAAGGCCGGGTCAAGAGCTGGCCGCTGAGG |
| | | AGGCCTCGGCCCTGGAACTGCAGATGAAGGTGGACTTCTTCCGGAAGCTGGGCT |
| | | ATTCATCCACGGAGATCCACAGCGTCCTGCAGAAGCTGGGCGTCCAGGCAGAC |
| | | ACCAACACGGTGCTGGGTGAGCTGGTGAAACACGGGACAGCCACCGAGCGGGA |
| | | GCGCCAGACCTCACCGGACCCCTGCCCTCAGCTCCCTCTAGTCCCGCGGGGTGG |
| | | TGGCACCCCTAAGGCTCCCAACCTGGAGCCTCCACTCCCAGAAGAGGAAAAGG |
| | | AGGGCAGCGACCTGAGACCAGTGGTCATCGATGGGAGCAACGTGGCCATGAGC |
| | | CATGGGAACAAGGAGGTCTTCTCCTGCCGGGGCATCCTGCTGGCAGTGAACTGG |
| | | TTTCTGGAGCGGGGCCACACAGACATCACAGTGTTTGTGCCATCCTGGAGGAAG |
| | | GAGCAGCCTCGGCCCGACGTGCCCATCACAGACCAGCACATCCTGCGGGAACT |
| | | GGAGAAGAAGAAGATCCTGGTGTTCACACCATCACGACGCGTGGGTGGCAAGC |
| | | GGGTGGTGTGCTATGACGACAGATTCATTGTGAAGCTGGCCTACGAGTCTGACG |
| | | GGATCGTGGTTTCCAACGACACATACCGTGACCTCCAAGGCGAGCGGCAGGAG |
| | | TGGAAGCGCTTCATCGAGGAGCGGCTGCTCATGTACTCCTTCGTCAATGACAAG |
| | | CTTGCCATCGGCCCCTTCTCAGCAATTAGAAGTCCCTGCATGGCTCCCACCTCCA |
| | | GAGAGTTCTTTGCACCCTCTGCAGGTTTATGCCCCCTGATGACCCACTGGGCCG |
| | | GCACGGGCCCAGCCTGGACAACTTCCTGCGTAAGAAGCCACTCACTTTGGAGCA |
| | | CAGGAAGCAGCCGTGTCCCTATGGAAGGAAATGCACCTATGGGATCAAGTGCC |
| | | GATTCTTCCACCCAGAGCGGCCAAGCTGCCCCCAGCGCTCTGTGGCAGATGAGC |
| | | TCCGTGCCAATGCTCTCCTCTCACCCCCCAGAGCCCCAAGCAAGGACAAAAATG |
| | | GCCGGCGGCCTTCACCTTCATCCCAGTCCAGCTCTCTGCTAACAGAGAGTGAGC |
| | | AGTGCAGCCTGGATGGGAAGAAGCTGGGGGCCCAGGCATCCCCAGGGTCCCGC |
| | | CAAGAGGGTCTAACACAGACCTATGCCCCATCAGGCAGGAGCCTCGCACCTAG |
| | | CGGGGGCAGTGGCAGCAGCTTTGGGCCCACAGACTGGCTCCCACAGACGCTGG |
| | | ACTCACTCCCGTACGTCTCCCAGGATTGCCTGGACTCGGGCATTGGCTCCCTGG |
| | | AGAGCCAGATGTCGGAACTTTGGGGGGTTCGAGGAGGAGGCCCTGGTGAGCCG |
| | | GGCCCACCCCGAGCCCCTTACACGGGCTACAGTCCCTATGGATCTGAGCTCCCA |
| | | GCCACCGCAGCCTTCTCTGCCTTTGGCCGGGCCATGGGTGCTGGCCACTTCAGT |
| | | GTCCCTGCCGACTACCCACCCGCGCCCCCTGCCTTTCCACCTCGAGAGTACTGG |
| | | TCTGAACCATACCCACTGCCCCCACCCACATCAGTCCTTCAGGAGCCCCCAGTG |
| | | CAGAGCCCAGGGGCTGGCAGGAGCCCGTGGGGCAGGGCAGGCAGCCTGGCCA |
| | | AGGAGCAGGCCAGCGTGTATACTAAGCTGTGTGGTGTGTTTCCCCCGCACCTGG |
| | | TGGAGGCTGTGATGGGGCGCTTCCCACAGCTCCTGGACCCCCAGCAGCTGGCTG |
| | | CCGAGATCCTCTCCTACAAGTCCCAGCACCCCAGTGAGTAAGCTGCCTGTGGCT |
| | | GGCAAGGGCAGCACCCCCAGCCTCCAAGGGCCGTCAGGCTGGGCTTTGGGCCA |
| | | TTGAGCAGCCCATTCCCAGCCCTGAGGCCCACCCCAGAGGCTGGACAGAGGGA |
| | | GGATTCAAGTCGGGAAGGAAACCCACAAACCAAAGATACTGTAGGATTGGTTC |
| | | TGGCCCATGCAGCACCTCTAGCTGTCTGCCTCAGTGGGTCAGAAGCGATCACCC |
| | | TGTTGATACACATTGTATCTCTGTAGTTTAAGGAGACGCTGCCGGTAACGGCGT |
| | | CGGTCCGTGGCTGAGGCCCAAACCGTCTTTTCTCTCAGAGGGTGGGGAGGGAG |
| | | GTGGGGGCAGCAGAGGCCTGGGCTGGGTGCCCTGTGCACGCCACCCCACTTCC |
| | | GCCCTACCCCTGGGACGTTGGCCTTGGCTGGCTAGTTGGGCACCGTGTGCCTGC |
| | | CCTCCAAGGGCCTCCTCTACGCCAATGAGGCCTCATCTGTGCTCTCGCTGGGCA |
| | | CGTGGCTTCATGTCAGTAAGCAAGATGCTTCTTAATAACCCACCTTCTGCCCCA |
| | | CTCTATTCCTTATCCTGCTGCCCCTGTAGGGGTCAAGGGCCCTCCGTCTACACCC |
| | | TCTTCTTCTCCTCCATCCTTTATTCAGAGTCATCTCGCCCTTCCCCATGGGTGGG |
| | | GGAACCTGTGTTTGTTTGTGTGCACATGTAAATTTTAAATATTTTAAGCAGAAA |
| | | GTCCTTACCTCCTGTAACACATCAATAAAGTACAATCATTGTGAGCCCTTTCAA |
| 12 | CD5 | CAUACCAGCUGAGCCGUCCG |
| | sgRNA* | |
| 13 | CBLB | AAAAUAUCAAGUAUAUAUGG |
| | sgRNA* | |
| 14 | CISH | GCCAAAGGUGCUGGACCCAG |
| | sgRNA* | |
| 15 | DGKA | UCCGGAUCUUUUUCUGACAG |
| | sgRNA* | |
| 16 | DGKZ | GGUAGCCCUACGGCGCAAGG |
| | sgRNA* | |
| 17 | DNMT3A | UGACACGCCAAAGGACCCUG |
| | sgRNA* | |
| 18 | MAP4K1 | AGAGGGUGCACAUCAAAGAG |
| | sgRNA* | |
| 19 | NR4A1 | CAUGGACCUGGCCAGCCCCG |
| | sgRNA* | |
| 20 | PTPN2 | UUGACAUAGAAGAGGCACAA |
| | sgRNA* | |
| 21 | TET2 | AAAGAGCAAGAGAUUCUGAA |
| | sgRNA* | |
| 22 | ZC3H12A | CAGCUCCCUCUAGUCCCGCG |
| | sgRNA* | |
| 23 | CBLB | GTATTCATCATCATCTTCCTCT |
| | shRNA 81 | |
| 24 | CBLB | TTTAACTTCATCATATGTGAGA |
| | shRNA 19 | |
| 25 | CBLB | TTTATAAACTGAATGAGAATGT |
| | shRNA 14:2 | |
| 26 | CBLB | TTCATCATCATCTTCCTCTACT |
| | shRNA 9 | |
| 27 | CBLB | TTTGACTTTTTCATAAGGCTAT |
| | shRNA 79 | |
| 28 | CBLB | TGAATACTTTCCATGGTACGAT |
| | shRNA 64 | |
| 29 | CBLB | TTAAAGTACTCATTCTCACTGA |
| | shRNA 11 | |
| 30 | CBLB | TATAATTGAGGATAAGTCCTTC |
| | shRNA 23:2 | |
| 31 | CBLB | TTAGAAAACTTTCTGTGGGTTC |
| | shRNA 27:2 | |
| 32 | CBLB | TAATATATTAGGAATGTTGCAA |
| | shRNA 28:2 | |
| 33 | CBLB | TGAAATAAGGGCTTGTTATGAG |
| | shRNA 65 | |
| 34 | CBLB | TTTAGAAAACTTTCTGTGGGTT |
| | shRNA 18:2 | |
| 35 | CBLB | TTTATTAAGAGTAATAACATGG |
| | shRNA 4:2 | |
| 36 | CBLB | TTTGATTTCTTCTCTGAAGATG |
| | shRNA 30 | |
| 37 | CBLB | TCTTCATACATTCTCTCCTTGC |
| | shRNA 48 | |
| 38 | CBLB | TAAATATATGCTTAAGTCAGGG |
| | shRNA 5 | |
| 39 | CBLB | TCTTGTATTCATCATCATCTTC |
| | shRNA 45 | |
| 40 | CBLB | TACATTATGACAATGAGATGGT |
| | shRNA 4 | |
| 41 | CBLB | TTAATTGAACATATGACTATTT |
| | shRNA 6:2 | |
| 42 | CBLB | TTTATTGACAAATAAATGCTTT |
| | shRNA 3:2 | |
| 43 | CBLB | TATGACAATGAGATGGTTGTGA |
| | shRNA 53 | |
| 44 | CBLB | TTCTAAAGGCAAATCATGGCGT |
| | shRNA 23 | |
| 45 | CBLB | TTACATTATGACAATGAGATGG |
| | shRNA 3 | |
| 46 | CBLB | TGTGAATTCAGGGAAACAGGGT |
| | shRNA 72 | |
| 47 | CD5 | TGCAGATCATAGTCACTGTCGG |
| | shRNA 64 | |
| 48 | CD5 | TTGATTTTCCTGAAGCAATGCT |
| | shRNA 25 | |
| 49 | CD5 | TGATTTTCCTGAAGCAATGCTC |
| | shRNA 77 | |
| 50 | CD5 | TTTTGGTTCATTCCCGTTGGGC |
| | shRNA 13 | |
| 51 | CD5 | TCCAAGTACTAGAGGAGTCTGT |
| | shRNA 75 | |
| 52 | CD5 | TGACACATGTCATTTCTGCTGT |
| | shRNA 74 | |
| 53 | CD5 | TTATTCAAACCAAAGGAACATG |
| | shRNA 8 | |
| 54 | CD5 | TGCAGATCATAGTCACTGTCGG |
| | shRNA 64 | |
| 55 | CD5 | TTCGTTATCCACGTGGGAGGCT |
| | shRNA 21 | |
| 56 | CD5 | TGATTGAGCTCTGAGGTGTGTA |
| | shRNA 98 | |
| 57 | CD5 | TTGGAAGGCTGCTTGTTTTGGG |
| | shRNA 47 | |
| 58 | CD5 | TAGAAGATGAACTGTGATGGAC |
| | shRNA 11 | |
| 59 | CD5 | TTGGTTTTTAAGTAGGCCATGG |
| | shRNA 7 | |
| 60 | CD5 | TTTTATTAAAAAAGGCACTGAT |
| | shRNA 1 | |
| 61 | CD5 | TTCACTAGCTTCTTGTAGGCAA |
| | shRNA 18 | |
| 62 | CD5 | TTGGAAGGCTGCTTGTTTTGGG |
| | shRNA 47 | |
| 63 | CD5 | TTGGGTTAGAAGATGAACTGTG |
| | shRNA 2 | |
| 64 | CD5 | TATTCAAACCAAAGGAACATGA |
| | shRNA 34 | |
| 65 | CD5 | TATTCAAAATGCACTCGCCCAG |
| | shRNA 5 | |
| 66 | CD5 | TGGGTTAGAAGATGAACTGTGA |
| | shRNA 12 | |
| 67 | CD5 | AAGATGAACTGTGATGGACTGA |
| | shRNA 51 | |
| 68 | CD5 | TCAAACCAAAGGAACATGACCA |
| | shRNA 35 | |
| 69 | CD5 | TGAGCTTCCTTGTCACTCCTTA |
| | shRNA 10 | |
| 70 | CD5 | TAGGGACATGATTTCAAGTGAA |
| | shRNA 23 | |
| 71 | CD5 | AAAACTATTCAAAATGCACTCG |
| | shRNA 9 | |
| 72 | CD5 | AAACTATTCAAAATGCACTCGC |
| | shRNA 3 | |
| 73 | CISH | ATAAATAAATAAATAGTGTATA |
| | shRNA | |
| | 123 | |
| 74 | CISH | TGAGACACAGGCTCTGCTGGGG |
| | shRNA 55 | |
| 75 | CISH | TAAAGGTATAATGTCATTACAG |
| | shRNA 5 | |
| 76 | CISH | TCACGTACTAAGAACGTGCCTT |
| | shRNA 10 | |
| 77 | CISH | TGCAGATACAGCTCTGTCCTGT |
| | shRNA 74 | |
| 78 | CISH | TAAAATAAAGAGGTAATAAAGG |
| | shRNA 111 | |
| 79 | CISH | TATCATTTTATTATAGAGGTAA |
| | shRNA 2 | |
| 80 | CISH | TAGGAGAAGGTCTTGGCTATGC |
| | shRNA 66 | |
| 81 | CISH | TTTCATGAAGTCTTATCAGACG |
| | shRNA 7 | |
| 82 | CISH | TCTATGCAGATACAGCTCTGTC |
| | shRNA 23 | |
| 83 | CISH | TAAAATAAAGAGGTAATAAAGG |
| | shRNA 111 | |
| 84 | CISH | TGAAGTCTTATCAGACGTGTAT |
| | shRNA 51 | |
| 85 | CISH | TAGAGGTAATAAAATAAAGAGG |
| | shRNA 84 | |
| 86 | CISH | TTCATGAAGTCTTATCAGACGT |
| | shRNA 15 | |
| 87 | CISH | GAAGGTAGGAGAAGGTCTTGGC |
| | shRNA 114 | |
| 88 | CISH | AGAGGTAATAAAATAAAGAGGT |
| | shRNA 131 | |
| 89 | CISH | TAAAGAGGTAATAAAGGTATAA |
| | shRNA 96 | |
| 90 | CISH | TATTATTTCATGAAGTCTTATC |
| | shRNA 3 | |
| 91 | CISH | TCTTATCAGACGTGTATTTCTC |
| | shRNA 77 | |
| 92 | CISH | TCATTACAGAAAAATCAGTGGA |
| | shRNA 8 | |
| 93 | CISH | AAAGGTATAATGTCATTACAGA |
| | shRNA 81 | |
| 94 | CISH | TTATCAGACGTGTATTTCTCTC |
| | shRNA 40 | |
| 95 | CISH | TGTCATTACAGAAAAATCAGTG |
| | shRNA 4 | |
| 96 | DNMT3A | TATTAGCGAAGAACATCTGGAG |
| | shRNA 30 | |
| 97 | DNMT3A | TTGGCATCAATCATCACAGGGT |
| | shRNA 7 | |
| 98 | DNMT3A | TGCTTTATGGAGTTTGACCTCG |
| | shRNA 79 | |
| 99 | DNMT3A | ATCAATCATCACAGGGTTGGAC |
| | shRNA 51 | |
| 100 | DNMT3A | TCTTCCACAGCATTCATTCCTG |
| | shRNA 45 | |
| 101 | DNMT3A | TTTGGCATCAATCATCACAGGG |
| | shRNA 6 | |
| 102 | DNMT3A | AGGGATTCTTCTCTTCTTCTGG |
| | shRNA 71 | |
| 103 | DNMT3A | TTCAATCATGGGCTTGTTCTGC |
| | shRNA 69 | |
| 104 | DNMT3A | TCAATCATGGGCTTGTTCTGCA |
| | shRNA 49 | |
| 105 | DNMT3A | TAGCGAAGAACATCTGGAGCCG |
| | shRNA 64 | |
| 106 | DNMT3A | ATGAAGACAGGAAAATGCTGGT |
| | shRNA 34 | |
| 107 | DNMT3A | TTATTAGCGAAGAACATCTGGA |
| | shRNA 2 | |
| 108 | DNMT3A | TTCATTCCTGCAATGACCTTGG |
| | shRNA 54 | |
| 109 | DNMT3A | TAGGGATTCTTCTCTTCTTCTG |
| | shRNA 26 | |
| 110 | DNMT3A | TTCCATCAAAGAGAGACAGCAC |
| | shRNA 66 | |
| 111 | DNMT3A | TCTTTGTAGGGATTCTTCTCTT |
| | shRNA 23 | |
| 112 | DNMT3A | GTTTGTTTAACTTTGTGTCGCT |
| | shRNA 76 | |
| 113 | DNMT3A | TCAATAATCTCCTTGACCTTGG |
| | shRNA 8 | |
| 114 | DNMT3A | TTCTCATTCATGAAGACAGGAA |
| | shRNA 3 | |
| 115 | DNMT3A | TTTCTCATTCATGAAGACAGGA |
| | shRNA 5 | |
| 116 | DNMT3A | TCTTCTCTTCTTCTGGTGGCTC |
| | shRNA 14 | |
| 117 | DNMT3A | TTAGCGAAGAACATCTGGAGCC |
| | shRNA 29 | |
| 118 | DNMT3A | TTATCATTCACAGTGGATGCCA |
| | shRNA 4 | |
| 119 | DNMT3A | TTGCTGAACTTGGCTATCCTGC |
| | shRNA 25 | |
| 120 | DNMT3A | TTTAACTTTGTGTCGCTACCTC |
| | shRNA 55 | |
| 121 | DNMT3A | TTGTTTAACTTTGTGTCGCTAC |
| | shRNA 9 | |
| 122 | DNMT3A | TTTGTTTAACTTTGTGTCGCTA |
| | shRNA 1 | |
| 123 | PTPN2 | TTAACAAAAATATTTAATGCTG |
| | shRNA 4 | |
| 124 | PTPN2 | TAACAAAAATATTTAATGCTGC |
| | shRNA 2 | |
| 125 | PTPN2 | TTAATATAATCATTCTCAGCAT |
| | shRNA 1 | |
| 126 | PTPN2 | TCTGCAAAGTCTTCTGCTGGTG |
| | shRNA 126 | |
| 127 | PTPN2 | TACTGTATAATACGACTTCACA |
| | shRNA 88 | |
| 128 | PTPN2 | TTTAGAGGAAAGTCCTGTACAT |
| | shRNA 108 | |
| 129 | PTPN2 | TTTTATGTTAATATCATCTCCT |
| | shRNA 3 | |
| 130 | PTPN2 | TAATACGACTTCACATCTTCTG |
| | shRNA 58 | |
| 131 | PTPN2 | TCTGTATACTAGAATCTCCCTT |
| | shRNA 23 | |
| 132 | PTPN2 | TAATTGTAGTAGATGTACTGTA |
| | shRNA 6 | |
| 133 | PTPN2 | ATGAGAATCTCAGTTGATCTGG |
| | shRNA 62 | |
| 134 | PTPN2 | TTTAACACGACTGTGATCATAT |
| | shRNA 104 | |
| 135 | PTPN2 | TCGAATTTCCAAGTACAGCGGC |
| | shRNA 61 | |
| 136 | PTPN2 | TAATATAATCATTCTCAGCATT |
| | shRNA 45 | |
| 137 | PTPN2 | TCTGTTTTCTGGAAACTTGGCC |
| | shRNA 48 | |
| 138 | PTPN2 | TTAACCGATTCTTTCTCCACAA |
| | shRNA 127 | |
| 139 | PTPN2 | TCATTAAAAACACTTAACACAT |
| | shRNA 118 | |
| 140 | PTPN2 | TAAAGGTTCTGCAAAGTCTTCT |
| | shRNA 67 | |
| 141 | PTPN2 | TCTGACAAGAGCTTCACACTGA |
| | shRNA 19 | |
| 142 | PTPN2 | ATACGACTTCACATCTTCTGAC |
| | shRNA 71 | |
| 143 | PTPN2 | TTCTATGTCAACTAAACTGGCA |
| | shRNA 7 | |
| 144 | PTPN2 | TTCTAGACCTATTCTGTTCCCA |
| | shRNA 56 | |
| 145 | PTPN2 | TACGACTTCACATCTTCTGACA |
| | shRNA 89 | |
| 146 | PTPN2 | TATAATACGACTTCACATCTTC |
| | shRNA 13 | |
| 147 | TET2 | TATATTTACAATTATAGATCTG |
| | shRNA 3 | |
| 148 | TET2 | TTATATTTACAATTATAGATCT |
| | shRNA 10 | |
| 149 | TET2 | TTTATTCTCTGAAACTAGGTGT |
| | shRNA 98 | |
| 150 | TET2 | TTGTTTGAACAAGAAACCTGTA |
| | shRNA 90 | |
| 151 | TET2 | AAGAATTCAAATATGAACCTGC |
| | shRNA 53 | |
| 152 | TET2 | TAACATTACAATACTTACAGTA |
| | shRNA 7 | |
| 153 | TET2 | TAAAATGAAAGTAAACTTGTTT |
| | shRNA 78 | |
| 154 | TET2 | TCATATTGAGTCTTGACAGGTG |
| | shRNA 9 | |
| 155 | TET2 | AAGTATTGAAAAAAGGTATGCT |
| | shRNA 91 | |
| 156 | TET2 | TTTATTAAAAACACTCACATAG |
| | shRNA 27 | |
| 157 | TET2 | TTATGTGTTGAAAAACTGGTGA |
| | shRNA 70 | |
| 158 | TET2 | TTACTATACAACTCTTTCACTT |
| | shRNA 75 | |
| 159 | TET2 | TTTGTTGAAAATGAAATCTAGT |
| | shRNA 59 | |
| 160 | TET2 | TATACAACTCTTTCACTTCTTT |
| | shRNA 101 | |
| 161 | TET2 | TCACATTTACATATCATCTCGT |
| | shRNA 5 | |
| 162 | TET2 | TTTAGATGTAAAACTCTGGCTA |
| | shRNA 6 | |
| 163 | TET2 | TTATAAACCAAACATCTCATGA |
| | shRNA 97 | |
| 164 | TET2 | TTAAGTAATACAATGTTCTTGT |
| | shRNA 2 | |
| 165 | TET2 | TTTATCAACTCAGTGCATTCTA |
| | shRNA 71 | |
| 166 | TET2 | TTAACTGACAGATTCACATGGA |
| | shRNA 1 | |
| 167 | TET2 | TTAAAAAGACAAAAGCCAGCTT |
| | shRNA 80 | |
| 168 | TET2 | TTAGATGTAAAACTCTGGCTAT |
| | shRNA 16 | |
| 169 | TET2 | TATATTAGCAAATTGTAGATCT |
| | shRNA 35 | |
| 170 | TET2 | TAAGCTAAGAAATGACAACGTC |
| | shRNA 106 | |
| 171 | TET2 | TAAGATTACAGTAAAAACTGCA |
| | shRNA 4 | |
| 172 | TET2 | TTTAATTTACTATACAACTCTT |
| | shRNA 8 | |
| 173 | TET2 | TTTGAATACTGATTTTCACCAT |
| | shRNA 31 | |
| 174 | TET2 | TTATGTATCAAGTACCTTTGCT |
| | shRNA 14 | |
| 175 | TET2 | TCTTATAAACCAAACATCTCAT |
| | shRNA 54 | |
| 176 | ZC3H12A | TTCCATAGGGACACGGCTGCTT |
| | shRNA 86 | |
| 177 | ZC3H12A | TCTGTTAGCAGAGAGCTGGACT |
| | shRNA 106 | |
| 178 | ZC3H12A | TTCACAATGAATCTGTCGTCAT |
| | shRNA 10 | |
| 179 | ZC3H12A | TACAGAGATACAATGTGTATCA |
| | shRNA 19 | |
| 180 | ZC3H12A | TGTATCAACAGGGTGATCGCTT |
| | shRNA 29 | |
| 181 | DGKA | TTAGGATTGACAAAGACGAGAA |
| | shRNA 14 | |
| 182 | DGKA | TCAGCTTGAGAGACAGAGCCAC |
| | shRNA 42 | |
| 183 | DGKA | TTACTCATCTCTAAATCCTTGA |
| | shRNA 3 | |
| 184 | DGKA | TCTGGTACACAGGTTTTCAGGA |
| | shRNA 96 | |
| 185 | DGKA | TTTAGCACTGCGATGCCTTCTA |
| | shRNA 25 | |
| 186 | DGKA | ATAATAAATACTGTGAGGGTGA |
| | shRNA 10 | |
| 187 | DGKA | TAGTAGTACTTCCGTTGCTTGG |
| | shRNA 77 | |
| 188 | DGKA | TTCGTAGTAGTACTTCCGTTGC |
| | shRNA 101 | |
| 189 | DGKA | TCTGGGAACATTATCCACTTCG |
| | shRNA 37 | |
| 190 | DGKA | TGAGAAGATGGATTCAGATGTG |
| | shRNA 87 | |
| 191 | DGKA | TAATAAATACTGTGAGGGTGAA |
| | shRNA 15 | |
| 192 | DGKA | TCTTTTGTCACATTTGTCTCAT |
| | shRNA 81 | |
| 193 | DGKA | TAGATTTTCAGGAATTGCTGGA |
| | shRNA 1 | |
| 194 | DGKA | TTTGCATGTTGAGAAGATGGAT |
| | shRNA 22 | |
| 195 | DGKA | TTTCATGGCACACTGGTCGTGA |
| | shRNA 76 | |
| 196 | DGKA | TCAAATTTAAATCATCCATGGT |
| | shRNA 4 | |
| 197 | DGKA | ATAGATTTTCAGGAATTGCTGG |
| | shRNA 49 | |
| 198 | DGKA | ATATGTACCACTTTACTCATCT |
| | shRNA 90 | |
| 199 | DGKA | TTGCATGTTGAGAAGATGGATT |
| | shRNA 95 | |
| 200 | DGKA | TTTAAATCATCCATGGTCTTCT |
| | shRNA 6 | |
| 201 | DGKA | TTTACTCATCTCTAAATCCTTG |
| | shRNA 13 | |
| 202 | DGKA | TAAATCATCCATGGTCTTCTGG |
| | shRNA 12 | |
| 203 | DGKA | TAGTCAATCTCTTTCATCATCT |
| | shRNA 11 | |
| 204 | DGKA | TTAGCTTTGTCAATGGTCTCTA |
| | shRNA 5 | |
| 205 | ZC3H12A | TTGACGAAGGAGTACATGAGCA |
| | shRNA 40 | |
| 206 | ZC3H12A | TGAATCTGTCGTCATAGCACAC |
| | shRNA 97 | |
| 207 | ZC3H12A | TACTGACATGAAGCCACGTGCC |
| | shRNA 99 | |
| 208 | fLuciferase | TAGATAAGCATTATAATTCCTA |
| | shRNA | |
| 209 | rLuciferase | TTAATCAGAGACTTCAGGCGGT |
| | shRNA | |
| 210 | FAS | ACTGCGTGCCCTGCCAAGAA |
| | sgRNA | |
| 211 | FAS | TTAAGAATCTTTTCAAACACTA |
| | shRNA | |
| 212 | MSLN | QVQLQQSGPELEKPGASVKISCKASGYSFTGYTMNWVKQSHGKSLEWIGLITPYNG |
| | CAR | ASSYNQKFRGKATLTVDKSSSTAYMDLLSLTSEDSAVYFCARGGYDGRGFDYWG |
| | | QGTTVTVSSGGGGSGGGGSGGGGSDIELTQSPAIMSASPGEKVTMTCSASSSVSYM |
| | | HWYQQKSGTSPKRWIYDTSKLASGVPGRFSGSGSGNSYSLTISSVEAEDDATYYCQ |
| | | QWSGYPLTFGAGTKLEIKAAATTTPAPRPPTPAPTIASQPLSLRPEACRPAAGGAVH |
| | | TRGLDFACDIYIWAPLAGTCGVLLLSLVITLYCKRGRKKLLYIFKQPFMRPVQTTQE |
| | | EDGCSCRFPEEEEGGCELRVKFSRSADAPAYQQGQNQLYNELNLGRREEYDVLDK |
| | | RRGRDPEMGGKPRRKNPQEGLYNELQKDKMAEAYSEIGMKGERRRGKGHDGLYQ |
| | | GLSTATKDTYDALHMQALPPR |
| 213 | ALPG | DIQMTQSPSSLSASVGDRVTITCRASENIYSYVAWYQQKPGKAPKLLIYNAKSLASG |
| | PrimeR | VPSRFSGSGSGTDFTLTISSLQPEDFATYYCQHHYVSPWTFGGGTKLEIKGGGGSGG |
| | | GGSGGGGSQVQLQESGPGLVKPSETLSLTCTVSGFSLTSYGVSWIRQPAGKGLEWI |
| | | GVIWEDGSTNYHSALISRVTMSVDTSKNQFSLKLSSVTAADTAVYYCARPHYGSSY |
| | | VGAMEYWGAGTTVTVSSATTTPAPRPPTPAPTIASQPLSLRPEACFMYVAAAAFVL |
| | | LFFVGCGVLLSRKRRRQHGQLWFPEGFKVSEASKKKRREPLGEDSVGLKPLKNAM |
| | | VSKLSQLQTELLAALLESGLSKEALLQALGEPGPYLLAGEGPLDKGESCGGGRGEL |
| | | AELPNGLGETRGSEDETDDDGEDFTPPILKELENLSPEEAAHQKAVVETLLQEDPW |
| | | RVAKMVKSYLQQHNIPQREVVDTTGLNQSHLSQHLNKGTPMKTQKRAALYTWYV |
| | | RKQREVAQQFTHAGQGGLIEEPTGDELPTKKGRRNRFKWGPASQQILFQAYERQK |
| | | NPSKEERETLVEECNRAECIQRGVSPSQAQGLGSNLVTEVRVYNWFANRRKEEAFR |
| | | HKLAMTCRDEFPTMVFPSGQISQASALAPAPPQVLPQAPAPAPAPAMVSALAQAPA |
| | | PVPVLAPGPPQAVAPPAPKPTQAGEGTLSEALLQLQFDDEDLGALLGNSTDPAVFT |
| | | DLASVDNSEFQQLLNQGIPVAPHTTEPMLMEYPEAITRLVTGAQRPPDPAPAPLGAP |
| | | GLPNGLLSGDEDFSSIADMDFSALLSQISS |

*first 3 nucleotides are 2′-o-methyl analogs and 3′-phosphorothioate internucleotide linkages

1. One or more recombinant nucleic acids comprising at least one sequence as set forth in any one of SEQ ID NOs: 12-207.

2. The one or more recombinant nucleic acids of claim 1, wherein the nucleic acid is a guide RNA.

3. The one or more recombinant nucleic acids of any one of claims 1-2, further comprising a protein comprising a nuclease domain, wherein the nucleic acid and protein form a ribonucleoprotein (RNP) complex.

4. The one or more recombinant nucleic acids of claim 3, wherein the nuclease domain comprises a CRISPR-associated endonuclease (Cas), optionally a Cas9 nuclease.

5. The recombinant nucleic acid of claim 3 or 4, wherein the ribonucleoprotein (RNP) complex reduces expression of one or more of CD5, CBLB, CISH, DGKA, DGKZ, DNMT3A, MAP4K1, NR4A1, PTPN2, TET2, and/or ZC3H12A in a cell by at least 50%, 55%, 60%, 65%, 75%, 80%, 85%, 90%, 95%, or 99% as compared to a control cell that does not comprise the RNP complex.

6. The one or more recombinant nucleic acids of any one of claims 1-5, comprising a first nucleic acid and a second nucleic acid, wherein the first and second nucleic acids are distinct.

7. The one or more recombinant nucleic acids of any one of claims 1-6, comprising a sequence set forth in SEQ ID NO: 12 and a sequence set forth in SEQ ID NO:13.

8. The one or more recombinant nucleic acids of any one of claims 1-6, comprising a sequence set forth in SEQ ID NO: 21 and a sequence set forth in SEQ ID NO: 13.

9. The one or more recombinant nucleic acids of any one of claims 1-6, comprising a sequence set forth in SEQ ID NO: 21 and a sequence set forth in SEQ ID NO: 12.

10. The one or more recombinant nucleic acids of any one of claims 1-6, comprising a sequence set forth in SEQ ID NO: 12 and a sequence set forth in SEQ ID NO: 14.

11. The one or more recombinant nucleic acids of any one of claims 1-6, comprising a sequence set forth in SEQ ID NO: 13 and a sequence set forth in SEQ ID NO: 20.

12. The one or more recombinant nucleic acids of any one of claims 1-6, comprising a sequence set forth in SEQ ID NO: 21 and a sequence set forth in SEQ ID NO: 20.

13. The one or more recombinant nucleic acids of any one of claims 1-6, comprising a sequence set forth in SEQ ID NO: 20 and a sequence set forth in SEQ ID NO: 22.

14. The one or more recombinant nucleic acids of any one of claims 1-6, comprising a sequence set forth in SEQ ID NO: 17 and a sequence set forth in SEQ ID NO: 13.

15. The one or more recombinant nucleic acids of any one of claims 1-6, comprising a sequence set forth in SEQ ID NO: 20 and a sequence set forth in SEQ ID NO: 14.

16. The one or more recombinant nucleic acids of any one of claims 1-6, comprising a sequence set forth in SEQ ID NO: 21 and a sequence set forth in SEQ ID NO: 22.

17. The one or more recombinant nucleic acids of claim 1, wherein the nucleic acid is a short hairpin RNA (shRNA).

18. The one or more recombinant nucleic acids of claim 17, wherein the shRNA reduces the expression of one or more of CD5, CBLB, CISH, DGKA, DNMT3A, PTPN2, TET2, and/or ZC3H12A in a cell by at least 50%, 55%, 60%, 65%, 75%, 80%, 85%, 90%, 95%, or 99% as compared to a control cell that does not comprise the shRNA.

19. The one or more recombinant nucleic acids of claim 1, 17, or 18, comprising a first nucleic acid and a second nucleic acid, wherein the first and second nucleic acids are distinct.

20. One or more recombinant nucleic acids comprising a nucleic acid sequence at least 15 nucleotides in length complementary to an mRNA encoding human CD5 comprising the sequence set forth in SEQ ID NO: 1.

21. One or more recombinant nucleic acids comprising a nucleic acid sequence at least 15 nucleotides in length complementary to an mRNA encoding human CBLB comprising the sequence set forth in SEQ ID NO: 2.

22. One or more recombinant nucleic acids comprising a nucleic acid sequence at least 15 nucleotides in length complementary to an mRNA encoding human CISH comprising the sequence set forth in SEQ ID NO: 3.

23. One or more recombinant nucleic acids comprising a nucleic acid sequence at least 15 nucleotides in length complementary to an mRNA encoding DGKA comprising the sequence set forth in SEQ ID NO: 4.

24. One or more recombinant nucleic acids comprising a nucleic acid sequence at least 15 nucleotides in length complementary to an mRNA encoding DGKZ comprising the sequence set forth in SEQ ID NO: 5.

25. One or more recombinant nucleic acids comprising a nucleic acid sequence at least 15 nucleotides in length complementary to an mRNA encoding DNMT3A comprising the sequence set forth in SEQ ID NO: 6.

26. One or more recombinant nucleic acids comprising a nucleic acid sequence at least 15 nucleotides in length complementary to an mRNA encoding MAP4K1 comprising the sequence set forth in SEQ ID NO: 7.

27. One or more recombinant nucleic acids comprising a nucleic acid sequence at least 15 nucleotides in length complementary to an mRNA encoding NR4A1 comprising the sequence set forth in SEQ ID NO: 8.

28. One or more recombinant nucleic acids comprising a nucleic acid sequence at least 15 nucleotides in length complementary to an mRNA encoding TET2 comprising the sequence set forth in SEQ ID NO: 10.

29. One or more recombinant nucleic acids comprising a nucleic acid sequence at least 15 nucleotides in length complementary to an mRNA encoding PTPN2 comprising the sequence set forth in SEQ ID NO: 9.

30. One or more recombinant nucleic acids comprising a nucleic acid sequence at least 15 nucleotides in length complementary to an mRNA encoding ZC3H12A comprising the sequence set forth in SEQ ID NO: 11.

31. One or more recombinant nucleic acids comprising at least two or more nucleic acids selected from the group consisting of: (1) a nucleic acid sequence at least 15 nucleotides in length complementary to an mRNA encoding human CD5 comprising the sequence set forth in SEQ ID NO: 1; (2) a nucleic acid sequence at least 15 nucleotides in length complementary to an mRNA encoding human CBLB comprising the sequence set forth in SEQ ID NO: 2; (3) a nucleic acid sequence at least 15 nucleotides in length complementary to an mRNA encoding human CISH comprising the sequence set forth in SEQ ID NO: 3; (4) a nucleic acid sequence at least 15 nucleotides in length complementary to an mRNA encoding DGKA comprising the sequence set forth in SEQ ID NO: 4; (5) a nucleic acid sequence at least 15 nucleotides in length complementary to an mRNA encoding DGKZ comprising the sequence set forth in SEQ ID NO: 5; (6) a nucleic acid sequence at least 15 nucleotides in length complementary to an mRNA encoding DNMT3A comprising the sequence set forth in SEQ ID NO: 6; (7) a nucleic acid sequence at least 15 nucleotides in length complementary to an mRNA encoding MAP4K1 comprising the sequence set forth in SEQ ID NO: 7; (9) a nucleic acid sequence at least 15 nucleotides in length complementary to an mRNA encoding TET2 comprising the sequence set forth in SEQ ID NO: 10; (10) a nucleic acid sequence at least 15 nucleotides in length complementary to an mRNA encoding human Protein Tyrosine Phosphatase Non-Receptor Type 2 (PTPN2) comprising the sequence set forth in SEQ ID NO: 9; (11) a nucleic acid sequence at least 15 nucleotides in length complementary to an mRNA encoding NR4A1 comprising the sequence set forth in SEQ ID NO: 8; or (12) a nucleic acid sequence at least 15 nucleotides in length complementary to an mRNA encoding ZC3H12A comprising the sequence set forth in SEQ ID NO: 11.

32. The one or more recombinant nucleic acids of any one of claims 20 to 31, wherein the nucleic acid sequence is at least 16, 17, 18, 19, 20, 21, or 22 nucleotides in length.

33. The one or more recombinant nucleic acids of any one of claims 20 to 32, wherein the nucleic acid is a short hairpin RNA (shRNA), a small interfering RNA (siRNA), a double stranded RNA (dsRNA), or an antisense oligonucleotide.

34. The one or more recombinant nucleic acids of claim 33, wherein the nucleic acid is an shRNA.

35. The one or more recombinant nucleic acids of any one of claims 20-34, wherein the nucleic acid reduces expression of CD5, CBLB, CISH, DGKA, DGKZ, DNMT3A, MAP4K1, NR4A1, PTPN2, TET2, and/or ZC3H12A in a cell by at least 50%, 55%, 60%, 65%, 75%, 80%, 85%, 90%, 95%, or 99% as compared to a control cell that does not comprise the nucleic acid.

36. The one or more recombinant nucleic acids of any one of claims 20 and 31-35, wherein the nucleic acid sequence is an shRNA complementary to the mRNA encoding CD5 and comprises a sequence set forth in any one of SEQ ID NOs: 47-72.

37. The one or more recombinant nucleic acids of any one of claims 21 and 31-35, wherein the nucleic acid sequence is an shRNA complementary to the mRNA encoding CBLB and comprises a sequence set forth in any one of SEQ ID NOs: 23-46.

38. The one or more recombinant nucleic acids of any one of claims 22 and 31-35, wherein the nucleic acid sequence is an shRNA complementary to the mRNA encoding CISH and comprises a sequence set forth in any one of SEQ ID NOs: 73-95.

39. The one or more recombinant nucleic acids of any one of claims 23 and 31-35, wherein the nucleic acid sequence is an shRNA complementary to the mRNA encoding DGKA and comprises a sequence set forth in any one of SEQ ID NOs: 181-204.

40. The one or more recombinant nucleic acids of any one of claims 25 and 31-35, wherein the nucleic acid sequence is an shRNA complementary to the mRNA encoding DNMT3A and comprises a sequence set forth in any one of SEQ ID NOs: 96-122.

41. The one or more recombinant nucleic acids of any one of claims 28 and 31-35, wherein the nucleic acid sequence is an shRNA complementary to the mRNA encoding TET2 and comprises a sequence set forth in any one of SEQ ID NOs: 147-175.

42. The one or more recombinant nucleic acids of any one of claims 29 and 31-35, wherein the nucleic acid sequence is an shRNA complementary to the mRNA encoding PTPN2 and comprises a sequence set forth in any one of SEQ ID NOs: 123-146.

43. The one or more recombinant nucleic acids of any one of claims 30-35, wherein the nucleic acid sequence is an shRNA complementary to the mRNA encoding ZC3H12A and comprises a sequence set forth in any one of SEQ ID NOs: 176-180 and 205-207.

44. The one or more recombinant nucleic acids of any one of claims 31-43, comprising at least a nucleic acid sequence at least 15 nucleotides in length complementary to an mRNA encoding human CD5 comprising the sequence set forth in SEQ ID NO: 1 and a nucleic acid sequence at least 15 nucleotides in length complementary to an mRNA encoding human CBLB comprising the sequence set forth in SEQ ID NO: 2.

45. The one or more recombinant nucleic acids of any one of claims 31-43, comprising at least a nucleic acid sequence at least 15 nucleotides in length complementary to an mRNA encoding TET2 comprising the sequence set forth in SEQ ID NO: 9 and a nucleic acid sequence at least 15 nucleotides in length complementary to an mRNA encoding human CBLB comprising the sequence set forth in SEQ ID NO: 2.

46. The one or more recombinant nucleic acids of any one of claims 31-43, comprising at least a nucleic acid sequence at least 15 nucleotides in length complementary to an mRNA encoding TET2 comprising the sequence set forth in SEQ ID NO: 9 and a nucleic acid sequence at least 15 nucleotides in length complementary to an mRNA encoding human CD5 comprising the sequence set forth in SEQ ID NO: 1.

47. The one or more recombinant nucleic acids of any one of claims 31-43, comprising at least a nucleic acid sequence at least 15 nucleotides in length complementary to an mRNA encoding human CD5 comprising the sequence set forth in SEQ ID NO: 1 and a nucleic acid sequence at least 15 nucleotides in length complementary to an mRNA encoding human CISH comprising the sequence set forth in SEQ ID NO: 3.

48. The one or more recombinant nucleic acids of any one of claims 20 to 47, wherein the recombinant nucleic acid further comprises one or more of: a nucleotide sequence encoding a priming receptor comprising a first extracellular antigen-binding domain that specifically binds to a first antigen; a nucleotide sequence encoding a chimeric antigen receptor (CAR) comprising a second extracellular antigen-binding domain that specifically binds to a second antigen; or a nucleotide sequence encoding a T cell receptor (TCR).

49. The one or more recombinant nucleic acids of claim 48, wherein the first antigen and the second antigen are distinct.

50. The one or more recombinant nucleic acids of claim 48 or 49, wherein the recombinant nucleic acid comprises, in a 5′ to 3′ direction
(a) the TCR;
(b) the nucleic acid of any one of claims 20 to 47.

51. The one or more recombinant nucleic acids of claim 48 or 49, wherein the recombinant nucleic acid comprises, in a 5′ to 3′ direction
(a) the nucleic acid of any one of claims 20 to 47;
(b) the TCR.

52. The one or more recombinant nucleic acids of claim 48 or 49, wherein the recombinant nucleic acid comprises, in a 5′ to 3′ direction
(a) the CAR;
(b) the nucleic acid of any one of claims 20 to 47; and
(c) the priming receptor.

53. The one or more recombinant nucleic acids of claim 48 or 49, wherein the nucleic acid comprises, in a 5′ to 3′ direction
(a) the priming receptor;
(b) the nucleic acid of any one of claims 20 to 47; and
(c) the CAR.

54. The one or more recombinant nucleic acids of any one of claims 20 to 53, wherein the recombinant nucleic acid further comprises a 5′ homology directed repair arm and/or a 3′ homology directed repair arm complementary to an insertion site in a host cell chromosome.

55. The one or more recombinant nucleic acids of claim 54, wherein the recombinant nucleic acid comprises the 5′ homology directed repair arm and the 3′ homology directed repair arm.

56. The one or more recombinant nucleic acids of any one of claims 20 to 55, wherein the recombinant nucleic acid is incorporated into an expression cassette or an expression vector.

57. The one or more recombinant nucleic acids of claim 56, wherein the expression cassette or the expression vector further comprises a constitutive promoter upstream of the recombinant nucleic acid.

58. The one or more recombinant nucleic acids of any one of claims 20 to 57, comprising a first nucleic acid and a second nucleic acid, wherein the first nucleic acid and the second nucleic acid are encoded on a single nucleic acid.

59. The one or more recombinant nucleic acids of claim 58, wherein the first nucleic acid comprises the 5′ homology directed repair arm and the second nucleic acid comprises the 3′ homology directed repair arm.

60. The one or more recombinant nucleic acids of any one of claim 58 or 59, wherein the first nucleic acid and the second nucleic acid are encoded on different nucleic acids.

61. The one or more recombinant nucleic acids of any one of claims 58 to 60, wherein the first nucleic acid and the second nucleic acid are incorporated into a single expression cassette or a single expression vector.

62. The one or more recombinant nucleic acids of claim 61, wherein the expression cassette or the expression vector further comprises a constitutive promoter upstream of the first nucleic acid and/or upstream of the second nucleic acid.

63. The one or more recombinant nucleic acids of any one of claims 56 to 62, wherein the expression vector is a non-viral vector.

64. An expression vector comprising the one or more recombinant nucleic acid(s) of any one of claims 1-63.

65. The expression vector of claim 64, wherein the expression vector is a non-viral vector.

66. The vector of claim 64 or 65, wherein the 5′ and 3′ ends of the recombinant nucleic acid(s) comprise one or more nucleotide sequences that are homologous to genomic sequences flanking an insertion site in a genome of a primary cell.

67. The vector of claim 66, wherein the insertion site is located at a T Cell Receptor Alpha Constant (TRAC) locus or a genomic safe harbor (GSH) locus.

68. The vector of claim 67, wherein the GSH locus is the GS94 locus.

69. An immune cell comprising at least one or more nucleic acids selected from the group consisting of: (1) a nucleic acid sequence at least 15 nucleotides in length complementary to an mRNA encoding human CD5 comprising the sequence set forth in SEQ ID NO: 1; (2) a nucleic acid sequence at least 15 nucleotides in length complementary to an mRNA encoding human CBLB comprising the sequence set forth in SEQ ID NO: 2; (3) a nucleic acid sequence at least 15 nucleotides in length complementary to an mRNA encoding human CISH comprising the sequence set forth in SEQ ID NO: 3; (4) a nucleic acid sequence at least 15 nucleotides in length complementary to an mRNA encoding DGKA comprising the sequence set forth in SEQ ID NO: 4; (5) a nucleic acid sequence at least 15 nucleotides in length complementary to an mRNA encoding DGKZ comprising the sequence set forth in SEQ ID NO: 5; (6) a nucleic acid sequence at least 15 nucleotides in length complementary to an mRNA encoding DNMT3A comprising the sequence set forth in SEQ ID NO: 6; (7) a nucleic acid sequence at least 15 nucleotides in length complementary to an mRNA encoding MAP4K1 comprising the sequence set forth in SEQ ID NO: 7; (9) a nucleic acid sequence at least 15 nucleotides in length complementary to an mRNA encoding TET2 comprising the sequence set forth in SEQ ID NO: 10; (10) a nucleic acid sequence at least 15 nucleotides in length complementary to an mRNA encoding human Protein Tyrosine Phosphatase Non-Receptor Type 2 (PTPN2) comprising the sequence set forth in SEQ ID NO: 9; (11) a nucleic acid sequence at least 15 nucleotides in length complementary to an mRNA encoding NR4A1 comprising the sequence set forth in SEQ ID NO: 8; or (12) a nucleic acid sequence at least 15 nucleotides in length complementary to an mRNA encoding ZC3H12A comprising the sequence set forth in SEQ ID NO: 11.

70. The immune cell of claim 69, wherein the one or more nucleic acids are an shRNA, an siRNA, a dsRNA, or an antisense oligonucleotide.

71. The immune cell of claim 70, wherein the one or more nucleic acids are shRNA.

72. The immune cell of claim 71, wherein the shRNA is complementary to the mRNA encoding CD5 and comprises a sequence set forth in any one of SEQ ID NOs: 47-72.

73. The immune cell of claim 71, wherein the shRNA is complementary to the mRNA encoding CBLB and comprises a sequence set forth in any one of SEQ ID NOs: 23-46.

74. The immune cell of claim 71, wherein the shRNA is complementary to the mRNA encoding CISH and comprises a sequence set forth in any one of SEQ ID NOs: 73-95.

75. The immune cell of claim 71, wherein the shRNA is complementary to the mRNA encoding DGKA and comprises a sequence set forth in any one of SEQ ID NOs: 181-204.

76. The immune cell of claim 71, wherein the shRNA is complementary to the mRNA encoding DNMT3A and comprises a sequence set forth in any one of SEQ ID NOs: 96-122.

77. The immune cell of claim 71, wherein the shRNA is complementary to the mRNA encoding TET2 and comprises a sequence set forth in any one of SEQ ID NOs: 147-175.

78. The immune cell of claim 71, wherein the shRNA is complementary to the mRNA encoding PTPN2 and comprises a sequence set forth in any one of SEQ ID NOs: 123-146.

79. The immune cell of claim 71, wherein the shRNA is complementary to the mRNA encoding ZC3H12A and comprises a sequence set forth in any one of SEQ ID NOs: 176-180 and 205-207.

80. The immune cell of any one of claims 69-79, wherein the cell further comprises a deletion of at least a first target gene selected from the group consisting of CD5, CBLB, CISH, DGKA, DGKZ, DNMT3A, MAP4K1, NR4A1, PTPN2, TET2, and ZC3H12A.

81. The immune cell of claim 80, further comprising deletion of at least a second target gene selected from the group consisting of CD5, CBLB, CISH, DGKA, DGKZ, DNMT3A, MAP4K1, NR4A1, PTPN2, TET2, and ZC3H12A, and wherein the first target gene and the second target gene are distinct.

82. The immune cell of claim 80 or 81, wherein the at least first or second target gene(s) are deleted via CRISPR-Cas9 gene editing.

83. The immune cell of any one of claims 69-82, wherein expression of at least one or more target gene selected from the group consisting of CD5, CBLB, CISH, DGKA, DGKZ, DNMT3A, MAP4K1, NR4A1, PTPN2, TET2, and ZC3H12A in the immune cell is reduced by at least 50%, 55%, 60%, 65%, 75%, 80%, 85%, 90%, 95%, or 99% as compared to a control cell that does not comprise the nucleic acids or does comprise the target gene.

84. An immune cell comprising a deletion of at least a first target gene selected from the group consisting of CD5, CBLB, CISH, DGKA, DGKZ, DNMT3A, MAP4K1, NR4A1, PTPN2, TET2, and ZC3H12A.

85. The immune cell of claim 84, further comprising deletion of at least a second target gene selected from the group consisting of CD5, CBLB, CISH, DGKA, DGKZ, DNMT3A, MAP4K1, NR4A1, PTPN2, TET2, and ZC3H12A, and wherein the first target gene and the second target gene are distinct.

86. The immune cell of claim 84 or 85, wherein the at least first or second target gene(s) are deleted via CRISPR-Cas9 gene editing.

87. The immune cell of any one of claims 84-86, wherein expression of the at least first or at least second target gene in the immune cell is reduced by at least 50%, 55%, 60%, 65%, 75%, 80%, 85%, 90%, 95%, or 99% as compared to a control cell that does not comprise the deletion of the at least first or at least second target gene.

88. An immune cell comprising a first guide RNA, wherein the first guide RNA comprises a sequence set forth in SEQ ID NOs: 12-22.

89. The immune cell of claim 67, further comprising a second guide RNA comprising a sequence set forth in SEQ ID NOs: 12-22.

90. The immune cell of claim 67 or 68, further comprising a protein comprising a nuclease domain, wherein the nucleic acid and protein form a ribonucleoprotein (RNP) complex.

91. The immune cell of any one of claims 67-69, wherein the nuclease domain comprises a CRISPR-associated endonuclease (Cas), optionally a Cas9 nuclease.

92. An immune cell comprising one or more nucleic acids comprising a first shRNA and a second shRNA, wherein the first shRNA and second shRNA each comprise a sequence set forth in any one of SEQ ID NOs: 23-207.

93. The immune cell of any one of claims 69-92, wherein the one or more nucleic acids reduce expression of CD5, CBLB, CISH, DGKA, DGKZ, DNMT3A, MAP4K1, NR4A1, PTPN2, TET2, or ZC3H12A in the immune cell by at least 50%, 55%, 60%, 65%, 75%, 80%, 85%, 90%, 95%, or 99% as compared to a control cell that does not comprise the one or more nucleic acids.

94. The immune cell of claim 93, wherein expression of CD5, CBLB, CISH, DGKA, DGKZ, DNMT3A, MAP4K1, NR4A1, PTPN2, TET2, and/or ZC3H12A in the immune cell is reduced by at least 50%, 55%, 60%, 65%, 75%, 80%, 85%, 90%, 95%, or 99% as compared to a control cell that does not comprise the first or second nucleic acid.

95. The immune cell of any one of claim 83, 87, 93 or 94, wherein expression of CD5, CBLB, CISH, DGKA, DGKZ, DNMT3A, MAP4K1, NR4A1, PTPN2, TET2, and/or ZC3H12A is determined by a nucleic acid assay or a protein assay.

96. The immune cell of claim 95, wherein the nucleic acid assay comprises at least one of polymerase chain reaction (PCR), quantitative PCR (qPCR), RT-qPCR, microarray, gene array, or RNAseq.

97. The immune cell of claim 95, wherein the protein assay comprises at least one of immunoblotting, fluorescence activated cell sorting, flow-cytometry, magnetic-activated cell sorting, or affinity-based cell separation.

98. The immune cell of any one of claims 69 to 97, wherein the cell further comprises one or more of: a priming receptor comprising a first extracellular antigen-binding domain that specifically binds to a first antigen; a chimeric antigen receptor (CAR) comprising a second extracellular antigen-binding domain that specifically binds to a second antigen; or a T cell receptor (TCR).

99. The immune cell of any one of claims 69 to 98, wherein the immune cell is a primary human immune cell.

100. The immune cell of any one of claims 69-99, wherein the primary immune cell is a natural killer (NK) cell, a natural killer T (NKT) cell, a T cell, a γδ T cell, a CD8+ T cell, a CD4+ T cell, a primary T cell, a T cell progenitor, or an induced pluripotent stem cell (iPSC).

101. The immune cell of any one of claims 69-100, wherein the primary immune cell is a primary T cell.

102. The immune cell of any one of claims 69-101, wherein the primary immune cell is a primary human T cell.

103. The immune cell of any one of claims 69-102, wherein the immune cell is virus-free.

104. The immune cell of any one of claims 69-103, wherein the immune cell is a viable, virus-free, primary cell.

105. The immune cell of any one of claims 69-104, wherein the immune cell is an autologous immune cell.

106. The immune cell of any one of claims 69-104, wherein the immune cell is an allogeneic immune cell.

107. A primary immune cell comprising at least one recombinant nucleic acid(s) comprising a first nucleic acid comprising a sequence as set forth in SEQ ID NOs: 12-207; and wherein the primary immune cell does not comprise a viral vector for introducing the recombinant nucleic acid(s) into the primary immune cell.

108. A viable, virus-free, primary cell comprising one or more ribonucleoprotein complex(es) (RNP), wherein the RNP comprises a nuclease domain and a guide RNA, wherein the guide RNA comprises a first nucleic acid comprising a sequence as set forth in SEQ ID NOs: 12-22.

109. The immune cell of claim 107 or 108, further comprising a second different nucleic acid comprising a sequence as set forth in SEQ ID NO: 12-207.

110. A primary immune cell comprising a ribonucleoprotein complex (RNP)-recombinant nucleic acid(s) complex, wherein the RNP comprises a nuclease domain and a guide RNA, wherein the recombinant nucleic acid(s) comprises at least a first nucleic acid comprising: (1) a nucleic acid sequence at least 15 nucleotides in length complementary to an mRNA encoding human CD5 comprising the sequence set forth in SEQ ID NO: 1; (2) a nucleic acid sequence at least 15 nucleotides in length complementary to an mRNA encoding human CBLB comprising the sequence set forth in SEQ ID NO: 2; (3) a nucleic acid sequence at least 15 nucleotides in length complementary to an mRNA encoding human CISH comprising the sequence set forth in SEQ ID NO: 3; (4) a nucleic acid sequence at least 15 nucleotides in length complementary to an mRNA encoding DGKA comprising the sequence set forth in SEQ ID NO: 4; (5) a nucleic acid sequence at least 15 nucleotides in length complementary to an mRNA encoding DGKZ comprising the sequence set forth in SEQ ID NO: 5; (6) a nucleic acid sequence at least 15 nucleotides in length complementary to an mRNA encoding DNMT3A comprising the sequence set forth in SEQ ID NO: 6; (7) a nucleic acid sequence at least 15 nucleotides in length complementary to an mRNA encoding MAP4K1 comprising the sequence set forth in SEQ ID NO: 7; (9) a nucleic acid sequence at least 15 nucleotides in length complementary to an mRNA encoding TET2 comprising the sequence set forth in SEQ ID NO: 10; (10) a nucleic acid sequence at least 15 nucleotides in length complementary to an mRNA encoding human Protein Tyrosine Phosphatase Non-Receptor Type 2 (PTPN2) comprising the sequence set forth in SEQ ID NO: 9; (11) a nucleic acid sequence at least 15 nucleotides in length complementary to an mRNA encoding NR4A1 comprising the sequence set forth in SEQ ID NO: 8; or (12) a nucleic acid sequence at least 15 nucleotides in length complementary to an mRNA encoding ZC3H12A comprising the sequence set forth in SEQ ID NO: 11, and wherein the 5′ and 3′ ends of the recombinant nucleic acid(s) comprise nucleotide sequences that are homologous to genomic sequences flanking an insertion site in the genome of the primary cell.

111. A viable, virus-free, primary cell comprising a ribonucleoprotein complex (RNP)-recombinant nucleic acid(s) complex, wherein the RNP comprises a nuclease domain and a guide RNA, wherein recombinant nucleic acid(s) comprises at least a first nucleic acid: (1) a nucleic acid sequence at least 15 nucleotides in length complementary to an mRNA encoding human CD5 comprising the sequence set forth in SEQ ID NO: 1; (2) a nucleic acid sequence at least 15 nucleotides in length complementary to an mRNA encoding human CBLB comprising the sequence set forth in SEQ ID NO: 2; (3) a nucleic acid sequence at least 15 nucleotides in length complementary to an mRNA encoding human CISH comprising the sequence set forth in SEQ ID NO: 3; (4) a nucleic acid sequence at least 15 nucleotides in length complementary to an mRNA encoding DGKA comprising the sequence set forth in SEQ ID NO: 4; (5) a nucleic acid sequence at least 15 nucleotides in length complementary to an mRNA encoding DGKZ comprising the sequence set forth in SEQ ID NO: 5; (6) a nucleic acid sequence at least 15 nucleotides in length complementary to an mRNA encoding DNMT3A comprising the sequence set forth in SEQ ID NO: 6; (7) a nucleic acid sequence at least 15 nucleotides in length complementary to an mRNA encoding MAP4K1 comprising the sequence set forth in SEQ ID NO: 7; (9) a nucleic acid sequence at least 15 nucleotides in length complementary to an mRNA encoding TET2 comprising the sequence set forth in SEQ ID NO: 10; (10) a nucleic acid sequence at least 15 nucleotides in length complementary to an mRNA encoding human Protein Tyrosine Phosphatase Non-Receptor Type 2 (PTPN2) comprising the sequence set forth in SEQ ID NO: 9; (11) a nucleic acid sequence at least 15 nucleotides in length complementary to an mRNA encoding NR4A1 comprising the sequence set forth in SEQ ID NO: 8; or (12) a nucleic acid sequence at least 15 nucleotides in length complementary to an mRNA encoding ZC3H12A comprising the sequence set forth in SEQ ID NO: 11, and wherein the 5′ and 3′ ends of the recombinant nucleic acid(s) comprise nucleotide sequences that are homologous to genomic sequences flanking an insertion site in the genome of the primary cell.

112. The cell of claim 110 or 111, wherein the nucleic acid sequence is an shRNA complementary to the mRNA encoding CD5 and comprises a sequence set forth in any one of SEQ ID NOs: 47-72.

113. The cell of claim 110 or 111, wherein the nucleic acid sequence is an shRNA complementary to the mRNA encoding CBLB and comprises a sequence set forth in any one of SEQ ID NOs: 23-46.

114. The cell of claim 110 or 111, wherein the nucleic acid sequence is an shRNA complementary to the mRNA encoding CISH and comprises a sequence set forth in any one of SEQ ID NOs: 73-95.

115. The cell of claim 110 or 111, wherein the nucleic acid sequence is an shRNA complementary to the mRNA encoding DGKA and comprises a sequence set forth in any one of SEQ ID NOs: 181-204.

116. The cell of claim 110 or 111, wherein the nucleic acid sequence is an shRNA complementary to the mRNA encoding DNMT3A and comprises a sequence set forth in any one of SEQ ID NOs: 96-122.

117. The cell of claim 110 or 111, wherein the nucleic acid sequence is an shRNA complementary to the mRNA encoding TET2 and comprises a sequence set forth in any one of SEQ ID NOs: 147-175.

118. The cell of claim 110 or 111, wherein the nucleic acid sequence is an shRNA complementary to the mRNA encoding PTPN2 and comprises a sequence set forth in any one of SEQ ID NOs: 123-146.

119. The cell of claim 110 or 111, wherein the nucleic acid sequence is an shRNA complementary to the mRNA encoding ZC3H12A and comprises a sequence set forth in any one of SEQ ID NOs: 176-180 and 205-207.

120. The cell of any one of claims 107-119, wherein the cell further comprises a priming receptor comprising a first extracellular antigen-binding domain that specifically binds to a first antigen and a chimeric antigen receptor (CAR) comprising a second extracellular antigen-binding domain that specifically binds to a second antigen, wherein the first antigen and the second antigen are distinct.

121. A population of cells comprising a plurality of immune cells of any one of claims 69-120.

122. A pharmaceutical composition comprising the immune cell of any one of claims 69-120 or the population of cells of claim 121, and a pharmaceutically acceptable excipient.

123. A pharmaceutical composition comprising the recombinant nucleic acid of any one of claims 1 to 63, or the vector of any one of claims 64-67, and a pharmaceutically acceptable excipient.

124. A method of editing an immune cell, comprising:
(a) providing a ribonucleoprotein (RNP) comprising a nuclease domain and a guide RNA, wherein the guide RNA comprises a sequence as set forth in SEQ ID NOs: 12-22;
(b) non-virally introducing the RNP into the immune cell, wherein the guide RNA specifically hybridizes to a target region of the genome of the primary immune cell, and wherein the nuclease domain cleaves the target region to create a double stranded break site in the genome of the immune cell.

125. A method of editing an immune cell, comprising:
(a) providing a ribonucleoprotein (RNP)-recombinant nucleic acid(s) complex, wherein the RNP comprises a nuclease domain and a guide RNA, wherein the recombinant nucleic acid(s) comprises the recombinant nucleic acid(s) of any one of claims 20-63, and wherein the 5′ and 3′ ends of the recombinant nucleic acid(s) comprise nucleotide sequences that are homologous to genomic sequences flanking an insertion site in the genome of the immune cell;
(b) non-virally introducing the RNP-recombinant nucleic acid(s) complex into the immune cell, wherein the guide RNA specifically hybridizes to a target region of the genome of the primary immune cell, and wherein the nuclease domain cleaves the target region to create the insertion site in the genome of the immune cell; and
(c) editing the immune cell via insertion of the recombinant nucleic acid(s) of any one of claims 20-63 into the insertion site in the genome of the immune cell.

126. The method of claim 124 or 125, wherein non-virally introducing comprises electroporation.

127. The method of any one of claims 124-126, wherein the nuclease domain comprises a CRISPR-associated endonuclease (Cas), optionally a Cas9 nuclease.

128. The method of any one of claims 125 to 127, wherein the target region of the genome of the cell is a T Cell Receptor Alpha Constant (TRAC) locus or a genomic safe harbor (GSH) locus.

129. The method of any one of claims 125 to 128, wherein the recombinant nucleic acid(s) is a double-stranded recombinant nucleic acid(s) or a single-stranded recombinant nucleic acid(s).

130. The method of any one of claims 125 to 129, wherein the recombinant nucleic acid(s) is a linear recombinant nucleic acid(s) or a circular recombinant nucleic acid(s), optionally wherein the circular recombinant nucleic acid(s) is a plasmid.

131. The method of any one of claims 124 to 130, wherein the immune cell is a primary human immune cell.

132. The method of any one of claims 124 to 131, wherein the immune cell is an autologous immune cell.

133. The method of any one of claims 124 to 131, wherein the immune cell is an allogeneic immune cell.

134. The method of any one of claims 124 to 133, wherein the immune cell is a natural killer (NK) cell, a natural killer T (NKT) cell, a T cell, a γδ T cell, a CD8+ T cell, a CD4+ T cell, a primary T cell, a T cell progenitor, or an induced pluripotent stem cell (iPSC).

135. The method of any one of claims 124 to 134, wherein the immune cell is a primary T cell.

136. The method of any one of claims 124 to 135, wherein the immune cell is a primary human T cell.

137. The method of any one of claims 124 to 136, wherein the immune cell is virus-free.

138. The method of any one of claims 124 to 137, further comprising obtaining the immune cell from a patient and introducing the recombinant nucleic acid in vitro.

139. A method of treating a disease in a subject comprising administering the immune cell(s) of any one of claims 69-121 or the pharmaceutical composition of claim 122 or 123 to the subject.

140. The method of claim 139, wherein the disease is cancer.

141. The method of claim 140, wherein the cancer is a solid cancer or a liquid cancer.

142. The method of claim 140 or 141, wherein the cancer is breast cancer, HER2-positive breast cancer, estrogen-receptor positive breast cancer, progesterone-receptor positive breast cancer, HER2-/estrogen-receptor-/progesterone-receptor-negative breast cancer, triple negative breast cancer, non-small cell lung cancer (NSCLC), lung adenocarcinoma, lung squamous cell carcinoma, lung adenosquamous carcinoma, prostate cancer, castration-resistant prostate cancer, colon cancer, rectal cancer, microsatellite instable (MSI) colon cancer, non-MSI colon cancer, or non-MSI or rectal cancer.

143. The method of any one of claims 139-142, wherein the administration of the cell(s) enhances an immune response.

144. The method of claim 143, wherein the enhanced immune response is an adaptive immune response.

145. The method of claim 144, wherein the enhanced immune response is increased T cell cytotoxicity.

146. The method of claim 144, wherein the enhanced immune response is increased T cell expansion and/or proliferation.

147. The method of claim 143, wherein the enhanced immune response is an innate immune response.

148. A method of enhancing an immune response in a subject comprising administering the immune cell(s) of any one of claims 69-121 or the pharmaceutical composition of claim 122 or 123 to the subject.

149. The method of claim 148, wherein the enhanced immune response is an adaptive immune response.

150. The method of claim 149, wherein the enhanced immune response is increased T cell cytotoxicity.

151. The method of claim 149, wherein the enhanced immune response is increased T cell expansion and/or proliferation.

152. The method of claim 149, wherein the enhanced immune response is an innate immune response.

153. The method of any one of claims 139-152, wherein expression of CD5, CBLB, CISH, DGKA, DGKZ, DNMT3A, MAP4K1, NR4A1, PTPN2, TET2, and/or ZC3H12A in the immune cell is reduced by at least 50%, 55%, 60%, 65%, 75%, 80%, 85%, 90%, 95%, or 99% as compared to a control cell that does not comprise the first nucleic acid or RNP complex.

154. The method of any one of claims 139-153, wherein expression of CD5, CBLB, CISH, DGKA, DGKZ, DNMT3A, MAP4K1, NR4A1, PTPN2, TET2, and/or ZC3H12A in the immune cell is reduced by at least 50%, 55%, 60%, 65%, 75%, 80%, 85%, 90%, 95%, or 99% as compared to a control cell that does not comprise the second nucleic acid or RNP complex.

155. The method of any one of claims 153-154, wherein expression of CD5, CBLB, CISH, DGKA, DGKZ, DNMT3A, MAP4K1, NR4A1, PTPN2, TET2, and/or ZC3H12A in the immune cell is determined by a nucleic acid assay or a protein assay.

156. The method of claim 155, wherein the nucleic acid assay comprises at least one of polymerase chain reaction (PCR), quantitative PCR (qPCR), RT-qPCR, microarray, gene array, or RNAseq.

157. The method of claim 155, wherein the protein assay comprises at least one of immunoblotting, fluorescence activated cell sorting, flow-cytometry, magnetic-activated cell sorting, or affinity-based cell separation.

158. The method of any one of claims 139-157, further comprising administering an immunotherapy to the subject concurrently with the immune cell or subsequently to the immune cell.
